(19) United States
(12) Patent Application Publication
(10) Pub. No.: US 2013/0072473 A1
(43) Pub. Date: Mar. 21, 2013

(54) COMPOUNDS FOR TREATING PROTEIN FOLDING DISORDERS

(71) Applicants: Bradley Tait, Malden, MA (US); Noel A. Powell, Westford, MA (US); Matthew Cullen, Braintree, MA (US)

(72) Inventors: Bradley Tait, Malden, MA (US); Noel A. Powell, Westford, MA (US); Matthew Cullen, Braintree, MA (US)

(73) Assignee: PROTEOSTASIS THERAPEUTICS, INC.

(21) Appl. No.: 13/467,949

(22) Filed: May 9, 2012

Related U.S. Application Data
(60) Provisional application No. 61/484,050, filed on May 9, 2011.

(51) Int. Cl. A61K 31/55; C07D 495/04; C07D 333/68; C07D 417/04; C07D 307/85; A61K 31/4743; A61K 31/4745

(52) U.S. Cl. CPC C07D 495/04; C07D 417/04; C07D 307/85; C07D 333/68
USPC 514/215; 514/301; 546/114; 514/367; 548/159; 540/593; 514/310; 546/143; 514/470; 549/467; 514/443; 549/57

(57) ABSTRACT

The present invention is directed to compounds of Formulae (I), (IIa-IIh), (IIIa-IIIe), (IVa-IVc), (Va-V1), (VIa-VII), (VII), (VIII) and (IX), pharmaceutical compositions thereof and methods of use thereof in the treatment of conditions associated with a dysfunction in proteostasis.

### RELATED APPLICATION

This application claims the benefit of U.S. Provisional Application No. 61/484,050 filed May 9, 2011. The entire teachings of the above application are incorporated herein by reference.

### BACKGROUND OF THE INVENTION

Cells normally maintain a balance between protein synthesis, folding, trafficking, aggregation, and degradation, referred to as protein homeostasis, utilizing sensors and networks of pathways [Sitia et al., Nature 426: 891-894, 2003; Ron et al., Nat Rev Mol Cell Biol 8: 519-529, 2007]. The cellular maintenance of protein homeostasis, or proteostasis, refers to controlling the conformation, binding interactions, location and concentration of individual proteins making up the proteome. Protein folding in vivo is accomplished through interactions between the folding polypeptide chain and macromolecular cellular components, including multiple classes of chaperones and folding enzymes, which minimize aggregation [Wiseman et al., Cell 131: 809-821, 2007]. Whether a given protein folds in a certain cell type depends on the distribution, concentration, and subcellular localization of chaperones, folding enzymes, metabolites and the like [Wiseman et al.]. Human loss of function diseases are often the result of a disruption of normal protein homeostasis, typically caused by a mutation in a given protein that compromises its cellular folding, leading to efficient degradation [Cohen et al., Nature 426: 905-909, 2003]. Human gain of function diseases are similarly the result of a disruption in protein homeostasis leading to protein aggregation [Balch et al. (2008), Science 319: 916-919].

Cystic Fibrosis (CF) is caused by mutations in the CFTR (Cystic Fibrosis Transmembrane conductance Regulator) gene' which encodes a multi-membrane spanning epithelial chloride channel. Ninety percent of patients have a deletion of Phenylalanine 508 (ΔF508) on at least one allele. This mutation results in disruption of the energetics of the protein fold² leading to efficient degradation of CFTR in the endoplasmic reticulum (ER). The loss of a functional CFTR channel at the plasma membrane disrupts ionic homeostasis (Cl⁻, Na⁺, HCO₃⁻) and airway surface hydration leading to reduced lung function'. Reduced periciliary liquid volume and increased mucus viscosity impede mucociliary clearance resulting in chronic infection and inflammation, phenotypic hallmarks of CF disease³. In addition to respiratory dysfunction, ΔF508 also impacts the normal function of additional organs (pancreas, intestine, gall bladder), suggesting that the loss-of-function impacts multiple downstream pathways that will require correction.

CF and other maladies of protein misfolding, including lysosomal storage diseases, type II diabetes, cardiovascular and neurological diseases, arise as a result of an imbalance in the capacity of the protein homeostasis (proteostasis) environment to handle the reduced energetic stability of misfolded, mutated proteins that are critical for normal physiology⁴⁻⁶. The cellular proteomic and metabolic environment is highly adaptable, and responds to stress and disease through numerous signaling pathways that include, among others, the unfolded protein response (UPR) and heat shock response (HSR). The latter respond to misfolding and/or aggregation of proteins by altering the transcriptional and post-translational regulation of synthesis, folding and trafficking components to restore function to the protein fold as well as cell, tissue and host physiology^{4,7}. There remains a need in the art for compounds and pharmaceutical compositions to treat conditions associated with proteostasis dysfunction.

### SUMMARY OF THE INVENTION

The present invention is directed to compounds encompassed by the Formulae (I) and (IV), compositions thereof, and methods for the treatment of a condition associated with a dysfunction in proteostasis in a patient in need of such treatment comprising administering to said patient an effective amount of these compounds.

In one embodiment, the invention is directed to a compound having the Formula (I):

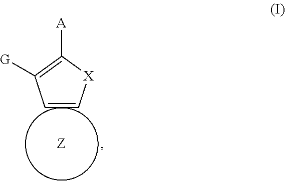

or a pharmaceutically acceptable salt, solvate, or prodrug of any of thereof; wherein:

X is O, S, or NRₐ;

Ring Z is a monocyclic or polycyclic ring system fused to the five-membered heteroaryl containing X, wherein Ring Z is selected from the group consisting of a C₄-C₁₂ cycloalkyl, C₄-C₁₂ cycloalkenyl, 4- to 12-membered heterocyclic, aryl and heteroaryl, each optionally substituted;

A is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙ R_{b}, N(R_{b})COOR_{b}, NR_{b}C(O)C(O)R_{b}, NR_{b}C(O)N(R_{b})₂,NR_{b}S(O)ₙN(R_{b})₂, and OC(O)OR_{b;}

G is selected from the group consisting of optionally substituted 3- to 12-membered heterocyclic, optionally substituted heteroaryl, C(O)N(R_{b})₂, NR_{b}C(O)R_{b}, NR_{b}S(O)ₙR_{b}, and C(R_{b})₂OR_{b};

Rₐ is selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl, C(O)R_{b}, C(O)C(O)R_{b}, C(O)NR_{b}R_{b}, OR_{b}, and S(O)ₙR_{b}; in certain embodiments, Ra is optionally substituted aryl-C₁-C₆-alkyl optionally substituted heteroaryl-C₁-C₆-alkyl, preferably optionally substituted arylmethyl or optionally substituted heteroarylmethyl;

Each R_{b} is independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl; and

n is 0, 1 or 2.

In an additional embodiment, the invention is directed to a compound having the Formula (IIa), (IIb), (IIc), (IId), (IIe), (IIf), (IIg), (IIh), or (IIi):

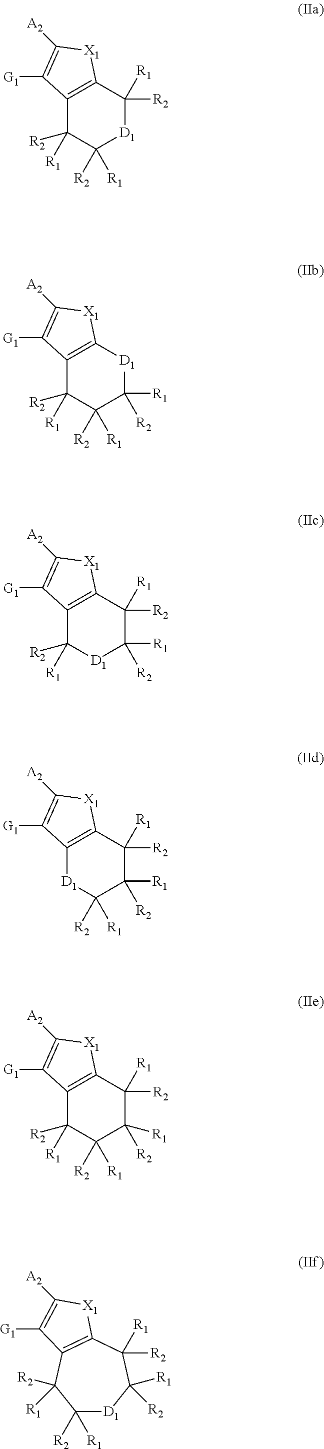

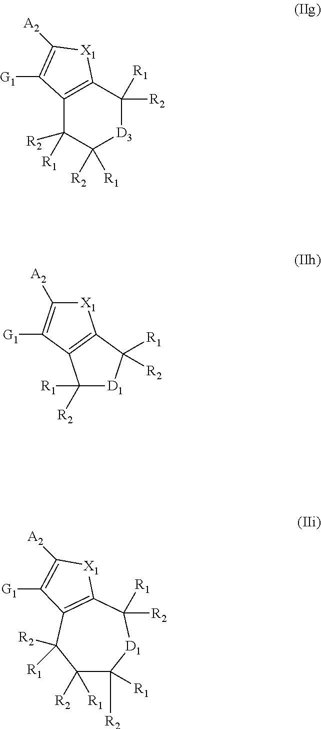

or a pharmaceutically acceptable salt, solvate, or prodrug of any of thereof; wherein:

X₁ is O, S, and NRₐ;

D₁ is selected from the group consisting of N(Rₐ), N(ORₐ), N(Rₐ)(O), N(+)(Rₐ)₂, O and S; in certain embodiments, D₁ is N(Rₐ) or D¹ is N(Rₐ), N(ORₐ), N(Rₐ)(O), O or S;

D₃ is selected from the group consisting of O and S;

A₂ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, N(R_{b})₂, C(O)OR_{b}, NO₂, CN, C(O)R_{b}, C(O)C(O)R_{b}, C(O)NR_{b}R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, and OC(O)OR_{b}; In certain embodiments of the compounds of Formula (Ia), A₂ is is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, N(R_{b})₂, NR_{b}C(O)N(R_{b})₂, NR_{b}C(O)R_{b}, NR_{b}S(O)ₙR_{b}, and OR_{b};

G₁ is selected from the group consisting of optionally substituted 3- to 12-membered heterocyclic, optionally substituted heteroaryl, C(O)N(R_{b})₂, NR_{b}C(O)R_{b}, NR_{b}S(O)ₙR_{b}, and C(R_{b})₂OR_{b}; in certain embodiments, G₁ can additionally be selected from H and optionally substituted aryl.

Each of R₁ and R₂ is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, SR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂,NR_{b}S(O)ₙN(R_{b})₂, S(O)ₙR_{b}, S(O)ₙNR_{b}R_{b}, OC(O)OR_{b} and (C═NR_{b})R_{b}; alternatively, two vicinal R₁ groups can be taken together with the atoms to which they are attached to form a fused, optionally substituted cyclic group selected from the group consisting of optionally substituted C₄-C₈ cycloalkyl, optionally substituted C₄-C₈ cycloalkenyl, optionally substituted 4- to 8-membered heterocyclic, optionally substituted aryl and optionally substituted heteroaryl; yet alternatively, two geminal R₁ and R₂ groups can be taken together with the carbon atom to which they are attached to form a spiro, optionally substituted cyclic group selected from the group consisting of optionally substituted C₃-C₈ cycloalkyl, optionally substituted C₃-C₈ cycloalkenyl, optionally substituted 3- to 8-membered heterocyclic, optionally substituted aryl and optionally substituted heteroaryl; in certain embodiments, two geminal R₁ and R₂ groups can be taken together with the carbon atom to which they are attached to form a carbonyl group; and

Rₐ, R_{b} and n are as previously defined in Formula (I).

In yet an additional embodiment, the invention is a compound having the Formula (IIa), (IIIb), (IIIc), (IIId), (IIIe), and (IIIf):

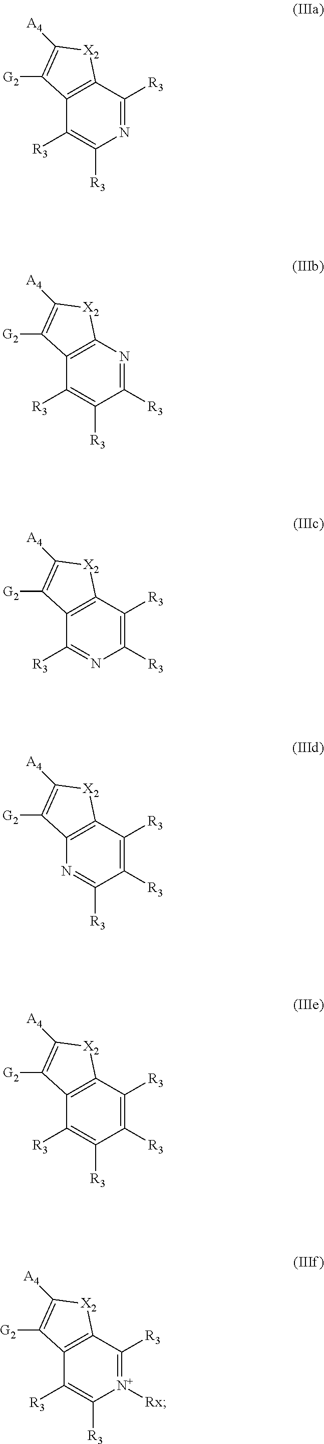

or a pharmaceutically acceptable salt, solvate, or prodrug of any of thereof; wherein:
X₂ is O, S, and NRₐ;

A₄ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{c}, NO₂, CN, C(O)R_{c}, C(O)C(O)R_{c}, C(O)NR_{c}R_{b}, NR_{c}C(O)R_{c}, NR_{b}S(O)ₙR_{c}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, and OC(O)OR_{b};

G₂ is selected from the group consisting of optionally substituted 3- to 12-membered heterocyclic and optionally substituted heteroaryl, C(O)N(R_{b})₂, NR_{b}C(O)R_{b}, NR_{b}S(O)ₙR_{b}, and C(R_{b})₂OR_{b};

Each R₃ is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, SR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, S(O)ₙR_{b}, S(O)ₙNR_{b}R_{b}, OC(O)OR_{b} and (C═NR_{b})R_{b}; alternatively, two vicinal R₃ groups can be taken together with the carbon atoms to which they are attached to form a fused, optionally substituted cyclic group selected from the group consisting of optionally substituted C₄-C₈ cycloalkyl, optionally substituted C₄-C₈ cycloalkenyl, optionally substituted 4- to 8-membered heterocyclic, optionally substituted aryl and optionally substituted heteroaryl;

Rx is selected from O(N-oxide) or optionally substituted C₁-C₁₀ alkyl; and

Rₐ, R_{b} and n are as previously defined.

In an additional aspect, the invention is directed to a compound having the Formula (IVa), (IVb) or (IVc):

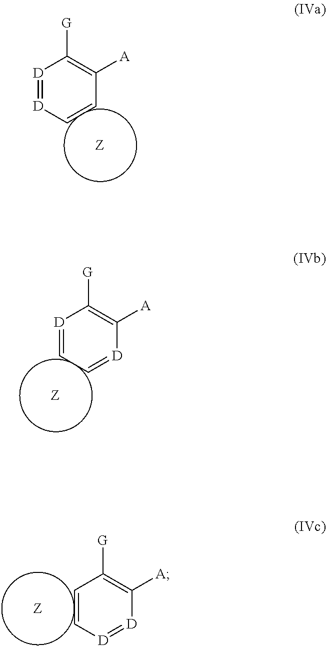

or a pharmaceutically acceptable salt, solvate, or prodrug of any of thereof; wherein:

the two D groups are each C(Rₐ), or alternatively, one of the D groups is C(Rₐ) and the other D group is N;

Ring Z is a monocyclic or polycyclic ring system fused to the six-membered aromatic ring containing the D groups, wherein Ring Z is a C₄-C₁₂ cycloalkyl, C₄-C₁₂ cycloalkenyl, 4- to 12-membered heterocyclic, aryl or heteroaryl, each optionally substituted;

A is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, and OC(O)OR_{b;}

G is selected from the group consisting of optionally substituted 3- to 12-membered heterocyclic, optionally substituted heteroaryl, C(O)N(R_{b})₂, NR_{b}C(O)R_{b}, NR_{b}S(O)ₙR_{b}, and C(R_{b})₂OR_{b}; and

Rₐ, R_{b} and n are as previously defined.

In yet an additional aspect, the invention is a compound having the Formula (Va), (Vb), (Vc), (Vd), (Ve), (Vf), (Vg), (Vh), (Vi), (Vj), (Vk), (Vl), (Vm), and (Vn):

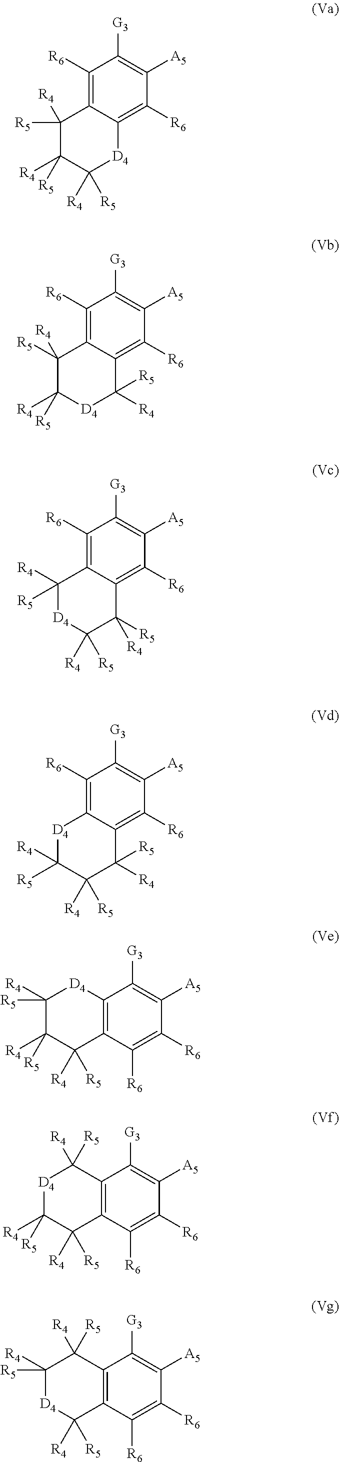

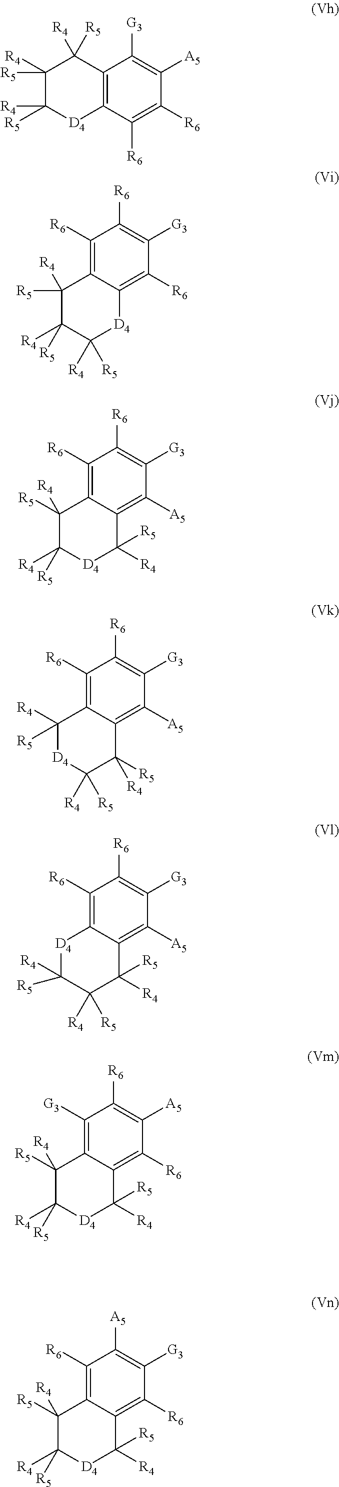

or a pharmaceutically acceptable salt, solvate, or prodrug of any of thereof; wherein: D₄ is selected from the group consisting of N(Rₐ), C(Rₐ)₂, N(ORₐ), N(Rₐ), (O), O and S;

A₅ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, and OC(O)OR_{b;}

G₃ is selected from the group consisting of optionally substituted 3- to 12-membered heterocyclic, optionally substituted heteroaryl, C(O)N(R_{b})₂, NR_{b}C(O)R_{b}, NR_{b}S(O)ₙR_{b}, and C(R_{b})₂OR_{b};

Each of R₄ and R₅ are independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, SR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, S(O)ₙR_{b}, S(O)ₙNR_{b}R_{b}, OC(O)OR_{b} and (C═NR_{b})R_{b}; alternatively, two vicinal R₄ or R₅ groups can be taken together with the carbon atoms to which they are attached to form a fused, optionally substituted cyclic group selected from the group consisting of optionally substituted C₄-C₈ cycloalkyl, optionally substituted C₄-C₈ cycloalkenyl, optionally substituted 4- to 8-membered heterocyclic, optionally substituted aryl and optionally substituted heteroaryl; yet alternatively, two geminal R₄ and R₅ groups can be taken together with the carbon atom to which they are attached to form a spiro, optionally substituted cyclic group selected from the group consisting of optionally substituted C₃-C₈ cycloalkyl, optionally substituted C₃-C₈ cycloalkenyl, optionally substituted 3- to 8-membered heterocyclic, optionally substituted aryl and optionally substituted heteroaryl;

Each R₆ is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, SR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, S(O)ₙR_{b}, S(O)ₙNR_{b}R_{b}, OC(O)OR_{b} and (C═NR_{b})R_{b}; and

Rₐ, R_{b} and n as previously defined.

The invention also encompasses a compound having the Formula (VIa), (VIb), (VIc), (VId), (VIe), (VIf), (VIg), (VIh), (VIi), (VIj), (VIk) and (VIl):

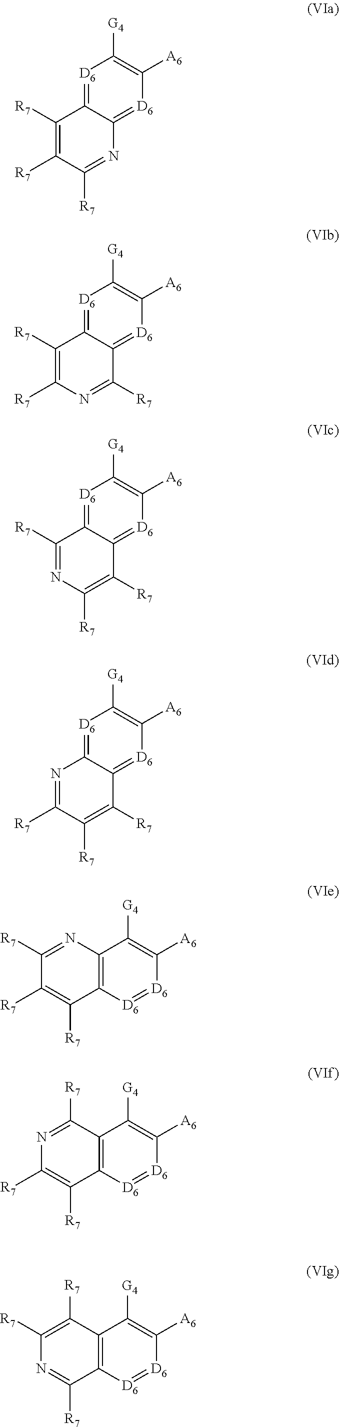

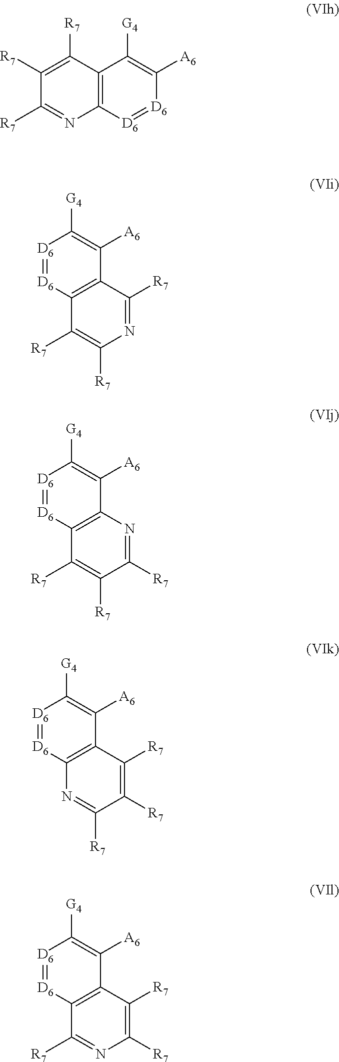

or a pharmaceutically acceptable salt, solvate, or prodrug of any of thereof; wherein:

the two D₆ groups (per ring) are each C(Rₐ); or alternative one of the D₆ groups is C(Rₐ) and the other D₆ group is N;

A₆ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, and OC(O)OR_{b;}

G₄ is selected from the group consisting of optionally substituted 3- to 12-membered heterocyclic, optionally substituted heteroaryl, C(O)N(R_{b})₂, NR_{b}C(O)R_{b}, NR_{b}S(O)ₙR_{b}, and C(R_{b})₂OR_{b};

Each R₇ is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, SR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, S(O)ₙR_{b}, S(O)ₙNR_{b}R_{b}, OC(O)OR_{b} and (C═NR_{b})R_{b}; alternatively, two vicinal R₇ groups can be taken together with the carbon atoms to which they are attached to form a fused, optionally substituted cyclic group selected from the group consisting of optionally substituted C₄-C₈ cycloalkyl, optionally substituted C₄-C₈ cycloalkenyl,optionally substituted 4- to 8-membered heterocyclic, optionally substituted aryl and optionally substituted heteroaryl; and

Rₐ, R_{b} and n as previously defined.

In one aspect, the invention is a pharmaceutical composition comprising an effective amount of a compound of Formula (I), (IIa-IIi), (IIIa-IIIf), (IVa-IVc), (Va-Vn), (VIa-VIl), (VII), (VIII), or (IX) and a pharmaceutically acceptable carrier.

The invention also includes a method of treating a patient suffering from a condition associated with a dysfunction in proteostasis comprising administering to said patient an effective amount of a compound of Formula (I), (IIa-IIe), (IIIa-IIIf), (IVa-IVc), (Va-Vn), (VIa-VIl), (VII), (VIII) or (IX) and a pharmaceutically acceptable carrier.

In another embodiment, the invention is a pharmaceutical composition comprising a pharmaceutically acceptable carrier and an effective amount of a compound having the Formula (IIb), (IIc), (IId), (IIe), (IIf), (IIg), (IIh) or (IIi):

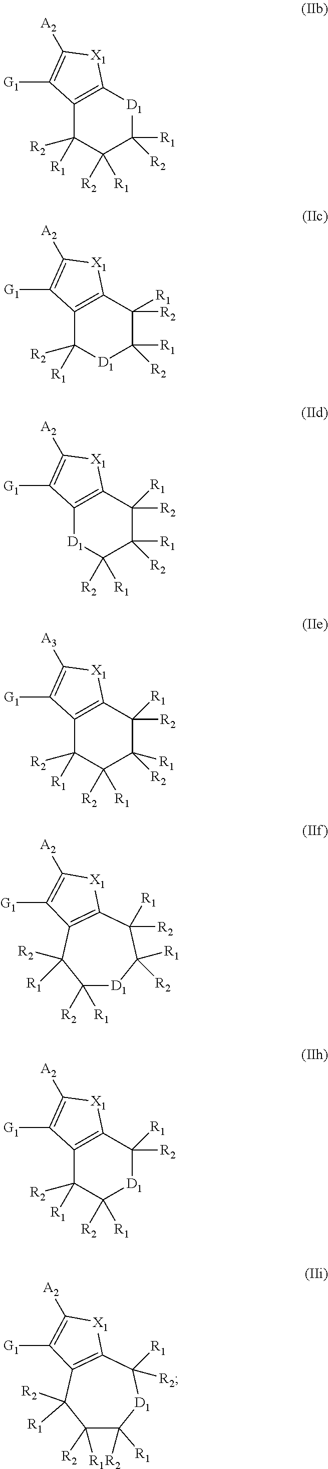

or a pharmaceutically acceptable salt, prodrug or solvate thereof;

X₁ is O, S, and NRₐ;

D₁ is selected from the group consisting of N(Rₐ), N(ORₐ), N(Rₐ)(O), O and S;

A₂ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂,NR_{b}S(O)ₙR_{b}, and OC(O)OR_{b};

G₁ is selected from the group consisting of optionally substituted 3- to 12-membered heterocyclic and optionally substituted heteroaryl, CON(R_{b})₂, NR_{b}C(O)R_{b}, NR_{b}S(O)ₙR_{b}, and C(R_{b})₂OR_{b};

Each of R₁ and R₂ are independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, SR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}S(O)ₙ(NR_{b})₂, S(O)ₙR_{b}, S(O)ₙNR_{b}R_{b}, OC(O)OR_{b} and (C═NR_{b})R_{b}; alternatively, two vicinal R₁ groups can be taken together with the carbon atoms to which they are attached to form a fused, optionally substituted cyclic group selected from the group consisting of optionally substituted C₄-C₈ cycloalkyl, optionally substituted C₄-C₈ cycloalkenyl, optionally substituted 4- to 8-membered heterocyclic, optionally substituted aryl and optionally substituted heteroaryl; yet alternatively, two geminal R₁ and R₂ groups can be taken together with the carbon to which they are attached to form a spiro, optionally substituted cyclic group selected from the group consisting of optionally substituted C₃-C₈ cycloalkyl, optionally substituted C₃-C₈ cycloalkenyl, optionally substituted 3- to 8-membered heterocyclic; optionally substituted aryl and optionally substituted heteroaryl and

Rₐ, R_{b} and n are as previously defined.

The invention further includes a method of treating a patient suffering from a condition associated with a dysfunction in proteostasis comprising administering to said patient a pharmaceutical composition comprising an effective amount of a compound of Formula (I), (IIa-IIi), (IIIc-IIIf), (IVa-IVc), (Va-Vn), (VIa-VIl), (VII), (VIII) or (IX), or a pharmaceutically acceptable salt, solvate or produg thereof, and a pharmaceutically acceptable carrier.

In an additional aspect, the invention is directed to a pharmaceutical composition comprising:

a pharmaceutically acceptable carrier or excipient;

an effective amount of a compound having the Formula (I), (IIa-IIi), (IIIa-IIIf), (IVa-IVc), (Va-Vn), (VIa-VIl), (VII), (VIII), or (IX) or a pharmaceutically acceptable salt, solvate, or prodrug of thereof; and

an effective amount of a second agent selected from the group consisting of a proteostasis regulator and a pharmacologic chaperone.

The invention additionally encompasses a method of treating cancer or a tumor comprising administering to a patient in need thereof an effective amount of a compound having the Formula (I), (IIa-IIi), (IIIa-IIIf), (IVa-IVc), (Va-Vn), (VIa-VIl), (VII), (VIII) or (IX) or a pharmaceutically acceptable salt, solvate, clathrate or prodrug of any of thereof

### BRIEF DESCRIPTION OF THE DRAWINGS

FIG. 1 is an immunoblot analysis showing enhanced levels of bands B and C when CFBE410- lung cells were cultured in the presence and absence of DMSO (lane 1) and compound 2 (lane 2) (in the assay described below in the Exemplification section).

FIG. 2 is a graph showing short circuit currents measured for compounds 2, 6, 7, 9, 13 and 40.

### DETAILED DESCRIPTION OF THE INVENTION

A description of preferred embodiments of the invention follows.

As used herein, the words “a” and “an” are meant to include one or more unless otherwise specified. For example, the term “a cell” encompasses both a single cell and a combination of two or more cells.

As discussed above, the present invention is directed to compounds of Formulae (I), (IVa-IVc), (Va-Vn) and (VIa-VIl), and pharmaceutically acceptable salts, solvates, and prodrugs thereof, pharmaceutical compositions thereof and methods of use thereof in the treatment of conditions associated with a dysfunction in proteostasis.

In some embodiments, the invention is directed to a compound of Formula (I), or a pharmaceutically acceptable salt, solvate, or prodrug of any of thereof. In certain embodiments, the compound has the Formula (I), wherein G is an optionally substituted 3- to 12-membered heterocyclic or heteroaryl.

In one embodiment, the invention is a compound of Formula (I), wherein X is S or O, or a pharmaceutically acceptable salt, solvate, or prodrug of any of thereof. In yet another aspect, Xis S.

In an additional embodiment, the compound has the Formula (I) wherein Ring Z is an optionally substituted 5 to 7-membered ring. In certain embodiments, Ring Z is an optionally substituted 6-membered ring. In a further aspect, Ring Z is an optionally substituted heterocyclic ring system. In yet another aspect, Ring Z is an optionally substituted heteroaryl ring system. In certain aspects, Ring Z is an optionally substituted aryl, such as optionally substituted phenyl. In an additional embodiment, the invention is a compound of Formula (I), wherein Ring Z is an optionally substituted, heteroaryl ring system containing one or more ring nitrogen atoms, for example, a 5- or 6-membered heteroaryl ring system containing one or more ring nitrogen atoms. In yet an additional aspect, Ring Z is selected from the group consisting of thiophenyl, furanyl, pyrrolyl, oxaxolyl, isoxazolyl, isoxazolinyl, thiazolyl, isothiazolyl, thiadiazolyl, triazolyl, tetrazolyl, thieno[2-3c]pyridinyl, benzothiophenyl, tetrahydrobenzothienyl, benzothiazolyl, benzoxazolyl, and benzofuranyl, each optionally substituted.

In certain aspects, the invention is a compound of Formula (I), wherein A is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, and OR_{b}. In another embodiment, A is selected from the group consisting of optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, and OR_{b}.

Non-limiting examples of compounds of Formula (I) include compounds of Formulae (IIa-IIg) and compounds of Formulae and pharmaceutically acceptable salts, solvates, or prodrugs of any of thereof.

In certain embodiments, the invention is a compound of Formula (IIa), (IIb), (IIc), (IId), (IIe), (IIf), (IIg), (IIh) or (IIi), wherein G₁ is an optionally substituted 3- to 12-membered heterocyclic or heteroaryl.

In one embodiment, the compound of the invention has the Formula (IIa), (IIb), (IIc), (II), (IIe), (IIf) or (IIg), wherein X₁ is S or O. In yet an additional embodiment, the compound of the invention has the Formula (IIa-IIi), wherein X₁ is S.

In certain aspects, the compound has the Formula (IIa), wherein A₁ is selected from the group consisting of hydrogen, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, N(R_{b})₂, NR_{b}C(O)N(R_{b})₂,NR_{b}C(O)R_{b},NR_{b}S(O)ₙR_{b}, and OR_{b}. In yet another embodiment, A₁ is selected from the group consisting of optionally substituted C₂-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, and OR_{b}.

In additional aspects, the compound has the Formula (IIb), (IIc), (IId), (IIe), (IIg), (IIh) or (IIi) wherein

A₂ is selected from the group consisting of optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{b}S(O)ₙR_{b}, N(R_{b})COOR_{b}, NR_{b}C(O)C(O)R_{b}, NR_{b}C(O)R_{b}, NR_{b}S(O)ₙNR_{b}NR_{b}, NR_{b}S(O)ₙR_{b}, and OC(O)OR_{b}.

In certain embodiment, the compound has the Formula (IIb), (IIc), (IId), (IIf), (IIg), (IIh) or (IIi), wherein D₁ is N(Rₐ). In certain embodiments, Rₐ is independently selected from the group consisting of hydrogen and optionally substituted C₁-C₁₀ alkyl.

In certain embodiments, the compound has the Formula (IIb), (IIc), (IId), (IIi), (IIg), (IIh) or (IIi), wherein D₁ is O.

In some embodiments, the compounds has the Formula (IIa), (IIb), (IIc), (IId), (IIe), (IIi), (IIg), (IIh) or (IIi), wherein G₁ is an optionally substituted heteroaryl. In certain additional aspects, G₁ is selected from the group consisting of thiophenyl, furanyl, pyrrolyl, oxazolyl, isoxazolyl, isoxazolinyl, thiazolyl, isothiazolyl, thiadiazolyl, triazolyl, tetrazolyl, thieno[2-3c]pyridinyl, benzothiophenyl, tetrahydrobenzothienyl, benzothiazolyl, benzoxazolyl, and benzofuranyl, each optionally substituted. In yet additional embodiments, G₁ is an optionally substituted 5/6-membered fused heteroaryl. In further aspects, G₁ is optionally substituted benzothiazolyl, benzoxazolyl, benzoimidazolyl, benzothiophenyl, and benzofuranyl.

In additional embodiments, the invention is directed to compounds having the Formula In some aspects, the invention is directed to compounds having the Formula (IIIa-IIIf), wherein G₂ is an optionally substituted 3- to 12-membered heterocyclic or heteroaryl.

In certain additional embodiment, compound has the Formula (IIIa), (IIIb), (IIIc), (IIId), or (IIIe), wherein X₂ is S.

In some embodiments, the compound has the Formula (IIIa), (IIIb), (IIIc), (IIId), (IIIe) or (IIIf) wherein A₄ is selected from the group consisting of optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, and OR_{b}.

In additional embodiments, the compound has the Formula (IIIa), (IIIb), (IIIc), (IIId), (IIIe) or (IIIf), wherein G₂ is an optionally substituted heteroaryl. In some embodiments, G₁ is selected from the group consisting of thiophenyl, furanyl, pyrrolyl, oxazolyl, isoxazolyl, isoxazolinyl, thiazolyl, isothiazolyl, thiadiazolyl, triazolyl, tetrazolyl, thieno[2-3c]pyridinyl, benzothiophenyl, tetrahydrobenzothienyl, benzothiazolyl, benzoxazolyl, and benzofuranyl, each optionally substituted.

A preferred subset of compounds of Formula I is represented by Formula (VII)

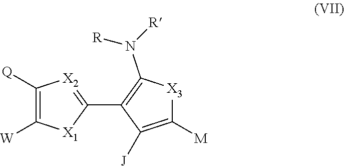

as well as pharmaceutically acceptable salts, solvates, prodrugs and N-oxides thereof, wherein:

X₁ and X₃ are each independently O, S or NR^{c}, where each R^{c} is independently H or substituted or unsubstituted C₁-C₆-alkyl, preferably methyl;

X₂ is N or CR^{c};

J and M, together with the carbon atoms to which they are attached, form an optionally substituted 5 to 7 membered carbocyclic or heterocyclic ring, wherein the heterocyclic ring comprises one or two heteroatoms independently selected from nitrogen and oxygen and the remainder of the ring atoms are carbon;

Q and W, together with the carbon atoms to which they are attached, form an optionally substituted 5 to 7 membered carbocyclic ring; and

R and R′ are each independently hydrogen, substituted or unsubstituted C₁-C₆-alkyl, substituted or unsubstituted C₂-C₆-alkenyl, substituted or unsubstituted C₂-C₆-alkynyl, —C(O)—R^{d} or —SO₂-R^{d}, where R^{d} is substituted or unsubstituted C₁-C₆-alkyl, substituted or unsubstituted aryl, or substituted or unsubstituted aryl-C₁-C₆-alkyl.

Preferably, X₁ is S and X₂ is N.

Preferably, X₃ is S.

Preferably, J, M and the carbon atoms to which they are attached form a benzo ring, a pyrido ring, an azacyclopentene ring, an azacyclohexene ring, an azacycloheptene ring, an oxacyclopentene ring, an oxacyclohexene ring or an oxaacycloheptene ring. The nitrogen atom of the azacyclopentene, azacyclohexene, or azacycloheptene ring is unsubstituted or substituted with a C₁-C₆-alkyl group; when this nitrogen atom is substituted, the compounds of Forumla VII further include the corresponding N-oxide.

Preferably, Q, W and the carbon atoms to which they are attached form a benzo ring, a cyclopentene ring, a cyclohexene ring or a cycloheptene ring, each of which is optionally substituted with C₁-C₆-alkyl or halogen. In certain embodiments, Q, W and the carbon atoms to which they are attached form a benzo ring which is unsubstituted or substituted with a methyl group or a halogen, such as chlorine or fluorine.

Preferably, R is H and R′ is H, C₁-C₃-alkyl, —C(O)—R^{d}, where R^{d} is C₁-C₆-alkyl, phenyl, or phenyl substituted with one to three halogen atoms. In certain embodiments, R is H and R′ is methyl or C(O)—R^{d}, where R^{d} is methyl, isopropyl, phenyl or 2-chlorophenyl.

Certain preferred embodiments of the compounds of Formula VII are of Formula VIII:

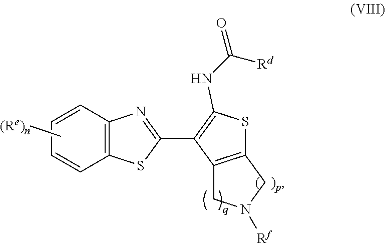

as well as pharmaceutically acceptable salts, solvates, prodrugs and N-oxides thereof, where:

R^{d} is substituted or unsubstituted C₁-C₆-alkyl, substituted or unsubstituted phenyl, such asr halogen-substituted phenyl; preferably R^{d} is methyl;

Each R^{e} is substituted or unsubstituted C₁-C₆-alkyl, such as halogenated C₁-C₆-alkyl, or halogen; preferably each R^{e} is methyl, trifluoromethyl or fluorine;

R^{f} is hydrogen or substituted or unsubstituted C₁-C₆-alkyl, preferably H, methyl, ethyl or isopropyl;

n is 0 to 3, preferably 0 or 1;

p and q are each independently 1 to 3, provided that the sum of p and q is 2 to 4; preferably, p is 1 and q is 2.

In a preferred embodiment, the compounds of Formula VII are represented by Formula (IX):

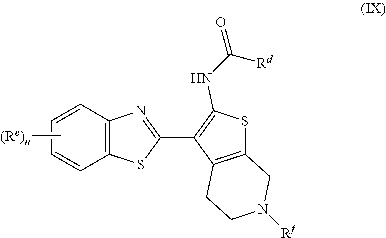

wherein:

R^{d} is substituted or unsubstituted C₁-C₆-alkyl, preferably methyl;

n is 0 or 1;

R^{e} is C₁-C₆-alkyl, substituted C₁-C₆-alkyl, such as halogenated C₁-C₆-alkyl, or halogen, preferably methyl, trifluoromethyl or fluorine; when n is 1, R^{e} is preferably at C₅ or C₆ of the benzothiazole group; and

R^{f} is C₁-C₆-alkyl or substituted C₁-C₆-alkyl, preferably methyl.

Non-limiting examples of compounds having the Formulae (I), (IIa-IIi), or (VII)-(IX) are shown below in Table la:

TABLE 1a

| Compound | |
|---|---|
| Number | Compound |
| 1 | 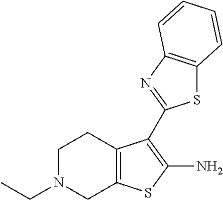 [SMILES: CCN1CCc2c(sc(N)c2-c2nc3ccccc3s2)C1] |
| 2 | 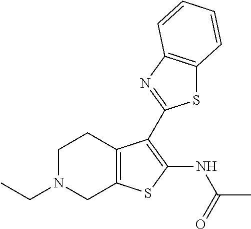 [SMILES: CCN1CCc2c(sc(NC(C)=O)c2-c2nc3ccccc3s2)C1] |
| 3 | 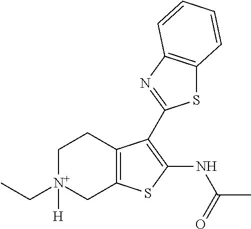 [SMILES: CC[NH+]1CCc2c(sc(NC(C)=O)c2-c2nc3ccccc3s2)C1] |
| 4 | 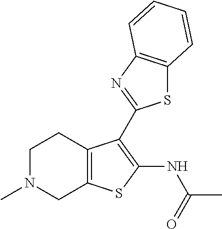 [SMILES: CC(=O)Nc1sc2c(c1-c1nc3ccccc3s1)CCN(C)C2] |
| 5 | 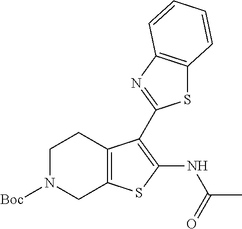 [SMILES: CC(=O)Nc1sc2c(c1-c1nc3ccccc3s1)CCN(C(=O)OC(C)(C)C)C2] |
| 6 | 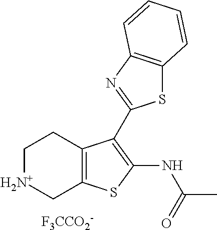 |
| 7 | 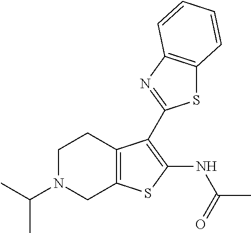 [SMILES: CC(=O)Nc1sc2c(c1-c1nc3ccccc3s1)CCN(C(C)C)C2] |
| 8 | 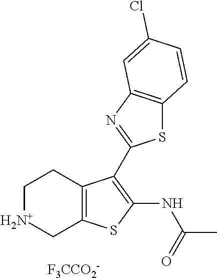 |
| 9 | 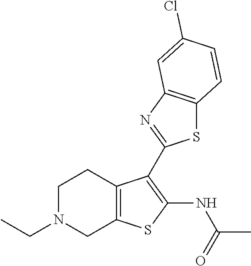 [SMILES: CCN1CCc2c(sc(NC(C)=O)c2-c2nc3cc(Cl)ccc3s2)C1] |
| 10 | 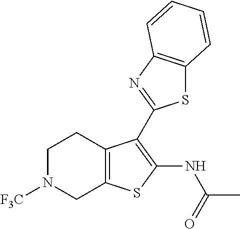 [SMILES: CC(=O)Nc1sc2c(c1-c1nc3ccccc3s1)CCN(C(F)(F)F)C2] |
| 11 | 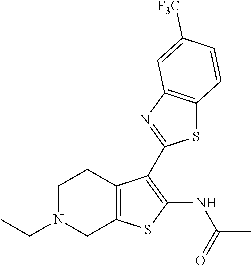 [SMILES: CCN1CCc2c(sc(NC(C)=O)c2-c2nc3cc(C(F)(F)F)ccc3s2)C1] |
| 12 | 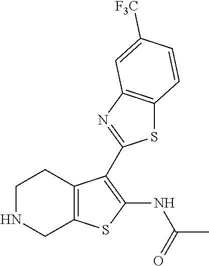 [SMILES: CC(=O)Nc1sc2c(c1-c1nc3cc(C(F)(F)F)ccc3s1)CCNC2] |
| 13 | 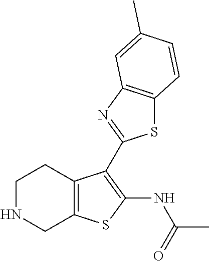 [SMILES: CC(=O)Nc1sc2c(c1-c1nc3cc(C)ccc3s1)CCNC2] |
| 14 | 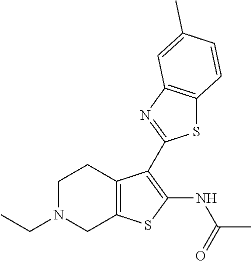 [SMILES: CCN1CCc2c(sc(NC(C)=O)c2-c2nc3cc(C)ccc3s2)C1] |
| 15 | 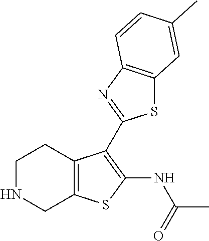 [SMILES: CC(=O)Nc1sc2c(c1-c1nc3ccc(C)cc3s1)CCNC2] |
| 16 | 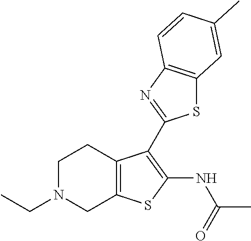 [SMILES: CCN1CCc2c(sc(NC(C)=O)c2-c2nc3ccc(C)cc3s2)C1] |
| 17 | 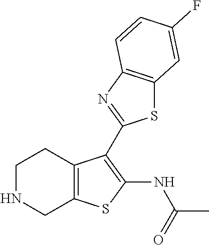 [SMILES: CC(=O)Nc1sc2c(c1-c1nc3ccc(F)cc3s1)CCNC2] |
| 18 | 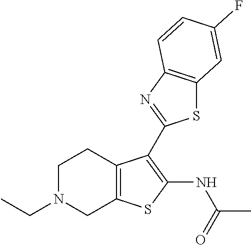 [SMILES: CCN1CCc2c(sc(NC(C)=O)c2-c2nc3ccc(F)cc3s2)C1] |
| 19 | 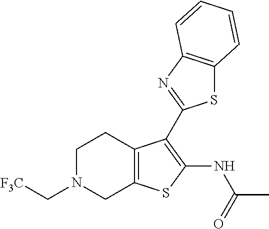 [SMILES: CC(=O)Nc1sc2c(c1-c1nc3ccccc3s1)CCN(CC(F)(F)F)C2] |
| 20 | 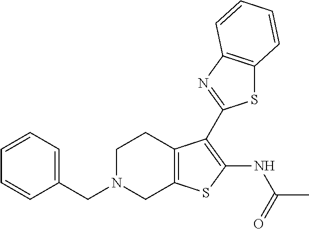 [SMILES: CC(=O)Nc1sc2c(c1-c1nc3ccccc3s1)CCN(Cc1ccccc1)C2] |
| 21 | 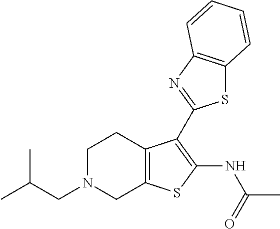 [SMILES: CC(=O)Nc1sc2c(c1-c1nc3ccccc3s1)CCN(CC(C)C)C2] |
| 22 | 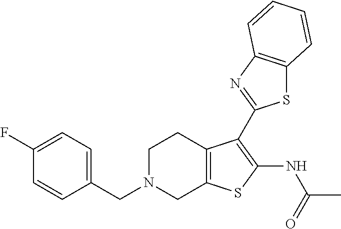 [SMILES: CC(=O)Nc1sc2c(c1-c1nc3ccccc3s1)CCN(Cc1ccc(F)cc1)C2] |
| 23 | 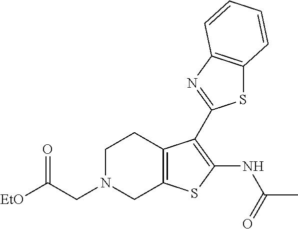 [SMILES: CCOC(=O)CN1CCc2c(sc(NC(C)=O)c2-c2nc3ccccc3s2)C1] |
| 24 | 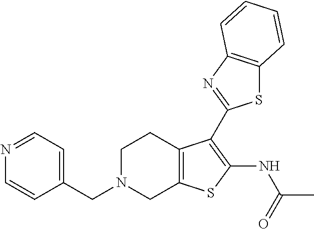 [SMILES: CC(=O)Nc1sc2c(c1-c1nc3ccccc3s1)CCN(Cc1ccncc1)C2] |
| 25 | 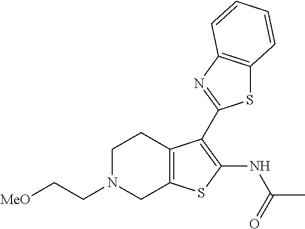 [SMILES: COCCN1CCc2c(sc(NC(C)=O)c2-c2nc3ccccc3s2)C1] |
| 26 | 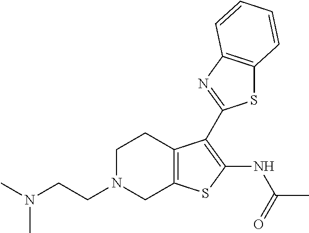 [SMILES: CC(=O)Nc1sc2c(c1-c1nc3ccccc3s1)CCN(CCN(C)C)C2] |
| 27 | 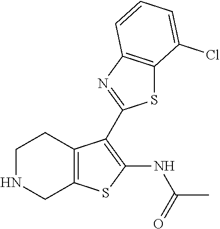 [SMILES: CC(=O)Nc1sc2c(c1-c1nc3cccc(Cl)c3s1)CCNC2] |
| 28 | 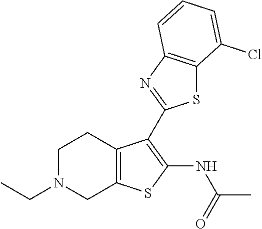 [SMILES: CCN1CCc2c(sc(NC(C)=O)c2-c2nc3cccc(Cl)c3s2)C1] |
| 29 | 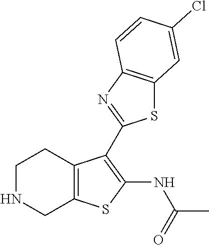 [SMILES: CC(=O)Nc1sc2c(c1-c1nc3ccc(Cl)cc3s1)CCNC2] |
| 30 | 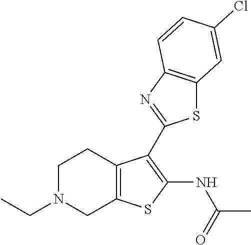 [SMILES: CCN1CCc2c(sc(NC(C)=O)c2-c2nc3ccc(Cl)cc3s2)C1] |
| 31 | 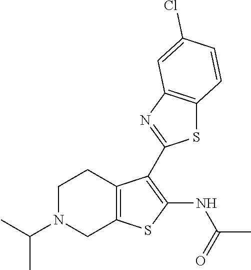 [SMILES: CC(=O)Nc1sc2c(c1-c1nc3cc(Cl)ccc3s1)CCN(C(C)C)C2] |
| 32 | 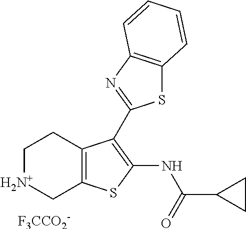 |
| 33 | 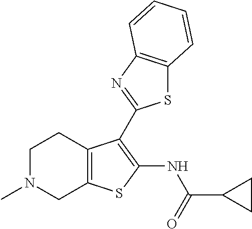 [SMILES: CN1CCc2c(sc(NC(=O)C3CC3)c2-c2nc3ccccc3s2)C1] |
| 34 | 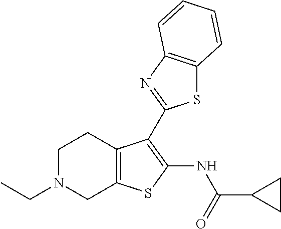 [SMILES: CCN1CCc2c(sc(NC(=O)C3CC3)c2-c2nc3ccccc3s2)C1] |
| 35 | 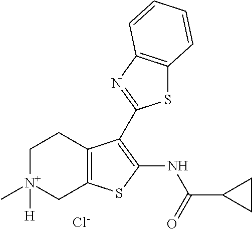 [SMILES: C[NH+]1CCc2c(sc(NC(=O)C3CC3)c2-c2nc3ccccc3s2)C1 ; [Cl-]] |
| 36 | 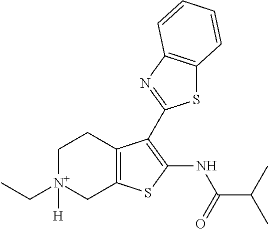 [SMILES: CC[NH+]1CCc2c(sc(NC(=O)C(C)C)c2-c2nc3ccccc3s2)C1] |
| 37 | 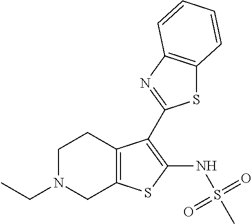 [SMILES: CCN1CCc2c(sc(NS(C)(=O)=O)c2-c2nc3ccccc3s2)C1] |
| 38 | 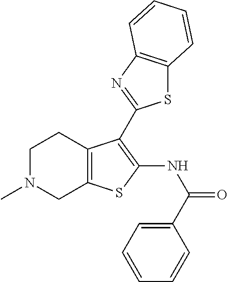 [SMILES: CN1CCc2c(sc(NC(=O)c3ccccc3)c2-c2nc3ccccc3s2)C1] |
| 39 | 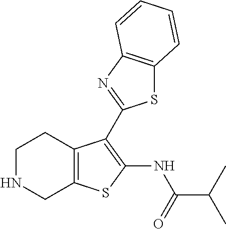 [SMILES: CC(C)C(=O)Nc1sc2c(c1-c1nc3ccccc3s1)CCNC2] |
| 40 | 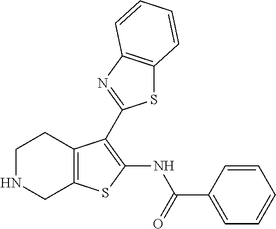 [SMILES: O=C(Nc1sc2c(c1-c1nc3ccccc3s1)CCNC2)c1ccccc1] |
| 41 | 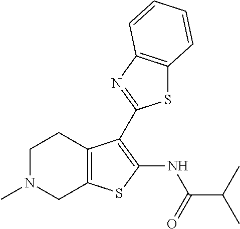 [SMILES: CC(C)C(=O)Nc1sc2c(c1-c1nc3ccccc3s1)CCN(C)C2] |
| 42 | 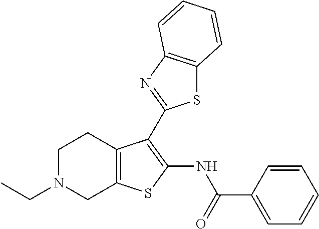 [SMILES: CCN1CCc2c(sc(NC(=O)c3ccccc3)c2-c2nc3ccccc3s2)C1] |
| 43 | 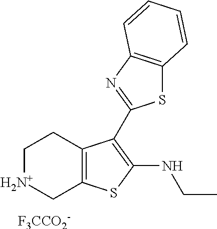 |
| 44 | 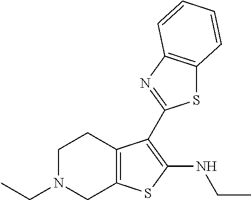 [SMILES: CCNc1sc2c(c1-c1nc3ccccc3s1)CCN(CC)C2] |
| 45 | 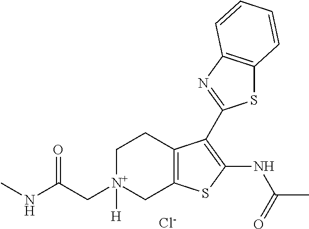 [SMILES: CNC(=O)C[NH+]1CCc2c(sc(NC(C)=O)c2-c2nc3ccccc3s2)C1 ; [Cl-]] |
| 46 | 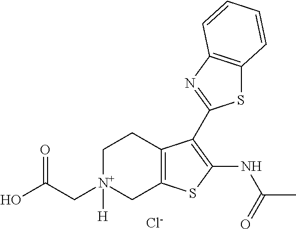 [SMILES: CC(=O)Nc1sc2c(c1-c1nc3ccccc3s1)CC[NH+](CC(=O)O)C2 ; [Cl-]] |
| 47 | 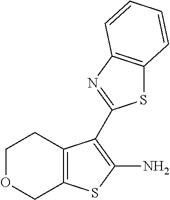 [SMILES: Nc1sc2c(c1-c1nc3ccccc3s1)CCOC2] |
| 48 | 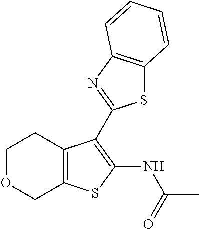 [SMILES: CC(=O)Nc1sc2c(c1-c1nc3ccccc3s1)CCOC2] |
| 51 | 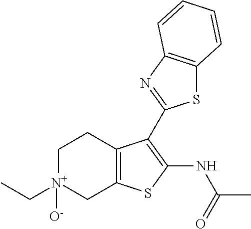 [SMILES: CC[N+]1([O-])CCc2c(sc(NC(C)=O)c2-c2nc3ccccc3s2)C1] |
| 52 | 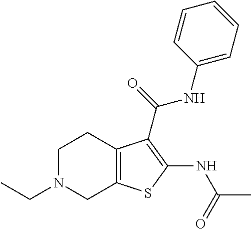 [SMILES: CCN1CCc2c(sc(NC(C)=O)c2C(=O)Nc2ccccc2)C1] |
| 53 | 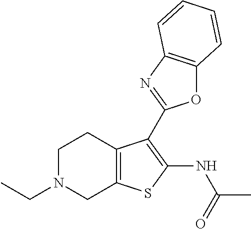 [SMILES: CCN1CCc2c(sc(NC(C)=O)c2-c2nc3ccccc3o2)C1] |
| 54 | 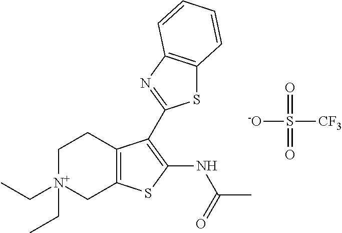 |
| 58 | 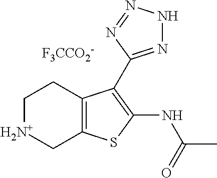 [SMILES: "F3CCO2-" ; CC(=O)Nc1sc2c(c1-c1nn[nH]n1)CC[NH2+]C2] |
| 59 | 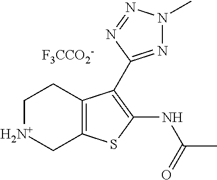 [SMILES: "F3CCO2-" ; CC(=O)Nc1sc2c(c1-c1nnn(C)n1)CC[NH2+]C2] |
| 60 | 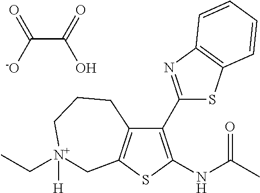 |
| 61 | 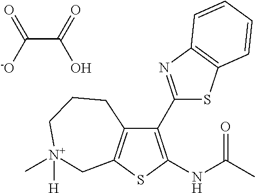 |
| 62 | 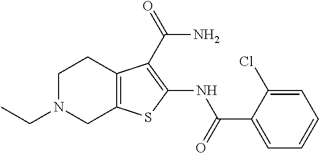 [SMILES: CCN1CCc2c(sc(NC(=O)c3ccccc3Cl)c2C(N)=O)C1] |
| 63 | 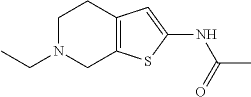 |
| 65 | 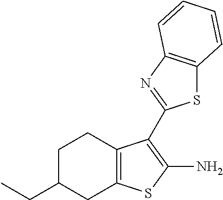 [SMILES: CCC1CCc2c(sc(N)c2-c2nc3ccccc3s2)C1] |
| 66 | 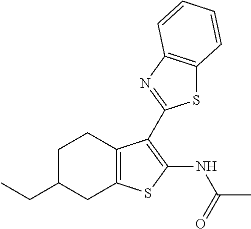 [SMILES: CCC1CCc2c(sc(NC(C)=O)c2-c2nc3ccccc3s2)C1] |
| 67 | 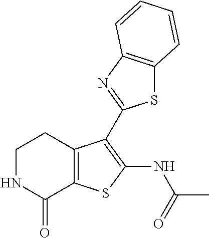 [SMILES: CC(=O)Nc1sc2c(c1-c1nc3ccccc3s1)CCNC2=O] |
| 68 | 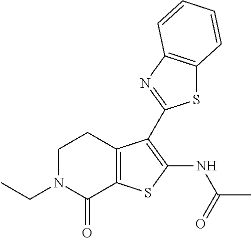 [SMILES: CCN1CCc2c(sc(NC(C)=O)c2-c2nc3ccccc3s2)C1=O] |
| 69 | 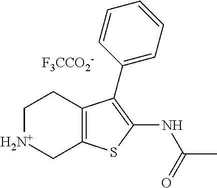 [SMILES: "F3CCO2-" ; CC(=O)Nc1sc2c(c1-c1ccccc1)CC[NH2+]C2] |
| 70 | 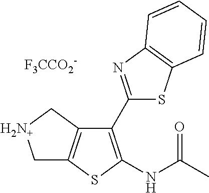 [SMILES: "F3CCO2-" ; CC(=O)Nc1sc2c(c1-c1nc3ccccc3s1)C[NH2+]C2] |
| 71 | 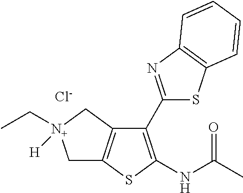 [SMILES: CC[NH+]1Cc2sc(NC(C)=O)c(-c3nc4ccccc4s3)c2C1 ; [Cl-]] |
| 72 | 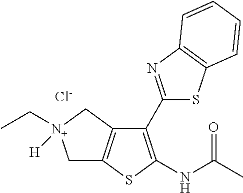 [SMILES: CC[NH+]1Cc2sc(NC(C)=O)c(-c3nc4ccccc4s3)c2C1 ; [Cl-]] |
| 77 | 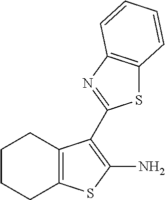 [SMILES: Nc1sc2c(c1-c1nc3ccccc3s1)CCCC2] |
| 78 | 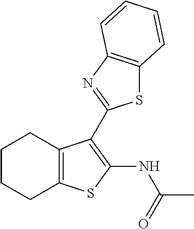 [SMILES: CC(=O)Nc1sc2c(c1-c1nc3ccccc3s1)CCCC2] |
| 80 | 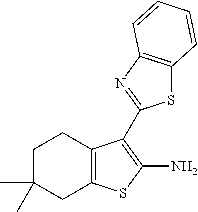 [SMILES: CC1(C)CCc2c(sc(N)c2-c2nc3ccccc3s2)C1] |
| 81 | 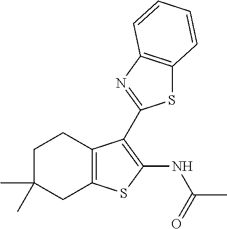 [SMILES: CC(=O)Nc1sc2c(c1-c1nc3ccccc3s1)CCC(C)(C)C2] |
| 82 | 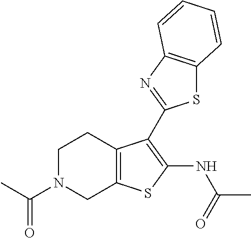 [SMILES: CC(=O)Nc1sc2c(c1-c1nc3ccccc3s1)CCN(C(C)=O)C2] |
| 83 | 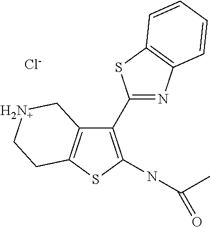 [SMILES: CC(=O)Nc1sc2c(c1-c1nc3ccccc3s1)C[NH2+]CC2 ; [Cl-]] |
| 84 | 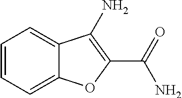 [SMILES: NC(=O)c1oc2ccccc2c1N] |
| 85 | 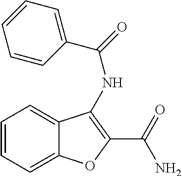 [SMILES: NC(=O)c1oc2ccccc2c1NC(=O)c1ccccc1] |
| 86 | 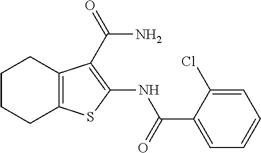 [SMILES: NC(=O)c1c(NC(=O)c2ccccc2Cl)sc2c1CCCC2] |
| 87 | 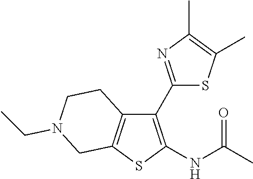 [SMILES: CCN1CCc2c(sc(NC(C)=O)c2-c2nc(C)c(C)s2)C1] |
| 88 | 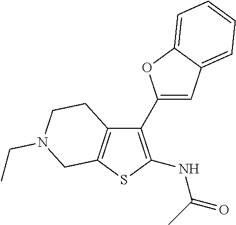 [SMILES: CCN1CCc2c(sc(NC(C)=O)c2-c2cc3ccccc3o2)C1] |
| 89 | 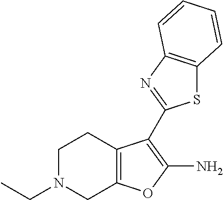 [SMILES: CCN1CCc2c(oc(N)c2-c2nc3ccccc3s2)C1] |
| 90 | 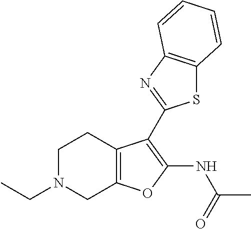 [SMILES: CCN1CCc2c(oc(NC(C)=O)c2-c2nc3ccccc3s2)C1] |
| 91 | 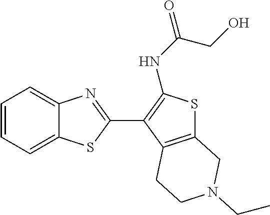 [SMILES: CCN1CCc2c(sc(NC(=O)CO)c2-c2nc3ccccc3s2)C1] |
| 92 | 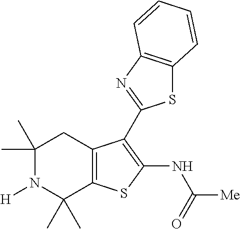 [SMILES: CC(=O)Nc1sc2c(c1-c1nc3ccccc3s1)CC(C)(C)NC2(C)C] |
| 93 | 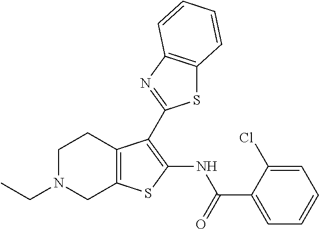 [SMILES: CCN1CCc2c(sc(NC(=O)c3ccccc3Cl)c2-c2nc3ccccc3s2)C1] |
| 94 | 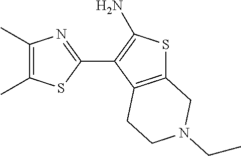 [SMILES: CCN1CCc2c(sc(N)c2-c2nc(C)c(C)s2)C1] |
| 95 | 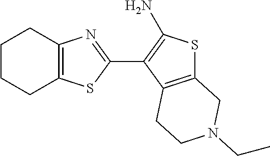 [SMILES: CCN1CCc2c(sc(N)c2-c2nc3c(s2)CCCC3)C1] |
| 96 | 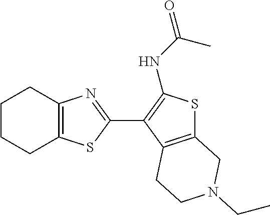 [SMILES: CCN1CCc2c(sc(NC(C)=O)c2-c2nc3c(s2)CCCC3)C1] |
| 97 | 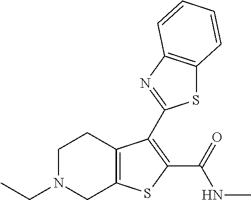 [SMILES: CCN1CCc2c(sc(C(=O)NC)c2-c2nc3ccccc3s2)C1] |
| 98 | 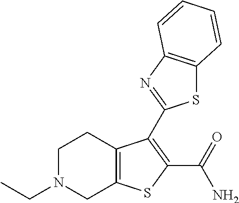 [SMILES: CCN1CCc2c(sc(C(N)=O)c2-c2nc3ccccc3s2)C1] |
| 99 | 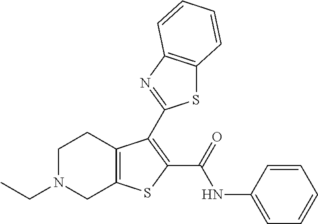 [SMILES: CCN1CCc2c(sc(C(=O)Nc3ccccc3)c2-c2nc3ccccc3s2)C1] |
| 100 | 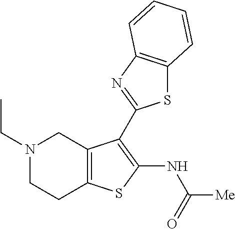 [SMILES: CCN1CCc2sc(NC(C)=O)c(-c3nc4ccccc4s3)c2C1] |
| 101 | 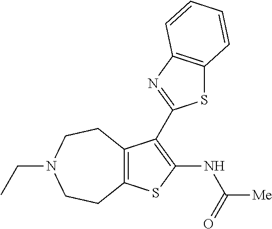 [SMILES: CCN1CCc2sc(NC(C)=O)c(-c3nc4ccccc4s3)c2CC1] |
| 102 | 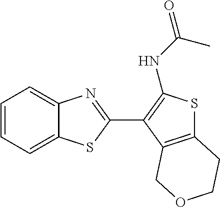 [SMILES: CC(=O)Nc1sc2c(c1-c1nc3ccccc3s1)COCC2] |
| 103 | 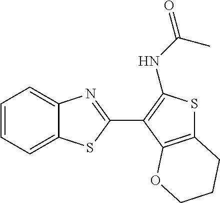 [SMILES: CC(=O)Nc1sc2c(c1-c1nc3ccccc3s1)OCCC2] |
| 104 | 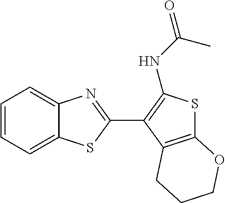 [SMILES: CC(=O)Nc1sc2c(c1-c1nc3ccccc3s1)CCCO2] |
| 105 | 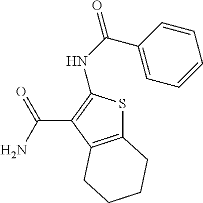 [SMILES: NC(=O)c1c(NC(=O)c2ccccc2)sc2c1CCCC2] |
| 106 | 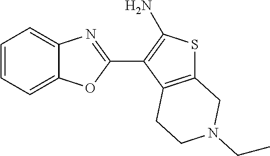 [SMILES: CCN1CCc2c(sc(N)c2-c2nc3ccccc3o2)C1] |
| 107 | 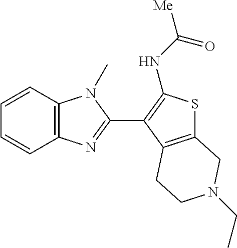 [SMILES: CCN1CCc2c(sc(NC(C)=O)c2-c2nc3ccccc3n2C)C1] |
| 108 | 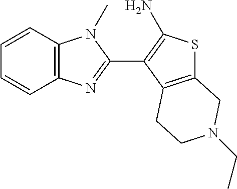 [SMILES: CCN1CCc2c(sc(N)c2-c2nc3ccccc3n2C)C1] |
| 109 | 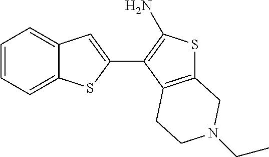 [SMILES: CCN1CCc2c(sc(N)c2-c2cc3ccccc3s2)C1] |
| 110 | 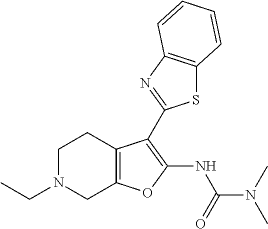 [SMILES: CCN1CCc2c(oc(NC(=O)N(C)C)c2-c2nc3ccccc3s2)C1] |
| 111 | 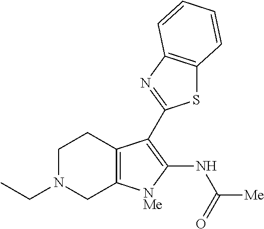 [SMILES: CCN1CCc2c(-c3nc4ccccc4s3)c(NC(C)=O)n(C)c2C1] |
| 112 | 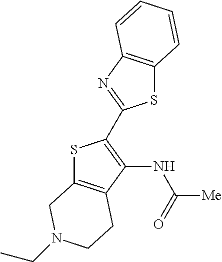 [SMILES: CCN1CCc2c(sc(-c3nc4ccccc4s3)c2NC(C)=O)C1] |
| 113 | 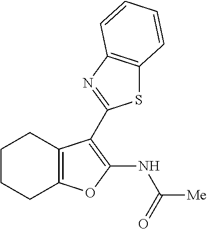 [SMILES: CC(=O)Nc1oc2c(c1-c1nc3ccccc3s1)CCCC2] |
| 114 | 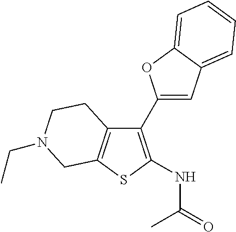 [SMILES: CCN1CCc2c(sc(NC(C)=O)c2-c2cc3ccccc3o2)C1] |
| 123 | 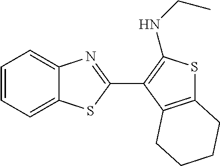 [SMILES: CCNc1sc2c(c1-c1nc3ccccc3s1)CCCC2] |

Non-limiting examples of compounds having the Formulae (IIIa-IIIe) are shown below in Table 1b:

TABLE 1b

| Compound | |
|---|---|
| Number | Compound |
| 49 | 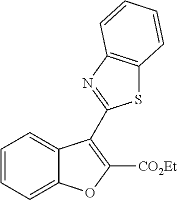 [SMILES: CCOC(=O)c1oc2ccccc2c1-c1nc2ccccc2s1] |
| 50 | 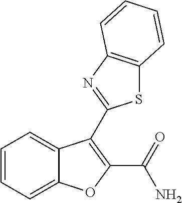 [SMILES: NC(=O)c1oc2ccccc2c1-c1nc2ccccc2s1] |
| 55 | 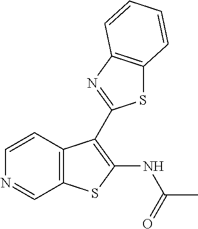 [SMILES: CC(=O)Nc1sc2cnccc2c1-c1nc2ccccc2s1] |
| 56 | 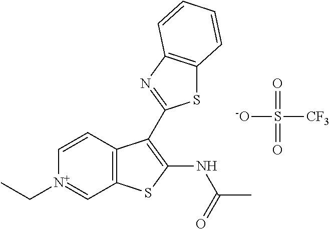 |
| 57 | 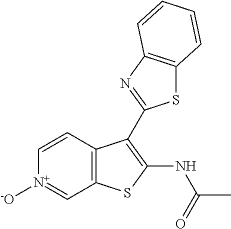 |
| 79 | 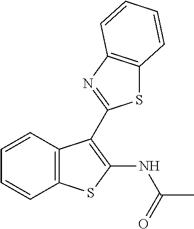 [SMILES: CC(=O)Nc1sc2ccccc2c1-c1nc2ccccc2s1] |
| 84 | 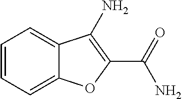 [SMILES: NC(=O)c1oc2ccccc2c1N] |
| 85 | 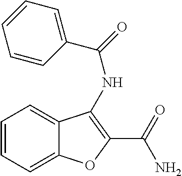 [SMILES: NC(=O)c1oc2ccccc2c1NC(=O)c1ccccc1] |
| 115 | 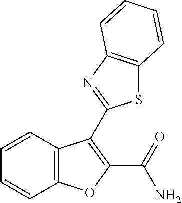 [SMILES: NC(=O)c1oc2ccccc2c1-c1nc2ccccc2s1] |
| 116 | 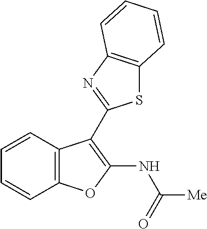 [SMILES: CC(=O)Nc1oc2ccccc2c1-c1nc2ccccc2s1] |
| 117 | 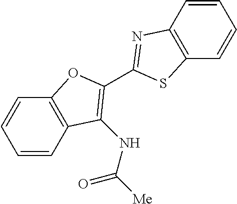 [SMILES: CC(=O)Nc1c(-c2nc3ccccc3s2)oc2ccccc12] |
| 118 | 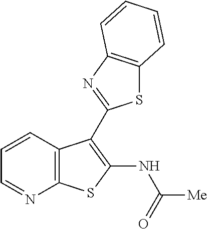 [SMILES: CC(=O)Nc1sc2ncccc2c1-c1nc2ccccc2s1] |
| 119 | 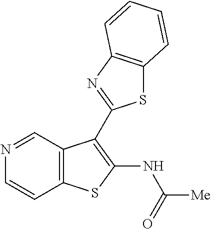 [SMILES: CC(=O)Nc1sc2ccncc2c1-c1nc2ccccc2s1] |
| 120 | 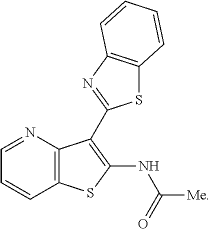 [SMILES: CC(=O)Nc1sc2cccnc2c1-c1nc2ccccc2s1] |

As discussed above, the invention encompasses compounds having the Formula (IVa-IVc), or pharmaceutically acceptable salts, prodrugs or solvates thereof. In certain embodiments, the invention is directed to compounds of Formula (IVa-IVc), wherein G is an optionally substituted 3- to 12-membered heterocyclic or heteroaryl. Non-limiting examples of compounds having the Formula (IVa-IVc) include compounds of Formula (Va-Vn) and (VIa-VIl). Further non-limiting examples of such compounds are shown below in Table 2.

TABLE 2

| Compound | |
|---|---|
| Number | Compound |
| 64 | 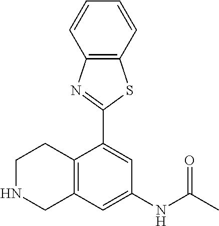 [SMILES: CC(=O)Nc1cc2c(c(-c3nc4ccccc4s3)c1)CCNC2] |
| 73 | 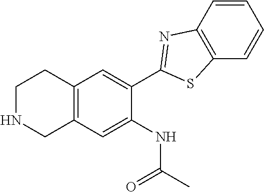 [SMILES: CC(=O)Nc1cc2c(cc1-c1nc3ccccc3s1)CCNC2] |
| 74 | 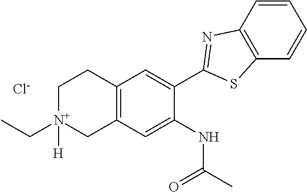 [SMILES: CC[NH+]1CCc2cc(-c3nc4ccccc4s3)c(NC(C)=O)cc2C1 ; [Cl-]] |
| 75 | 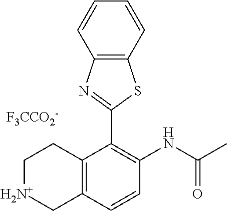 [SMILES: "F3CCO2-" ; CC(=O)Nc1ccc2c(c1-c1nc3ccccc3s1)CC[NH2+]C2] |
| 76 | 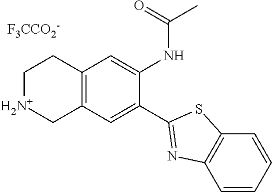 [SMILES: "F3CCO2-" ; CC(=O)Nc1cc2c(cc1-c1nc3ccccc3s1)C[NH2+]CC2] |
| 121 | 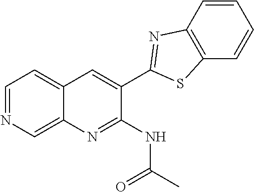 [SMILES: CC(=O)Nc1nc2cnccc2cc1-c1nc2ccccc2s1] |
| 122 | 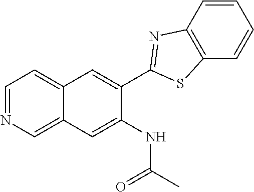 [SMILES: CC(=O)Nc1cc2cnccc2cc1-c1nc2ccccc2s1] |

In certain embodiments, the compounds of the invention do not include the compounds of Formula X below, wherein for each compound R^{d} and R^{f} are set forth in Table 3.

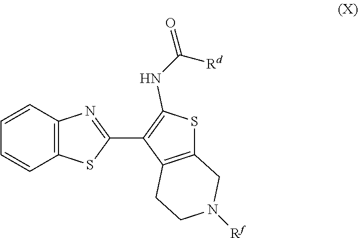

TABLE 3

| R^{d} | R^{f} | R^{d} | R^{f} |
|---|---|---|---|
| cyclohexyl | methyl | 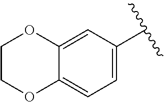 [SMILES: *c1ccc2c(c1)OCCO2] | Acetyl |
| Ethyl | Methyl | 2-furanyl | Methyl |
| cyclopropyl | Methyl | cyclohexyl | Methyl |
| 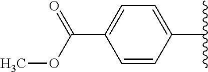 [SMILES: *c1ccc(C(=O)OC)cc1] | Methyl | benzyl | Methyl |
| 4-chlorophenyl | Ethyl | 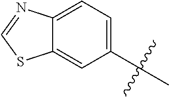 [SMILES: *c1ccc2ncsc2c1] | methyl |
| 2-thienyl | Ethyl | 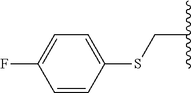 [SMILES: *CSc1ccc(F)cc1] | methyl |
| 4-methoxyphenyl | Ethyl | methyl | 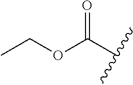 [SMILES: *C(=O)OCC] |
| Methyl | Benzyl | 4-methoxyphenyl | 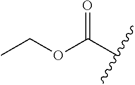 [SMILES: *C(=O)OCC] |
| isopropyl | Benzyl | ethyl | 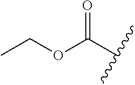 [SMILES: *C(=O)OCC] |
| 2-furanyl | Benzyl | 3-methoxyphenyl | 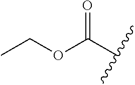 [SMILES: *C(=O)OCC] |
| 4-fluorophenyl | Isopropyl | 2-thienyl | 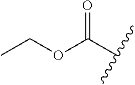 [SMILES: *C(=O)OCC] |
| Methyl | Isopropyl | 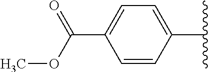 [SMILES: *c1ccc(C(=O)OC)cc1] | Isopropyl |
| 2-thienyl | Isopropyl | 3-(trifluoromethyl)phenyl | methyl |

In certain embodiments, the compounds of the invention do not include the compounds of Formula X where R^{d} is methyl and R^{f} is selected from the group consisting of isopropyl, hydrogen, t-butoxycarbonyl, methyl, benzyl and acetyl.

As discussed above, the invention additionally encompasses pharmaceutical compositions comprising an effective amount of a compound of the invention and a pharmaceutically acceptable carrier. For example, pharmaceutical compositions comprising a pharmaceutically acceptable carrier and an effective amount of a compound of Formulae (I), (IIa-IIh), (IIIa-IIIe), (IVa-IVc), (Va-Vn), (VIa-VIl), (VII), (VIII) or (IX) are encompassed by the invention.

In additional embodiment, the pharmaceutical composition comprises an effective amount of a compound shown above in Tables 1, 1b, 2 or 3 and a pharmaceutically acceptable carrier.

It is to be understood that the specific embodiments described herein can be taken in combination with other specific embodiments delineated herein. For example, for compounds of Formula (IIa), X₁ was defined as sulfur (S) in one embodiment described above and G₁ was defined as optionally substituted heteroaryl in an additional embodiment above. It is to be understood that the invention thus encompasses compounds of Formula (IIa), wherein X₁ is sulfur and G₁ is optionally substituted heteroaryl.

The term “alkyl”, as used herein, unless otherwise indicated, refers to both branched and straight-chain saturated aliphatic hydrocarbon groups having the specified number of carbon atoms; for example, “C₁-C₁₀ alkyl” denotes alkyl having 1 to 10 carbon atoms. Examples of alkyl include, but are not limited to, methyl, ethyl, n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl.

The term, “alkenyl”, as used herein, refers to both straight and branched-chain moieties having the specified number of carbon atoms and having at least one carbon-carbon double bond.

The term, “alkynyl”, as used herein, refers to both straight and branched-chain moieties having the specified number or carbon atoms and having at least one carbon-carbon triple bond.

The term “cycloalkyl,” as used herein, refers to cyclic alkyl moieties having 3 or more carbon atoms. Examples of cycloalkyl include, but are not limited to, cyclopropyl, cyclobutyl, cyclopentyl, cyclohexyl, cycloheptyl and adamantyl.

The term “cycloalkenyl,” as used herein, refers to cyclic alkenyl moieties having 3 or more carbon atoms.

The term “cycloalkynyl,” as used herein, refers to cyclic alkynyl moieties having 5 or more carbon atoms.

The term “heterocyclic” encompasses heterocycloalkyl, heterocycloalkenyl, heterobicycloalkyl, heterobicycloalkenyl, heteropolycycloalkyl, heteropolycycloalkenyl and the like. Heterocycloalkyl refers to cycloalkyl groups containing one or more heteroatoms (O, S, or N) within the ring. Heterocycloalkenyl as used herein refers to cycloalkenyl groups containing one or more heteroatoms (O, S or N) within the ring. Heterobicycloalkyl refers to bicycloalkyl groups containing one or more heteroatoms (O, S or N) within a ring. Heterobicycloalkenyl as used herein refers to bicycloalkenyl groups containing one or more heteroatoms (O, S or N) within a ring.

Cycloalkyl, cycloalkenyl, heterocyclic, groups also include groups similar to those described above for each of these respective categories, but which are substituted with one or more oxo moieties.

The term “aryl”, as used herein, refers to mono- or polycyclic aromatic carbocyclic ring systems. A polycyclic aryl is a polycyclic ring system that comprises at least one aromatic ring. Polycyclic aryls can comprise fused rings, covalently attached rings or a combination thereof. The term “aryl” embraces aromatic radicals, such as, phenyl, naphthyl, indenyl, tetrahydronaphthyl, and indanyl. An aryl group may be substituted or unsubstituted.

The term “heteroaryl”, as used herein, refers to aromatic carbocyclic groups containing one or more heteroatoms (O, S, or N) within a ring. A heteroaryl group can be monocyclic or polycyclic. A heteroaryl group may additionally be substituted or unsubstituted. The heteroaryl groups of this invention can also include ring systems substituted with one or more oxo moieties. A polycyclic heteroaryl can comprise fused rings, covalently attached rings or a combination thereof. Examples of heteroaryl groups include, but are not limited to, pyridinyl, pyridazinyl, imidazolyl, pyrimidinyl, pyrazolyl, triazolyl, pyrazinyl, quinolyl, isoquinolyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, thiazolopyridinyl, oxazolopyridinyl and azaindolyl. The foregoing heteroaryl groups may be C-attached or heteroatom-attached (where such is possible). For instance, a group derived from pyrrole may be pyrrol-1-yl (N-attached) or pyrrol-3-yl (C-attached).

The term “substituted” refers to substitution by independent replacement of one, two, or three or more of the hydrogen atoms with substituents including, but not limited to, —C₁-C₁₂ alkyl, —C₂-C₁₂ alkenyl, —C₂-C₁₂ alkynyl, —C₃-C₁₂ cycloalkyl, —C₃-C₁₂ cycloalkenyl, C₃-C₁₂ cycloalkynyl, -heterocyclic, —F, —Cl, —Br, —I, —OH, —NO₂, —N₃, —CN, —NH₂, oxo, thioxo, —NHRₓ, —NRₓRₓ, dialkylamino, -diarylamino, -diheteroarylamino, —ORₓ, —C(O)R_{y}, —C(O)C(O)R_{y}, —OCO₂R_{y}, —OC(O)R_{y}, OC(O)C(O)R_{y}, —NHC(O)R_{y}, —NHCO₂R_{y}, —NHC(O)C(O)R_{y}, NHC(S)NH₂, —NHC(S)NHRₓ, —NHC(NH)NH₂, —NHC(NH)NHR_{X}, —NHC(NH)R_{X}, —C(NH)NHRₓ, and (C═NRₓ)Rₓ; —NRxC(O)Rₓ, —NRxCO₂R_{y}, —NRxC(O)C(O)R_{y}, —NRxC(S)NH₂, —NRₓC(S)NHRₓ, —NRₓC(NH)NH₂, —NRₓC(NH)NHRₓ, —NRxC(NH)Rₓ, —C(NRx)NHRₓ—S(O)R_{y}, —NHSO₂Rₓ, —CH₂NH₂, —CH₂SO₂CH₃, -aryl, -arylalkyl, -heteroaryl, -heteroarylalkyl, —heterocycloalkyl, —C₃-C₁₂-cycloalkyl, -polyalkoxyalkyl, -polyalkoxy, -methoxymethoxy, -methoxyethoxy, —SH, —S—Rₓ, or -methylthiomethyl, wherein Rₓ is selected from the group consisting of —C₁-C₁₂ alkyl, —C₂-C₁₂ alkenyl, —C₂-C₁₂ alkynyl, —C₃-C₁₂ cycloalkyl, -aryl, -heteroaryl and -heterocyclic and -R_{y} is selected from the group consisting of —C₁-C₁₂ alkyl, —C₂-C₁₂ alkenyl, —C₂-C₁₂ alkynyl, —C₃-C₁₂ cycloalkyl, -aryl, -heteroaryl, -heterocyclic, —NH₂, —NH—C₁-C₁₂ alkyl, —NH—C₂-C₁₂ alkenyl, —NH—C₂-C₁₂-alkynyl, —NH—C₃-C₁₂ cycloalkyl, —NH-aryl, —NH-heteroaryl and —NH-heterocyclic. It is understood that the aryls, heteroaryls, alkyls, and the like can be further substituted.

The term “haloalkyl” as used herein refers to an alkyl group having 1 to (2n+1) subsistent(s) independently selected from F, Cl, Br or I, where n is the maximum number of carbon atoms in the alkyl group.

As will be understood by the skilled artisan, “H” is the symbol for hydrogen, “N” is the symbol for nitrogen, “S” is the symbol for sulfur, “O” is the symbol for oxygen.

“Me” is an abbreviation for methyl.

Non-limiting examples of optionally substituted aryl are phenyl, substituted phenyl, napthyl and substituted naphthyl.

In some embodiments, two R_{b} groups when attached to the same atom, can be taken together with the atom to which they are attached to form an optionally substituted C₃-C₁₂ cycloalkyl, an optionally substituted C₃-C₁₂ cycloalkenyl, an optionally substituted heterocyclic, an optionally substituted aryl, and an optionally substituted heteroaryl.

Certain of the compounds described herein contain one or more asymmetric centers and may thus give rise to enantiomers, diastereomers, and other stereoisomeric forms that may be defined, in terms of absolute stereochemistry, as (R)— or (S)—. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. “Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. When the compounds described herein contain olefinic double bonds or other centers of geometric asymmetry, and unless specified otherwise, it is intended that the compounds include both E and Z geometric isomers. Likewise, all tautomeric forms are also intended to be included.

Where a particular stereochemistry is described or depicted it is intended to mean that a particular enantiomer is present in excess relative to the other enantiomer. A compound has an R-configuration at a specific position when it is present in excess compared to the compound having an S-configuration at that position. A compound has an S-configuration at a specific position when it is present in excess compared to the compound having an R-configuration at that position.

It is to be understood that atoms making up the compounds of the present invention are intended to include isotopic forms of such atoms. Isotopes, as used herein, include those atoms having the same atomic number but different mass numbers. Isotopes of hydrogen include, for example, tritium and deuterium, and isotopes of carbon include, for example, ¹³C and ¹⁴C. The invention therefore encompasses embodiments in which one or more of the hydrogen atoms in Formulae (I), (IIa-IIi), (IIIa-IIIf), (IVa-IVc), (Va-Vn), (VIa-VIl), (VII), (VIII), or (IX) are replaced with deuterium. The invention also encompasses embodiments wherein one or more of the carbon atoms in Formulae (I), (IIa-IIi), (IIIa-IIIf), (IVa-IVc), (Va-VI), (VIa-VII), (VII), (VIII) or (IX) is replaced with silicon atoms.

The invention additionally encompasses embodiments wherein one or more of the nitrogen atoms in Formulae (I), (IIa-IIi), (IIIa-IIIf), (IVa-IVc), (Va-Vn), (VIa-VIl ), (VII), (VIII) or (IX) is oxidized to N-oxide.

Exemplary synthetic routes for the preparation of compounds of the invention are shown below as Schemes 1-4 below. Methods that can be used to synthesize compounds of the invention have also been discussed in the literature, for example, in some of the references listed below. As will be understood by the skilled artisan, diastereomers can be separated from the reaction mixture using column chromatography.

The synthesis of 2-aminothiophenes such as examples 1 can be synthesized via the chemistry depicted in Scheme 1.⁸ The resulting amine can be reacted with appropriate acetylating⁹ or sulfonylatine _{g}roups to form amides and sulfonylamides such as examples 2 and 37, respectively.

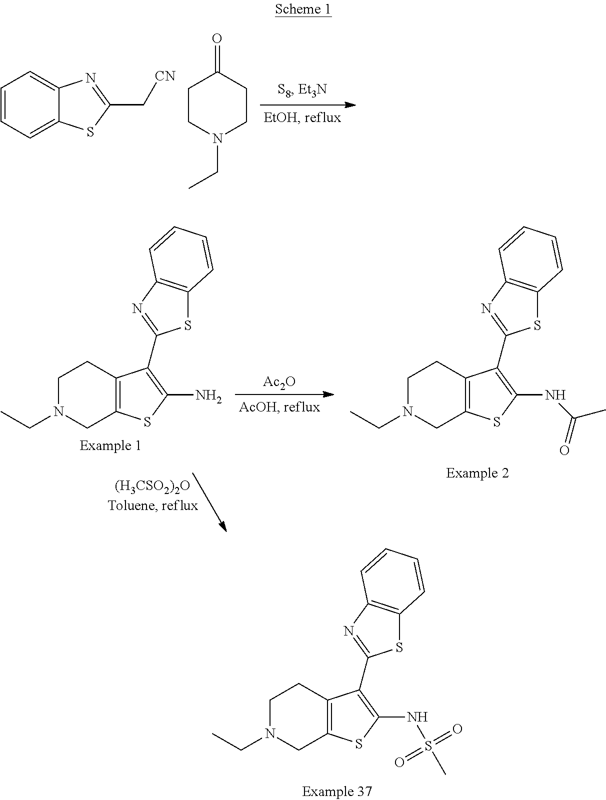

The synthesis of 2-aminothiophenes such as compound B can be synthesized as shown in Scheme 2. The appropriate ketone is reacted with sulfur using the Gewald¹¹ conditions affording compound A. The resulting ester can be reacted with appropriate aniline to afford benzthiazoles, benzoxazoles, and benzimidazoles¹². The resulting amine can be acetylated as above to compound C. There are a number of reports available for the oxidation of the tetrahydropiperidine embodied by C to pyridines with either manganese dioxide¹³ or DDQ.¹⁴

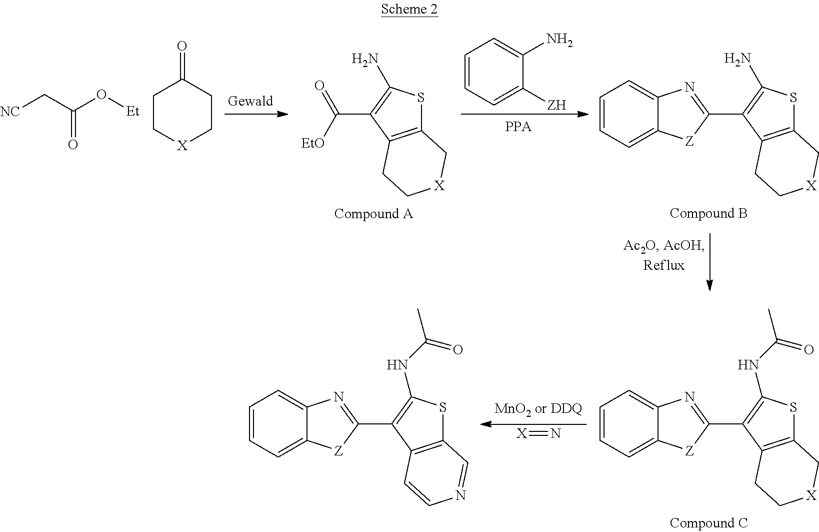

The synthesis of 2-aminothiophenes such as compound E can be synthesized by reductive amination of the 2-aminothiophenes.

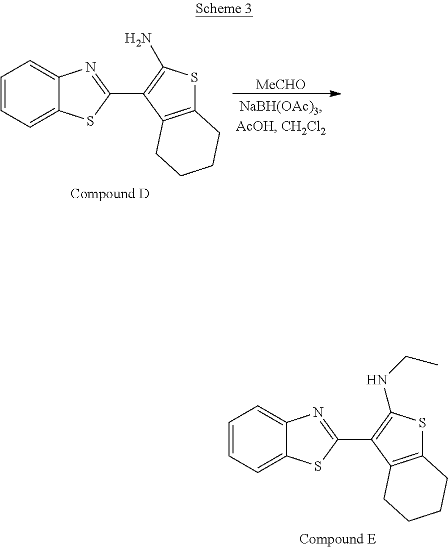 [SMILES: "MeCHO" ; Nc1sc2c(c1-c1nc3ccccc3s1)CCCC2 ; CCNc1sc2c(c1-c1nc3ccccc3s1)CCCC2]

Compound I can be prepared using the chemistry outlined below. The preparation of intermediate G is described with a carbamoyl group on the piperidine nitrogen instead of an ethyl group.¹⁵ Condensation of compound G with the benzothiazole acetonitrile should afford compound H, which when acetylated will give compound I.

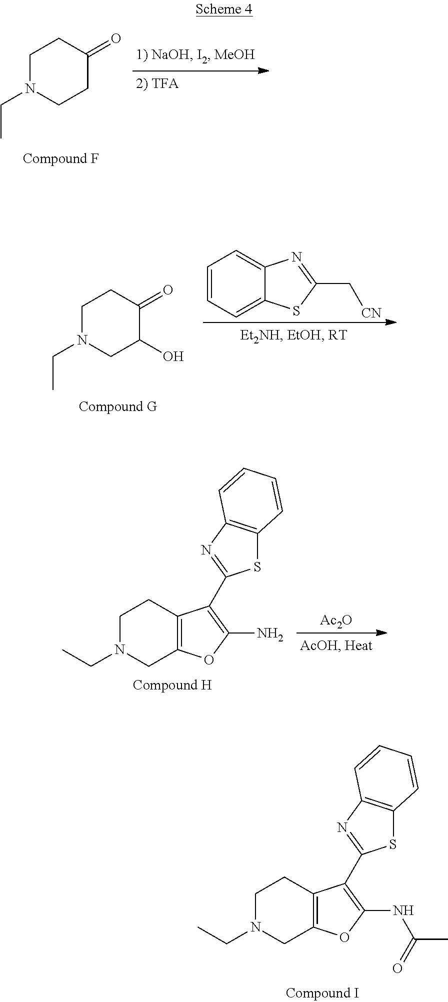

The invention encompasses pharmaceutically acceptable salts of the compounds described herein. Thus, in certain aspects, the invention is directed to pharmaceutically salts of compounds of Formulae (I), (IIa-IIh), (IIIa-IIIe), (IVa-IVc), (Va-Vl), (VIa-VIl), (VII), (VIII) or (IX) and the compounds disclosed herein. As used herein, a “pharmaceutically acceptable salt” includes an ionic bond-containing product of the reaction between the disclosed compound with either an acid or a base, suitable for administering to a subject. Pharmaceutically acceptable salts are well known in the art and are described, for example, in Berge et al (1977), Pharmaceutical Salts. Journal of Pharmaceutical Sciences, 69(1): 1-19, the contents of which are herein incorporated by reference. A non-limiting example of a pharmaceutically acceptable salt is an acid salt of a compound containing an amine or other basic group which can be obtained by reacting the compound with a suitable organic or inorganic acid. Examples of pharmaceutically acceptable salts also can be metallic salts including, but not limited to, sodium, magnesium, calcium, lithium and aluminum salts. Further examples of pharmaceutically acceptable salts include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures), succinates, benzoates and salts with amino acids such as glutamic acid. Salts can also be formed with suitable organic bases when the compound comprises an acid functional group such as —COOH or —SO₃H. Such bases suitable for the formation of a pharmaceutically acceptable base addition salts with compounds of the present invention include organic bases that are nontoxic and strong enough to react with the acid functional group. Such organic bases are well known in the art and include amino acids such as arginine and lysine, mono-, di-, and triethanolamine, choline, mono-, di-, and trialkylamine, such as methylamine, dimethylamine, and trimethylamine, guanidine, N-benzylphenethylamine, N-methylglucosamine, N-methylpiperazine, morpholine, ethylendiamine, tris(hydroxymethyl)aminomethane and the like.

The invention also includes hydrates of the compounds described herein, including for example solvates of the compounds described herein. In some embodiments, the invention encompasses solvates of a compound of Formulae (I), (IIa-IIh), (IIIa-IIIe), (IVa-IVc), (Va-VI), (VIa-VIl), (VII), (VIII) or (IX) or a compound specifically disclosed herein.

Also included in the present invention are prodrugs of the compounds described herein, for example, prodrugs of a compound of Formulae (I), (IIa-IIh), (IIIa-IIIe), (IVa-IVc), (Va-Vl), (VIa-VIl), (VII), (VIII), or (IX) and of a compound described herein.

The invention additionally includes clathrates of the compounds described herein. In some embodiments, the invention is directed to clathrates of a compound of Formulae (I), (IIa-IIh), (IIIa-IIIe), (IVa-IVc), (Va-Vl), (VIa-VIl), (VII), (VIII) and (IX).

As discussed above, the invention includes pharmaceutical compositions comprising a pharmaceutically acceptable carrier or excipient and a compound described herein. The inventive compound or a compound of one of Formulae (I), (IIa-IIh), (IIIa-IIIe), (IVa-IVc), (Va-Vl), (VIa-VIl), (VII), (VIII), or (IX) or a pharmaceutically acceptable salt, solvate, clathrate or prodrug of any of thereof, can be administered in pharmaceutical compositions comprising a pharmaceutically acceptable carrier or excipient. The excipient can be chosen based on the expected route of administration of the composition in therapeutic applications. The route of administration of the composition depends on the condition to be treated. For example, intravenous injection may be preferred for treatment of a systemic disorder and oral administration may be preferred to treat a gastrointestinal disorder. The route of administration and the dosage of the composition to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms.

Pharmaceutical compositions comprising compounds described herein, or a pharmaceutically acceptable salt, solvate, clathrate or prodrug of any of thereof, can be administered by a variety of routes including, but not limited to, parenteral, oral, pulmonary, ophthalmic, nasal, rectal, vaginal, aural, topical, buccal, transdermal, intravenous, intramuscular, subcutaneous, intradermal, intraocular, intracerebral, intralymphatic, intraarticular, intrathecal and intraperitoneal.

The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the pharmacologic agent or composition. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Pharmaceutical compositions can also include large, slowly metabolized macromolecules such as proteins, polysaccharides such as chitosan, polylactic acids, polyglycolic acids and copolymers (such as latex functionalized SEPHAROSETM, agarose, cellulose, and the like), polymeric amino acids, amino acid copolymers, and lipid aggregates (such as oil droplets or liposomes).

The compositions can be administered parenterally such as, for example, by intravenous, intramuscular, intrathecal or subcutaneous injection. Parenteral administration can be accomplished by incorporating a composition into a solution or suspension. Such solutions or suspensions may also include sterile diluents such as water for injection, saline solution, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

Injectable formulations can be prepared either as liquid solutions or suspensions; solid forms suitable for solution in, or suspension in, liquid vehicles prior to injection can also be prepared. The preparation also can also be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The compositions and pharmacologic agents described herein can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications. For suppositories, binders and carriers include, for example, polyalkylene glycols or triglycerides; such suppositories can be formed from mixtures containing the active ingredient in the range of about 0.5% to about 10%, preferably about 1%- to about 2%. Oral formulations include excipients, such as pharmaceutical grades of mannitol, lactose, starch, magnesium stearate, sodium saccharine, cellulose, and magnesium carbonate. Topical application can result in transdermal or intradermal delivery. Transdermal delivery can be achieved using a skin patch or using transferosomes. [Paul et al., Eur. J. Immunol. 25: 3521-24, 1995; Cevc et al., Biochem. Biophys. Acta 1368: 201-15, 1998].

For the purpose of oral therapeutic administration, the pharmaceutical compositions can be incorporated with excipients and used in the form of tablets, troches, capsules, elixirs, suspensions, syrups, wafers, chewing gums and the like. Tablets, pills, capsules, troches and the like may also contain binders, excipients, disintegrating agent, lubricants, glidants, sweetening agents, and flavoring agents. Some examples of binders include microcrystalline cellulose, gum tragacanth or gelatin. Examples of excipients include starch or lactose. Some examples of disintegrating agents include alginic acid, corn starch and the like. Examples of lubricants include magnesium stearate or potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and non-toxic in the amounts used. In another embodiment, the composition is administered as a tablet or a capsule.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like. For vaginal administration, a pharmaceutical composition may be presented as pessaries, tampons, creams, gels, pastes, foams or spray.

The pharmaceutical composition can also be administered by nasal administration. As used herein, nasally administering or nasal administration includes administering the composition to the mucus membranes of the nasal passage or nasal cavity of the patient. As used herein, pharmaceutical compositions for nasal administration of a composition include therapeutically effective amounts of the compounds prepared by well-known methods to be administered, for example, as a nasal spray, nasal drop, suspension, gel, ointment, cream or powder. Administration of the composition may also take place using a nasal tampon or nasal sponge.

For topical administration, suitable formulations may include biocompatible oil, wax, gel, powder, polymer, or other liquid or solid carriers. Such formulations may be administered by applying directly to affected tissues, for example, a liquid formulation to treat infection of conjunctival tissue can be administered dropwise to the subject's eye, or a cream formulation can be administered to the skin.

Rectal administration includes administering the pharmaceutical compositions into the rectum or large intestine. This can be accomplished using suppositories or enemas. Suppository formulations can easily be made by methods known in the art. For example, suppository formulations can be prepared by heating glycerin to about 120° C., dissolving the pharmaceutical composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

In addition to the usual meaning of administering the formulations described herein to any part, tissue or organ whose primary function is gas exchange with the external environment, for purposes of the present invention, “pulmonary” will also mean to include a tissue or cavity that is contingent to the respiratory tract, in particular, the sinuses. For pulmonary administration, an aerosol formulation containing the active agent, a manual pump spray, nebulizer or pressurized metered-dose inhaler as well as dry powder formulations are contemplated. Suitable formulations of this type can also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a head space representing greater than about 15% of the total volume of the canister. Often, the compound intended for pulmonary administration is dissolved, suspended or emulsified in a mixture of a solvent, surfactant and propellant. The mixture is maintained under pressure in a canister that has been sealed with a metering valve.

The invention encompasses a method of treating a patient suffering from a condition associated with a dysfunction in protein homeostasis comprising administering to said patient a therapeutically effective amount of a compound described herein. In certain aspects, the condition is cystic fibrosis.

“Treating” or “treatment” includes preventing or delaying the onset of the symptoms, complications, or biochemical indicia of a disease, alleviating or ameliorating the symptoms or arresting or inhibiting further development of the disease, condition, or disorder. A “patient” is a human subject in need of treatment.

An “effective amount” refers to that amount of the therapeutic agent that is sufficient to ameliorate of one or more symptoms of a disorder and/or prevent advancement of a disorder, cause regression of the disorder and/or to achieve a desired effect.

As used herein, the term “inhibiting” or “decreasing” encompasses causing a net decrease by either direct or indirect means. The term “increasing” means to cause a net gain by either direct or indirect means.

The invention encompasses the treatment of a condition associated with a dysfunction in proteostasis. Proteostasis refers to protein homeostasis. Dysfunction in protein homeostasis is a result of protein misfolding, protein aggregation, defective protein trafficking or protein degradation. Exemplary proteins of which there can be a dysfunction in proteostasis, for example that can exist in a misfolded state, include, but are not limited to, glucocerebrosidase, hexosamine A, cystic fibrosis transmembrane conductance regulator, aspartylglucsaminidase, α-galactosidase A, cysteine transporter, acid ceremidase, acid α-L-fucosidase, protective protein, cathepsin A, acid β-glucosidase, acid β-galactosidase, iduronate 2-sulfatase, α-L-iduronidase, galactocerebrosidase, acid α-mannosidase, acid β-mannosidase, arylsulfatase B, arylsulfatase A, N-acetylgalactosamine-6-sulfate sulfatase, acid β-galactosidase, N-acetylglucosamine-1-phosphotransferase, acid sphingmyelinase, NPC-1, acid α-glucosidase, β-hexosamine B, heparin N-sulfatase, α-N-acetylglucosaminidase, α-glucosaminide N-acetyltransferase, N-acetylglucosamine-6-sulfate sulfatase, α-N-acetylgalactosaminidase, α-neuramidase, β-glucuronidase, β-hexosamine A and acid lipase, polyglutamine, α-synuclein, Ab peptide, tau protein transthyretin and insulin.

Protein conformational diseases encompass gain of function disorders and loss of function disorders. In one embodiment, the protein conformational disease is a gain of function disorder. The terms “gain of function disorder,” “gain of function disease,” “gain of toxic function disorder” and “gain of toxic function disease” are used interchangeably herein. A gain of function disorder is a disease characterized by increased aggregation-associated proteotoxicity. In these diseases, aggregation exceeds clearance inside and/or outside of the cell. Gain of function diseases include, but are not limited to neurodegenerative diseases associated with aggregation of polyglutamine, Lewy body diseases, amyotrophic lateral sclerosis, transthyretin-associated aggregation diseases, Alzheimer's disease and prion diseases. Neurodegenerative diseases associated with aggregation of polyglutamine include, but are not limited to, Huntington's disease, dentatorubral and pallidoluysian atrophy, several forms of spino-cerebellar ataxia, and spinal and bulbar muscular atrophy. Alzheimer's disease is characterized by the formation of two types of aggregates: extracellular aggregates of Aβ peptide and intracellular aggregates of the microtubule associated protein tau. Transthyretin-associated aggregation diseases include, for example, senile systemic amyloidoses and familial amyloidotic neuropathy. Lewy body diseases are characterized by an aggregation of α-synuclein protein and include, for example, Parkinson's disease. Prion diseases (also known as transmissible spongiform encephalopathies or TSEs) are characterized by aggregation of prion proteins. Exemplary human prion diseases are Creutzfeldt-Jakob Disease (CJD), Variant Creutzfeldt-Jakob Disease, Gerstmann-Straussler-Scheinker Syndrome, Fatal Familial Insomnia and Kuru.

In a further embodiment, the protein conformation disease is a loss of function disorder. The terms “loss of function disease” and “loss of function disorder” are used interchangeably herein. Loss of function diseases are a group of diseases characterized by inefficient folding of a protein resulting in excessive degradation of the protein. Loss of function diseases include, for example, cystic fibrosis and lysosomal storage diseases. In cystic fibrosis, the mutated or defective enzyme is the cystic fibrosis transmembrane conductance regulator (CFTR). One of the most common mutations of this protein is ΔF508 which is a deletion (A) of three nucleotides resulting in a loss of the amino acid phenylalanine (F) at the 508th (508) position on the protein. Lysosomal storage diseases are a group of diseases characterized by a specific lysosomal enzyme deficiency which may occur in a variety of tissues, resulting in the build-up of molecules normally degraded by the deficient enzyme. The lysosomal enzyme deficiency can be in a lysosomal hydrolase or a protein involved in the lysosomal trafficking Lysosomal storage diseases include, but are not limited to, aspartylglucosaminuria, Fabry's disease, Batten disease, Cystinosis, Farber, Fucosidosis, Galactasidosialidosis, Gaucher's disease (including Types 1, 2 and 3), Gml gangliosidosis, Hunter's disease, Hurler-Scheie's disease, Krabbe's disease, a-Mannosidosis, B-Mannosidosis, Maroteaux-Lamy's disease, Metachromatic Leukodystrophy, Morquio A syndrome, Morquio B syndrome, Mucolipidosis II, Mucolipidosis III, Neimann-Pick Disease (including Types A, B and C), Pompe's disease, Sandhoff disease, Sanfilippo syndrome (including Types A, B, C and D), Schindler disease, Schindler-Kanzaki disease, Sialidosis, Sly syndrome, Tay-Sach's disease and Wolman disease.

In another embodiment, the disease associated with a dysfunction in proteostasis is a cardiovascular disease. Cardiovascular diseases include, but are not limited to coronary artery disease, myocardial infarction, stroke, restenosis and arteriosclerosis. Conditions associated with a dysfunction of proteostasis also include ischemic conditions, such as, ischemia/reperfusion injury, myocardial ischemia, stable angina, unstable angina, stroke, ischemic heart disease and cerebral ischemia.

In yet another embodiment, the disease associated with a dysfunction in proteostasis is diabetes or diabetic retinopathy.

In some embodiments, the condition is selected from the group consisting of Huntington's disease, Alzheimer's disease, Parkinson's disease, amyotrophic lateral sclerosis, diabetic retinopathy, diabetes, and cystic fibrosis.

The present invention also encompasses methods of treating a patient suffering from a condition associated with a dysfunction in proteostasis comprising administering an effective amount of a compound having one of the Formula (I), (IIa-IIh), (IIIa-IIIe), (IVa-IVc), (Va-Vl), (VIa-VIl), (VII), (VIII) or (IX) or a pharmaceutically acceptable salt, prodrug, clathrate or solvate of any of thereof

In an additional embodiment, the invention is directed to a pharmaceutical composition comprising a compound described herein and a second agent, wherein the second agent is selected from the group consisting of a pharmacologic chaperone and a proteostasis regulator. In certain embodiments, the invention is directed to a pharmaceutical composition comprising a compound having one of the Formula (I), (IIa-IIh), (IIIa-IIIe), (IVa-IVc), (Va-Vh), (VIa-VIl), (VII), (VIII) or (IX) and a second agent, wherein the second agent is selected from the group consisting of a pharmacologic chaperone and a proteostasis regulator. The invention also encompasses a method of treating a patient suffering from a condition associated with a dysfunction in proteostasis comprising administering a therapeutically effective amount of a compound of the invention and a second agent, wherein the second agent is a pharmacologic chaperone. Pharmacologic chaperones or kinetic stabilizers refer to compounds that bind an existing steady state level of the folded mutant protein and chemically enhance the folding equilibrium by stabilizing the fold [Bouvier, Chem Biol14: 241-242, 2007; Fan et al., Nat Med 5: 112-115, 1999; Sawkar et al., Proc Natl Acad Sci USA 99:15428-15433, 2002; Johnson and Kelly, Accounts of Chemical Research 38: 911-921, 2005]. The pharmacologic chaperone is administered in amount that in combination with a compound described herein in an amount that is sufficient to treat a patient suffering from a condition associated with a dysfunction in proteostasis. Exemplary pharmacologic chaperones are described in U.S. Patent Publication No's. 20080056994, 20080009516, 20070281975, 20050130972, 20050137223, 20050203019, 20060264467 and 20060287358, the contents of which are incorporated by reference herein.

In another embodiment, the invention is a method of treating a patient suffering from a condition associated with a dysfunction in proteostasis comprising administering therapeutically effective amount of a compound of the invention and a second agent, wherein the second agent is a proteostasis regulator. The term “proteostasis regulator” refers to small molecules, siRNA and biologicals (including, for example, proteins) that enhance cellular protein homeostasis. For example, proteostasis regulators can be agents that influence protein synthesis, folding, trafficking and degradation pathways. Proteostasis regulators encompass pharmacologic agents that stimulate the HSR signaling activity. Proteostasis regulators function by manipulating signaling pathways, including, but not limited to, the heat shock response or the unfolded protein response, or both, resulting in transcription and translation of proteostasis network components. Proteostasis regulators can enhance the folding, trafficking and function of proteins (for example, mutated proteins). Proteostasis regulators can also regulate protein chaperones by upregulating transcription or translation of the protein chaperone, or inhibiting degradation of the protein chaperone. Proteostasis regulators can influence the biology of folding, often by the coordinated increase in chaperone and folding enzyme levels and macromolecules that bind to partially folded conformational ensembles, thus enabling their progression to intermediates with more native structure and ultimately increasing the concentration of folded mutant protein for export. In one aspect, the proteostasis regulator is distinct from a chaperone in that the proteostasis regulator can enhance the homeostasis of a mutated protein but does not bind the mutated protein. In addition, proteostasis regulators can upregulate an aggregation pathway or a disaggregase activity. Exemplary proteostasis regulators are the celastrols, MG-132 and L-type Ca²⁺ channel blockers (e.g., dilitiazem and verapamil). The term “celastrols” refers to celastrol and derivatives or analogs thereof, including, but not limited to, those celastrol derivatives described in Westerheide et al., J Biol Chem, 2004. 279(53): p. 56053-60, the contents of which are expressly incorporated by reference herein. Celastrol derivatives include, for example, celastrol methyl ester, dihydrocelastrol diacetate, celastrol butyl ether, dihydrocelastrol, celastrol benzyl ester, primesterol, primesterol diacetate and triacetate of celastrol. In certain aspects, the proteostasis regulator is a heat shock response activator. A heat shock response activator is an agent that indirectly or directly activates the heat shock response, for example, by directly or indirectly activating heat shock transcription factor 1 (HSF1), inhibiting Hsp90, and/or activating chaperone expression (Westerheide et al., J Biol Chem, 2004. 279(53): p. 56053-60, the contents of which are expressly incorporated by reference herein). The terms “heat shock response activator,” “heat shock activator,” “heat shock response inducer,” and “heat shock inducer” are used interchangeably herein. Non-limiting examples of heat shock response activators are celastrols, non-steroidal anti-inflammatory drugs, ansamycin, geldenamycin, radiciol, glucuronic acid, and tributylin. Heat shock response activators have also been described, for example, in U.S. Patent Application Publication No's. 20070259820, 20070207992, 20070179087, 20060148767, the contents of each of which are expressly incorporated by reference herein. In some embodiments, the heat shock response activator is a small molecule heat shock response activator.

The invention also encompasses a method of treating cancer or a tumor in a patient in need thereof comprising administering to said patient an effective amount of a compound described herein. Cancers that can be treated according to methods of the present invention include, but are not limited to, breast cancer, colon cancer, pancreatic cancer, prostate cancer, lung cancer, ovarian cancer, cervical cancer, multiple myeloma, basal cell carcinoma, neuroblastoma, hematologic cancer, rhabdomyosarcoma, liver cancer, skin cancer, leukemia, basal cell carcinoma, bladder cancer, endometrial cancer, glioma, lymphoma, and gastrointestinal cancer. In another embodiment, the invention is a method of treating cancer or a tumor comprising administering an effective amount of a compound described herein in combination with the administration of a chemotherapeutic agent. Chemotherapeutic agents that can be utilized include, but are not limited to, alkylating agents such as cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®; Aventis Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; and pharmaceutically acceptable salts, acids or derivatives of any of the above. In a further embodiment, the invention is a method of treating cancer or a tumor comprising administering to a patient in need thereof an effective amount of a compound described herein in combination with radiation therapy.

The invention is illustrated by the following examples which are not meant to be limiting in any way.

### EXAMPLES

### Examples 1-3

### 3-(Benzo[d]thiazol-2-yl)-6-ethyl-4,5,6,7-tetrahydrothieno[2,3-c]pyridin-2-amine, N-(3-(Benzo[d]thiazol-2-yl)-6-ethyl-4,5,6,7-tetrahydrothieno[2,3-c]pyridin-2-yl)acetamide, and 2-Acetamido-3-(benzo[d]thiazol-2-yl)-6-ethyl-4,5,6,7-tetrahydrothieno[2,3-c]pyridin-6-ium chloride

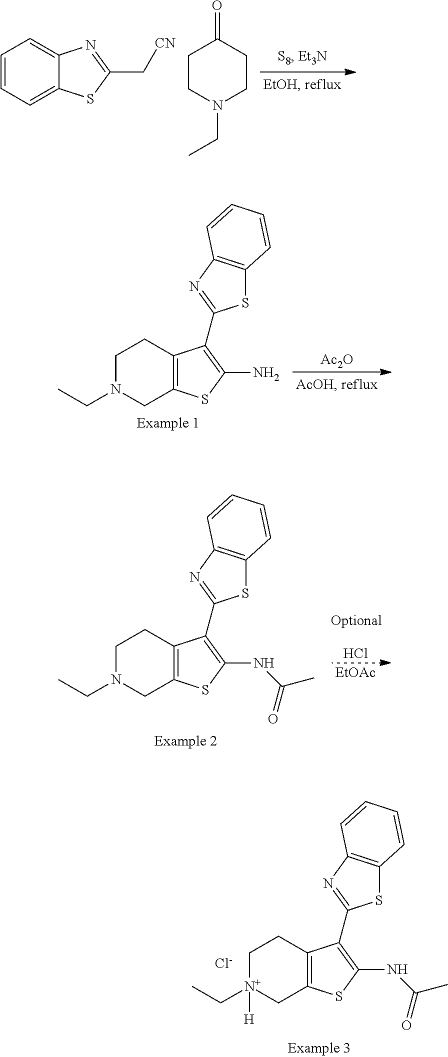

A mixture of (1,3-benzothiazole-2-yl)acetonitrile (24.1 g, 0.14 mol), 1-ethylpiperidine-4 one (17.8 g, 0.14 mol), sulfur (4.5 g, 0.14 mol), triethylamine (29.4 mL, 0.21 mol) and absolute ethanol (400 mL) was heated at reflux for 3 h. After cooling to room temperature the reaction mixture was filtered and the filtrate was concentrated under reduced pressure. The remaining dark oily residue was dissolved in methylene chloride (250 mL) and washed with water (3×30 mL), dried over magnesium sulfate and concentrated under vacuum. The remaining residue was next treated with 6M hydrochloric acid solution (50 mL). The precipitated brown solid was filtered off and washed with diethyl ether (1×50 mL). The solid was then treated with 4M sodium hydroxide (60 mL) and the solution was washed with dichloromethane (4×50 mL). The combined organic layer was washed with water (1×30 mL) and brine (1×50 mL), followed by drying over magnesium sulfate. The solvent was evaporated under reduced pressure to give 17.6 g of crude 3-(benzo[d]thiazol-2-yl)-6-ethyl-4,5,6,7-tetrahydrothieno[2,3-c]pyridin-2-amine as a brown solid. A sample suitable for biological testing and analytical analysis was obtained via column chromatography on silica using an ethyl acetate/hexanes gradient (9-50%). Yield: 40%; m/z (ESI⁺) 316 (MH⁺); ¹H NMR (500 MHz, CDCl₃) δ 7.88 (d, J=8.5 Hz, 1H), 7.82 (d, J=8.5 Hz, 1H), 7.42 (t, J=7 Hz, 1H), 7.26 (t, J=7 Hz, 1H), 6.77 (bs, 2H), 3.53 (s, 2H), 3.03-3.01 (m, 2H), 2.86 (t, J=6 Hz, 2H), 2.64 (q, J=7 Hz, 2H), 1.20 (t, J=7 Hz, 3H).

A mixture of 3-(benzo[c]thiazol-2-yl)-6-ethyl-4,5,6,7-tetrahydrothieno[2,3-c]pyridin-2-amine (10 g, 0.032 mol), acetic anhydride (4.5 mL, 0.048 mol) and glacial acetic acid (400 mL) was heated at reflux for 2 h. The mixture was allowed to cool to room temperature and then 4M sodium hydroxide was added to adjust the pH to approximately 9. The mixture was filtered and the aqueous filtrate was washed with dichloromethane (3×50 mL). The organic layer was combined with the filtered solid material and the resulting mixture was then washed with water (1×30 mL) and brine (1×30 mL). The organic phase was dried over magnesium sulfate and concentrated to dryness under vacuum to give crude product as a brown oil. The crude product was purified by column chromatography on silica using an ethyl acetate/hexanes gradient (9-50%). Fractions containing the product were concentrated in vacuo to afford 5.6 g of N-(3-(benzo[c]thiazol-2-yl)-6-ethyl-4,5,6,7-tetrahydrothieno[2,3-c]pyridin-2-yl)acetamide as a dark yellow powder. Yield: 49%; m/z (ESI⁻) 356 (M⁻−H); ¹H NMR (500 MHz, CDCl₃) δ 12.99 (s, 1H) 7.94 (d, J=8 Hz, 1H), 7.88 (d, J=8 Hz, 1H), 7.50 (t, J=7 Hz, 1H), 7.37 (t, J=7.5 Hz, 1H), 3.65 (s, 2H), 3.07-3.04 (m, 2H), 2.89 (t, J=5.5 Hz, 2H), 2.67 (q, J=7 Hz, 2H), 2.35 (s, 3H), 1.22 (t, J=7 Hz, 3H).

To solid N-(3-(benzo[c]thiazol-2-yl)-6-ethyl-4,5,6,7-tetrahydrothieno[2,3-c]pyridin-2-yl)acetamide (2.2 g, 0.0062 mol) was added a solution of hydrochloric acid in ethyl acetate (4M, 60 mL). After stirring, a precipitate formed and the precipitate was filtered from the mixture. The filtered solid was washed with diethyl ether (2×50 mL) and allowed to dry in a stream of air to afford an orange solid. The solid was recrystallized from methanol (70 mL) to afford 1.2 g of 2-acetamido-3-(benzo[c]thiazol-2-yl)-6-ethyl-4,5,6,7-tetrahydrothieno[2,3-c]pyridin-6-ium chloride as a pale yellow solid. Yield: 48% m/z (ESI⁻) 358 (MH⁺); ¹H NMR (200 MHz, D₂O) δ 7.75 (bs, 1H), 7.57 (bs, 1H), 7.34 (bs, 1H), 7.27 (bs,1H), 4.32 (bs, 1H), 3.82 (bs, 1H), 3.53 (bs, 1H), 3.21 (bs, 2H), 2.76 (bs, 3H), 2.02 (bs, 3H), 1.32 (bs, 3H).

### Example 4

### N-(3-(Benzo[d]thiazol-2-yl)-6-methyl-4,5,6,7-tetrahydrothieno[2,3-c]pyridin-2-yl)acetamide

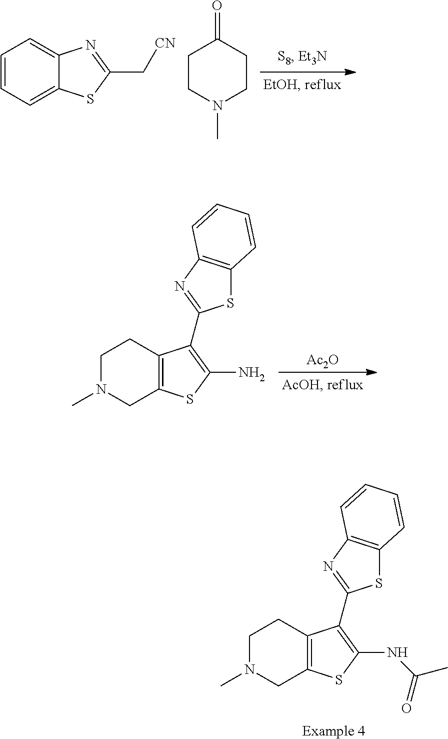

A mixture of (1,3-benzothiazole-2-yl)acetonitrile (1 g, 0.0057 mol), 1-methylpiperdine-4-one (0.66 mL, 0.0057 mol), sulfur (0.18 g, 0.0057 mol), and triethylamine (1.14 mL, 0.0037 mol) in anhydrous ethanol (30 mL) was heated at reflux for 3 h. The reaction mixture was allowed to cool to room temperature and was then condensed in vacuo. The remaining residue was triturated with water and neutralized with concentrated hydrochloric acid. The heterogeneous mixture was filtered and the filtered solids were purified by column chromatography on silica to afford 320 mg of 3-(benzo[d]thiazol-2-yl)-6-methyl-4,5,6,7-tetrahydrothieno[2,3-c]pyridin-2-amine as a red solid. Yield: 19%; m/z (ESI⁺) 302 (MH⁺)

A mixture of 3-(benzo[d]thiazol-2-yl)-6-methyl-4,5,6,7-tetrahydrothieno[2,3-c]pyridin-2-amine (0.25 g, 0.00083 mol), acetic anhydride (0.117 mL, 0.00124 mol), and glacial acetic acid (30 mL) was heated at reflux for 5 h and then allowed to cool to room temperature. The reaction mixture was diluted with water (100 mL) and then neutralized with 5% sodium bicarbonate solution. The precipitated solids were collected via filtration and then dissolved in dichloromethane. The dichloromethane solution was washed with water, dried over magnesium sulfate, and filtered. The filtrate was condensed in vacuo and the remaining residue was triturated with diethyl ether. The mixture was filtered and allowed to dry in a stream of air to afford 210 mg of N-(3-(Benzo[d]thiazol-2-yl)-6-methyl-4,5,6,7-tetrahydrothieno[2,3-c]pyridin-2-yl)acetamide as a brown solid. Yield: 74%; m/z (ESI⁻) 342 (M⁻−H); ¹H NMR (500 MHz, CDCl₃) δ 13.02 (s, 1H), 7.97 (d, J=7 Hz, 1H), 7.90 (d, J=7 Hz, 1H), 7.52 (t, J=8 Hz, 1H), 7.39 (t, J=8 Hz, 1H), 3.64 (s, 2H), 3.09 (s, 2H), 2.89-2.86 (m, 2H), 2.50 (s, 3H), 2.38 (s, 3H)

### Examples 5-7

### tert-Butyl 2-Amino-3-(benzo[d]thiazol-2-yl)-4,5-dihydrothieno[2,3-c]pyridine-6(7H)-carboxylate, 2-Acetamido-3-(benzo[d]thiazol-2-yl)-4,5,6,7-tetrahydrothieno[2,3-c]pyridin-6-ium 2,2,2-Trifluoroacetate, and N-(3-(Benzo[d]thiazol-2-yl)-6-isopropyl-4,5,6,7-tetrahydrothieno[2,3-c]pyridin-2-yl)acetamide

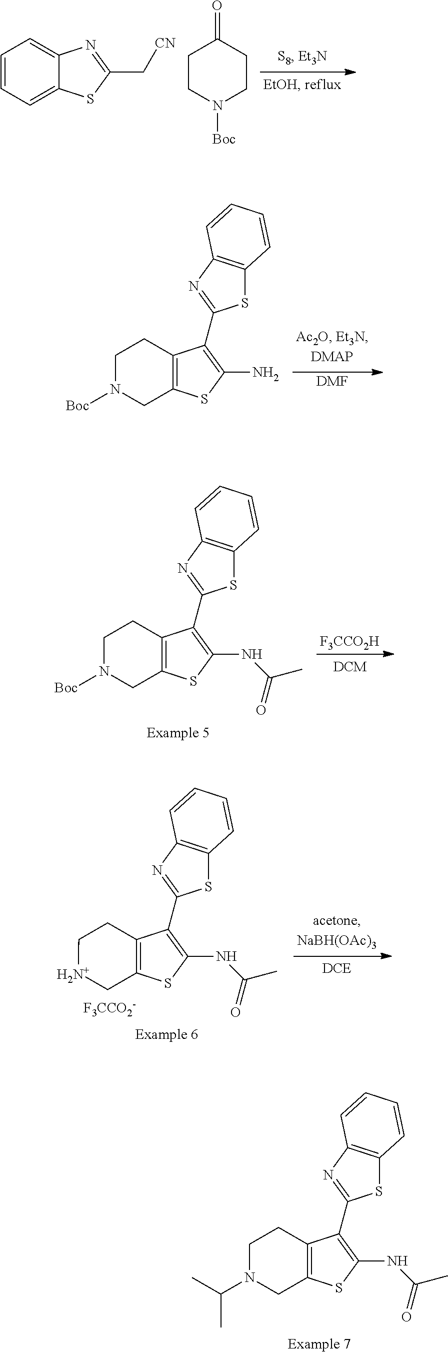

A mixture of N-Boc-4-piperidone (6.85 g, 34.4 mmol), (1,3-benzothiazol-2-yl)acetonitrile (6 g, 34.4 mmol), sulfur (1.1 g, 34.4 mmol), and triethylamine (7.2 mL, 51.7 mmol) in anhydrous ethanol (80 mL) was heated at reflux or 3 h. The reaction mixture was allowed to cool to room temperature, which resulted in the formation of a precipitate. The precipitate was collected via filtration and was then washed with methanol to afford a brown solid. The crude product was purified by column chromatography on silica using an ethyl acetate/hexanes mixture as the eluant. Fractions were combined and condensed in vacuo to afford a solid, which was then triturated with ethyl acetate. The mixture was filtered and allowed dry in a stream of air to afford 8.66 g of tert-butyl 2-amino-3-(benzo[d]thiazol-2-yl)-4,5-dihydrothieno[2,3-c]pyridine-6(7H)-carboxylate. Yield: 65%; m/z (ESI⁺) 388 (MH⁺); ¹H NMR (500 MHz, d₆-DMSO) δ 8.15 (s, 2H), 7.99 (d, J=8 Hz, 1H), 7.88 (d, J=8 Hz, 1H), 7.44 (t, J=7.5 Hz, 1H), 7.30 (t, J=6.5 Hz, 1H), 4.35 (s, 2H), 3.65 (s, 2H), 2.83 (s, 2H), 1.42 (s, 9H).

A mixture of tert-butyl 2-amino-3-(benzo[d]thiazol-2-yl)-4,5-dihydrothieno[2,3-c]pyridine-6(7H)-carboxylate (3 g; 7.74 mmol), triethyamine (3.2 mL, 23.2 mmol) and N,N-dimethyl-4-aminopyridine (50 mg) in N,N-dimethylformamide (15 mL) was treated with acetic anhydride (1.1 mL, 11.6 mmol). The reaction mixture was allowed to stir at room temperature for 24 h, after which a large amount of precipitate had formed in the flask. The precipitate was collected by filtration and then washed with water and methanol. The crude product was purified by crystallization from methanol to afford 1.5 g of tert-butyl 2-acetamido-3-(benzo[d]thiazol-2-yl)-4,5-dihydrothieno[2,3-c]pyridine-6(7H)-carboxylate as a pale yellow solid. Yield: 45%; m/z (ESI⁺) 452 (MNa⁺), 430 (MH⁺); ¹H NMR (500 MHz, CDCl₃) δ 13.00 (s, 1H), 7.96 (d, J=8.5 Hz, 1H), 7.90 (d, J=8 Hz, 1H), 7.51 (t, J=7.5 Hz, 1H), 7.38 (t, J=7.5 Hz, 1H), 4.58 (s, 2H), 3.78 (s, 2H), 3.00 (s, 2H), 2.37 (s, 3H), 1.50 (s, 9H).

A solution of tert-butyl 2-acetamido-3-(benzo[d]thiazol-2-yl)-4,5-dihydrothieno[2,3-c]pyridine-6(7H)-carboxylate (1 g, 2.33 mmol) in dichloromethane (20 mL) was treated with trifluoroacetic acid (1.5 mL). The reaction mixture was allowed to stir at room temperature for 24 h. The reaction mixture was filtered and the filtered solids were washed dichloromethane, followed by drying in a stream of air to afford 351 mg of 2-acetamido-3-(benzo[c]thiazol-2-yl)-4,5,6,7-tetrahydrothieno[2,3-c]pyridin-6-ium 2,2,2-trifluoroacetate as a pale yellow solid. Yield: 34%; m/z (ESI⁺) 330 (MH⁺); ¹H NMR (500 MHz, d₆-DMSO) δ 12.50 (s, 1H), 9.31 (s, 2H), 8.17 (d, J=8.5 Hz, 1H), 8.16 (d, J=8.5 Hz, 1H), 7.60 (t, J=8 Hz, 1H), 7.49 (t, J=8 Hz, 1H), 4.34 (s, 2H), 3.50 (s, 2H), 3.15 (s, 2H), 2.33 (s, 3H).

To a solution of 2-acetamido-3-(benzo[c]thiazol-2-yl)-4,5,6,7-tetrahydrothieno[2,3-c]pyridin-6-ium 2,2,2-trifluoroacetate (130 mg, 0.39 mmol) in dichloroethane (3 mL) was added acetic acid (0.13 mL, 2.36 mmol) and acetone (0.32 mL, 4.33 mmol). The mixture was allowed to stir at room temperature for 3 hours and then sodium triacetoxyborohydride (165 mg, 0.78 mmol) was added portionwise to the mixture. The reaction mixture was allowed to stir at room temperature for 24 h. The reaction mixture was diluted with dichloromethane (20 mL) and then washed with a 5% sodium bicarbonate solution and brine. The organic phase was dried over magnesium sulfate, filtered, and then concentrated in vacuo. The crude product was triturated with methanol and then diethyl ether to afford 44 mg of N-(3-(benzo[d]thiazol-2-yl)-6-isopropyl-4,5,6,7-tetrahydrothieno[2,3-c]pyridin-2-yl)acetamide as pale yellow solid after drying in a stream of air. Yield: 32%; m/z (ESI⁺) 372 (MH⁺); ¹H NMR (600 MHz, CDCl₃) δ 13.00 (s, 1H), 7.96 (d, J=8.4 Hz, 1H), 7.89 (d, J=7.8 Hz, 1H), 7.51 (t, J=7.8 Hz, 1H), 7.38 (t, J=7.8 Hz, 1H), 3.80 (s, 2H), 3.10-3.02 (m, 5H), 2.37 (s, 3H), 1.25-1.19 (m, 6H).

### 5 Examples 8-9

### 2-Acetamido-3-(5-chlorobenzo[dd]thiazol-2-yl)-4,5,6,7-tetrahydrothieno[2,3-c]pyridin-6-ium 2,2,2-Trifluoroacetate and N-(3-(5-Chlorobenzo[d]thiazol-2-yl)-6-ethyl-4,5,6,7-tetrahydrothieno[2,3-c]pyridin-2-yl)acetamide

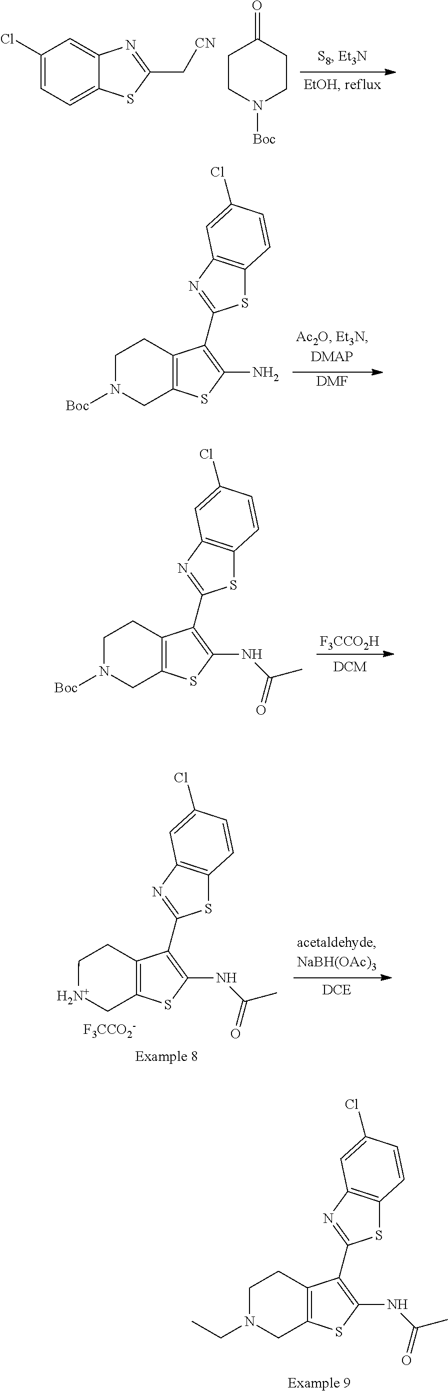

A mixture of 2-(5-chlorobenzo[d]thiazol-2-yl)acetonitrile (2 g, 9.61 mmol), N-Boc-4-piperidone (1.91 g, 9.61 mmol), sulfur (307 mg, 9.61 mmol), and triethylamine (2 mL, 14.4 mmol) in anhydrous ethanol (20 mL) was heated at reflux for 20 min. The reaction mixture was allowed to cool to room temperature and a solid precipitate formed. The precipitate was collected by filtration and was then washed with methanol to afford 2.56 g of tert-butyl 2-amino-3-(5-chlorobenzo[d]thiazol-2-yl)-4,5-dihydrothieno[2,3-c]pyridine-6(7H)-carboxylate. Yield: 63%; m/z (ESI⁺) 424/422 (Cl³⁷:MH⁺/Cl³⁵:MH⁺).

Acetic anhydride (834 μL, 8.82 mmol) was added to a solution of tert-butyl 2-amino-3-(5-chlorobenzo[d]thiazol-2-yl)-4,5-dihydrothieno[2,3-c]pyridine-6(7H) carboxylate (2.48 g, 5.88 mmol), triethylamine (2.45 mL, 17.6 mmol), and N,N-dimethyl-4-aminopyridine (50 mg) in N,N-dimethylformamide (25 mL). The resulting mixture was allowed to stir at room temperature for 2 d. The precipitate that formed in the reaction mixture was collected by filtration and then washed with water and methanol to afford 2.5 g of tert-butyl 2-acetamido-3-(5-chlorobenzo[d]thiazol-2-yl)-4,5-dihydrothieno[2,3-c]pyridine-6(7H)-carboxylate. Yield: 91%; m/z (ESI⁻) 466/464 (Cl³⁷:MH⁺/Cl³⁵:MH⁺).

Trifluoroacetic acid (5 mL) was added to a solution of tert-butyl 2-acetamido-3-(5-chlorobenzo[d]thiazol-2-yl)-4,5-dihydrothieno[2,3-c]pyridine-6(7H)-carboxylate (2.4 g, 5.17 mmol) in dichloromethane (15 mL). The mixture was stirred at room temperature for 24 h. The precipitate that formed was collected by filtration and then washed with dichloromethane to afford 870 mg of 2-acetamido-3-(5-chlorobenzo[d]thiazol-2-yl)-4,5,6,7-tetrahydrothieno[2,3-c]pyridin-6-ium 2,2,2-trifluoroacetate as a white powder. Additional 2-acetamido-3-(5-chlorobenzo[c]thiazol-2-yl)-4,5,6,7-tetrahydrothieno[2,3-c]pyridin-6-ium 2,2,2-trifluoroacetate (1.4 g) was obtained as a pale brown powder by concentrating the dichloromethane filtrate and recrystallizing the residue from methanol. Yield: 92%; m/z (ESI⁺) 364/362 (Cl³⁷:MH⁺/Cl³⁵:MH⁺); ¹H NMR (300 MHz, d₆-DMSO) δ 9.19 (bs, 2H), 8.37 (s, 1H), 8.23 (d, J=8.4 Hz, 1H), 7.54 (d, J=8.4 Hz, 1H), 4.35 (bs, 2H), 3.52 (bs, 2H), 3.15 (bs, 2H), 2.37 (s, 3H).

Acetaldehyde (178 μL, 3.13 mmol) was added to a suspension of 2-acetamido-3-(5-chlorobenzo[d]thiazol-2-yl)-4,5,6,7-tetrahydrothieno[2,3-c]pyridin-6-ium 2,2,2-trifluoroacetate (800 mg, 1.67 mmol) in dichloroethane (8 mL). The mixture was allowed to stir at room temperature for 1.5 h and then sodium triacetoxyborohydride (707 mg, 3.34 mmol) was added portionwise. The resulting mixture was allowed stirred at room temperature for 30 min. The reaction was quenched through the addition of saturated aqueous sodium bicarbonate solution and the mixture was diluted with dichloromethane. The phases were separated and the organic layer was washed with a 5% sodium bicarbonate solution, water, and then brine. The organic layer was dried over magnesium sulfate, filtered, and condensed in vacuo to give an orange solid. Trituration with methanol and filtering afforded 600 mg of N-(3-(5-chlorobenzo[c]thiazol-2-yl)-6-ethyl-4,5,6,7-tetrahydrothieno[2,3-c]pyridin-2-yl)acetamide. Yield: 92%; m/z (ESI⁺) 394/392 (Cl³⁷:MH⁺/Cl³⁵:MH⁺); ¹H NMR (600 MHz, d₆-DMSO) δ 12.45 (s, 1H), 8.23 (s, 1H), 8.10 (d, J=8.4 Hz, 1H), 7.44 (d, J=8.4 Hz, 1H), 3.49 (s, 2H), 2.84 (s, 2H), 2.75-2.72 (m, 2H), 2.52-2.46 (m, 2H), 2.29 (s, 3H), 1.06 (t, J=7.2 Hz, 3H).

### Example 10

### N-(3-(Benzo[d]thiazol-2-yl)-6-(trifluoromethyl)-4,5,6,7-tetrahydrothieno[2,3-c]pyridin-2-yl)acetamide

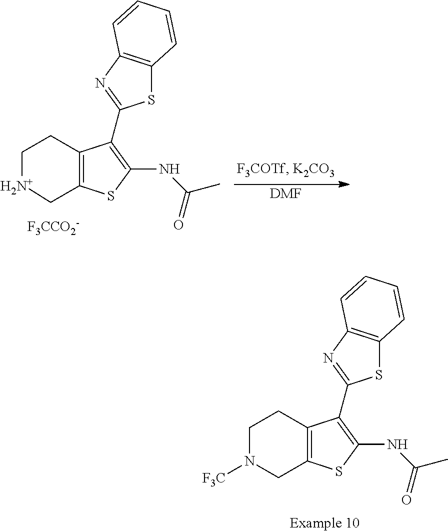

Trifluoromethyl trifluoromethanesulfonate (72.9 mg) was added to a mixture of 2-acetamido-3-(benzo[c]thiazol-2-yl)-4,5,6,7-tetrahydrothieno[2,3-c]pyridin-6-ium 2,2,2-trifluoroacetate (from Example 6; 100 mg, 0.304 mmol) and potassium carbonate (84 mg, 0.608 mmol) in N,N-dimethylformamide (1 mL). The reaction mixture was allowed to stir at room temperature for 24 h. The reaction mixture was filtered and the remaining solids were washed with water and methanol. The solids were dissolved in dichloromethane and washed further with water. The organic phase was dried over magnesium sulfate, filtered, and condensed in vacuo to afford 60 mg of N-(3-(benzo[d]thiazol-2-yl)-6-(trifluoromethyl)-4,5,6,7-tetrahydrothieno[2,3-c]pyridin-2-yl)acetamide as a pale brown solid. Yield: 42%; m/z (ESI⁺) 398 (MH⁺); ¹H NMR (500 MHz, CDCl₃) δ 13.03 (d, J=9.5 Hz, 1H), 7.98 (d, J=8 Hz, 1H), 7.92 (dd, J=8, 3 Hz, 1H), 7.54 (t, J=7.5 Hz, 1H), 7.42 (t, J=7.5 Hz, 1H), 4.65 (d, J=9.5 Hz, 2H), 3.86 (t, J=6 Hz, 1H), 3.83 (t, J=6 Hz, 1H), 3.12-3.07 (m, 2H), 2.38 (s, 3H).

TABLE 4
Further Examples that were prepared according to general methods of Examples 1-10.
Salt forms were prepared as described in Examples 3 (Chloride), 6 (Trifluoroacetate), and 60 (Oxalate).

| | | | m/z ESI⁺ HCl, | |
|---|---|---|---|---|
| | | m/z | TFA, or | |
| Example | Compound | ESI⁺/ESI⁻ | Oxalate Salt | ¹H NMR |
| 11 | 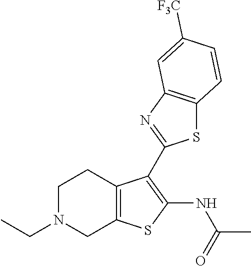 [SMILES: CCN1CCc2c(sc(NC(C)=O)c2-c2nc3cc(C(F)(F)F)ccc3s2)C1] | 426 (MH⁺) | 426 (M⁺, Oxalate) | (600 MHz, d₆-DMSO) δ 12.42 (s, 1H), 8.51 (s, 1H), 8.31 (d, J = 7.2 Hz, 1H), 7.70 (d, J = 7.2 Hz, 1H), 3.47 (bs, 2H), 2.84 (bs, 2H), 2.72 (bs, 2H), 2.31 (s, 3H), 1.05 (t, J = 7.8 Hz, 3H) |
| 12 | 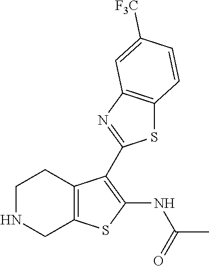 [SMILES: CC(=O)Nc1sc2c(c1-c1nc3cc(C(F)(F)F)ccc3s1)CCNC2] | N/A | 397 (M⁺, TFA) | (600 MHz, d₆-DMSO) δ 12.39 (s, 1H), 9.33 (s, 2H), 8.60 (s, 1H), 8.38 (d, J = 8.4 Hz, 1H), 7.77 (d, J = 8.4 Hz, 1H), 4.32 (s, 2H), 3.48 (bs, 2H), 3.13 (bs, 2H), 2.35 (s, 3H) |
| 13 | 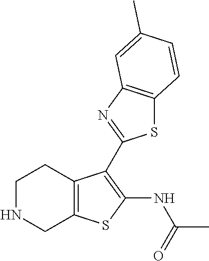 [SMILES: CC(=O)Nc1sc2c(c1-c1nc3cc(C)ccc3s1)CCNC2] | N/A | 344 (M⁺, TFA) | (600 MHz, d₆-DMSO) δ 12.55 (s, 1H), 9.23 (s, 2H), 8.01 (d, J = 7.8 Hz, 1H), 7.97 (s, 1H), 7.30 (d, J = 7.8 Hz, 1H), 4.31 (s, 2H), 3.74 (bs, 2H), 3.13 (s, 3H), 3.13-3.10 (m, 2H), 2.31 (s, 3H) |
| 14 | 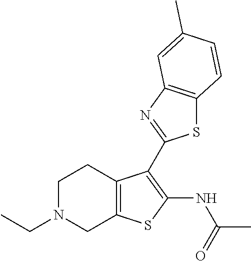 [SMILES: CCN1CCc2c(sc(NC(C)=O)c2-c2nc3cc(C)ccc3s2)C1] | 372 (MH⁺) | N/A | (600 MHz, d₆-DMS) δ 12.60 (bs, 1H), 7.98 (d, J = 10.2 Hz, 1H), 7.93 (s, 1H), 7.27 (d, J = 10.2 Hz, 1H), 3.51 (s, 2H), 2.88 (s, 2H), 2.76 (t, J = 6.6 Hz, 2H), 2.52 (q, J = 8.0 Hz, 2H), 2.50 (s, 3H), 2.30 (s, 3H), 1.07 (t, J = 8.0 Hz, 3H) |
| 15 | 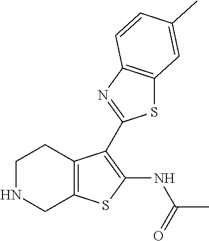 [SMILES: CC(=O)Nc1sc2c(c1-c1nc3ccc(C)cc3s1)CCNC2] | N/A | 344 (M⁺, TFA) | (600 MHz, d₆-DMSO) δ 12.49 (s, 1H), 9.24 (s, 2H), 8.02 (d, J = 8.4 Hz, 1H), 7.93 (s, 1H), 7.38 (d, J = 8.4 Hz, 1), 4.31 (s, 2H), 3.47 (bs, 2H), 3.11 (bs, 2H), 2.44 (s, 3H), 2.30 (s, 3H) |
| 16 | 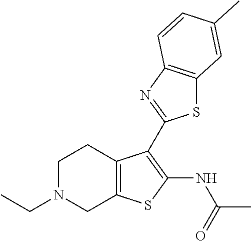 [SMILES: CCN1CCc2c(sc(NC(C)=O)c2-c2nc3ccc(C)cc3s2)C1] | 372 (MH⁺) | 372 (M⁺, HCl) | (600 MHz, d₆-DMSO) δ 12.55 (s, 1H), 11.24 (s, 1H), 8.04 (d, J = 7.2 Hz, 1H), 7.96 (s, 1H), 7.41 (d, J = 7.2 Hz, 1H), 4.58 (d, J = 14.4 Hz, 1H), 4.27 (bs, 1H), 3.90 (bs, 3H), 3.79 (bs, 1H), 3.38 (bs, 1H), 2.47 (s, 3H), 2.34 (s, 3H), 1.35 (s, 3H |
| 17 | 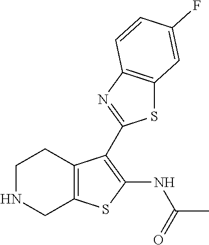 [SMILES: CC(=O)Nc1sc2c(c1-c1nc3ccc(F)cc3s1)CCNC2] | N/A | 348 (M⁺, TFA) | (600 MHz, d₆-DMSO) δ 12.33 (s, 1H), 9.30 (s, 2H), 8.23-8.20 (m, 1H), 8.11 (dd, J = 8.4, 2.4 Hz, 1H), 7.49 (s, 1H), 4.35 (s, 2H), 3.51 (s, 2H), 3.14 (s, 2H), 2.34 (s, 3H) |
| 18 | 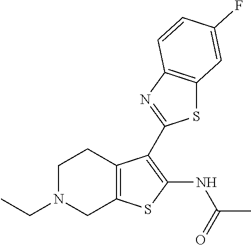 [SMILES: CCN1CCc2c(sc(NC(C)=O)c2-c2nc3ccc(F)cc3s2)C1] | 376 (MH⁺) | 376 (M⁺, HCl) | (600 MHz, d₆-DMSO) δ 12.34 (s, 1H), 10.94 (bs, 1H), 8.22 (dd, J = 9.6, 3.2 Hz, 1H), 8.12 (dd, J = 9.6, 2.4 Hz, 1H), 7.48 (dt, J = 9.6, 2.4 Hz, 1H), 4.60 (d, J = 13.8 Hz, 1H), 4.30-4.26 (m, 1H), 3.90 (d, J = 7.2 Hz, 1H), 3.80 (d, J = 12.6 Hz, 1H), 3.36-3.24 (m, 4H), 2.34 (s, 3H), 1.34 (t, J = 7.2 Hz, 3H) |
| 19 | 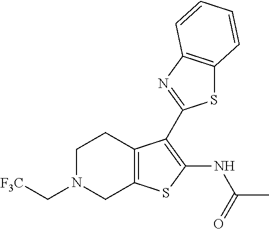 [SMILES: CC(=O)Nc1sc2c(c1-c1nc3ccccc3s1)CCN(CC(F)(F)F)C2] | 412 (MH⁺) | N/A | (600 MHz, CDCl₃) δ 13.04 (s, 1H), 7.98 (d, J = 8.0 Hz, 1H), 7.91 (d, J = 7.5 Hz, 1H), 7.53 (t, J = 7.5 Hz, 1H), 7.40 (t, J = 8 Hz, 1 H), 4.10 (s, 2H), 3.44 (bs, 2H), 3.33 (bs, 2H), 3.20 (bs, 2H), 2.38 (s, 3H) |
| 20 | 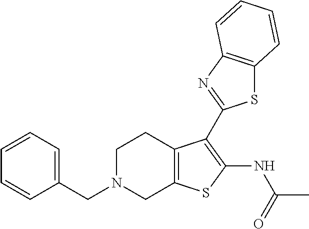 [SMILES: CC(=O)Nc1sc2c(c1-c1nc3ccccc3s1)CCN(Cc1ccccc1)C2] | 420 (MH⁺) | 420 (M⁺, HCl) | (600 MHz, d₆-DMSO) δ 12.50 (s, 1H), 11.36 (s, 1H), 8.14 (t, J = 6.6 Hz, 2H), 7.62 (d, J = 3.6 Hz, 2H), 7.56 (t, J = 7.8 Hz, 1H), 7.47-7.44 (m, 4H), 4.51 (bs, 1H), 4.44 (bs, 1H), 4.31 (bs, 2H), 3.74 (bs, 1H), 3.37 (bs, 1H), 3.26 (bs, 2H), 2.30 (s, 3H) |
| 21 | 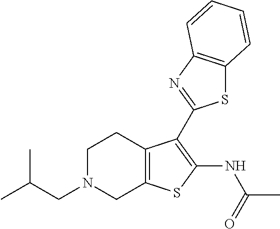 [SMILES: CC(=O)Nc1sc2c(c1-c1nc3ccccc3s1)CCN(CC(C)C)C2] | 386 (MH⁺) | N/A | (600 MHz, d₆-DMSO) δ 12.56 (s, 1H), 8.11 (d, J = 7.8 Hz, 2H), 7.54 (t, J = 7.8 Hz, 1H), 7.43 (t, J = 7.8 Hz, 1H), 3.49 (s, 2H), 2.89 (bs, 2H), 2.74 (t, J = 5.4 Hz, 2H), 2.29 (s, 3H), 2.26 (d, J = 9.8 Hz, 2H), 1.86-1.82 (m, 1H), 0.86 (d, J = 6.6 Hz, 6H) |
| 22 | 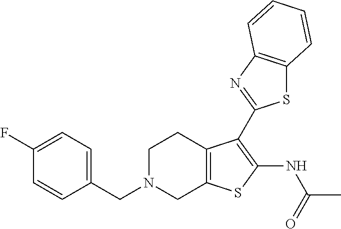 [SMILES: CC(=O)Nc1sc2c(c1-c1nc3ccccc3s1)CCN(Cc1ccc(F)cc1)C2] | 438 (MH⁺) | N/A | (600 MHz, CDCl₃) δ 13.01 (s, 1H), 7.98 (d, J = 9.6 Hz, 1H), 7.91 (d, J = 9.6 Hz, 1H), 7.53 (t, J = 9.6 Hz, 1H), 7.40 (bs, 3H), 7.06 (t, J = 9.6 Hz, 2H), 3.74 (s, 2H), 3.66 (s, 2H), 3.05 (bs, 2H), 2.93 (bs, 2H), 2.38 (s, 3H) |
| 23 | 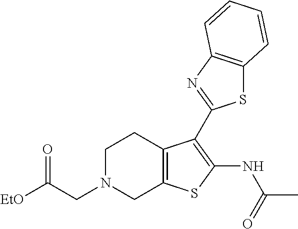 [SMILES: CCOC(=O)CN1CCc2c(sc(NC(C)=O)c2-c2nc3ccccc3s2)C1] | 416 (MH⁺) | N/A | (600 MHz, CDCl₃) δ 13.02 (s, 1H), 7.98 (d, J = 9.6 Hz, 1H), 7.91 (d, J = 9.6 Hz, 1H), 7.53 (t, J = 9 Hz, 1H), 7.40 (t, J = 9 Hz, 1H), 4.26 (q, J = 8.4 Hz, 2H), 3.88 (s, 2H), 3.52 (s, 2H), 3.11 (bs, 4H), 2.38 (s, 3H), 1.33 (t, J = 8.4 Hz, 3H) |
| 24 | 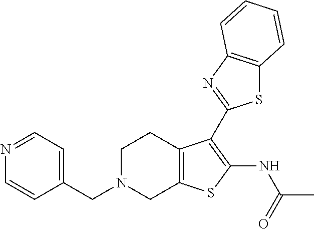 [SMILES: CC(=O)Nc1sc2c(c1-c1nc3ccccc3s1)CCN(Cc1ccncc1)C2] | 421 (MH⁺) | N/A | (600 MHz, d₆-DMSO) δ 12.56 (s, 1H), 8.50 (s, 2H), 8.10 (s, 2H), 7.54 (t, J = 7.8 Hz, 1H), 7.42 (t, J = 7.8 Hz, 1H), 7.36 (s, 2H), 3.71 (s, 2H), 3.56 (s, 2H), 2.91 (s, 2H), 2.79 (s, 2H), 2.28 (s, 3H) |
| 25 | 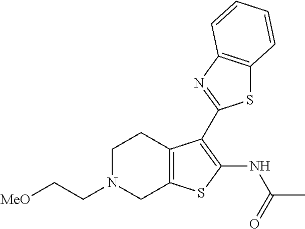 [SMILES: COCCN1CCc2c(sc(NC(C)=O)c2-c2nc3ccccc3s2)C1] | 388 (MH⁺) | 388 (M⁺, HCl) | (600 MHz, d₆-DMSO) δ 12.49 (s, 1H), 10.89 (bs, 1H), 8.15 (d, J = 6.6 Hz, 1H), 8.14 (d, J = 6.6 Hz, 1H), 7.57 (t, J = 7.8 Hz, 1H), 7.46 (t, J = 7.8 Hz, 1H), 4.56 (broad d, J = 15 Hz, 1H), 4.35 (broad d, J = 15 Hz, 1H), 3.78 (bs, 2H), 3.75 (bs, 2H), 3.47 (bs, 2H), 3.36 (s, 3H), 3.24 (bs, 2H), 2.31 (s, 3H) |
| 26 | 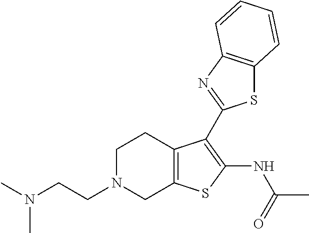 [SMILES: CC(=O)Nc1sc2c(c1-c1nc3ccccc3s1)CCN(CCN(C)C)C2] | 401 (MH⁺) | 401 (M⁺, HCl) | (600 MHz, d₆-DMSO) δ 12.49 (s, 1H), 11.49 (s, 1H), 10.77 (s, 1H), 8.17 (d, J = 3.6 Hz, 1H), 8.15 (d, J = 3.6 Hz, 1H), 7.57 (t, J = 8.4 Hz, 1H), 7.46 (t, J = 8.4 Hz, 1H), 3.95- 3.65 (broad m, 6H), 3.63 (bs, 2H), 3.30 (bs, 2H), 2.84 (s, 6H), 2.32 (s, 3H) |
| 27 | 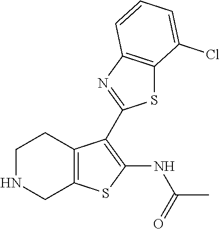 [SMILES: CC(=O)Nc1sc2c(c1-c1nc3cccc(Cl)c3s1)CCNC2] | N/A | 366/364 (M⁺ ³⁷Cl/³⁵Cl, TFA) | (600 MHz, d₆-DMSO) δ 12.31 (s, 1H), 9.33 (bs, 2H), 8.16 (d, J = 8.4 Hz, 1H), 7.64 (t, J = 7.2 Hz, 1H), 7.61 (t, J = 7.2 Hz, 1H), 4.35 (s, 2H), 3.51 (bs, 2H), 3.16 (bs, 2H), 2.34 (s, 3H) |
| 28 | 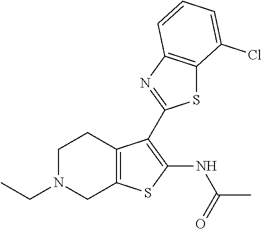 [SMILES: CCN1CCc2c(sc(NC(C)=O)c2-c2nc3cccc(Cl)c3s2)C1] | 394/392 (MH⁺ ³⁷Cl/³⁵Cl) | 394/392 (M⁺ ³⁷Cl/³⁵Cl, HCl) | (500 MHz, d-TFA) δ 7.70 (d, J = 8 Hz, 1H), 7.45-7.40 (m, 2H), 4.42 (d, J = 15.5 Hz, 1H), 3.96 (d, J = 15.5 Hz, 1H), 3.58 (bs, 1H), 3.21-3.10 (m, 4H), 2.76 (d, J = 13 Hz, 1H), 1.92 (s, 3H), 1.15 (t, J = 7.5 Hz, 3H) |
| 29 | 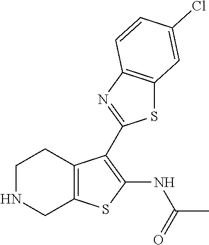 [SMILES: CC(=O)Nc1sc2c(c1-c1nc3ccc(Cl)cc3s1)CCNC2] | N/A | 366/364 (M⁺ ³⁷Cl/³⁵Cl, TFA) | (600 MHz, d₆-DMSO) δ 12.38 (s, 1H), 9.30 (bs, 2H), 8.34 (s, 1H), 8.17 (d, J = 9 Hz, 1H), 7.63 (d, J = 9 Hz, 1H), 4.35 (s, 2H), 3.51 (bs, 2H), 3.14 (bs, 2H), 2.34 (s, 3H) |
| 30 | 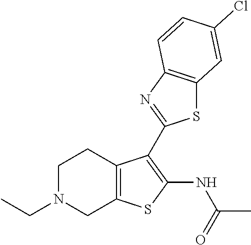 [SMILES: CCN1CCc2c(sc(NC(C)=O)c2-c2nc3ccc(Cl)cc3s2)C1] | 394/392 (MH⁺ ³⁷Cl/³⁵Cl) | 394/392 (M⁺ ³⁷Cl/³⁵Cl, HCl) | (600 MHz, d₆-DMSO) δ 12.38 (s, 1H), 11.23 (bs, 1H), 8.32 (s, 1H), 8.14 (d, J = 9 Hz, 1H), 7.60 (d, J = 9 Hz, 1H), 4.57 (d, J = 18 Hz, 1H), 4.25 (m, 1H), 3.77 (bs, 1H), 3.36- 3.22 (m, 5H), 2.32 (s, 3H), 1.33 (t, J = 9 Hz, 3H) |
| 31 | 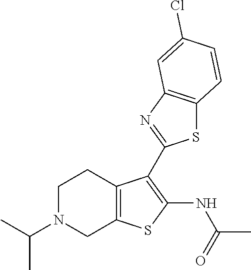 [SMILES: CC(=O)Nc1sc2c(c1-c1nc3cc(Cl)ccc3s1)CCN(C(C)C)C2] | 406/404 (MH⁺ ³⁷Cl/³⁵Cl) | 406/404 (M⁺ ³⁷Cl/³⁵Cl, HCl) | (600 MHz, d₆-DMSO) δ 12.44 (s, 1H), 10.82 (bs, 1H), 8.33 (s, 1H), 8.20 (d, J = 9.6 Hz, 1H), 7.51 (d, J = 9.6 Hz, 1H), 4.46 (bs, 1H), 4.35 (bs, 1H), 3.79 (bs, 1H), 3.65 (bs, 1H), 3.37 (bs, 2H), 3.25 (bs, 1H), 2.35 (s, 3H), 1.34 (bs, 6H) |

### Examples 32-35

### 3-(Benzo[d]thiazol-2-yl)-2-(cyclopropanecarboxamido)-4,5,6,7-tetrahydrothieno[2,3-c]pyridin-6-ium 2,2,2-Trifluoroacetate, N-(3-(benzo[d]thiazol-2-yl)-6-methyl-4,5,6,7-tetrahydrothieno[2,3-c]pyridin-2-yl)cyclopropanecarboxamide, N-(3-(benzo[d]thiazol-2-yl)-6-ethyl-4,5,6,7-tetrahydrothieno[2,3-c]pyridin-2-yl)cyclopropanecarboxamide, and 3-(Benzo[d]thiazol-2-yl)-2-(cyclopropanecarboxamido)-6-methyl-4,5,6,7-tetrahydrothieno[2,3-c]pyridin-6-ium Chloride

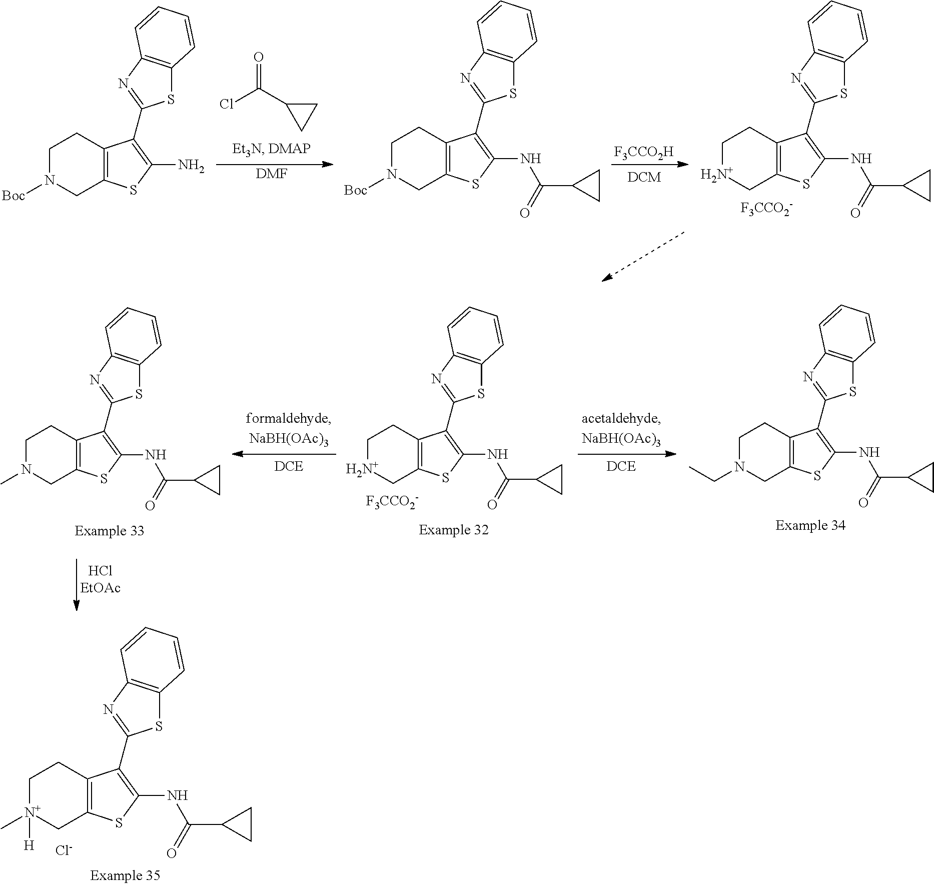

To a solution of tert-butyl 2-amino-3-(benzo[d]thiazol-2-yl)-4,5-dihydrothieno[2,3-c]pyridine-6(7H)-carboxylate (from Example 5; 0.5 g, 1.29 mmol) in dichloromethane (10 mL) were added triethylamine (0.27 mL, 1.94 mmol) and cyclopropanecarbonyl chloride (0.13mL, 1.42 mmol), followed by N,N-dimethyl-4-aminopyridine (32 mg, 0.26 mmol). The reaction mixture was allowed to stir for 24 h at ambient temperature. The reaction mixture was diluted with dichloromethane and then washed with 1M hydrochloric acid solution, 5% sodium bicarbonate solution, and brine. The organic layer was dried over magnesium sulfate, filtered, and concentrated in vacuo. The crude product was purified by column chromatography on silica using an ethyl acetate/hexanes mixture (1:7) as the eluant to afford 0.30 g of tert-butyl 3-(benzo[c]thiazol-2-yl)-2-(cyclopropanecarboxamido)-4,5-dihydrothieno[2,3-c]pyridine-6(7H)-carboxylate as an off-white solid. Yield: 51%; m/z (ESI⁻) 454 (M⁻−H).

To a solution of tert-butyl 3-(benzo[d]thiazol-2-yl)-2-(cyclopropanecarboxamido)-4,5-dihydrothieno[2,3-c]pyridine-6(7H)-carboxylate (0.3 g, 0.66 mmol) in dichloromethane (3 mL) was added trifluoroacetic acid (0.5 mL). The reaction mixture was allowed to stir for 24 h at ambient temperature. The reaction mixture was then diluted with diethyl ether to cause a solid to precipitate. The precipitate was collected by filtration and then washed with diethyl ether to give 0.30 g of 3-(benzo[d]thiazol-2-yl)-2-(cyclopropanecarboxamido)-4,5,6,7-tetrahydrothieno[2,3-c]pyridin-6-ium 2,2,2-trifluoroacetate as an off-white solid. Yield: 97%; m/z (ESI⁺) 356 (MH⁺); ¹H NMR (600 MHz, d₆-DMSO) δ 12.81 (s, 1H), 9.23 (s, 2H), 8.18 (d, J=8.4 Hz, 1H), 8.16 (d, J=7.8 Hz, 1H), 7.57 (t, J=7.2 Hz, 1H), 7.47 (t, J=7.2 Hz, 1H), 4.31 (s, 2H), 3.48 (s, 2H), 3.13 (s, 2H), 2.13-2.11 (m, 1H), 0.99-0.95 (m, 4H).

To a suspension of 3-(benzo[d]thiazol-2-yl)-2-(cyclopropanecarboxamido)-4,5,6,7-tetrahydrothieno[2,3-c]pyridin-6-ium 2,2,2-trifluoroacetate (0.15 g, 0.32 mmol) in dichloroethane (4 mL) were added a 36% solution of formaldehyde in water (108 mL, 1.4 mmol) and acetic acid (0.073 mL, 1.28 mmol). The reaction mixture was allowed to stir for 1 h at ambient temperature before sodium triacetoxyborohydride (0.38 g, 1.80 mmol) was added portionwise over 5 min. The resulting mixture was allowed to stir for 24 h at ambient temperature. The reaction mixture was quenched through the addition of a 5% sodium bicarbonate solution. The reaction mixture was then extracted with dichloromethane and the organic phase was washed with a 5% sodium bicarbonate solution and brine. The organic phase was then dried over magnesium sulfate, filtered, and concentrated in vacuo. The crude product was triturated with diethyl ether and filtered to afford 61 mg of N-(3-(benzo[d]thiazol-2-yl)-6-methyl-4,5,6,7-tetrahydrothieno[2,3-c]pyridin-2-yl)cyclopropanecarboxamide as a yellow solid. Yield: 51%; m/z (ESI⁺) 370 (MH⁺).

To a suspension of N-(3-(benzo[d]thiazol-2-yl)-6-methyl-4,5,6,7-tetrahydrothieno[2,3-c]pyridin-2-yl)cyclopropanecarboxamide (55 mg, 0.15 mmol) in ethyl acetate (0.5 mL) was added a solution of 4.5M hydrochloric acid in ethyl acetate (1 mL). The reaction mixture was allowed to stir for 10 min at ambient temperature before it was diluted with diethyl ether. The precipitate formed was collected by filtration, washed with diethyl ether, and the allowed to dry in a stream of air to afford 58 mg of 3-(benzo[d]thiazol-2-yl)-2-(cyclopropanecarboxamido)-6-methyl-4,5,6,7-tetrahydrothieno[2,3-c]pyridin-6-ium chloride as an off-white solid. Yield: 97%; m/z (ESI⁺) 370 (MH⁺); ¹H NMR (600 MHz, d₆-DMSO) δ 12.85 (s, 1H), 11.08 (s, 1H), 8.21 (d, J=8.4 Hz, 1H), 8.18 (d, J=7.8 Hz, 1H), 7.60 (t, J=7.8 Hz, 1H), 7.49 (t, J=7.8 Hz, 1H), 4.54 (d, J=15 Hz, 1H), 4.30-4.27 (bm, 1H), 3.75 (bs, 1H), 3.42 (bs, 1H), 3.27 (bs, 2H), 2.93 (s, 3H), 2.15 (bs, 1H), 1.02-0.98 (m, 4H).

To a suspension of 3-(benzo[d]thiazol-2-yl)-2-(cyclopropanecarboxamido)-4,5,6,7-tetrahydrothieno[2,3-c]pyridin-6-ium 2,2,2-trifluoroacetate (0.14 g, 0.30 mmol) in dichloroethane (4 mL) was added acetaldehyde (0.032 mL, 0.57 mmol) and acetic acid (0.10 mL, 1.79 mmol). The reaction mixture was allowed to stir for 1 h at ambient temperature before sodium triacetoxyborohydride (0.18 g, 0.84 mmol) was added portionwise over 5 min. The resulting mixture was allowed to stir for 24 h at ambient temperature. The reaction was quenched through the addition of a 5% sodium bicarbonate solution and the resulting mixture was then extracted with dichloromethane. The organic layer was washed with a 5% sodium bicarbonate solution and brine, dried over magnesium sulfate, filtered and concentrated in vacuo. The crude product was triturated with methanol, filtered, and then allowed to dry in a stream of air to afford 61 mg of N-(3-(benzo[c]thiazol-2-yl)-6-ethyl-4,5,6,7-tetrahydrothieno[2,3-c]pyridin-2-yl)cyclopropanecarboxamide as a yellow solid. Yield: 55%; m/z (ESI⁺) 384 (MH⁺); ¹H NMR (600 MHz, d₆-DMSO) δ 12.70 (s, 1H), 8.09 (t, J=8.4 Hz, 2H), 7.54 (t, J=7.2 Hz, 1H), 7.42 (t, J=7.2 Hz, 1H), 3.55 (s, 2H), 2.92 (t, J=6 Hz, 2H), 2.80 (t, J=6 Hz, 2H), 2.56 (q, J=7.2 Hz, 2H), 2.01-1.99 (m, 1H), 1.08 (t, J=7.2 Hz, 3H), 0.98-0.96 (m, 4H).

### Example 36

### 3-(Benzo[d]thiazol-2-yl)-6-ethyl-2-isobutyramido-4,5,6,7-tetrahydrothieno[2,3-c]pyridin-6-ium Chloride

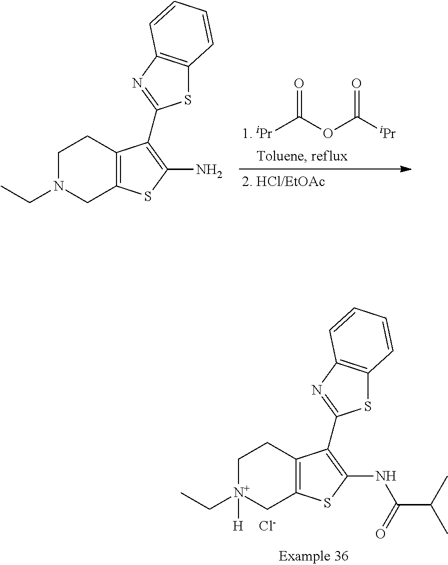 [SMILES: CCN1CCc2c(sc(N)c2-c2nc3ccccc3s2)C1 ; CC(C)C(=O)OC(=O)C(C)C ; CC[NH+]1CCc2c(sc(NC(=O)C(C)C)c2-c2nc3ccccc3s2)C1 ; [Cl-]]

A solution of 3-(benzo[c]thiazol-2-yl)-6-ethyl-4,5,6,7-tetrahydrothieno[2,3-c]pyridin-2-amine (from example 1, 0.4 g, 1.27 mmol) in toluene (7 mL) was treated with isobutyric anhydride (0.32 mL, 1.90 mmol). The mixture was heated at reflux for 12 h and then allowed to cool to room temperature. The mixture was condensed in vacuo and the crude product was purified by column chromatography on silica using a dichloromethane/methanol mixture. The acylated product was then dissolved in ethyl acetate (5 mL) and treated with a solution of 4.5M hydrochloric acid in ethyl acetate (5 mL). The mixture was allowed to stir at ambient temperature for 10 min and was then filtered. The filtered solids were recrystallized from isopropanol to afford 193 mg of 3-(benzo[d]thiazol-2-yl)-6-ethyl-2-isobutyramido-4,5,6,7-tetrahydrothieno[2,3-c]pyridin-6-ium chloride. Yield: 36%; m/z (ESI⁻) 386 (MH⁺); ¹H NMR (200 MHz, d₆-DMSO) δ 12.87 (s, 1H), 11.22 (bs, 1H), 8.18 (d, J=7.6 Hz, 1H), 8.04 (d, J=7.6 Hz, 1H), 7.59 (t, J=7 Hz, 1H), 7.47 (t, J=7 Hz, 1H), 4.64-4.53 (m, 1H), 4.30 (bs, 1H), 3.76 (bs, 1H), 3.27 (bs, 5H), 2.86 (p, J=7 Hz, 1H), 1.37-1.27 (m, 9H).

### Example 37

### N-(3-(Benzo[c]thiazol-2-yl)-6-ethyl-4,5,6,7-tetrahydrothieno[2,3-c]pyridin-2-yl)methanesulfonamide

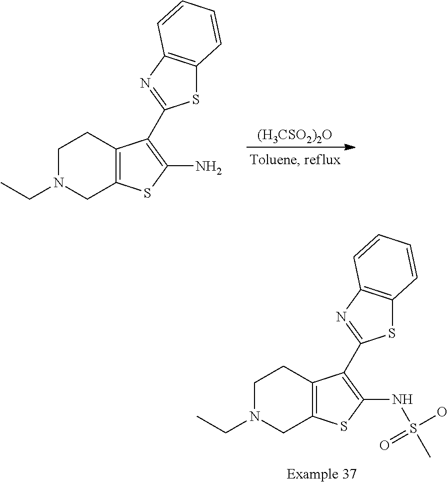

A mixture of 3-(benzo[c]thiazol-2-yl)-6-ethyl-4,5,6,7-tetrahydrothieno[2,3-c]pyridin-2 amine (from example 1, 1 g, 3.17 mmol), methanesulfonic anhydride (0.83 g, 4.76 mmol), and toluene (20 mL) was heated at reflux for 3 h. After cooling to room temperature, the solvent was removed under reduced pressure and the remaining residue was dissolved in dichloromethane (100 ml). The mixture was washed with a 5% sodium bicarbonate solution (2×10 mL) and brine (1×10 mL), dried over magnesium sulfate, filtered and condensed in vacuo. The crude product was purified twice by column chromatography on silica first using a mixture of dichloromethane/methanol and then ethyl acetate/hexanes. The product was then purified by preparative HPLC on a C18 reverse phase column to afford 7 mg of N-(3-(benzo[c]thiazol-2-yl)-6-ethyl-4,5,6,7-tetrahydrothieno[2,3-c]pyridin-2-yl)methanesulfonamide as a green solid. Yield: 0.6%; m/z (ESI⁺) 394 (MH⁺); ¹H NMR (500 MHz, d₆-DMSO) δ 9.78 (s, 1H), 7.95 (s, 1H), 7.75 (s, 1H), 7.37 (t, J=7.5 Hz, 1H), 7.23 (t, J=7 Hz, 1H), 4.42 (bs, 1H), 4.19 (bs, 1H), 3.76 (bs, 1H), 3.57 (bs, 1H), 3.30-3.26 (m, 4H), 2.76 (s, 3H), 1.30 (t, J=7.5 Hz, 3H).

TABLE 5
Further Examples that were prepared according to Examples 32-37. Salt forms were
prepared as described in Examples 3 (Chloride), 6 (Trifluoroacetate), and 60 (Oxalate).

| | | | m/z ESI⁺ | |
|---|---|---|---|---|
| | | | HCl, TFA, | |
| | | m/z | or Oxalate | |
| Example | Compound | ESI⁺/ESI⁻ | Salt | ¹H NMR |
| 38 | 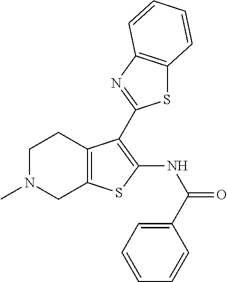 [SMILES: CN1CCc2c(sc(NC(=O)c3ccccc3)c2-c2nc3ccccc3s2)C1] | 406 (MH⁺) | N/A | (600 MHz, d-TFA) δ 7.97 (t, J = 9.6 Hz, 2H), 7.70 (t, J = 7.8 Hz, 1H), 7.66-7.59 (m, 3H), 7.44 (t, J = 7.8 Hz, 1H), 7.28 (t, J = 7.8 Hz, 2H), 4.65 (d, J = 15 Hz, 1H), 4.26 (d, J = 15 Hz, 1H), 3.80 (bs, 1H), 3.40 (bs, 1H), 3.34 (bs, 1H), 3.07 (s, 3H), 3.00 (d, J = 15 Hz, 1H) |
| 39 | 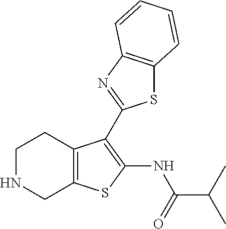 [SMILES: CC(C)C(=O)Nc1sc2c(c1-c1nc3ccccc3s1)CCNC2] | N/A | 358 (M⁺, TFA) | (600 MHz, d₆-DMSO) δ 12.88 (s, 1H), 9.31 (s, 2H), 8.20 (d, J = 7.8 Hz, 1H), 8.07 (d, J = 7.8 Hz, 1H), 7.62 (t, J = 7.8 Hz, 1H), 7.50 (t, J = 7.8 Hz, 1H), 4.36 (s, 2H), 3.52 (s, 2H), 3.18 (s, 2H), 2.87 (sep, J = 6.6 Hz, 1H), 1.31 (d, J = 6.6 Hz, 6H) |
| 40 | 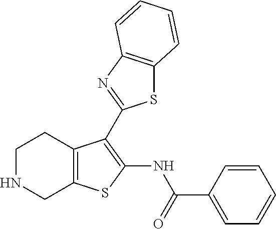 [SMILES: O=C(Nc1sc2c(c1-c1nc3ccccc3s1)CCNC2)c1ccccc1] | N/A | 392 (M⁺, TFA) | (600 MHz, d₆-DMSO) δ 13.89 (s, 1H), 9.33 (bs, 2H), 8.19 (s, 1H), 8.10 (bs, 3H), 7.74 (bs, 3H), 7.63 (s, 1H), 7.50 (s, 1H), 4.39 (bs, 2H), 3.46 (bs, 2H), 3.20 (bs, 2H) |
| 41 | 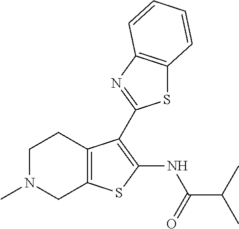 [SMILES: CC(C)C(=O)Nc1sc2c(c1-c1nc3ccccc3s1)CCN(C)C2] | 372 (MH⁺) | N/A | (600 MHz, CDCl₃) δ 13.19 (s, 1H), 7.93 (d, J = 9.6 Hz, 1H), 7.89 (d, J = 9.6 Hz, 1H), 7.50 (t, J = 8.4 Hz, 1H), 7.37 (t, J = 9 Hz, 1H), 3.62 (s, 2H), 3.07 (t, J = 6.6 Hz, 2H), 2.85 (t, J = 6.6 Hz, 2H), 2.78 (sept, J = 8.4 Hz, 1H), 1.42 (d, J = 8.4 Hz, 6H) |
| 42 | 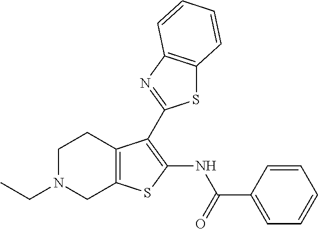 [SMILES: CCN1CCc2c(sc(NC(=O)c3ccccc3)c2-c2nc3ccccc3s2)C1] | 420 (MH⁺) | N/A | (600 MHz, d-TFA) δ 7.97 (t, J = 6.6 Hz, 2H), 7.70 (t, J = 7.8 Hz, 1H), 7.64 (t, J = 7.8 Hz, 1H), 7.60 (d, J = 7.8 Hz, 2H), 7.44 (t, J = 7.8 Hz, 1H), 7.28 (t, J = 7.8 Hz, 2H), 4.68 (broad d, J = 15 Hz, 1H), 4.22 (broad d, J = 15 Hz, 1H), 3.83 (bs, 1H), 3.42 (bs, 2H), 3.36 (bs, 2H), 2.98 (bs, 1H), 1.38 (t, J = 6.6 Hz, 3H) |

### Examples 43-44

### 3-(Benzo[dlthiazol-2-yl)-2-(ethylamino)-4,5,6,7-tetrahydrothieno[2,3-c]pyridin-6-ium 2,2,2-Trifluoroacetate and 3-(Benzo[d]thiazol-2-yl)-6-ethyl-2-(ethylamino)-4,5,6,7-tetrahydrothieno[2,3-c]pyridin-6-ium Carboxyformate

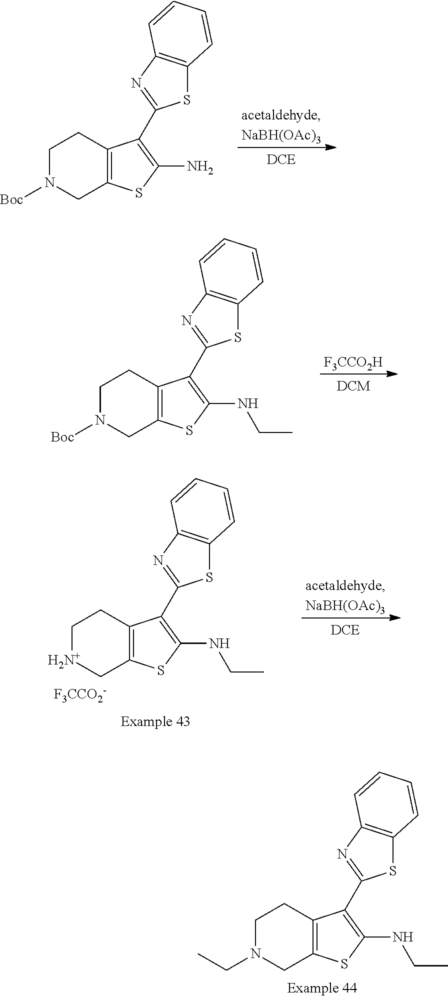

Acetaldehyde (148 μL, 2.45 mmol) was added to a solution of tert-butyl 2-amino-3-(benzo[d]thiazol-2-yl)-4,5-dihydrothieno[2,3-c]pyridine-6(7H)-carboxylate (from example 5, 500 mg, 1. 29 mmol) in dichloromethane (5 mL). The mixture was allowed to stir at room temperature for 2 h and then acetic acid (443 μL; 7.74 mmol) was added, followed by portionwise addition of sodium triacetoxyborohydride (1.04 g, 4.90 mmol). The reaction mixture was allowed to stir at room temperature for 24 h. The reaction was quenched with a saturated aqueous solution of sodium bicarbonate and the resulting mixture was then extracted dichloromethane. The organic phase was dried over magnesium sulfate, filtered, and evaporated. The crude product was purified by column chromatography on silica using dichloromethane as the eluant to afford 150 mg of tert-butyl 3-(benzo[d]thiazol-2-yl)-2-(ethylamino)-4,5-dihydrothieno[2,3-c]pyridine-6(7H)-carboxylate as a yellow-brown oil. Yield: 28%; m/z (ESI⁺) 360 (MH⁺−C₄H₈), 316 (MH⁺−C₅H₉O₂).

tert-Butyl 3-(benzo[d]thiazol-2-yl)-2-(ethylamino)-4,5-dihydrothieno[2,3-c]pyridine-6(7H)-carboxylate (150 mg, 0.361 mmol) was dissolved in dichloromethane (2 mL) and treated trifluoroacetic acid (300 μL). The solution was allowed to stir at room temperature for 24 h. The reaction mixture was concentrated under reduced pressure and the remaining residue was triturated with diethyl ether. The mixture was filtered and the solid was allowed to dry in a stream of air to afford 160 mg of 3-(benzo[d]thiazol-2-yl)-2-(ethylamino)-4,5,6,7-tetrahydrothieno[2,3-c]pyridin-6-ium 2,2,2-trifluoroacetate as a yellow solid. Yield: 99%; m/z (ESI⁺) 316 (MH⁺); ¹H NMR (500 MHz, d₆-DMSO) δ 9.40 (bs, 1H), 9.33 (bs, 2H), 8.09 (d, J=7.5 Hz, 1H), 7.99 (d, J=7.5 Hz, 1H), 7.55 (t, J=7.5 Hz, 1H), 7.40 (t, J=7.5 Hz, 1H), 4.29 (s, 2H), 3.43 (s, 2H), 3.17 (s, 2H), 1.40 (t, J=7 Hz, 3H).

Acetaldehyde (203 μL, 3.62 mmol) was added to a solution of 3-(benzo[d]thiazol-2-yl)-2-(ethylamino)-4,5,6,7-tetrahydrothieno[2,3-c]pyridin-6-ium 2,2,2-trifluoroacetate (850 mg; 1.90 mmol) in dichloroethane (10 mL). The reaction mixture was allowed to stir at room temperature for 2 h and then sodium triacetoxyborohydride (1.53 g, 7.24 mmol) was added portionwise. The reaction mixture was allowed to stir at room temperature for 24 h. The reaction was quenched through the addition of a saturated aqueous solution of sodium bicarbonate and then the mixture was extracted with dichloromethane. The organic phase was dried over magnesium sulfate, filtered, and condensed in vacuo. The crude product was purified by column chromatography using a mixture of chloroform and methanol as the eluant. The product obtained after column chromatography was converted to the oxalate salt by dissolving the solid in ethyl acetate (10 mL), followed by the addition of a 0.3M solution of oxalic acid in ethyl acetate (7 mL). The mixture was allowed to stir for 15 min and then the resulting precipitate was collected by filtration. The filtered solids were triturated with warm methanol and allowed to dry in a stream of air to give 160 mg of 3-(benzo[d]thiazol-2-yl)-6-ethyl-2-(ethylamino)-4,5,6,7-tetrahydrothieno[2,3-c]pyridin-6-ium carboxyformate as a yellow solid. Yield: 25%; m/z (ESI⁺) 345 (MH⁺), ¹H NMR (200 MHz, d₆-DMSO) δ 9.28 (t, J=3.2 Hz, 1H), 7.99 (d, J=7.6 Hz, 1H), 7.89 (d, J=8.2 Hz, 1H), 7.45 (t, J=7.6 Hz, 1H), 7.30 (t, J=7.2 Hz, 1H), 4.16 (bs, 2H), 3.42-3.29 (m, 4H), 3.12 (bs, 2H), 1.34-1.23 (m, 6H).

### Example 45

### 2-Acetamido-3-(benzo[d]thiazol-2-yl)-6-(2-(methylamino)-2-oxoethyl)-4,5,6,7-tetrahydrothieno[2,3-c]pyridin-6-ium Chloride

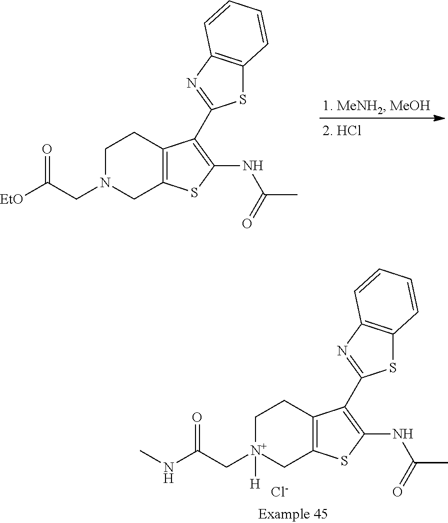

To a suspension of ethyl 2-(2-acetamido-3-(benzo[d]thiazol-2-yl)-4,5-dihydrothieno[2,3-c]pyridin-6(7H)-yl)acetate (from Example 23, 110 mg, 0.26 mmol) in methanol (6 mL) was added a 40% solution of methylamine in water (2.5 mL). The reaction mixture was allowed to stir for 24 h at room temperature. The precipitate from the reaction mixture was collected by filtration. The crude product was purified by flash chromatography on silica using a mixture of methanol/dichloromethane (1:100) as the eluant to afford 80 mg of 2-(2-acetamido-3-(benzo[d]thiazol-2-yl)-4,5-dihydrothieno[2,3-c]pyridin-6(7H)-yl)-N-methylacetamide as a yellow solid. Yield: 73%; m/z (ESI⁺) 401 (MH⁺).

To a suspension of 2-(2-acetamido-3-(benzo[d]thiazol-2-yl)-4,5-dihydrothieno[2,3-c]pyridin-6(7H)-yl)-N-methylacetamide (80 mg, 0.20 mmol) in ethyl acetate (1 mL) was added a solution of 4.5M hydrochloric acid in ethyl acetate (2 mL). The reaction mixture was allowed to stir for 10 min at ambient temperature before it was diluted with diethyl ether. The precipitate that formed was collected by filtration and then washed with diethyl ether to afford 68 mg of 2-acetamido-3-(benzo[d]thiazol-2-yl)-6-(2-(methylamino)-2-oxoethyl)-4,5,6,7-tetrahydrothieno[2,3-c]pyridin-6-ium chloride as a yellow solid. Yield: 78%; m/z (ESI⁺) 401 (MH⁺); ¹H NMR (600 MHz, d₆-DMSO) δ 12.50 (s, 1H), 10.83 (bs, 1H), 8.61 (d, J=4.8 Hz, 1H), 8.18 (t, J=7.2 Hz, 2H), 7.60 (t, J=7.8 Hz, 1H), 7.49 (t, J=7.2 Hz, 1H), 4.58 (bs, 1H), 4.46 (bs, 1H), 3.77 (bs, 1H), 3.58 (bs, 1H), 3.25 (s, 2H), 2.69 (s, 3H), 2.34 (s, 3H).

### Example 46

### 2-Acetamido-3-(benzo[d]thiazol-2-yl)-6-(carboxymethyl)-4,5,6,7-tetrahydrothienol[2,3-c]pyridin-6-ium Chloride

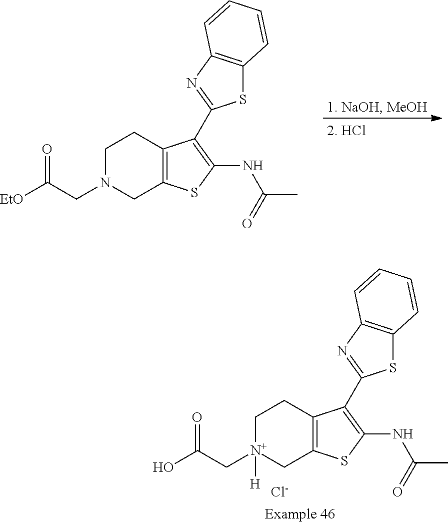 [SMILES: CCOC(=O)CN1CCc2c(sc(NC(C)=O)c2-c2nc3ccccc3s2)C1 ; "1. NaOH, MeOH" ; CC(=O)Nc1sc2c(c1-c1nc3ccccc3s1)CC[NH+](CC(=O)O)C2 ; [Cl-]]

To a suspension of ethyl 2-(2-acetamido-3-(benzo[d]thiazol-2-yl)-4,5-dihydrothieno[2,3-c]pyridin-6(7H)-yl)acetate (from Example 23, 90 mg, 0.22 mmol) in methanol (4 mL) was added 3 drops of a 1M sodium hydroxide solution. The reaction mixture was allowed to stir for 24 h at ambient temperature. The reaction mixture was condensed in vacuo and the residue was acidified with a 1M hydrochloric acid solution to pH 3-4. The mixture was diluted with ethyl acetate and the yellow precipitate that formed was collected by filtration. The filtered solids were washed with methanol and diethyl ether to afford 75 mg of 2-acetamido-3-(benzo[d]thiazol-2-yl)-6-(carboxymethyl)-4,5,6,7-tetrahydrothieno[2,3-c]pyridin-6-ium chloride as a yellowish solid after drying in a stream of air. Yield: 81%; m/z (ESI⁺) 388 (MH⁺); ¹H NMR (600 MHz, d₆-DMSO) δ 12.56 (s, 1H), 8.11 (s, 2H), 7.54 (s, 1H), 7.42 (s, 1H), 3.70 (s, 2H), 3.35 (s, 2H), 2.91 (bs, 4H), 2.29 (s, 3H).

### Examples 47-48

### 3-(Benzo[d]thiazol-2-yl)-5,7-dihydro-4H-thieno[2,3-c]pyran-2-amine and N-(3-(Benzo[d]thiazol-2-yl)-5,7-dihydro-4H-thieno[2,3-c]pyran-2-yl)acetamide

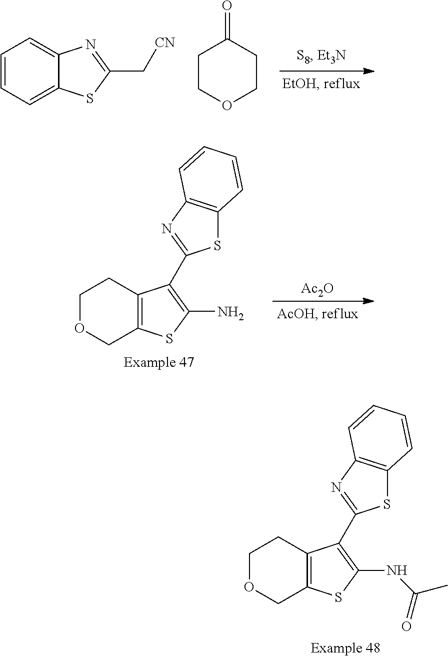

A mixture of (1,3-benzothiazole-2-yl)acetonitrile (1 g, 5.70 mmol), dihydro-2H-pyran-4(31)-one (0.53 mL, 5.70 mmol), sulfur (0.18 g, 5.70 mmol), triethylamine (1.14 mL) and absolute ethanol (30 mL) was heated at reflux for 3 h. After cooling to room temperature the reaction mixture was concentrated under reduced pressure. The remaining residue was triturated with water and then neutralized with concentrated hydrochloric acid. The precipitated solid was filtered and then purified by column chromatography on silica using an ethyl acetate/hexanes gradient (9-50%) to afford 378 mg of 3-(benzo[d]thiazol-2-yl)-5,7-dihydro-4H-thieno[2,3-c]pyran-2-amine. Yield: 23%; m/z (ESI⁻) 289 (MH⁺); ¹H NMR (500 MHz, d₆-DMSO) δ 8.15 (s, 2H), 8.01 (d, J=8 Hz, 1H), 7.90 (d, J=8 Hz, 1H), 7.46 (t, J=8 Hz, 1H), 7.32 (t, J=8 Hz, 1H), 4.56 (s, 2H), 3.94 (t, J=5 Hz, 2H), 2.88 (bs, 2H).

A mixture of 3-(benzo[d]thiazol-2-yl)-5,7-dihydro-4H-thieno[2,3-c]pyran-2-amine (350 mg, 1.2 mmol), acetic anhydride (0.17 mL, 1.8 mmol) and glacial acetic acid (20 mL) was heated at reflux for 5 h. The mixture was allowed to cool to room temperature and then diluted with water (100 mL). The mixture was neutralized through the addition of a 5% sodium bicarbonate solution and the precipitate that formed was collected by filtration. The filtered solid was dissolved in dichloromethane and washed with water (1×30 mL). The organic phase was dried over magnesium sulfate, filtered, and concentrated to dryness under vacuum. The crude product was triturated with diethyl ether and the collected by filtration to afford 44 mg of N-(3-(benzo[c]thiazol-2-yl)-5,7-dihydro-4H-thieno[2,3-c]pyran-2-yl)acetamide. Yield: 11%; m/z (ESI⁺) 331 (MH⁺); ¹H NMR (200 MHz, d₆-DMSO) δ 12.56 (s, 1H), 8.13 (d, J=8 Hz, 2H), 7.58 (t, J=7 Hz, 1H), 7.45 (t, J=7.6 Hz, 1H), 4.69 (s, 2H), 3.96 (t, J=5.4 Hz, 2H), 2.93 (bs, 2H), 2.32 (s, 3H).

### Examples 49-50

### Ethyl 3-(Benzo[d]thiazol-2-yl)benzofuran-2-carboxylate and 3-(Benzo[d]thiazol-2-yl)benzofuran-2-carboxamide

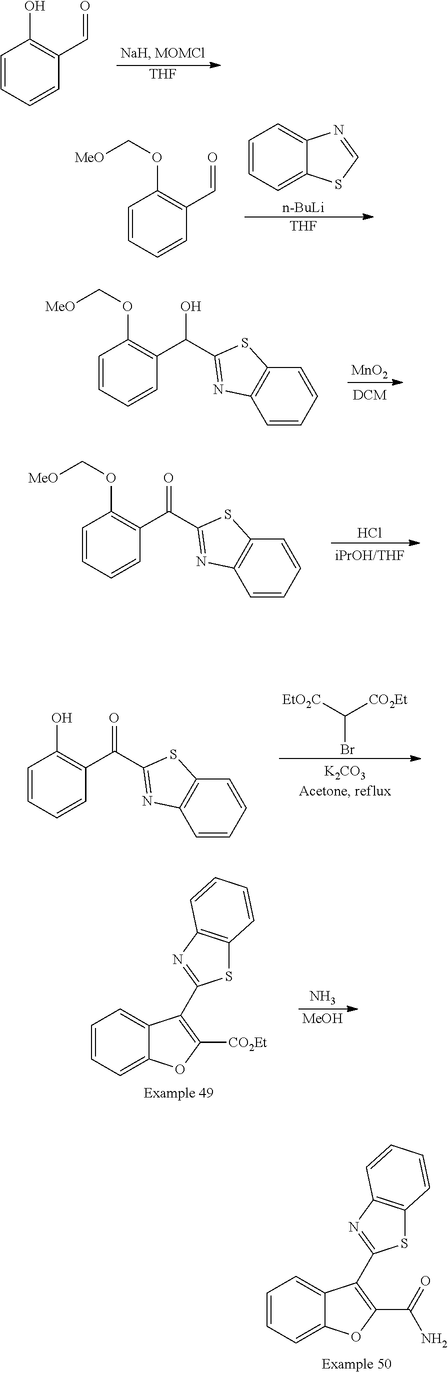

A suspension of sodium hydride (3.92 g, 98.2 mmol) in anhydrous tetrahydrofuran (60 mL) was cooled to 0° C. and then solution of salicylaldehyde (8.62 mL, 82.0 mmol) in tetrahydrofuran (30 mL) was slowly added, taking care to maintain the internal temperature below 5° C. The reaction mixture was allowed to stir for 20 min and then a solution of methoxymethyl chloride (7.33 mL, 98.2 mmol) in tetrahydrofuran (30 mL) was added. The reaction mixture was allowed to warm to room temperature and stir for 64 h. The reaction mixture was poured into water and then condensed in vacuo to half of the original volume. The remaining mixture was extracted with ethyl acetate and the organic phase was washed with brine, dried over magnesium sulfate, filtered, and condensed in vacuo. The crude product was purified by column chromatography with a dichloromethane/methanol mixture (100:1) as eluant affording 11.4 g of 2-(methoxymethoxy)benzaldehyde as a clear oil. Yield=84%.

A stirred solution of benzothiazole (2.18 mL, 19.2 mmol) in dry tetrahydrofuran (50 mL), under an argon atmosphere, was cooled to -78° C. A 2.5M solution of n-butyllithium in hexane (8.42 mL, 21.1 mmol) was added dropwise to the reaction mixture, followed by the dropwise addition of a 2-(methoxymethoxy)benzaldehyde solution (3.5 g, 21.1 mmol) in dry tetrahydrofuran (50 mL) over 1 h. The cold bath was removed and the mixture was allowed to stir an additional 20 min. The reaction mixture was diluted with water (100 mL) and then extracted with diethyl ether. The organic phase was dried over magnesium sulfate, filtered, and then condensed in vacuo to afford a solid. The solid was triturated with diethyl ether and filtered to afford 1.4 g of benzo[d]thiazol-2-yl(2-(methoxymethoxy)phenyl)methanol. Yield=25%; m/z (ESI⁻) 346.

To a mixture of the benzo[d]thiazol-2-yl(2-(methoxymethoxy)phenyl)methanol (1.39 g, 4.61 mmol) in dichloromethane (40 mL) was added activated manganese dioxide (3.10 g, 32.1 mmol). The reaction mixture was allowed to stir at room temperature for 20 h. The reaction mixture was filtered and the filtered precipitate was washed with hot ethyl acetate. The filtrate was condensed in vacuo to afford 1.35 g of crude benzo[d]thiazol-2-yl(2-(methoxymethoxy)phenyl)methanone. Yield=98%.

To a solution of benzo[d]thiazol-2-yl(2-(methoxymethoxy)phenyl)methanone (1.35 g, 4.51 mmol) in a 1:1 mixture of isopropanol/tetrahydrofuran (50 mL) was added concentrated hydrochloric acid (1 mL). The reaction mixture was allowed to stir at 55° C. for 2.5 h. The reaction mixture was condensed to 50% of the original volume in vacuo and was then partitioned between diethyl ether and a 5% sodium bicarbonate solution (50 mL). The organic phase was dried over magnesium sulfate, filtered, and then condensed in vacuo to afford a solid. The solid was triturated with methanol (2×4 mL), and filtered to afford 1.06 g of benzo[d]thiazol-2-yl(2-hydroxyphenyl)methanone. Yield=92%; m/z (ESI ⁺) 256 (MH⁺).

To a mixture of benzo[d]thiazol-2-yl(2-hydroxyphenyl)methanone (0.93 g, 3.64 mmol) and potassium carbonate (5.03 g, 36.4 mmol) in anhydrous acetone (90 mL) was added diethyl bromomalonate (0.675 mL, 3.64 mmol). The mixture was heated at reflux for 5 h and then condensed in vacuo. The remaining residue was portioned between water and dichloromethane. The organic phase was dried over magnesium sulfate, filtered, and condensed in vacuo to afford a solid. The crude product was triturated with methanol (4 mL) and filtered to afford 0.76 g of ethyl 3-(benzo[d]thiazol-2-yl)benzofuran-2-carboxylate. Yield=68%; m/z (ESI⁺) 324 (MH⁺); ¹H NMR (200 MHz, CDCl₃) δ 8.64 (d, J=7 Hz, 1H); 8.20 (d, J=7.4 Hz, 1H), 7.99 (d, J=6.4 Hz, 1H), 7.71-7.44 (m, 5H), 4.55 (q, J=7 Hz, 2H), 1.47 (t, J=7 Hz, 3H).

Ethyl 3-(benzo[d]thiazol-2-yl)benzofuran-2-carboxylate (0.750 g, 2.32 mmol) was dissolved in a solution of methanol that was saturated with ammonia (150 mL). The reaction mixture was allowed to stir at room temperature for 20 h. The reaction mixture was condensed in vacuo and the remaining residue was triturated with diethyl ether to afford 0.66 g of 3-(benzo[d]thiazol-2-yl)benzofuran-2-carboxamide. Yield=96%; m/z (ESI⁺) 295 (MH⁺); ¹H NMR (500 MHz, d₆-DMSO) δ 8.79 (d, J=6 Hz, 1H), 8.60 (s, 1H), 8.25-8.16 (m, 3H), 7.77 (d, J=7.5 Hz, 1H), 7.64 (t, J=8 Hz, 1H), 7.61 (t, J=7.5 Hz, 1H), 7.58-7.51 (m, 2H).

### Example 51

### 2-Acetamideo-3-(benzo[d]thiazol-2-yl)-6-ethyl-4,5,6,7-tetrahydrothieno[2,3-c]pyridine 6-oxide

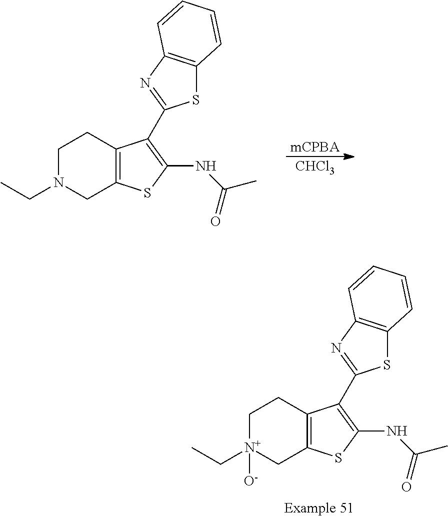

A solution of N-(3-(benzo[d]thiazol-2-yl)-6-ethyl-4,5,6,7-tetrahydrothieno[2,3-c]pyridin-2-yl)acetamide (from Example 2, 80mg, 0.220 mmol) in chloroform (10 mL) was cooled to 0° C. Solid 3-chloro-peroxybenzoic acid (57 mg, 0.33 mmol) was added to the flask in one portion. The reaction mixture was allowed to warm to room temperature and stir for 3 h. The reaction mixture was diluted with dichloromethane (50 mL) and then washed with a 5% sodium bicarbonate solution (2 x 10 mL) and brine (1 x 10 mL). The organic phase was dried over magnesium sulfate, filtered, and concentrated in vacuum to afford a brown oil. The oil was triturated with diethyl ether (2 x 0.5 mL) and then filtered to afford 35 mg of 2-Acetamido-3-(benzo[c]thiazol-2-yl)-6-ethyl-4,5,6,7-tetrahydrothieno[2,3-c]pyridine 6-oxide as a brown solid. Yield: 42%; m/z (ESI⁺) 374 (MH⁺); ¹H NMR (500 MHz, CDCl₃) δ 13.07 (s, 1H), 8.01 (d, J=8 Hz, 1H), 7.95 (d, J=8.5 Hz, 1H), 7.59 (t, J=7.5 Hz, 1H), 7.46 (t, J=7.5 Hz, 1H), 4.55 (q, J=7.5 Hz, 2H), 3.82-3.74 (m, 2H), 3.59-3.54 (m, 3H), 3.21-3.17 (m, 1H), 1.57 (t, J=7.5 Hz, 3H).

### Example 52

### 2-Acetamido-6-ethyl-N-phenyl-4,5,6,7-tetrahydrothieno[2,3-c]pyridine-3-carboxamide

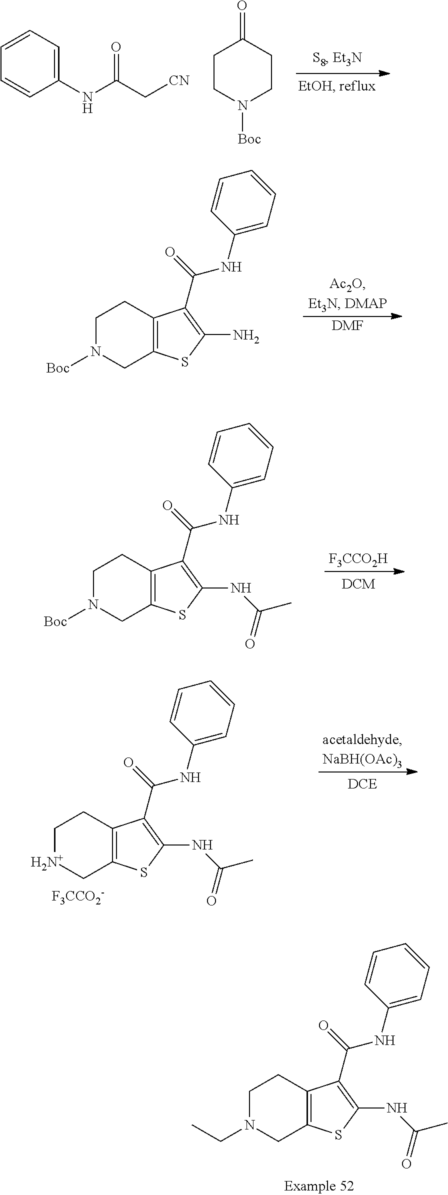

Prepared according to Example 5 using 2-cyano-N-phenylacetamide (5 g, 31.3 mmol), N-Boc-piperidone (6.1 g, 31.3 mmol), sulfur (1 g, 31.3 mmol), triethylamine (6.6 mL, 46.9 mmol), and ethanol (100 mL). The crude product was purified by column chromatography on silica using and ethyl acetate/hexanes gradient (1:10-1:8) to afford 2 g of tert-butyl 2-amino-3-(phenylcarbamoyl)-4,5-dihydrothieno[2,3-c]pyridine-6(7H)-carboxylate as a yellow solid. Yield: 17%; m/z (ESI⁻) 372 (M⁻−H).

Prepared according to Example 5 using tert-butyl 2-amino-3-(phenylcarbamoyl)-4,5-dihydrothieno[2,3-c]pyridine-6(7H)-carboxylate (1 g, 2.67 mmol), N,N-dimethyl-4-aminopyridine (10 mg), acetic anhydride (0.379 mL, 4.02 mmol), triethylamine (1.12 mL, 8.03 mmol), and N,N-dimethylformamide (7 mL). The crude product was crystallized from hexane/ether to afford 800 mg of tert-butyl 2-acetamido-3-(phenylcarbamoyl)-4,5-dihydrothieno[2,3-c]pyridine-6(7H)-carboxylate as an off-white solid. Yield: 72%; m/z (ESI⁺) 416 (MH⁺), 438 (MNa⁺).

Prepared according to Example 6 using tert-butyl 2-acetamido-3-(phenylcarbamoyl)-4,5-dihydrothieno[2,3-c]pyridine-6(7H)-carboxylate (440 mg, 1.06 mmol), trifluoroacetic acid (0.4 mL, 5.30 mmol), and dichloromethane (5 mL). The crude product was washed with diethyl ether and allowed to dry in a stream of air to afford 440 mg of 2-acetamido-3-(phenylcarbamoyl)-4,5,6,7-tetrahydrothieno[2,3-c]pyridin-6-ium 2,2,2-trifluoroacetate. Yield: 99%; m/z (ESI⁻) 317 (MH⁺), 339 (MNa⁺).

Prepared according to Example 7 using 2-acetamido-3-(phenylcarbamoyl)-4,5,6,7-tetrahydrothieno[2,3-c]pyridin-6-ium 2,2,2-trifluoroacetate (0.3 g, 0.698 mmol), acetaldehyde (0.063 mL, 1.12 mmol), sodium triacetoxyborohydride (297 mg, 1.40 mmol), and dichloroethane (5 mL). The crude product was crystallized from hexanes/diethyl ether to afford 61 mg of 2-acetamido-6-ethyl-N-phenyl-4,5,6,7-tetrahydrothieno[2,3-c]pyridine-3-carboxamide as a yellow solid. Yield: 42%; m/z (ESI⁺) 344 (MH⁻), 366 (MNa⁺); ¹H NMR (500 MHz, CDCl₃) δ 11.76 (s, 1H), 7.55 (s, 1H), 7.50 (d, J=8.5 Hz, 2H), 7.39 (t, J=8 Hz, 2H), 7.18 (t, J=7.5 Hz, 1H), 3.62 (s, 2H), 2.99 (s, 2H), 2.85 (t, J=5.5 Hz, 2H), 2.65 (q, J=7 Hz, 2H), 2.24 (s, 3H), 1.21 (t, J=7 Hz, 3H)

### Example 53

### N-(3-(Benzo[d]oxazol-2-yl)-6-ethyl-4,5,6,7-tetrahydrothieno[2,3-c]pyridin-2-yl)acetamide

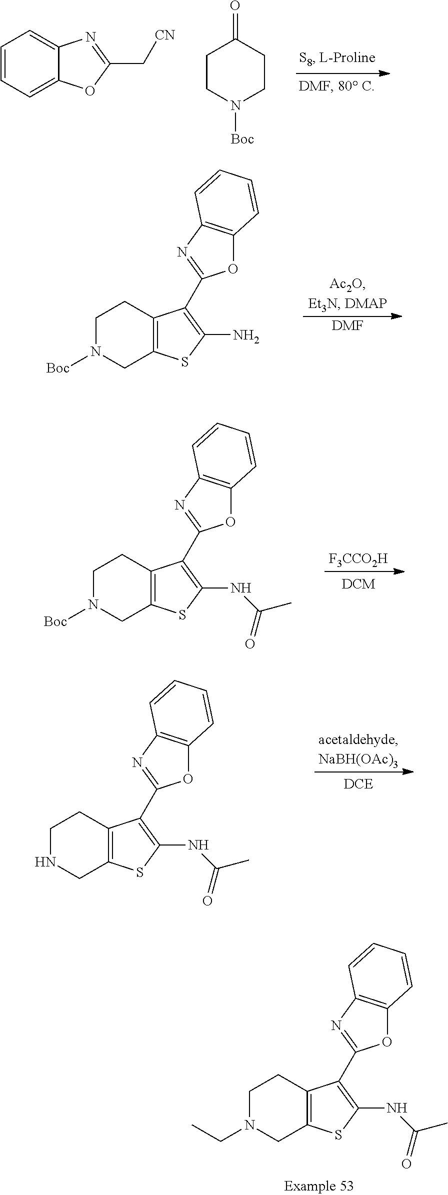

A mixture of (1,3-benzoxazol-2-yl)acetonitrile (1.01 g, 6.3 mmol), N-Boc-4-piperidone (1.26 g, 6.3 mmol), sulfur (300 mg, 10 mmol), and L-proline (72 mg, 0.63 mmol) in N,N-dimethylformamide (25 mL) was allowed to stir at 80° C. for 12 h. After cooling to room temperature the reaction mixture was condensed in vacuo and the remaining residue was purified by column chromatography on silica using a mixture of ethyl acetate/hexanes (1:4) as the eluant to obtain 0.899 g of tert-butyl 2-amino-3-(benzo[d]oxazol-2-yl)-4,5-dihydrothieno[2,3-c]pyridine-6(7H)-carboxylate. Yield: 40%; m/z (ESI⁺) 372 (MH⁺)

A mixture of tert-butyl 2-amino-3-(benzo[c/]oxazol-2-yl)-4,5-dihydrothieno[2,3-c]pyridine-6(7H)-carboxylate (1.05 g, 2.82 mmol), N,N-dimethyl-4-aminopyridine (35 mg, 0.282 mmol), triethylamine (1.3 mL, 8.46 mmol), and acetic anhydride (0.4 mL, 0.431 mmol) in N,N-dimethylformamide (20 mL) was allowed to stir at 80° C. for 2 d. After cooling to room temperature the reaction mixture was condensed in vacuo. The remaining residue was purified by column chromatography on silica using a mixture of ethyl acetate/hexanes (1:5) as the eluant to afford 0.728 g of tert-butyl 2-acetamido-3-(benzo[c/]oxazol-2-yl)-4,5-dihydrothieno[2,3-c]pyridine-6(7H)-carboxylate. Yield: 62%; m/z (ESI⁺) 436 (MNa⁺).

A solution of tert-butyl 2-acetamido-3-(benzo[c/]oxazol-2-yl)-4,5-dihydrothieno[2,3-c]pyridine-6(7H)-carboxylate (0.728 g, 1.76 mmol) in dichloromethane (8 mL) was treated with trifluoroacetic acid (8 mL). The reaction mixture was allowed to stir for 4 h and then the reaction was quenched through the addition of a 4M sodium hydroxide solution (final pH=10). The mixture was extracted with dichloromethane (3×10 mL) and the combined organic phases were washed with brine, dried over magnesium sulfate, filtered, and condensed to afford 0.550 g of N-(3-(benzo[c/]oxazol-2-yl)-4,5,6,7-tetrahydrothieno[2,3-c]pyridin-2-yl)acetamide. Yield: 99%; m/z (ESI⁺) 314 (MH⁺).

A solution of N-(3-(benzo[c/]oxazol-2-yl)-4,5,6,7-tetrahydrothieno[2,3-c]pyridin-2-yl)acetamide (0.215 g, 0.686 mmol) in dichloroethane (5 mL) was sparged with argon for 30 min to remove oxygen. Acetic acid (0.6 mL, 1.03 mmol) and acetaldehyde (0.120 mL, 2.06 mmol) were added, followed by sodium triacetoxyborohydride (0.218 g, 1.03 mmol). The reaction mixture was allowed to stir for 12 h at room temperature. The reaction was quenched through the addition of a degassed solution of saturated aqueous sodium bicarbonate (5 mL) and the resulting mixture was allowed to stir for 30 min. The phases were separated and the aqueous layer was extracted with dichloromethane (3 x 10 mL). The combined organic phases were washed with brine, dried over magnesium sulfate, filtered, and condensed in vacuo to afford 0.254 g of pure N-(3-(benzo[c/]oxazol-2-yl)-6-ethyl-4,5,6,7-tetrahydrothieno[2,3-c]pyridin-2-yl)acetamide. Yield: 85%; m/z (ESI⁺) 342 (MH⁺); ¹H NMR (500 MHz, CDCl₃) δ 12.04 (s,1H), 7.69 (d, J=7.5 Hz, 1H), 7.55 (d, J=15 Hz, 1H), 7.37-7.31 (m, 2H), 3.65 (s, 2H), 3.19 (t, J=6 Hz, 2H), 2.88 (t, J=6 Hz, 2H), 2.67 (q, J=7.5 Hz, 2H), 2.39 (s, 3H), 1.22 (t, J=7.5 Hz, 3H).

### Example 54

### 2-Acetamido-3-(benzo[d]thiazol-2-yl)-6,6-diethyl-4,5,6,7-tetrahydrothieno[2,3-c]pyridin-6-ium Trifluoromethanesulfonate

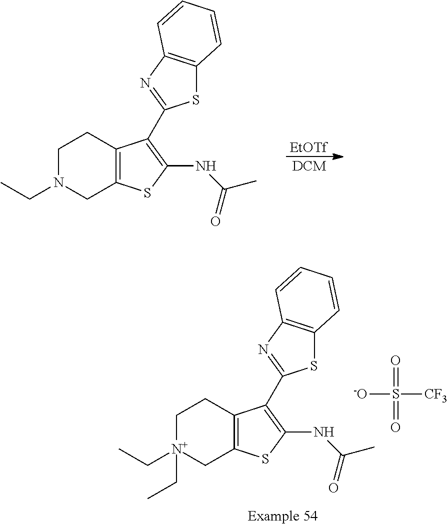

A solution of N-(3-(benzo[c]thiazol-2-yl)-6-ethyl-4,5,6,7-tetrahydrothieno[2,3-c]pyridin-2-yl)acetamide (from Example 2, 200 mg, 0.560 mmol) in dichloromethane (10 mL) was cooled to 0° C. and then ethyl trifluoromethanesulfonate (0.08 mL, 0.590 mmol) was added. The reaction mixture was allowed to warm to room temperature and stir for 2 h. The precipitate that formed was collected by filtration and was washed with dichloromethane (2×0.5 mL). The solid was dried under vacuum to afford 254 mg of 2-acetamido-3-(benzo[d]thiazol-2-yl)-6,6-diethyl-4,5,6,7-tetrahydrothieno[2,3-c]pyridin-6-ium trifluoromethanesulfonate as a grey solid. Yield: 85%; m/z (ESI⁺) 386 (M⁺); ¹H NMR (500 MHz, d₆-DMSO) δ 12.56 (s, 1H), 8.21 (dd, J=Hz, 2H), 7.62 (t, J=Hz, 1H), 7.52 (t, J=Hz, 1H), 4.68 (s, 2H), 3.80 (t, J=Hz, 2H), 3.48 (t, J=Hz, 2H), 2.37 (s, 3H), 1.31 (t, J=Hz, 3H)

### Examples 55-56

### N-(3-(Benzo[d]thiazol-2-yl)thieno[2,3-c]pyridin-2-yl)acetamide and 2-Acetamido-3-(benzo[c]thiazol-2-yl)-6-ethylthieno[2,3-c]pyridin-6-ium Trifluoromethanesulfonate

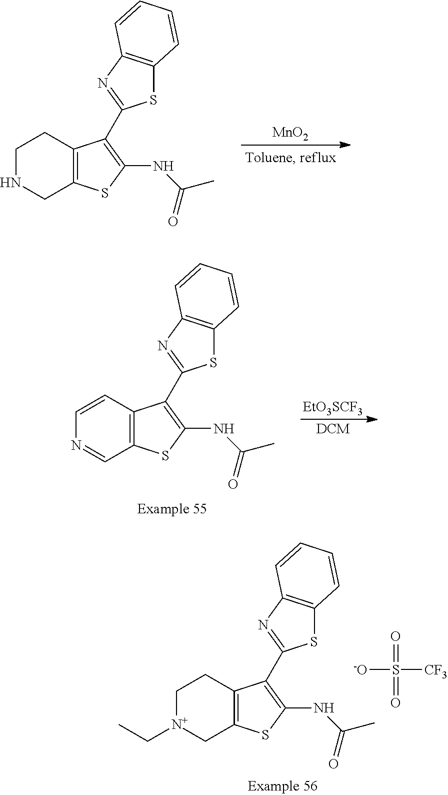

A solution of N-(3-(benzo[c]thiazol-2-yl)-4,5,6,7-tetrahydrothieno[2,3-c]pyridin-2-yl)acetamide (from Example 6, 100 mg, 0.304 mmol) in toluene (5 mL) was treated with manganese dioxide (400 mg, 4.56 mmol). The reaction mixture was heated at reflux for 6 h. The reaction mixture was filtered through a pad of Celite and the filtrate was condensed in vacuo to afford 80 mg of N-(3-(benzo[c/]thiazol-2-yl)thieno[2,3-c]pyridin-2-yl)acetamide as a brown solid. Yield: 80%; m/z (ESI⁺) 326; ¹H NMR (200 MHz, d₆-DMSO) δ 9.36 (s, 1H), 8.62 (s, 1H), 8.26 (d, J=6 Hz, 1H), 8.19 (d, J=7.6 Hz, 1H), 8.17 (d, J=7.6 Hz, 1H), 7.64-7.45 (m, 2H), 2.42 (s, 3H).

Ethyl trifluoromethanesulfonate (90 mg, 0.508 mmol) was added to a solution of N-(3-(benzo[d]thiazol-2-yl)thieno[2,3-c]pyridin-2-yl)acetamide in dichloromethane (2 mL) that had been cooled to 0° C. The mixture was allowed to warm to room temperature and stir for 2 h. The precipitate that formed was collected by filtration and then washed with dichloromethane and diethyl ether. Drying the solid in a stream of nitrogen afforded 187 mg of 2-acetamido-3-(benzo[d]thiazol-2-yl)-6-ethylthieno[2,3-c]pyridin-6-ium trifluoromethanesulfonate as a yellow/green solid. Yield: 70%; m/z (ESI⁺) 354 (M⁺); ¹H NMR (500 MHz, d₆-DMSO) δ 13.19 (s, 1H), 9.71 (s, 1H), 8.85 (s, 1H), 8.49 (s, 1H), 8.26 (s, 2H), 7.67 (s, 1H), 7.58 (s, 1H), 4.64 (s, 2H), 1.58 (t, J=7.5 Hz, 3H).

### Example 57

### 2-Acetamido-3-(benzo[c]thiazol-2-yl)thieno[2,3-c]pyridine 6-oxide

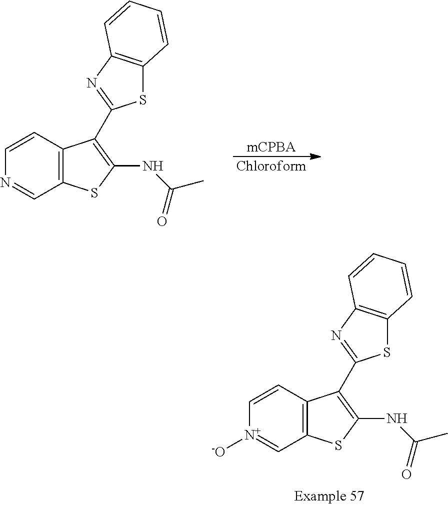

A solution of N-(3-(benzo[c]thiazol-2-yl)thieno[2,3-c]pyridin-2-yl)acetamide (from Example 55, 80 mg, 0.246 mmol) in chloroform (1 mL) was cooled to 0° C. and then treated with 3-chloroperoxybenzoic acid (80 mg, 0.464 mmol). The mixture was stirred at the reduced temperature for 3 h. The mixture was diluted with chloroform and washed with a 5% solution of aqueous sodium bicarbonate. The organic phase was dried over magnesium sulfate, filtered, and condensed in vacuo to afford 80 mg of 2-acetamido-3-(benzo[d]thiazol-2-yl)thieno[2,3-c]pyridine 6-oxide as a yellow solid. Yield: 93%; m/z (ESI⁺) 352 (MH⁺); ¹H NMR (500 MHz, CDCl₃) δ 8.66 (s, 1H), 8.28 (d, J=7 Hz, 1H), 8.05 (d, J=7.5 Hz, 1H), 8.03 (d, J=9 Hz, 1H), 7.99 (d, J=7.5 Hz, 1H), 7.60 (t, J=7.5 Hz, 1H), 7.49 (t, J=7.5 Hz, 1H), 2.48 (s, 3H).

### Example 58-59

### 2-Acetamido-3-(2H-tetrazol-5-yl)-4,5,6,7-tetrahydrothieno[2,3-c]pyridin-6-ium 2,2,2-Trifluoroacetate and 2-Acetamido-3-(2-methyl-2H-tetrazol-5-yl)-4,5,6,7-tetrahydrothieno[2,3-c]pyridin-6-ium 2,2,2-Trifluoroacetate

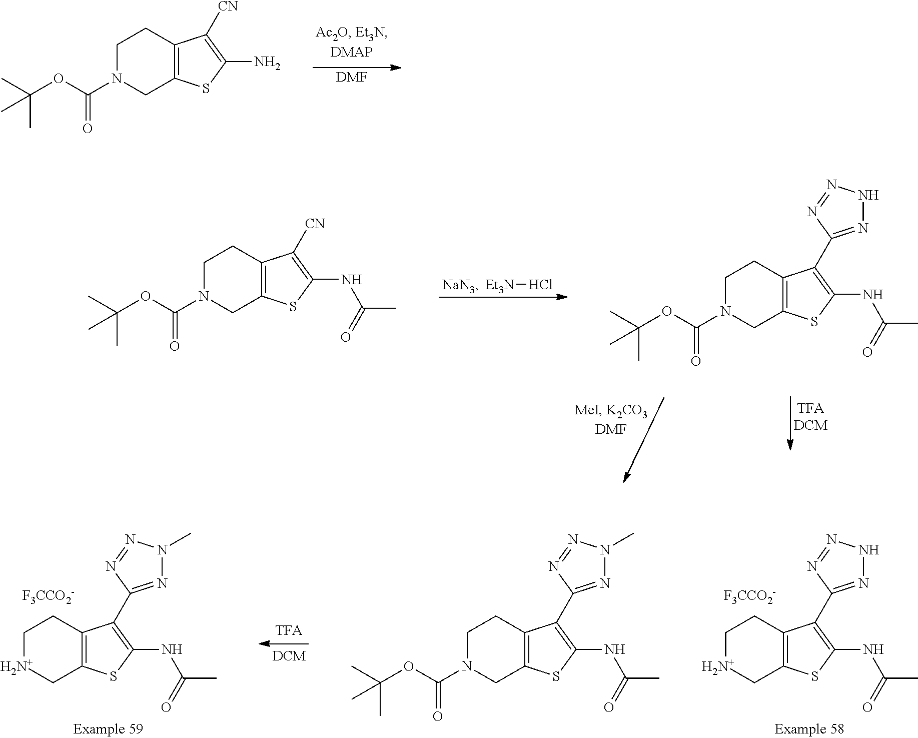

A mixture of tert-butyl 2-amino-3-cyano-4,5-dihydrothieno[2,3-c]pyridine-6(7H)-carboxylate (purchased from Enamine/catalog # EN300-23852, 4 g, 14.0 mmol), triethylamine (6 mL, 43.0 mmol), N,N-dimethyl-4-amino-pyridine (0.35 g, 2.80 mmol), and acetic anhydride (7.1 mL, 70.0 mmol) in N,N-dimethylformamide (20 mL) was allowed to stir at room temperature for 24 h. The reaction mixture was condensed in vacuo and the remaining residue was dissolved in dichloromethane. The mixture was washed with water, 1M hydrochloric acid, and a 5% aqueous sodium bicarbonate solution. The organic phase was dried over magnesium sulfate, filtered, and condensed in vacuo. The remaining black oil was triturated with diethyl ether and the solid that formed was collected by filtration. The solid was washed with additional diethyl ether and allowed to dry in a stream to afford 1.5 g of tert-butyl 2-acetamido-3-cyano-4,5-dihydrothieno[2,3-c]pyridine-6(7H)-carboxylate as a gray solid. Yield: 35%; m/z (ESI⁻) 320 (M⁻−H).

A mixture of tert-butyl 2-acetamido-3-cyano-4,5-dihydrothieno[2,3-c]pyridine-6(7H)-carboxylate (0.5 g, 1.55 mmol), sodium azide (1 g, 15.5 mmol), and triethylamine hydrochloride (0.95 g, 1.86 mmol), in N,N-dimethylformamide (5 mL) was heated at 150° C. for 4 h. The reaction mixture was condensed in vacuo and the remaining residue was dissolved in dichloromethane. The mixture was washed with water, dried over magnesium sulfate, filtered, and condensed in vacuo to afford 300 mg of tert-butyl 2-acetamido-3-(2H-tetrazol-5-yl)-4,5-dihydrothieno[2,3-c]pyridine-6(7H)-carboxylate as a light yellow solid. Yield: 66%; m/z (ESI⁻) 363 (M⁻−H).

A solution of tert-butyl 2-acetamido-3-(2H-tetrazol-5-yl)-4,5-dihydrothieno[2,3-c]pyridine-6(7H)-carboxylate (60 mg, 0.165 mmol) in dichloromethane (2 mL) was treated with trifluoroacetic acid (0.5 mL). The reaction mixture was allowed to stir at room temperature for 3 h. The reaction mixure was condensed in vacuo and the remaining residue was triturated with diethyl ether. The solid was collected by filtration and allowed to dry in a stream of air to afford 10 mg of 2-acetamido-3-(2H-tetrazol-5-yl)-4,5,6,7-tetrahydrothieno[2,3-c]pyridin-6-ium 2,2,2-trifluoroacetate as an off white solid. Yield 16%; m/z (ESI⁺) 265 (M⁺); ¹H NMR (600 MHz, d₆-DMSO) δ 11.86 (s, 1H), 11.29 (s, 1H), 9.30 (bs, 2H), 4.27 (bs, 2H), 3.39 (bs, 2H), 3.12 (bs, 2H), 2.19 (s, 3H).

A mixture of tert-butyl 2-acetamido-3-(2H-tetrazol-5-yl)-4,5-dihydrothieno[2,3-c]pyridine-6(7H)-carboxylate (0.2 g, 0.550 mmol), potassium carbonate (0.15 g, 1.10 mmol), and methyl iodide (0.18 mL, 2.95 mmol) in N,N-dimethylformamide (3 mL) was allowed to stir at room temperature for 24 h. The reaction mixture was condensed in vacuo and the remaining residue was dissolved in dichloromethane. The mixture was washed with water, dried over magnesium sulfate, filtered, and condensed in vacuo to afford 130 mg of tert-butyl 2-acetamido-3-(2-methyl-2H-tetrazol-5-yl)-4,5-dihydrothieno[2,3-c]pyridine-6(7H)-carboxylate as an off-white solid. Yield: 62%; m/z (ESI⁺) 401 (MNa⁺), 379 (MH⁺).

A solution of tert-butyl 2-acetamido-3-(2-methyl-2H-tetrazol-5-yl)-4,5-dihydrothieno[2,3-c]pyridine-6(7H)-carboxylate (120 mg, 0.317 mmol) in dichloromethane (2 mL) was treated with trifluoroacetic acid (0.5 mL). The reaction mixture was allowed to stir at room temperature for 2 h and was then concentrated in vacuo. The remaining residue was triturated with diethyl ether and the resulting precipitate was collected via filtration to afford 120 mg of 2-acetamido-3-(2-methyl-2H-tetrazol-5-yl)-4,5,6,7-tetrahydrothieno[2,3-c]pyridin-6-ium 2,2,2-trifluoroacetate as an off-white solid. Yield: 96%; m/z (ESI⁺) 279 (MH⁺); ¹H NMR (600 MHz, d₆-DMSO) δ 10.73 (s, 1H), 9.39 (bs, 2H), 4.44 (s, 3H), 4.30 (s, 2H), 3.40 (t, J=4.9 Hz, 2H), 3.02 (t, J=5.9 Hz, 2H), 2.22 (s, 3H).

### Examples 60-61

### 2-Acetamido-3-(benzo[d]thiazol-2-yl)-7-ethyl-5,6,7,8-tetrahydro-4H-thieno[2,3-c]azepin-7-ium Carboxyformate and 2-Acetamido-3-(benzo[d]thiazol-2-yl)-7-methyl-5,6,7,8-tetrahydro-4H-thieno[2,3-c]azepin-7-ium Carboxyformate

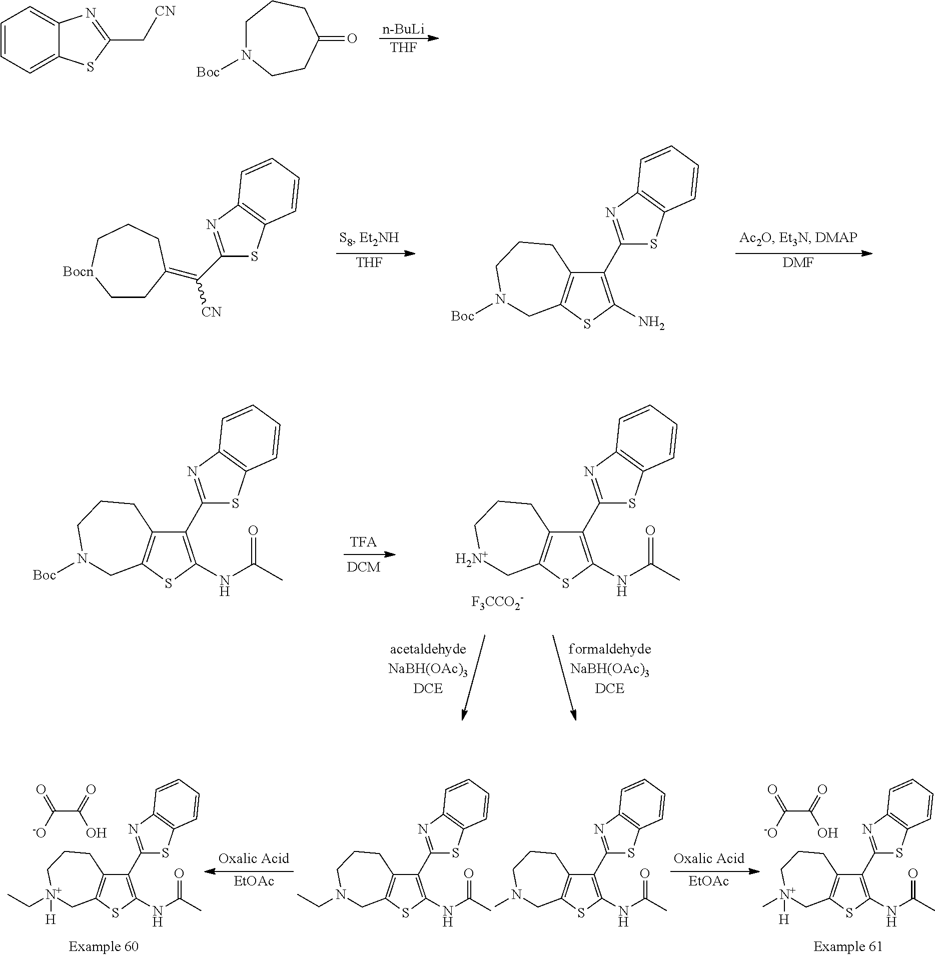

Solid 2-(benzo[d]thiazol-2-yl)acetonitrile (0.5 g, 2.87 mmol) was dissolved in anhydrous tetrahydrofuran (5 mL) and the solution was cooled to −70° C. In a dropwise fashion, the solution was treated with a solution of n-butyllithium (2.5M in hexane, 1.15 mL, 2.87 mmol) and, after 20 min, a solution of tert-butyl 4-oxoazepane-l-carboxylate (0.6 g, 2.87 mmol) in anhydrous tetrahydrofuran (3 mL) was added in the same manner. The reaction mixture was allowed to warm to room temperature and stir for 1 h. The reaction mixture was diluted with ethyl acetate (10 mL) and 1M hydrochloric acid and the resulting mixture was stirred vigorously for 10 min. The organic layer was washed with brine, dried over magnesium sulfate, filtered, and condensed in vacuo. The remaining residue was purified by column chromatography on silica using a mixture of ethyl acetate:hexanes (1:50) as the eluant to afford 0.8 g of tert-butyl 4-(benzo[d]thiazol-2-yl(cyano)methylene)azepane-1-carboxylate. Yield: 80%; m/z (ESI⁺) 392 (MNa⁺), 370 (MH⁺).

A mixture of tert-butyl 4-(benzo[d]thiazol-2-yl(cyano)methylene)azepane-1-carboxylate (0.5 g, 1.35 mmol), sulfur (44 mg, 1.35 mmol), and diethylamine (0.49 mL, 4.74 mmol) in tetrahydrofuran (4 mL) was allowed to stir at room temperature. The progress of the reaction was followed by thin layer chromatography and when all of the nitrile had been consumed the reaction mixture was filtered. The filtrate was evaporated to dryness and product the remaining residue was triturated with hexanes. The solids were collected by filtration and then recrystallized from methanol to afford 0.5 g of tert-butyl 2-amino-3-(benzo[c]thiazol-2-yl)-5,6-dihydro-4H-thieno[2,3-c]azepine-7(8H) carboxylate. Yield: 92%; m/z (ESI⁺) 402 (MH⁺), 346 (MH⁺−C₄H₈).

To a solution of tert-butyl 2-amino-3-(benzo[d]thiazol-2-yl)-5,6-dihydro-4H-thieno[2,3-c]azepine-7(8H)-carboxylate (180 mg, 0.45 mmol) in N,N-dimethylformamide (3 mL) was added acetic anhydride (0.21 mL, 2.25 mmol), triethylamine (0.17 mL, 1.35 mmol), and N,N-dimethyl-4-aminopyridine (50 mg, 0.45 mmol). The mixture was allowed to stir at room temperature for 2 d before it was concentrated in vacuo. The remaining residue was dissolved in dichloromethane and washed with water, 1M hydrochloric acid, and 1M sodium hydroxide. The organic phase was dried over magnesium sulfate, filtered, and concentrated in vacuum. The residue was purified by column chromatography on silica using a hexane/ethyl acetate mixture and the eluant to affod 120 mg of tert-butyl 2-acetamido-3-(benzo[d]thiazol-2-yl)-5,6-dihydro-4H-thieno[2,3-c]azepine-7(8H)-carboxylate as a yellow precipitate. Yield=60%; m/z (ESI⁻) 444 (MH⁺).

To a solution of tert-butyl 2-acetamido-3-(benzo[d]thiazol-2-yl)-5,6-dihydro-4H-thieno[2,3-c]azepine-7(8H)-carboxylate (120 mg, 0.27 mmol) in dichloromethane (3 mL) was added trifluoroacetic acid (0.2 mL, 2.7 mmol). The mixture was allowed to stir at room temperature for 24 h. The reaction mixture was concentrated in vacuo and the remaining solid was triturated with diethyl ether. The solid was collected by filtration and allowed to dry in a stream of air to afford 77 mg of 2-acetamido-3-(benzo[c]thiazol-2-yl)-5,6,7,8-tetrahydro-4H-thieno[2,3-c]azepin-7-ium 2,2,2-trifluoroacetate. Material was used immediately in the next reaction. Yield=62%.

To a solution of 2-acetamido-3-(benzo[c]thiazol-2-yl)-5,6,7,8-tetrahydro-4H-thieno[2,3-c]azepin-7-ium (80 mg, 0.170 mmol) in 1,2-dichloroethane (3 mL) was added acetaldehyde (0.02 mL, 0.340 mmol). The reaction mixture was allowed to stir at room temperature for 1 h and then sodium triacetoxyborohydride (50 mg, 0.240 mmol) was added portionwise over 5 min. The reaction mixture was allowed to stir at room temperature for 2 d.

The reaction mixture was then diluted with dichloromethane (20 mL) and washed with a 5% sodium bicarbonate solution and brine. The organic phase was dried over magnesium sulfate, filtered, and then concentrated in vacuo to afford 39 mg of N-(3-(benzo[d]thiazol-2-yl)-7-ethyl-5,6,7,8-tetrahydro-4H-thieno[2,3-c]azepin-2-yl)acetamide. Yield: 62%; m/z (ESI⁺) 372 (MH⁺).

To a solution of N-(3-(benzo[c]thiazol-2-yl)-7-ethyl-5,6,7,8-tetrahydro-4H-thieno[2,3-c]azepin-2-yl)acetamide (53 mg, 0.140 mmol) in ethyl acetate (2 mL) was added solution of oxalic acid (13 mg, 0.140 mmol) in ethyl acetate. The reaction mixture was allowed to stir at room temperature for 10 minutes. The precipitate that formed was collected by filtration and then recrystallized from methanol/diethyl ether to afford 12 mg of 2-acetamido-3-(benzo[c]thiazol-2-yl)-7-ethyl-5,6,7,8-tetrahydro-4H-thieno[2,3-c]azepin-7-ium carboxyformate as a pale yellow solid. Yield: 39%; m/z (ESI⁺) 372 (M⁺); ¹H NMR (600 MHz, d₆-DMSO) δ 11.80 (s, 1H), 8.16 (d, J=10.2 Hz, 1H), 8.14 (d, J=10.8 Hz, 1H), 7.58 (t, J=9 Hz, 1H), 7.49 (t, J=9 Hz, 1H), 4.49 (s, 2H), 3.45 (s, 2H), 3.10 (s, 2H), 3.01 (s, 2H), 2.21 (s, 3H), 1.95 (s, 2H), 1.20 (bs, 3H).

To a solution of 2-acetamido-3-(benzo[c]thiazol-2-yl)-5,6,7,8-tetrahydro-4H-thieno[2,3-c]azepin-7-ium (150 mg, 0.430 mmol) in dichloroethane (3 mL) was added a 36% aqueous formaldehyde solution (0.05 mL, 1.74 mmol). This reaction mixture was allowed to stir at room temperature for 2 h and then sodium triacetoxyborohydride (130 mg, 0.610 mmol) was added portionwise over 5 min. The resulting mixture was allowed to stir at room temperature for 24 h. The reaction mixture was concentrated in vacuo and the remaining reissue was dissolved in dichloromethane. The mixture was washed with water, a 5% aqueous sodium bicarbonate solution, and brine. The organic phase was dried over magnesium sulfate, filtered and condensed in vacuo to afford 117 mg of N-(3-(benzo[d]thiazol-2-yl)-7-methyl-5,6,7,8-tetrahydro-4H-thieno[2,3-c]azepin-2-yl)acetamide. Material was immediately used in the next step. Yield=76%.

To a solution of N-(3-(benzo[c]thiazol-2-yl)-7-methyl-5,6,7,8-tetrahydro-4H-thieno[2,3-c]azepin-2-yl)acetamide (140 mg, 0.39 mmol) in ethyl acetate (3 mL) was added a solution of oxalic acid (40 mg, 0.43 mmol) in ethyl acetate (1 mL). The reaction mixture was allowed to stir at room temperature for 10 minutes. The precipitate that formed was collected via filtration and then washed with ethyl acetate and diethyl ether. The solid was recrystallized from methanol/diethyl ether to afford 100 mg of 2-acetamido-3-(benzo [c]thiazol-2-yl)-7-methyl-5,6,7,8-tetrahydro-4H-thieno[2,3-c]azepin-7-ium carboxyformate as a pale yellow solid. Yield=58%; m/z (ESI⁺) 358 (M⁺); ¹H NMR (200 MHz, d₆-DMSO) δ 11.77 (s, 1H), 8.16-8.10 (m, 2H), 7.60-7.43 (m, 2H), 4.45 (s, 2H), 4.01 (s, 2H), 3.43 (s, 2H), 2.81 (s, 3H), 2.17 (s, 3H), 1.93 (s, 2H).

### Example 62

### 2-(2-Chlorobenzamido)-6-ethyl-4,5,6,7-tetrahydrothieno[2,3-c]pyridine-3-carboxamide

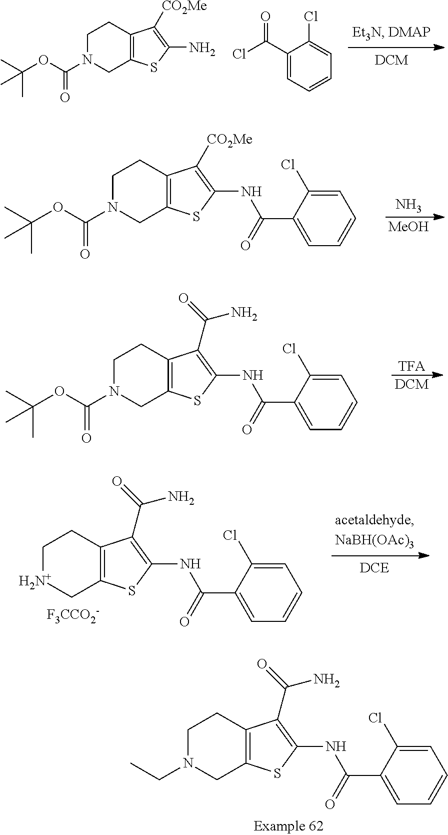

A mixture of 6-tert-butyl 3-methyl 2-amino-4,5-dihydrothieno[2,3-c]pyridine-3,6(7H)-dicarboxylate (purchased from Enamine/catalog number EN300-23853, 2 g, 6.40 mmol), triethylamine (1.34 mL, 9.60 mmol), and N,N-dimethyl-4-aminopyridine (0.16 g, 1.30 mmol) in dichloromethane (20 mL) was cooled to 0° C. Neat 2-chlorobenzoyl chloride (0.97 mL, 7.70 mmol) was added dropwise to the reaction mixture and at the end of the addition the mixture was allowed to warm to room temperature. The progress of the reaction was monitored by thin layer chromatography. When analyses showed that the starting amine had been consumed the reaction mixture was diluted with dichloromethane (150 mL) and washed with water, 0.5M hydrochloric acid, a 5% aqueous solution of sodium bicarbonate, and brine. The organic phase was dried over magnesium sulfate, filtered, and condensed in vacuo. The remaining residue was purified by column chromatography on silica using an ethyl acetate/hexane (1:100) mixture as the eluant to afford 1.58 g of 6-tert-butyl 3-methyl 2-(2-chlorobenzamido)-4,5-dihydrothieno[2,3-c]pyridine-3,6(7H)-dicarboxylate as a white solid. Yield: 55%; m/z (ESI⁺) 475/473 (Cl³⁷ MNa⁺/Cl³⁵ MNa⁺).

Solid 6-tert-butyl 3-methyl 2-(2-chlorobenzamido)-4,5-dihydrothieno[2,3-c]pyridine-3,6(7H)-dicarboxylate (1.2 g, 2.66 mmol) was dissolved in a 5.8M solution of ammonia in methanol (40 mL) and was heated at 60° C. The reaction mixture was allowed to stir at the elevated temperature for 6 d and was then condensed in vacuo. The remaining solid was triturated with dichloromethane and collected by filtration to afford 0.845 g of tert-butyl 3-carbamoyl-2-(2-chlorobenzamido)-4,5-dihydrothieno[2,3-c]pyridine-6(7H)-carboxylate as a white solid. Yield: 73%; m/z (ESI⁺) 460/458 (Cl³⁷MNa⁺/Cl³⁵ MNa⁺).

Solid tert-butyl 3-carbamoyl-2-(2-chlorobenzamido)-4,5-dihydrothieno[2,3-c]pyridine-6(7H)-carboxylate (0.6 g, 1.46 mmol) was suspended in dichloromethane (10 mL) and then treated with trifluoroacetic acid (0.5 mL). The resulting mixture was allowed to stir for 3 d at room temperature and was then condensed in vacuo. The remaining solid was triturated with diethyl ether and then collected by filtration to afford 0.53 g of 3-carbamoyl-2-(2-chlorobenzamido)-4,5,6,7-tetrahydrothieno[2,3-c]pyridin-6-ium 2,2,2-trifluoroacetate as a white solid. Yield 82%; m/z (ESI⁺) 338/336 (Cl³⁷MNa⁺/Cl³⁵ MNa⁺).

Solid 3-carbamoyl-2-(2-chlorobenzamido)-4,5,6,7-tetrahydrothieno[2,3-c]pyridin-6-ium 2,2,2-trifluoroacetate (0.37 g, 0.80 mmol) was suspended in 1,2-dichloroethane (6 mL) and then treated with acetaldehyde (0.07 mL, 1.30 mmol). The resulting mixture was allowed to stir for 4 h at room temperature and then an additional amount of acetaldehyde (0.046 mL) was added. After 5 min, solid sodium triacetoxyborohydride (0.24 g, 1.10 mmol) was added portionwise to the reaction mixture. The resulting mixture was allowed to stir at room temperature for 24 h. The reaction mixture was diluted with dichloromethane and was washed with a 5% aqueous sodium bicarbonate solution. The organic phase was dried over magnesium sulfate, filtered, and condensed in vacuo. The remaining solid was triturated with diethyl ether and collected by filtration to afford 0.186 g of 2-(2-chlorobenzamido)-6-ethyl-4,5,6,7-tetrahydrothieno[2,3-c]pyridine-3-carboxamide as a brown solid. Yield: 64%; m/z (ESI⁺) 366/364 (Cl³⁷MH⁺/Cl³⁵ MH⁺); ¹H NMR (600 MHz, d₆-DMSO) δ 12.35 (s, 1H), 7.68 (d, J=7.8 Hz, 1H), 7.59-7.50 (bm, 2H), 7.46 (t, J=8.4 Hz, 1H), 3.52 (s, 2H), 2.80 (s, 2H), 2.67 (s, 2H), 2.51 (s, 2H), 1.07-1.04 (m, 3H).

### Example 63

### N-(6-Ethyl-4,5,6,7-tetrahydrothieno[2,3-c]pyridin-2-yl)acetamide

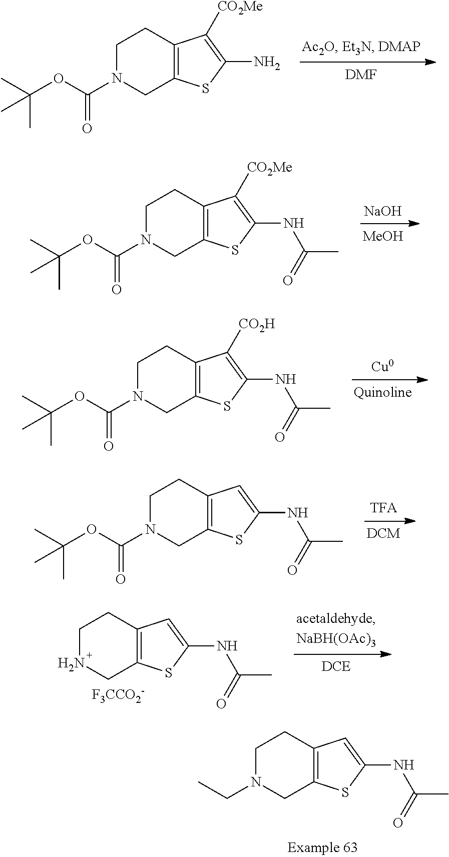

A mixture of 6-tert-butyl 3-methyl 2-amino-4,5-dihydrothieno[2,3-c]pyridine-3,6(7H)-dicarboxylate (purchased from Enamine/catalog number EN300-23853, 5 g, 0.016 mol), N,N-dimethyl-4-aminopyridine (0.39 g, 0.003 mol), triethylamine (6.7 mL, 0.048 mol), and acetic anhydride (2.27 mL, 0.024 mol) in N,N-dimethylformamide (30 mL) was allowed to stir at room temperature for 24 h. The reaction mixture was condensed in vacuo and the remaining residue was dissolved in dichloromethane. The mixture was washed with water, 0.5M hydrochloric acid, and a 5% aqueous solution of sodium bicarbonate. The organic phase was dried over magnesium sulfate, filtered, and condensed in vacuo to afford 5.6 g of crude 6-tert-butyl 3-methyl 2-acetamido-4,5-dihydrothieno[2,3-c]pyridine-3,6(7H)-dicarboxylate as a brown residue. The material was taken onto the next reaction without further purification. Yield: 99%; m/z (ESI⁺) 377 (MNa⁺).

Crude 6-tert-butyl 3-methyl 2-acetamido-4,5-dihydrothieno[2,3-c]pyridine-3,6(7H)-dicarboxylate (5.6 g, 0.016 mol) was suspended in methanol (100 mL) and a 1M aqueous solution of sodium hydroxide (100 mL) was added. The resulting mixture was allowed to stir at room temperature for 2 d and was then heated at 40° C. for 4 h. The reaction mixture was condensed to half of the original volume under reduced pressure and the remaining aqueous mixture was washed with diethyl ether. The aqueous phase was then neutralized with 1M hydrochloric acid and the precipitate that formed was collected via filtration. After drying in a stream of air the 3.27 g of crude 2-acetamido-6-(tert-butoxycarbonyl)-4,5,6,7-tetrahydrothieno[2,3-c]pyridine-3-carboxylic acid was obtained as a brown solid. Yield: 60%; m/z (ESI⁻) 339 (M⁻−H).

A mixture of crude 2-acetamido-6-(tert-butoxycarbonyl)-4,5,6,7-tetrahydrothieno[2,3-c]pyridine-3-carboxylic acid (1.2 g , 3.50 mmol) and copper powder (0.29 g, 4.58 mmol) in quinoline (12 mL) was heated at 190° C. for 1.5 h. After cooling to room temperature, the reaction mixture was diluted with dichloromethane (100 mL) and then washed with 2M hydrochloric acid, a 5% aqueous solution of sodium bicarbonate, and brine. The organic phase was dried over magnesium sulfate, filtered, and concentrated in vacuo. The remaining residue was purified by column chromatography using a mixture of ethyl acetate/hexanes (1:5) as the eluant to afford 0.49 g of tert-butyl 2-acetamido-4,5-dihydrothieno[2,3-c]pyridine-6(7H)-carboxylate. Yield: 48%; m/z (ESI⁺) 297 (MH⁺).

A solution of tert-butyl 2-acetamido-4,5-dihydrothieno[2,3-c]pyridine-6(7H)-carboxylate (0.25 g, 0.84 mmol) in dichloromethane (3 mL) was treated with trifluoroacetic acid (1 mL). The mixture was allowed to stir for 2.5 h at room temperature and was then evaporated to dryness. The remaining solid was triturated with diethyl ether and then collected by filtration to afford 0.26 g of 2-acetamido-4,5,6,7-tetrahydrothieno[2,3-c]pyridin-6-ium 2,2,2-trifluoroacetate as a light brown solid. Yield: 99%; m/z (ESI⁺) 197 (M⁺)

A mixture of 2-acetamido-4,5,6,7-tetrahydrothieno[2,3-c]pyridin-6-ium 2,2,2-trifluoroacetate (0.26 g, 8.38 mmol), and acetaldehyde (0.075 mL, 1.34 mmol) in dichloroethane (5 mL) was allowed to stir for 1 h at room temperature. Next sodium triacetoxyborohydride (0.44 g, 2.09 mmol) was added in two portions over 5 min. The reaction mixture was allowed to stir for 24 h at room temperature. The reaction was quenched through the addition of a 5% aqueous sodium bicarbonate solution. The phases were separated and the aqueous phase was extracted with dichloromethane. The combined organic layers were dried over magnesium sulfate, filtered, and then concentrated in vacuo to afford 0.16 g of N-(6-ethyl-4,5,6,7-tetrahydrothieno[2,3-c]pyridin-2-yl)acetamide as a yellow solid. Yield 85%; m/z (ESI⁻) 225 (MH⁺); ¹H NMR (600 MHz, CDCl₃) δ 8.28 (s, 1H), 6.33 (s, 1H), 5.39 (s, 2H), 2.76 (t, J=5.4 Hz, 2H), 2.64-2.61 (m, 4H), 2.14 (s, 3H), 1.17 (t, J=7.2 Hz, 3H).

### Example 64

### N-(5-(Benzo[d]thiazol-2-yl)-1,2,3,4-tetrahydroisoquinolin-7-yl)acetamide

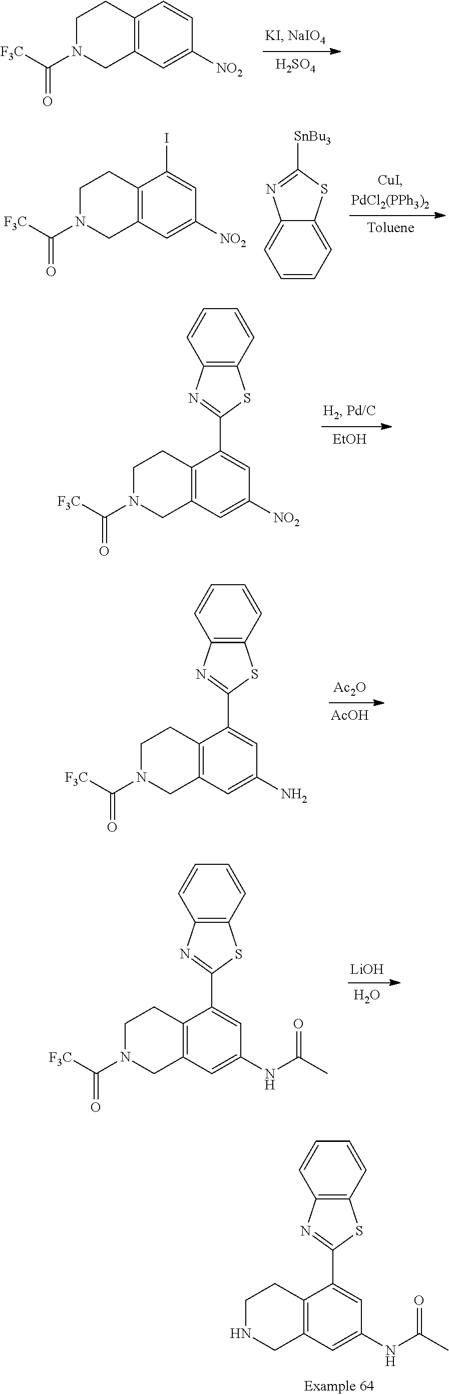

2,2,2-trifluoro-1-(7-nitro-3,4-dihydroisoquinolin-2(1H)-yl)ethanone was prepared via the teachings described in US Patent Application Publication US 2006/0040966 A1 (Examples 1 and 2, Pages 71-72).

Solid sodium periodate (0.38 g, 1.80 mmol) was added slowly to a solution of potassium iodide (0.80 g, 5.50 mmol) in sulfuric acid (20 mL). The resulting mixture was stirred for 0.5 h and then solid 2,2,2-trifluoro-1-(7-nitro-3,4-dihydroisoquinolin-2(1H)-yl)ethanone (1.0 g, 3.70 mmol) was added in one portion. The reaction mixture was allowed to stir for 1 h at 30° C. The reaction mixture was poured into iced water and the resulting mixture was extracted with dichloromethane. The organic phase was washed with a saturated aqueous solution of sodium thiosulfate. The organic phase was dried over magnesium sulfate and evaporated to afford 800 mg of 2,2,2-trifluoro-1-(5-iodo-7-nitro-3,4-dihydroisoquinolin-2(1H)-yl)ethanone. Yield 54%.

To a solution of 2,2,2-trifluoro-1-(5-iodo-7-nitro-3,4-dihydroisoquinolin-2(1H)-yl)ethanone (0.6 g, 1.50 mmol) in toluene (10 mL) being maintained under an argon atmosphere was added 3-(tributylstannyl)benzothiazole (0.76 g, 1.80 mmol), copper(I) iodide (0.014 g, 0.075 mmol), and dichloro-bis-triphenylphine-palladium(II) (0.042 g, 0.075 mmol). The resulting mixture was heated at 90° C. for 4 h and then allowed to stir for 12 h at room temperature. The precipitate that formed was collected by filtration and then washed with diethyl ether. Drying the solid in a stream of air afforded 400 mg of 1-(5-(benzo[d]thiazol-2-yl)-7-nitro-3,4-dihydroisoquinolin-2(1H)-yl)-2,2,2-trifluoroethanone. Yield 75%; m/z (ESI⁺) 408 (MH⁺).

To a suspension of 1-(5-(benzo[d]thiazol-2-yl)-7-nitro-3,4-dihydroisoquinolin-2(1H)-yl)-2,2,2-trifluoroethanone (0.35 g, 0.859 mmol) in ethanol (30 mL) was added 10% palladium on carbon (0.1 g). A balloon of hydrogen was attached to the flask and the mixture was heated at 40° C. for 4 h. The reaction mixture was filtered through a pad of Celite and the filtrate was condensed in vacuo to afford 0.26 g of 1-(7-amino-5-(benzo[d]thiazol-2-yl)-3,4-dihydroisoquinolin-2(1H)-yl)-2,2,2-trifluoroethanone as a light yellow solid. Yield: 81%; m/z (ESI⁺) 378 (MH⁺).

To a solution of 1-(7-amino-5-(benzo[d]thiazol-2-yl)-3,4-dihydroisoquinolin-2(1H)-yl)-2,2,2-trifluoroethanone (0.26 g, 0.70 mmol) in acetic acid (5 mL) was added acetic anhydride (0.5 mL). The resulting mixture was heated at reflux for 2 h and was then concentrated in vacuo. The remaining residue was dissolved in dichloromethane and washed with a 5% aqueous sodium bicarbonate solution and brine. The organic layer was dried over magnesium sulfate, filtered, and then concentrated in vacuo to afford 0.26 g of N-(5-(benzo[d]thiazol-2-yl)-2-(2,2,2-trifluoroacetyl)-1,2,3,4-tetrahydroisoquinolin-7-yl)acetamide. Yield: 89%; m/z (ESI⁺) 420 (MH⁺).

To a mixture of N-(5-(benzo[d]thiazol-2-yl)-2-(2,2,2-trifluoroacetyl)-1,2,3,4-tetrahydroisoquinolin-7-yl)acetamide (0.26 g, 0.620 mmol) in methanol (2 mL), dichloromethane (0.35 mL), and water (0.75 mL) was added lithium hydroxide (0.17 g, 7.0 mmol). The resulting mixture was allowed to stir at room temperature for 10 min and was then concentrated in vacuo. The remaining residue was dissolved in dichloromethane and the organic layer was washed with water, dried over magnesium sulfate, filtered, and then concentrated in vacuo. The remaining residue was triturated with a mixture of dichloromethane and water, which after filtration and drying, afforded 120 mg of N-(5-(benzo[d]thiazol-2-yl)-1,2,3,4-tetrahydroisoquinolin-7-yl)acetamide as a gray solid. Yield: 52%; m/z (ESI⁺) 324 (MH⁺); ¹H NMR (600 MHz, CDCl₃, mixture of rotamers) δ 8.18 (d, 1H), 8.16-8.12 (m, 2H), 7.99 (d, 1H), 7.58 (t, J=7.8 Hz, 1H), 7.50 (t, J=7.8 Hz, 1H), 4.97 (d, 2H), 3.95-3.90 (m, 2H), 3.55-3.51 (m, 2H).

### Examples 65-66

### 3-(Benzo[d]thiazol-2-yl)-6-ethyl-4,5 ,6,7-tetrahydrobenzo[b]thiophen-2-amine and N-(3-(Benzo[d]thiazol-2-yl)-6-ethyl-4,5,6,7-tetrahydrobenzo[b]thiophen-2-yl)acetamide

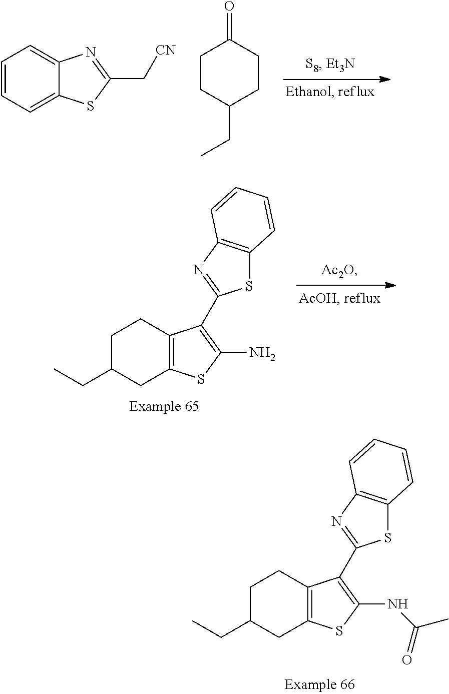

3-(Benzo[d]thiazol-2-yl)-6-ethyl-4,5,6,7-tetrahydrobenzo[b]thiophen-2-amine was prepared according to the method described in example 1 using (1,3-benzothiazol-2-yl)acetonitrile (1 g, 0.0057 mol), ethanol (17 mL), 4-ethylcyclohexanone (0.81 mL, 0.0057 mol), sulfur (0.18 g, 0.0057 mol), and triethylamine (1 mL). The crude product was purified by column chromatography on silica using hexanes as the eluant. The product obtained after chromatography was further triturated with hexanes and then collected by filtration to afford 0.446 g of 3-(benzo[d]thiazol-2-yl)-6-ethyl-4,5,6,7-tetrahydrobenzo[b]thiophen-2-amine as a beige solid. Yield: 25%; m/z (ESI⁺) 315 (MH⁺); ¹H NMR (500 MHz, CDCl₃) δ 7.90 (d, J=8 Hz, 1H), 7.82 (d, J=9 Hz, 1H), 7.42 (t, J=8 Hz, 1H), 7.28 (t, J=7.5 Hz, 1H), 6.89 (bs, 1H), 3.01 (d, J=16 Hz, 1H), 2.78 (bs, 1H), 2.68 (dd, J=16, 5 Hz, 1H), 2.25 (bt, J=12.5 Hz, 1H), 2.04 (d, J=12.5 Hz, 1H), 1.72 (bs, 1H), 1.48-1.42 (m, 3H), 0.99 (t, J=7.5 Hz, 3H).

N-(3-(Benzo[d]thiazol-2-yl)-6-ethyl-4,5,6,7-tetrahydrobenzo[b]thiophen-2-yl)acetamide was prepared according to the method described in example 2 using 3-(benzo[d]thiazol-2-yl)-6-ethyl-4,5,6,7-tetrahydrobenzo[b]thiophen-2-amine (0.17 g, 0.540 mmol), acetic anhydride (0.077 ml, 0.810 mmol), and acetic acid (5 mL). The crude product was purified by column chromatography on silica using hexanes as the eluant to afford 0.096 g of N-(3-(benzo[d]thiazol-2-yl)-6-ethyl-4,5,6,7-tetrahydrobenzo[b]thiophen-2-yl)acetamide as a yellow solid. Yield: 50%; m/z (ESI⁺) 379 (MNa⁺), 357 (MH⁺); ¹H NMR (500 MHz, CDCl₃) δ 13.04 (s, 1H), 7.95 (d, J=8 Hz, 1H), 7.89 (d, J=8 Hz, 1H), 7.50 (t, J=8 Hz, 1H), 7.37 (t, J=8 Hz, 1H), 3.05 (broad d, J=16 Hz, 1H), 2.84 (d, J=4.5 Hz, 1H), 2.80 (d, J=4.5 Hz, 1H), 2.35-2.30 (m, 4H), 2.08 (d, J=11 Hz, 1H), 1.71 (bs, 1H), 1.53-1.39 (m, 3H), 1.00 (t, J=8 Hz, 3H).

### Examples 67-68

### N-(3-(Benzo[d]thiazol-2-yl)-7-oxo-4,5,6,7-tetrahydrothieno[2,3-c]pyridin-2-yl)acetamide and N-(3-(Benzo[d]thiazol-2-yl)-6-ethyl-7-oxo-4,5,6,7-tetrahydrothieno[2,3-c]pyridin-2-yl)acetamide

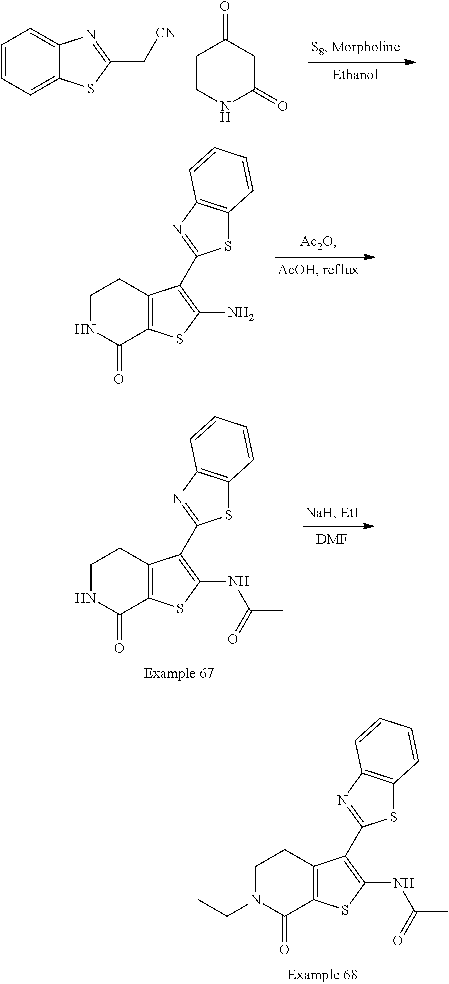

A mixture of sulfur (623 mg, 19.5 mmol), piperidine-2,4-dione (2 g, 17.7 mmol), and (1,3-benzothiazol-2-yl)acetonitrile (3.08 g, 17.7 mmol) in anhydrous ethanol was heated at 45° C. under an argon atmosphere. Then to the reaction mixture was added neat morpholine (1.54 mL, 17.7 mmol). The reaction mixture was allowed to stir at 50° C., under an argon atmosphere, for 24 h. The precipitate that formed was collected via filtration and was washed with methanol. The solid was further triturated with diethyl ether and pentane to afford 2.0 g of 2-amino-3-(benzo[d]thiazol-2-yl)-5,6-dihydrothieno[2,3-c]pyridin-7(4H)-one as an orange solid. Yield: 38% (90% purity); m/z (ESI⁺) 302 (MH⁺).

To a solution of 2-amino-3-(benzo[d]thiazol-2-yl)-5,6-dihydrothieno[2,3-c]pyridin-7(4H)-one (35 mg, 0.116 mmol) in acetic acid (0.5 mL) was added acetic anhydride (33 μL, 0.349 mmol). The reaction mixture was heated at reflux under an argon atmosphere for 4 h. The reaction mixture was concentrated under reduced pressure and the remaining solid recrystallized from methanol to give pale yellow solid, which was further triturated with methanol and diethyl ether, to afford 27 mg of N-(3-(benzo[d]thiazol-2-yl)-7-oxo-4,5,6,7-tetrahydrothieno[2,3-c]pyridin-2-yl)acetamide as an off-white solid. Yield: 68%; m/z (ESI⁺) 344 (MH⁺); ¹H NMR (600 MHz, d₆-DMSO) δ 12.71 (s, 1H), 8.14 (d, J=8.4 Hz, 2H), 7.73 (s, 1H), 7.57 (t, J=7.8 Hz, 1H), 7.46 (t, J=7.8 Hz, 1H), 3.50 (dd, J=6.6, 4.8 Hz, 2H), 3.07 (t, J=7.2 Hz, 2H), 2.32 (s, 3H).

Solid sodium hydride (64 mg, 1.60 mmol) was added to a solution of N-(3-(benzo[d]thiazol-2-yl)-7-oxo-4,5,6,7-tetrahydrothieno[2,3-c]pyridin-2-yl)acetamide (220 mg, 0.641 mmol) in N,N-dimethylformamide (1 mL). The mixture was allowed to stir, under an argon atmosphere, for 30 min at room temperature. Neat ethyl iodide (184 μL, 1.92 mmol) was added to the flask and the reaction mixture was allowed to stir at room temperature for 24 h. After the first 24 h additional sodium hydride (64 mg, 1.60 mmol) and ethyl iodide (184 μL 1.92 mmol) were added to the reaction mixture. The reaction mixture was allowed to stir, under an argon atmosphere, for another 24 h. The reaction mixture was diluted with dichloromethane and then washed with water, 1M hydrochloric acid, a 5% aqueous sodium bicarbonate solution, and brine. The organic layer was dried over magnesium sulfate, filtered, and condensed in vacuo. The crude product was purified by column chromatography on silica using a mixture of chloroform/methanol as the eluant. The product obtained chromatography was recrystallized from methanol to afford 70 mg of N-(3-(benzo[d]thiazol-2-yl)-6-ethyl-7-oxo-4,5,6,7-tetrahydrothieno[2,3-c]pyridin-2-yl)acetamide. Yield: 29%; m/z (ESI⁺) 372 (MH⁺); ¹H NMR (600 MHz, d₆-DMSO) δ 12.72 (s, 1H), 8.17 (t, J=7.2 Hz, 2H), 7.60 (t, J=7.8 Hz, 1H), 7.49 (t, J=7.8 Hz, 1H), 3.68 (t, J=7.2 Hz, 2H), 3.45 (q, J=7.2 Hz, 2H), 3.18 (t, J=7.2 Hz, 2H), 2.35 (s, 3H), 1.11 (t, J=7.2 Hz, 3H).

### Example 69

### 2-Acetamido-3-phenyl-4,5,6,7-tetrahydrothieno[2,3-c]pyridin-6-ium 2,2,2-Trifluoroacetate

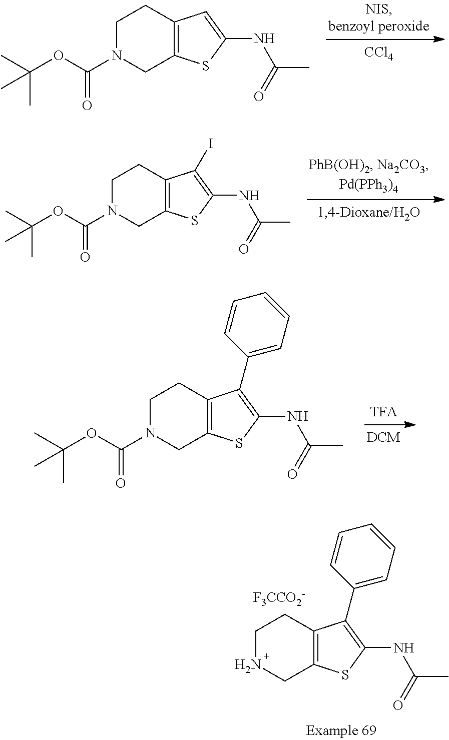

A mixture of tert-butyl 2-acetamido-4,5-dihydrothieno[2,3-c]pyridine-6(7H)-carboxylate (from Example 63, 1 g, 3.40 mmol), N-iodosuccinimide (0.92 g, 4.10 mmol), and benzoyl peroxide (15 mg) in carbon tetrachloride (10 mL) was heated at reflux for 4 h. The reaction mixture was diluted with dichloromethane and then washed with water, 1M sodium hydroxide, and brine. The organic phase was dried over magnesium sulfate, filtered, and condensed in vacuo. The crude product was purified by column chromatography on silica using a mixture of dichloromethane/methanol as the eluant to afford 0.7 g of tert-butyl 2-acetamido-3-iodo-4,5-dihydrothieno[2,3-c]pyridine-6(7H)-carboxylate as a pale yellow solid. Yield 49%.

To a mixture of tert-butyl 2-acetamido-3-iodo-4,5-dihydrothieno[2,3-c]pyridine-6(7H)-carboxylate (0.2 g, 0.470 mmol) in 1,4-dioxane (4 mL) and water (1 mL), being maintained under an agron atmosphere, were added sodium carbonate (0.055 g, 0.940 mmol), phenyl boronic acid (0.07 g, 0.570 mmol), and tetrakis(triphenylphosphine)palladium(0) (0.055 g, 0.047 mmol). The reaction mixture was flushed with argone for 10 min and then the vessel was sealed. The system was heated at 95° C. for 5 h. The reaction mixture was diluted with dichloromethane and water. The organic phase was washed with 1M hydrochloric acid, 1M sodium hydroxide, dried over magnesium sulfate, and evaporated. The crude product was purified by column chromatography on silica using a mixture of dichloromethane/methanol as the eluant to afford 100 mg of tert-butyl 2-acetamido-3-phenyl-4,5-dihydrothieno[2,3-c]pyridine-6(7H)-carboxylate as an off-white solid. Yield 22%; m/z (ESI⁺) 395 (MNa⁺).

A solution of tert-butyl 2-acetamido-3-phenyl-4,5-dihydrothieno[2,3-c]pyridine-6(7H)-carboxylate (100 mg, 0.268 mmol) in dichloromethane (3 mL) was treated with trifluoroacetic acid (0.5 mL). The resulting mixture was allowed to stir at room temperature for 2 h. The reaction mixture was condensed in vacuo and the remaining residue was triturated with diethyl ether. The solid was collected by filtration and allowed to dry in a stream of air to afford 95 mg of 2-acetamido-3-phenyl-4,5,6,7-tetrahydrothieno[2,3-c]pyridin-6-ium 2,2,2-trifluoroacetate as a cream-colored solid. Yield: 95%; m/z (ESI⁺) 273 (M⁺); ¹H NMR (600 MHz, d₆-DMSO) δ 10.10 (s, 1H), 9.27 (bs, 2H), 7.46 (t, J=9 Hz, 2H), 7.37 (t, J=9 Hz, 1H), 7.24 (d, J=9 Hz, 2H), 4.28 (s, 2H), 3.31 (t, J=6.6 Hz, 2H), 2.53 (t, J=6.6 Hz, 2H), 1.99 (s, 3H).

### Examples 70-72

### 2-Acetamido-3-(benzo[d]thiazol-2-yl)-5,6-dihydro-4H-thieno[2,3-c]pyrrol-5-ium 2,2,2-Trifluoroacetate, 2-Acetamido-3-(benzo[d]thiazol-2-yl)-5-ethyl-5 ,6-dihydro-4H-thieno[2,3-c]pyrrol-5-ium Chloride, and 2-Acetamido-3-(benzo[d]thiazol-2-yl)-5-isopropyl-5,6-dihydro-4H-thieno[2,3-c]pyrrol-5-ium Chloride

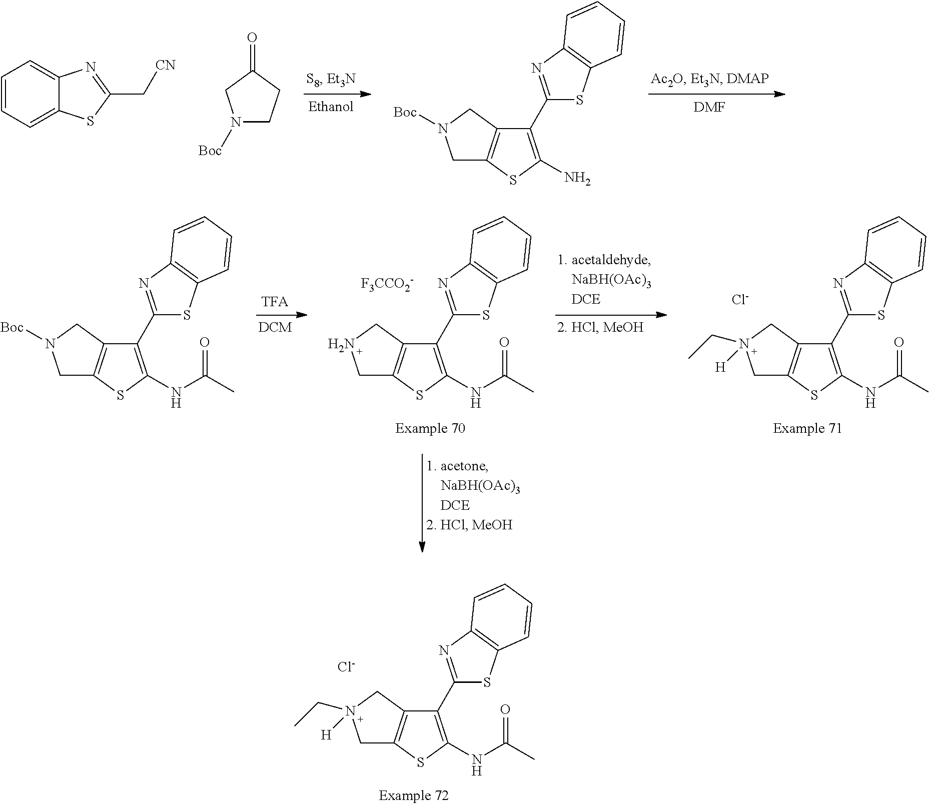

A mixture of tert-butyl 3-oxopyrrolidine-l-carboxylate (1 g, 5.40 mmol), 1,3-benzothiazol-2-ylacetonitrile (0.94 g, 5.40 mmol), sulfur (0.173 g, 5.40 mmol), and triethylamine (1.13 mL, 8.10 mmol) in ethanol (15 mL) was heated at reflux for 5 h. The reaction mixture was allowed to cool to room temperature and was then condensed in vacuo. The remaining residue was dissolved in dichloromethane and washed with water. The organic phase was dried over magnesium sulfate, filtered, and concentrated in vacuo to afford 2.0 g of crude tert-butyl 2-amino-3-(benzo[d]thiazol-2-yl)-4H-thieno[2,3-c]pyrrole-5(6H)-carboxylate as a black, amorphous solid, which was used in the next reaction without any further purification.

To a solution of crude tert-butyl 2-amino-3-(benzo[d]thiazol-2-yl)-4H-thieno[2,3-c]pyrrole-5(6H)-carboxylate (2.0 g, 5.40 mmol) in N,N-dimethylformamide (20 mL) were added triethylamine (2.2 mL, 16.0 mmol), acetic anhydride (0.82 mL, 8.10 mmol), N,N-dimethyl-4-aminopyridine (10 mg). The reaction mixture was allowed to stir at room temperature for 16 h. The reaction mixture was condensed in vacuo and the remaining residue was dissolved in dichloromethane. The mixture was washed with water and the organic phase was dried over magnesium sulfate, filtered, and condensed in vacuo. The crude product was purified by column chromatography on silica using a mixture of ethyl acetate/hexanes as the eluant to afford 120 mg of tert-butyl 2-acetamido-3-(benzo[d]thiazol-2-yl)-4H-thieno[2,3-c]pyrrole-5(6H)-carboxylate as a dark gray solid. Yield: 5% over 2 steps; m/z (ESI⁺) 416 (MH⁺)

To a solution of tert-butyl 2-acetamido-3-(benzo[d]thiazol-2-yl)-4H-thieno[2,3-c]pyrrole-5(6H)-carboxylate (120 mg, 0.289 mmol) in dichloromethane (3 mL) was added trifluoroacetic acid (0.5 mL). The reaction mixture was allowed to stir for 2 h at room temperature and was then condensed in vacuo. The remaining residue was triturated with diethyl ether, filtered, and allowed to dry in a stream of air to afford 130 mg of 2-acetamido-3-(benzo[d]thiazol-2-yl)-5,6-dihydro-4H-thieno[2,3-c]pyrrol-5-ium 2,2,2-trifluoroacetate as a brown solid. Yield: 95%; m/z (ESI⁺) 316 (M⁺); ¹H NMR (600 MHz, d₆-DMSO) δ 12.00 (s, 1H), 9.92 (s, 2H), 8.13 (t, J=9.6 Hz, 2H), 7.57 (t, J=7.2 Hz, 1H), 7.46 (t, J=7.8 Hz, 1H), 4.64 (s, 2H), 4.51 (s, 2H), 2.32 (s, 3H).

A mixture of 2-acetamido-3-(benzo[d]thiazol-2-yl)-5,6-dihydro-4H-thieno[2,3-c]pyrrol-5-ium 2,2,2-trifluoroacetate (0.085 g, 0.300 mmol), acetaldehyde (0.05 mL, 0.900 mmol), and acetic acid (0.1 mL, 1.80 mol) in dichloroethane (5 mL) was allowed to stir at room temperature for 2 h. Solid sodium triacetoxyborohydride (0.25 g, 1.20 mmol) was added after 2 h and the resulting mixture was allowed to stir at room temperature for 24 h. The reaction mixture was diluted with dichloromethane and then washed with a 5% aqueous solution of sodium bicarbonate and brine. The organic phase was dried over magnesium sulfate, filtered, and condensed in vacuo. The crude product was purified by column chromatography on silica using a mixture of dichloromethane/methanol as the eluant. The solid obtained after chromatography was dissolved in methanol saturated with dry hydrochloric acid. The mixture was allowed to stir for 15 min and was then condensed in vacuo. The remaining residue was triturated with diethyl ether, filtered, and allowed to dry in a stream of air to afford 30 mg of 2-acetamido-3-(benzo[d]thiazol-2-yl)-5-ethyl-5,6-dihydro-4H-thieno[2,3-c]pyrrol-5-ium chloride. Yield 17%; m/z (ESI⁺) 344 (M⁺); ¹H NMR (600 MHz, d-TFA) δ 8.02 (bs, 2H), 7.76 (s, 1H), 7.69 (s, 1H), 5.12 (bs, 2H), 4.60 (bs, 2H), 3.60 (bs, 2H), 2.24 (bs, 3H), 1.13 (bs, 3H).

2-Acetamido-3-(benzo[d]thiazol-2-yl)-5-isopropyl-5,6-dihydro-4H-thieno[2,3-c]pyrrol-5-ium chloride was prepared according to the procedure described for 2-acetamido-3-(benzo[d]thiazol-2-yl)-5-ethyl-5 ,6-dihydro-4H-thieno[2,3-c]pyrrol-5-ium chloride (Example 71), except that acetone was used in place of acetaldehyde. Yield: 67%; m/z (ESI⁺) 358 (MH⁺); ¹H NMR (600 MHz, d-TFA) δ 7.69 (bs, 2H), 7.44 (bs, 1H), 7.37 (s, 1H), 4.76 (bs, 1H), 4.64 (bs, 1H), 4.93 (bs, 2H), 3.49 (bs, 1H), 1.91 (s, 3H), 1.09 (bs, 6H).

### Examples 73-74

### 7-Acetamido-6-(benzo[d]thiazol-2-yl)-1,2,3,4-tetrahydroisoquinolin-2-ium 2,2,2-Trifluoroacetate and 7-Acetamido-6-(benzo[d]thiazol-2-yl)-2-ethyl-1,2,3,4-tetrahydroisoquinolin-2-ium Chloride

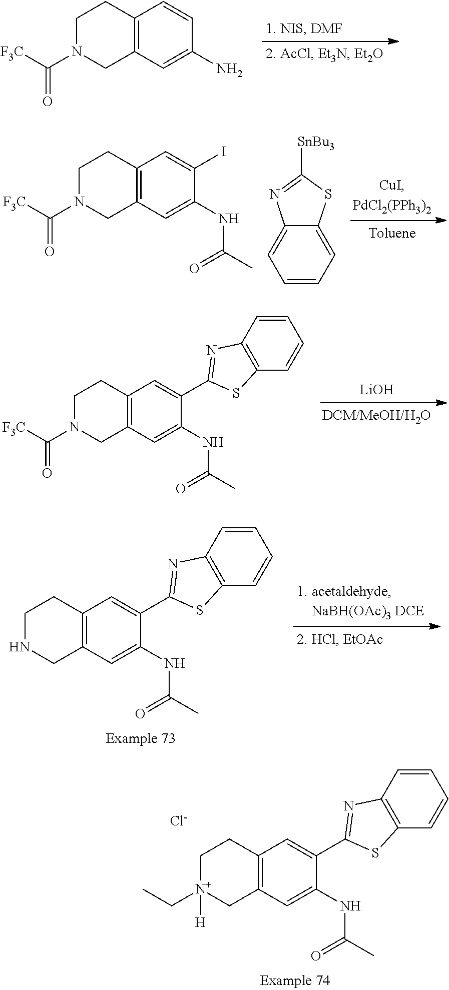

1-(7-Amino-3,4-dihydroisoquinolin-2(1H)-yl)-2,2,2-trifluoroethanone was prepared via the teachings described in US Patent Application Publication US 2006/0040966 A1 (Examples 1, 2, and 3, Pages 71-72).

To a solution of 1-(7-amino-3,4-dihydroisoquinolin-2(1H)-yl)-2,2,2-trifluoroethanone (1.10 g, 4.50 mmol) in N,N-dimethylformamide (2 mL) cooled to -20° C. was added a solution of N-iodosuccinimide (1.11 g, 4.95 mmol) in N,N-dimethylformamide (4 mL) in a dropwise fashion. Once the addition was complete the reaction mixture was allowed to warm to room temperature and stir for 24 h. The reaction mixture was condensed in vacuo and the remaining residue was redissolved in dichloromethane. The mixture was washed with a 1M aqueous solution of sodium hydroxide, dried over magnesium sulfate, filtered, and condensed in vacuo. The 1.55 g of crude, black N-(6-iodo-2-(2,2,2-trifluoroacetyl)-1,2,3,4-tetrahydroisoquinolin-7-yl)acetamide was used in the next reaction without any further purification.

A mixture of crude N-(6-iodo-2-(2,2,2-trifluoroacetyl)-1,2,3,4-tetrahydroisoquinolin-7-yl)acetamide (1.55 g, 4.19 mmol), triethylamine (0.87 mL, 6.29 mmol), and acetyl chloride (0.39 mL, 5.45 mmol) in anhydrous diethyl ether (10 mL) was allowed to stir at room temperature for 24 h. The reaction mixture was condensed in vacuo and the remaining residue was dissolved in dichloromethane. The mixture was washed with water, a 5% aqueous solution of sodium bicarbonate, and brine. The organic phase was dried over magnesium sulfate, filtered, and concentrated in vacuo. The crude product was purified by column chromatography on silica using a mixture of ethyl acetate/hexanes (1:2) as the eluant to afford 0.22 g of N-(6-(benzo[d]thiazol-2-yl)-2-(2,2,2-trifluoroacetyl)-1,2,3,4-tetrahydroisoquinolin-7-yl)acetamide as a yellow solid. Yield: 13% over two steps; m/z (ESI⁺) 413 (MH⁺).

To a solution of N-(6-(benzo[d]thiazol-2-yl)-2-(2,2,2-trifluoroacetyl)-1,2,3,4-tetrahydroisoquinolin-7-yl)acetamide (0.21 g, 0.510 mmol) in toluene (5 mL), being maintained under an argon atmosphere, were added dichloro-bis-triphenylphosphine palladium(II) (0.018 g, 0.0255 mmol) and copper(I) iodide (0.0049 g, 0.0255 mmol), followed by 2-(tributylstannyl)-1,3-benzathiazole (0.26 g, 0.612 mmol). The reaction mixture heated at 80° C., under an argon atmosphere, for 24 h. The reaction mixture was filtered through a pad of Celite and the filtrate was concentrated in vacuo. The remaining residue was dissolved in dichloromethane and washed with water, a 0.5M aqueous solution of N,N,N′,N′-ethylenediaminetetraacetic acid, and brine. The organic phase was dried over magnesium sulfate, filtered, and concentrated in vacuo. The crude product was first triturated with diethyl ether and then methanol. Collection of the solid and drying in a stream of air afforded 0.18 g of N-(6-(benzo[d]thiazol-2-yl)-2-(2,2,2-trifluoroacetyl)-1,2,3,4-tetrahydroisoquinolin-7-yl)acetamide as a beige solid. Yield: 86%; m/z (ESI⁺) 420 (MH⁺).

To a mixture of N-(6-(benzo[d]thiazol-2-yl)-2-(2,2,2-trifluoroacetyl)-1,2,3,4-tetrahydroisoquinolin-7-yl)acetamide (0.18 g, 0.429 mmol) in methanol (1.50 mL), dichloromethane (0.26 mL), and water (0.55 mL) was added solid lithium hydroxide hydrate (0.12 g, 2.79 mmol). The reaction mixture was allowed to stir at room temperature for 25 min and was then condensed in vacuo to one-third of the original volume. The remaining material was diluted with water and extracted with dichloromethane. The organic phase was washed with water, dried over magnesium sulfate, filtered, and condensed in vacuo to afford 0.100 g of N-(6-(benzo[d]thiazol-2-yl)-1,2,3,4-tetrahydroisoquinolin-7-yl)acetamide as a yellow solid. Yield: 71% For biological testing, a small portion of N-(6-(benzo[d]thiazol-2-yl)-1,2,3,4-tetrahydroisoquinolin-7-yl)acetamide was converted to the hydrochloride salt via the following procedure. A solution of N-(6-(benzo[d]thiazol-2-yl)-1,2,3,4-tetrahydroisoquinolin-7-yl)acetamide (30 mg) in ethyl acetate (0.50 mL) was treated with a 5.6M solution of hydrochloric acid in ethyl acetate (2 mL1). The mixture was allowed to stir at room temperature for 10 min and was then concentrated in vacuo. The remaining solid was triturated with diethyl ether, filtered, and allowed to dry in a stream of air to afford 25 mg of 7-acetamido-6-(benzo[d]thiazol-2-yl)-1,2,3,4-tetrahydroisoquinolin-2-ium 2,2,2-trifluoroacetate as a yellow solid. Yield: 76%; m/z (ESI⁺) 324 (M⁺); ¹H NMR (600 MHz, d₆-DMSO) δ 11.60 (s, 1H), 9.51 (bs, 2H), 8.16-8.15 (m, 2H), 8.11 (d, J=7.8 Hz, 1H), 7.56 (t, J=7.8 Hz, 1H), 7.49 (t, J=7.8 Hz, 1H), 4.30 (bs, 2H), 3.34 (bs, 2H), 3.06 (bs, 2H), 2.19 (s, 3H).

A mixture of N-(6-(benzo[d]thiazol-2-yl)-1,2,3,4-tetrahydroisoquinolin-7-yl)acetamide (0.070 g, 0.216 mmol), acetaldehyde (0.023 mL, 0.411 mmol), and acetic acid (0.074 mL, 1.30 mmol) in dichloroethane (2 mL) was allowed to stir at room temperature for 45 min. Sodium triacetoxyborohydride (0.046 g, 0.605 mmol) was then added to the flask and the reaction mixture was allowed to stir at room temperature for 24 h. The reaction mixture was condensed in vacuo and the remaining residue was diluted with water. The aqueous mixture was made basic in pH through the addition of a 5% aqueous solution of sodium bicarbonate and was then extracted with dichloromethane. The organic phase was washed with brine, dried over magnesium sulfate, filtered, and condensed in vacuo. The crude product was purified by column chromatography on silica using a mixture of dichloromethane/methanol mixture (90:1) as the eluant to afford 0.027 g of N-(6-(benzo[d]thiazol-2-yl)-2-ethyl-1,2,3,4-tetrahydroisoquinolin-7-yl)acetamide as a yellow solid. Yield: 35%; m/z (ESI⁺) 352 (MH⁺). To a solution of N-(6-(benzo[d]thiazol-2-yl)-2-ethyl-1,2,3,4-tetrahydroisoquinolin-7-yl)acetamide (0.027 g, 0.077 mmol) in ethyl acetate (0.20 mL) was added a 5.6M solution of hydrochloric acid in ethyl acetate (2 mL). The reaction mixture was allowed to stir at room temperature for 15 min and was then condensed in vacuo. The remaining residue was triturated with diethyl ether, filtered, and allowed to dry in a stream of air to afford 0.0075 g of 7-acetamido-6-(benzo[d]thiazol-2-yl)-2-ethyl-1,2,3,4-tetrahydroisoquinolin-2-ium chloride as a yellow solid. Yield: 25%; m/z (ESI⁺) 352 (M⁺); ¹H NMR (600 MHz, d-TFA) δ 7.74 (bs, 2H), 7.47 (s, 1H), 7.40 (bs, 2H), 7.08 (bs, 1H), 4.44 (bs, 1H), 4.04 (bs, 1H), 3.59 (bs, 1H), 3.12 (bs, 4H), 2.88 (bs, 1H), 1.85 (s, 3H), 1.16 (s, 3H).

### Example 75-76

### 6-Acetamido-5-(benzordithiazol-2-yl)-1,2,3,4-tetrahydroisoquinolin-2-ium 2,2,2-Trifluoroacetate and 6-Acetamido-7-(benzo[d]thiazol-2-yl)-1,2,3,4-tetrahydroisoquinolin-2-ium 2,2,2-Trifluoroacetate

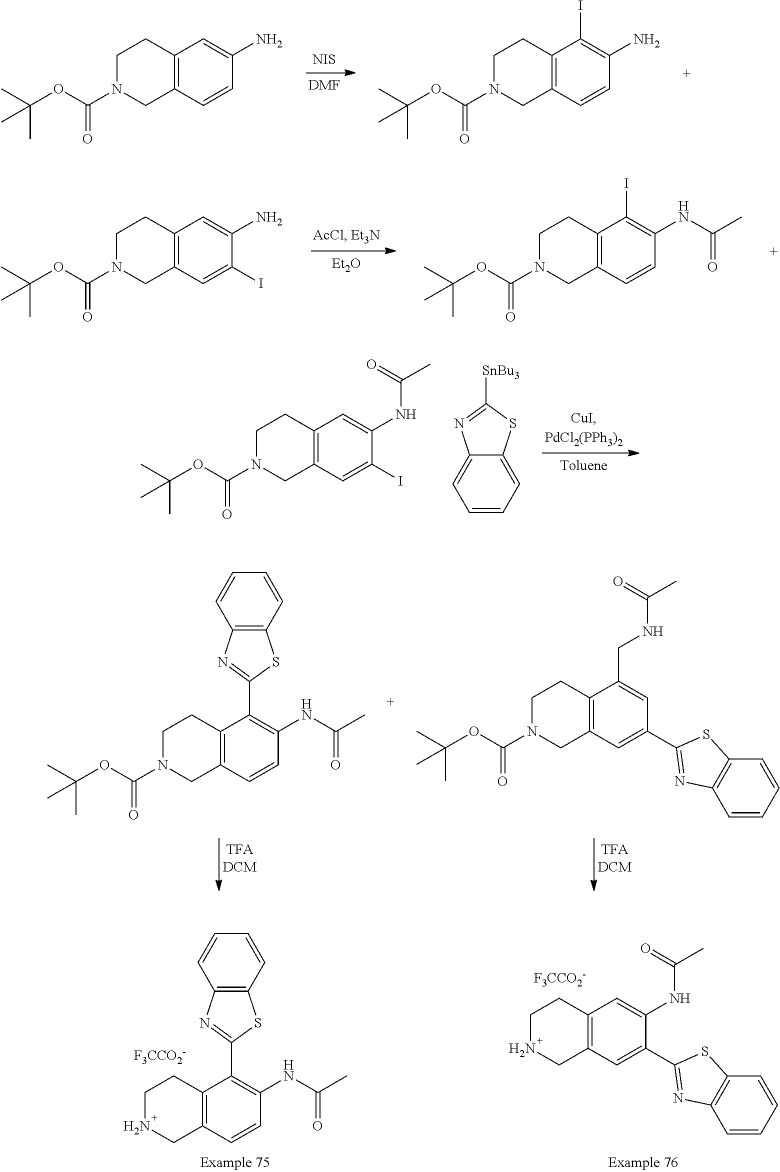

To a solution of tert-butyl 6-amino-3,4-dihydroisoquinoline-2(1H)-carboxylate (purchased from Oakwood Products/ catalog# 011348; 0.6 g, 2.42 mmol) in N,N-dimethylformamide (4 mL), cooled to −20° C., was added a solution of N-iodo succinimide (0.6 g, 2.66 mmol) in N,N-dimethylformamide (6 mL) in a dropwise manner. The reaction was stirred overnight at ambient temperature. The reaction mixture was concentrated in vacuo and the the remaining residue was dissolved in ethyl acetate. The mixture was washed with a 1M sodium hydroxide solution and brine. The organic phase was dried over magnesium sulfate, filtered, and concentrated in vacuo. The crude product was purified by column chromatography on silica using dichloromethane as eluant to afford a 0.5 g mixture of tert-butyl 6-amino-5-iodo-3,4-dihydroisoquinoline-2(1H)-carboxylate and tert-butyl 6-amino-7-iodo-3,4-dihydroisoquinoline-2(1H)-carboxylate as an orange solid. Yield: 55%; m/z (ESI⁺) 375 (MH⁺).

A mixture of tert-butyl 6-amino-5-iodo-3,4-dihydroisoquinoline-2(1H)-carboxylate and tert-butyl 6-amino-7-iodo-3,4-dihydroisoquinoline-2(1H)-carboxylate (0.48 g, 1.28 mmol), triethylamine (0.5 mL, 3.59 mmol), and acetyl chloride (0.17 mL, 2.31 mmol) in diethyl ether (10 mL) was allowed to stir at ambient temperature for 24 h. The reaction mixture was concentrated to dryness and the crude product was purified by column chromatography on silica using a mixture of dichloromethane/methanol (1:200) as the eluant to afford a 0.27 g mixture of tert-butyl 6-acetamido-5-iodo-3,4-dihydroisoquinoline-2(1H)-carboxylate and tert-butyl 6-acetamido-7-iodo-3,4-dihydroisoquinoline-2(1H)-carboxylate as an orange solid. Yield: 55%; m/z (ESI⁺) 439 (MNa⁺).

To a mixture of tert-butyl 6-acetamido-5-iodo-3,4-dihydroisoquinoline-2(1H)-carboxylate and tert-butyl 6-acetamido-7-iodo-3,4-dihydroisoquinoline-2(1H)-carboxylate (0.27 g, 0.65 mmol) in toluene (5 mL), under an atmosphere of argon, were added dichloro-bis-triphenylphosphine palladium(II) (23 mg, 0.032 mmol) and copper(I) iodide (6 mg, 0.032 mmol). To the reaction mixture was then added 2-(tributylstannyl)-1,3-benzothiazole (0.33 g, 0.780 mmol). The reaction mixture was heated at 80° C. for 24 h. The reaction was diluted with dichloromethane and then filtered. The filtrate was condensed in vacuo and the remaining residue was suspended in dichloromethane. The mixture was washed with an aqueous solution of N,N,N′,N′-ethylenediaminetetraacetic acid and brine. The organic phase was dried over magnesium sulfate, filtered, and condensed in vacuo. The crude product was purified by column chromatography on silica using a mixture of dichloromethane/methanol (1:150) as the eluent to afford 50 mg of tert-butyl 6-acetamido-5-(benzo[d]thiazol-2-yl)-3,4-dihydroisoquinoline-2(1H)-carboxylate as a white solid and 70 mg of tert-butyl 6-acetamido-7-(benzo[d]thiazol-2-yl)-3,4-dihydroisoquinoline-2(1H)-carboxylate. Combined yield: 44%; m/z (ESI⁺) 446 (MNa⁺), 424 (MH⁺).

To a solution of tert-butyl 6-acetamido-5-(benzo[d]thiazol-2-yl)-3,4-dihydroisoquinoline-2(1H)-carboxylate (50 mg, 1.04 mmol) in dichloromethane (2 mL) was added trifluoroacetic acid (0.4 mL). The reaction mixture was allowed to stir at ambient temperature for 24 h. The reaction mixture was diluted with diethyl ether and the precipitate that formed was collected by filtration. The solid was washed with diethyl ether and allowed to dry in a stream of air to afford 47 mg of 6-acetamido-5-(benzo[d]thiazol-2-yl)-1,2,3,4-tetrahydroisoquinolin-2-ium 2,2,2-trifluoroacetate as a white solid. Yield: 90%; m/z (ESI⁺) 324 (M⁺); ¹H NMR (600 MHz, d₆-DMSO) δ 9.53 (s, 1H), 9.03 (s, 2H), 8.16 (d, J=8.4 Hz, 1H), 8.07 (d, J=7.8 Hz, 1H), 7.55 (t, J=7.8 Hz, 1H), 7.51-7.47 (m, 2H), 7.37 (d, J=8.4 Hz, 1H), 4.31 (s, 2H), 3.27 (s, 2H), 2.81 (s, 2H), 1.84 (s, 3H).

To a solution of tert-butyl 6-acetamido-7-(benzo[d]thiazol-2-yl)-3,4-dihydroisoquinoline-2(1H)-carboxylate (70 mg, 0.16 mmol) in dichloromethane (1 mL) was added trifluoroacetic acid (0.2 mL). The reaction mixture was allowed to stir at ambient temperature for 24 h. The reaction mixture was diluted with diethyl ether and the precipitate that formed was collected by filtration. The filtered solids were washed with diethyl ether and allowed to dry in a stream of air to afford 67 mg of the desired product 6-acetamido-7-(benzo[d]thiazol-2-yl)-1,2,3,4-tetrahydroisoquinolin-2-ium 2,2,2-trifluoroacetate as a white solid. Yield: 93%; m/z (ESI⁺) 324 (M⁺); ¹H NMR (600 MHz, d₆-DMSO) δ 11.65 (s, 1H), 9.07 (s, 2H), 8.20 (s, 1H), 8.16 (d, J=7.8 Hz, 1H), 8.11 (d, J=7.8 Hz, 1H), 7.90 (s, 1H), 7.57 (t, J=8.4 Hz, 1H), 7.49 (t, J=8.4 Hz, 1H), 4.34 (s, 2H), 3.39 (bs, 2H), 3.03 (t, J=6.6 Hz, 2H), 2.19 (s, 3H).

### Examples 77-79

### 3-(Benzo[d]thiazol-2-yl)-4,5,6,7-tetrahydrobenzo[b]thiophen-2-aminium

Chloride, N-(3-(Benzo[d]thiazol-2-yl)-4,5,6,7-tetrahydrobenzo[b]thiophen-2-yl)acetamide, and N-(3-(Benzo[d]thiazol-2-yl)benz[b]thiophen-2-yl)acetamide

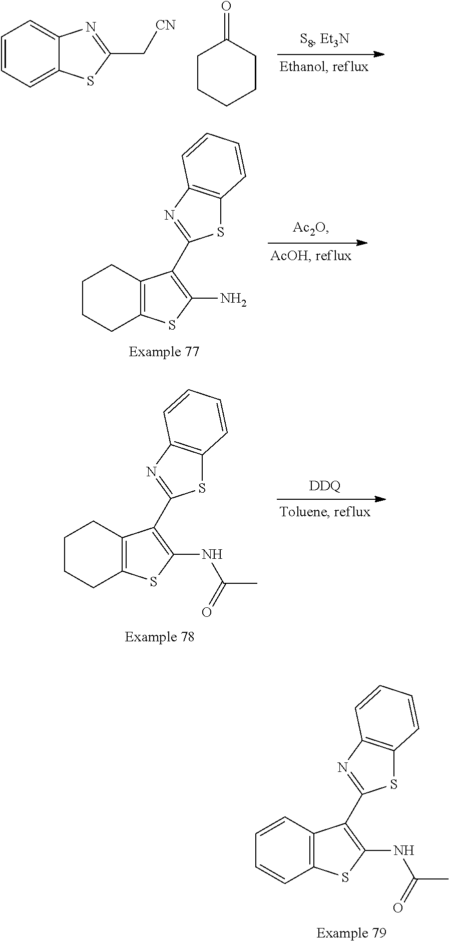

A mixture of (1,3-benzothiazol-2-yl)acetonitrile (1 g, 0.0057 mol) cyclohexanone (0.59 mL, 0.0057 mol), sulfur (0.18 g, 0.0057 mol), and triethylamine (1 mL) in ethanol (17mL) was heated at reflux for 5 h. The reaction mixture was condensed in vacuo and the remaining residue was triturated with water, and then neutralized with concentrated hydrochloric acid. The mixture was filtered and the solid was purified by column chromatography on silica using hexanes as the eluant to afford 0.375 g of 3-(benzo[d]thiazol-2-yl)-4,5,6,7-tetrahydrobenzo[b]thiophen-2-amine. A portion of the product (100 mg) was then dissolved in ethyl acetate (2 mL) and treated with a 5M hydrochloric acid solution in ethyl acetate to afford 3-(benzo[d]thiazol-2-yl)-4,5,6,7-tetrahydrobenzo[b]thiophen-2-aminium chloride as a yellow solid. Yield: 25%; m/z (ESI⁺) 287 (MH⁺); ¹H NMR (500 MHz, CD₃OD) δ 8.04 (t, J=7.5 Hz, 2H), 7.61 (t, J=7.5 Hz, 1H), 7.50 (t, J=7.5 Hz, 1H), 2.87 (bs, 2H), 2.71 (bs, 2H), 1.92 (bs, 4H).

A mixture of 3-(benzo[c]thiazol-2-yl)-4,5,6,7-tetrahydrobenzo[b]thiophen-2-amine (0.15 g, 0.00052 mol) and acetic anhydride (0.074 mL, 0.00078 mol) in acetic acid (10 mL) was heated at reflux for 4 h. The reaction mixture was condensed in vacuo and the remaining residue was dissolved in water. The mixture was adjusted to a basic pH with an aqueous 4M sodium hydroxide solution. The precipitate that formed was collected by filtration and washed with both methanol and diethyl ether. The solid was then further purified by column chromatography on silica using hexanes as the eluant to afford 99 mg of N-(3-(benzo[d]thiazol-2-yl)-4,5,6,7-tetrahydrobenzo[b]thiophen-2-yl)acetamide as a yellow solid. Yield 58%; m/z (ESI⁺) 351 (MNa⁺), 329 (MH⁺); ¹H NMR (500 MHz, CDCl₃) δ 10.05 (s, 1H), 7.95 (d, J=8.5 Hz, 1H), 7.89 (d, J=7.5 Hz, 1H), 7.50 (t, J=7.5 Hz, 1H), 7.37 (t, J=7.5 Hz, 1H), 2.91 (bs, 2H), 2.73 (bs, 2H), 2.35 (s, 3H), 1.93 (bs, 2H), 1.87 (bs, 2H).

A mixture of N-(3-(benzo[d]thiazol-2-yl)-4,5,6,7-tetrahydrobenzo[b]thiophen-2-yl)acetamide (0.3 g, 0.0009 mol) and 2,3-dichloro-5,6-dicyano-p-benzoquinone (0.4 g, 0.0018 mol) in toluene (20 mL) was heated at reflux for 7.5 h. The insoluble solids were filtered and washed with dichloromethane. The filtrate was condensed in vacuo and the remaining residue was purified by column chromatography on silica using dichloromethane as the eluant to afford 0.099 g of N-(3-(benzo[d]thiazol-2-yl)benzo[b]thiophen-2-yl)acetamide as a yellow solid. Yield: 34%; m/z (ESI⁺) 325 (MH⁺); ¹H NMR (200 MHz, CDCl₃) δ 13.52 (s, 1H), 8.33 (d, J=8.2 Hz, 1H), 8.01 (t, J=7.2 Hz, 2H), 7.85 (d, J=8.2 Hz, 1H), 7.58-7.30 (m, 4H), 2.46 (s, 3H).

### Examples 80-81

### 3-(Benzo[d]thiazol-2-yl)-6,6-dimethyl-4,5 ,6,7-tetrahydrobenzo[b]thiophen-2-amine and N-(3-(Benzo[d]thiazol-2-yl)-6,6-dimethyl-4,5,6,7-tetrahydrobenzo[b]thiophen-2-yl)acetamide

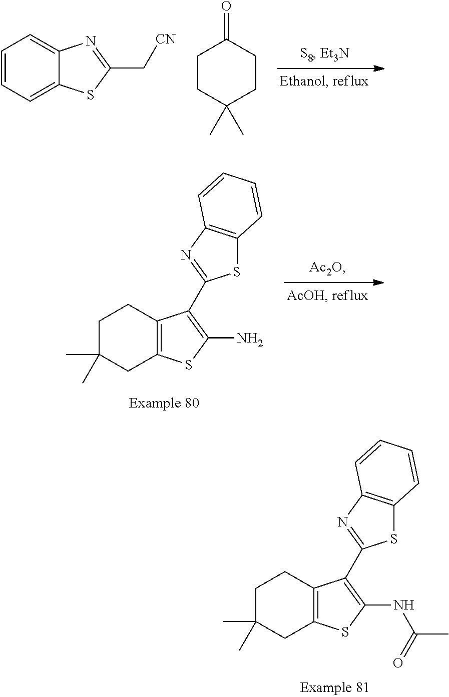

A mixture of (1,3-benzothiazol-2-yl)acetonitrile (1 g, 0.0057 mol), 4,4-dimethylcyclohexanone (0.72 g, 0.0057 mol), sulfur (0.18 g, 0.0057 mol), and triethylamine (1 mL) in ethanol (17 mL) was heated at reflux for 4 h. The reaction mixture was condensed in vacuo and the remaining residue was dissolved in dichloromethane. The mixture was washed with water, dried over magnesium sulfate, and condensed in vacuo. The crude product was purified by column chromatography on silica using hexanes as the eluant and then recrystallized from methanol to give afford 0.357 g of 3-(benzo[d]thiazol-2-yl)-6,6-dimethyl-4,5,6,7-tetrahydrobenzo[b]thiophen-2-amine as a beige solid. Yield: 20%; m/z (ESI⁺) 315 (MH⁺); ¹H NMR (200 MHz, CDCl₃) δ 7.89 (d, J=8.2 Hz, 1H), 7.83 (d, J=8.4 Hz, 1H), 7.42 (t, J=8.2 Hz, 1H), 7.27 (t, J=8.2 Hz, 1H), 6.81 (bs, 1H), 2.87 (t, J=6.4 Hz, 2H), 2.38 (s, 2H), 1.64 (t, J=6.4 Hz, 2H), 1.05 (s, 6H).

A mixture of 3-(benzo[d]thiazol-2-yl)-6,6-dimethyl-4,5,6,7-tetrahydrobenzo[b]thiophen-2-amine (0.3 g, 0.0061 mol) and acetic anhydride (0.14 mL, 0.0014 mol) in acetic acid (10 mL) was heated at reflux for 3 h. The precipitate that formed was collected by filtration and washed with water, methanol, and diethyl ether. The solid was then recrystallized from a mixture of boiling hexanes and diethyl ether to afford 0.185 g of N-(3-(benzo[d]thiazol-2-yl)-6,6-dimethyl-4,5,6,7-tetrahydrobenzo[b]thiophen-2-yl)acetamide as a yellow solid. Yield: 57%; m/z (ESI⁺) 379 (MNa⁺), 357 (MH⁺); ¹H NMR (500 MHz, CDCl₃) δ 13.07 (s, 1H), 7.96 (d, J=7.5 Hz, 1H), 7.90 (d, J=7.5 Hz, 1H), 7.51 (t, J=7.5 Hz, 1H), 7.38 (t, J=7.5 Hz, 1H), 2.92 (s, 2H), 2.50 (s, 2H), 2.36 (s, 3H), 1.68 (t, J=6.5 Hz, 2H), 1.05 (s, 6H).

### Example 82

### N-(6-Acetyl-3-(benzo[c]thiazol-2-yl)-4,5,6,7-tetrahydrothieno[2,3-c]pyridin-2-yl)acetamide

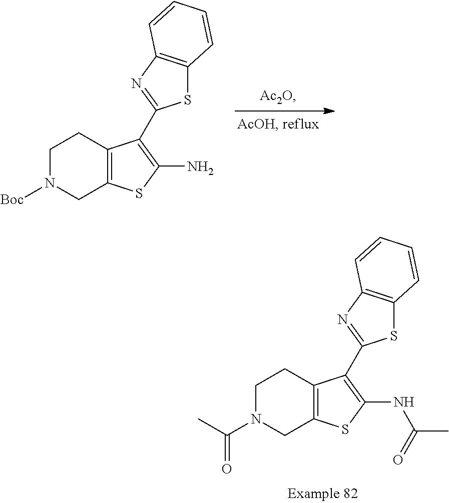 [SMILES: CC(C)(C)OC(=O)N1CCc2c(sc(N)c2-c2nc3ccccc3s2)C1 ; CC(=O)Nc1sc2c(c1-c1nc3ccccc3s1)CCN(C(C)=O)C2]

A solution of tert-butyl 2-amino-3-(benzo[d]thiazol-2-yl)-4,5-dihydrothieno[2,3-c]pyridine-6(7H)-carboxylate (from example 5; 500 mg, 1.29 mmol) and acetic anhydride (0.37 mL, 3.87 mmol) in acetic acid (5 mL) was heated at reflux for 4 h. The reaction mixture was condensed in vacuo and the remaining residue was dissolved in dichloromethane. The mixture was washed with a 5% aqueous sodium bicarbonate solution, water, and brine. The organic layer was dried over magnesium sulfate, filtered, and condensed in vacuo. The crude product was purified by column chromatography on silica using a mixture of dichloromethane/methanol as the eluant and then recrystallized from methanol to afford 110 mg of N-(6-acetyl-3-(benzo[d]thiazol-2-yl)-4,5,6,7-tetrahydrothieno[2,3-c]pyridin-2-yl)acetamide as a beige solid. Yield: 23%; m/z (ESI⁻) 370 (M⁻−H); ¹H NMR (500 MHz, d₆-DMSO, 80° C.) δ 12.32 (s, 1H); 8.10 (d, J=8.5 Hz, 2H), 7.57 (t, J=7.5 Hz, 1H), 7.45 (t, J=7.5 Hz, 1H), 4.65 (s, 2H), 3.82 (t, J=5.5 Hz, 2H), 3.20 (s, 2H), 2.30 (s, 3H), 2.12 (s, 3H).

### Example 83

### 2-Acetamido-3-(benzo[d]thiazol-2-yl)-4,5,6,7-tetrahydrothieno[3,2-c]pyridin-5-ium Chloride

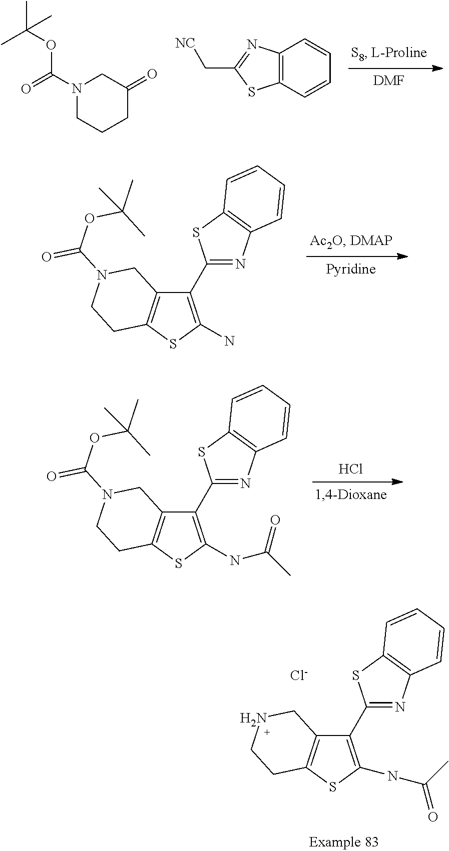

L-Proline (0.098 g, 0.85 mmol) was added in portions to a mixture of tert-butyl 3-oxopiperidine-1-carboxylate (1.8 g, 8.50 mmol), benzothiazole-2-acetonitrile (1.5 g, 8.50 mmol), and sulfur (0.36 g, 11.1 mmol) in N,N-dimethylformamide (10 mL) that had been cooled to 0° C. After the proline addition the reaction mixture was allowed to warm to room temperature and was then heated at 60° C. for 3 h. The reaction mixture was diluted with ethyl acetate (100 mL) and washed with water and brine. The organic phase was dried over sodium sulfate, filtered, and concentrated under vacuum. The crude product was subjected to column chromatography on neutral alumina using a mixture of ethyl acetate/hexanes (1:9) as the eluant to afford a mixture of isomeric products. The isomeric mixture were further separated and purified by preparative HPLC to obtained 0.08 g of tert-butyl 2-amino-3-(benzo[d]thiazol-2-yl)-6,7-dihydrothieno[3,2-c]pyridine-5(4H)-carboxylate as a yellow solid. Yield: 2.4%; m/z (ESI⁺) 388 (MH⁺).

Acetic anhydride (0.023 mL, 0.250 mmol) was added to a mixture of tert-butyl 2-amino-3-(benzo[d]thiazol-2-yl)-6,7-dihydrothieno[3,2-c]pyridine-5(4H)-carboxylate (0.08 g, 0.200 mmol) and N,N-dimethyl-4-aminopyridine (5 mg), in pyridine (5 mL), that had been cooled to 0° C. After the addition of the anhydride was complete the reaction mixture was allowed to warm to room temperature and stir for 24 h. The volatiles were removed under vacuum and th remaining residue was purified by column chromatography on silica using a mixture of ethyl acetate/hexanes (1:4) as the eluant to afford 0.06 g of tert-butyl 2-acetamido-3-(benzo[d]thiazol-2-yl)-6,7-dihydrothieno[3,2-c]pyridine-5(4H)-carboxylate as an-off white solid. Yield: 68%; m/z (ESI⁺) 430 (MH⁺).

A 2M solution of hydrochloric acid in 1,4-dioxane (1 mL) was added to a solution of tert-butyl 2-acetamido-3-(benzo[d]thiazol-2-yl)-6,7-dihydrothieno[3,2-c]pyridine-5(4H)-carboxylate (0.06 g, 0.140 mmol) in 1,4-dioxane (5 mL) at 0° C. At the end of the addition the reaction mixture was allowed to warm to room temperature and stir for 1 h. The volatiles were removed under vacuum and the remaining residue was triturated with diethyl ether. The solid was collected by filtration and allowed to dry in a stream of air to afford 0.048 g of 2-acetamido-3-(benzo[d]thiazol-2-yl)-4,5,6,7-tetrahydrothieno[3,2-c]pyridin-5-ium chloride as a light yellow solid. Yield: 93%; m/z (ESI⁺) 330 (M⁺); ¹H NMR (400 MHz, d₆-DMSO): 6 11.87 (s, 1 H), 8.20 (d, J=7.6 Hz, 1 H), 8.14 (d, J=7.6 Hz, 1 H), 7.60 (t, J=7.2 Hz, 1 H), 7.50 (t, J=7.2 Hz, 1 H), 4.48 (s, 2 H), 3.47 (bs, 2 H), 3.03 (bs, 2 H), 2.28 (s, 3 H).

### Examples 84-85

### 3-Aminobenzofuran-2-carboxamide and 3-Benzamidobenzofuran-2-carboxamide

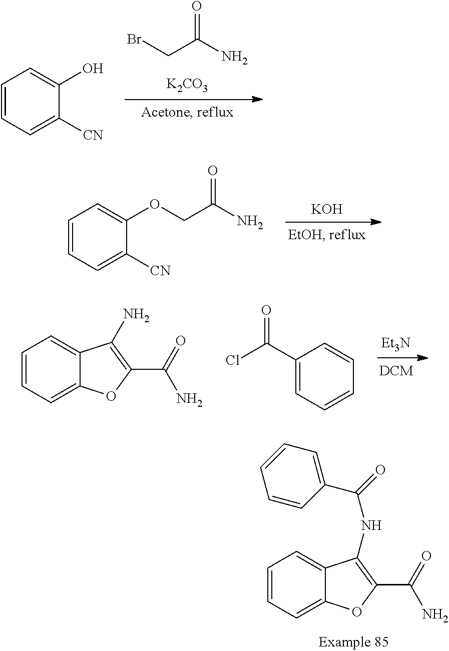

A mixture of bromoacetamide (3 g, 21.7 mmol), 2-hydroxybenzonitrile (2.58 g, 21.7 mmol), and potassium carbonate (6 g, 43.4 mmol) in acetone (10 mL) was heated at reflux for 16 h. The reaction mixture was allowed to cool to room temperature and the precipitate that formed was collected by filtration. The filtered solid was washed with copious amounts of water and then allowed to dry in a stream of air to afford 3.13 g of 2-(2-cyanophenoxy)acetamide as a white solid. Yield: 82%; m/z (ESI⁺) 177 (MH⁺).

To a suspension of 2-(2-cyanophenoxy)acetamide (3.13 g, 18.0 mmol) in ethanol (100 mL) was added solid potassium hydroxide (3.2 g, 57.0 mmol). The reaction mixture was heated at reflux for 1 h and was then condensed in vacuo. The remaining residue was diluted with water and then filtered. The filtered solids were washed with water and cold methanol. Drying the remaining material in a stream of air afforded 2 g of 3-aminobenzofuran-2-carboxamide as a white solid. Yield: 64%; m/z (ESI⁺) 177 (MH⁺); ¹H NMR (200 MHz, d₆-DMSO) δ 7.80 (d, J=7.6 Hz, 1H), 7.36 (s, 2H), 7.23-7.16 (m, 2H), 5.96 (bs, 2H).

A mixture of 3-aminobenzofuran-2-carboxamide (0.3 g, 1.7 mmol) and triethylamine (0.5 mL, 3.80 mmol) in dichloromethane (10 mL) was treated with neat benzoyl chloride (0.30 mL, 2.90 mmol) in a dropwise fashion. The reaction mixture was allowed to stir a room temperature for 24 h. The reaction mixture was diluted with dichloromethane and then washed with water, 2M hydrochloric acid, and a 5% aqueous sodium bicarbonate. The organic phase was dried over magnesium sulfate, filtered, and condensed in vacuo to afford yellow oil. The oil was triturated with diethyl ether and the solid that formed was collected by filtration and then recrystallized from methanol to afford 130 mg of 3-benzamidobenzofuran-2-carboxamide as a white solid. Yield: 27%; m/z (ESI⁺) 303 (MNa⁺); ¹H NMR (500 MHz, d6-DMSO) δ 11.04 (s, H), 8.25 (d, J=8 Hz, 2H), 8.01 (d, J=7.5 Hz, 2H), 7.96 (s, 1H), 7.67 (t, J=7.5 Hz, 1H), 7.62-7.58 (m, 3H), 7.54 (t, J=7.5 Hz, 1H), 7.36 (t, J=7.5 Hz, 1H).

### Example 86

### 2-(2-Chlorobenzamido)-4,5,6,7-tetrahydrobenzo[b]thiophene-3-carboxamide

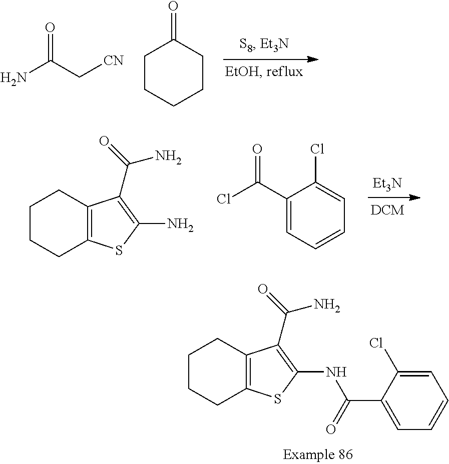

A mixture of cyanoacetamide (2 g, 24.0 mmol), cyclohexanone (2.48 mL, 24.0 mmol), sulfur (0.77 g, 24.0 mmol), and triethylamine (0.77 g, 24.0 mmol) in ethanol (30 mL) was heated at reflux for 5 h and then concentrated in vacuo. The crude product was purified by column chromatography on silica using a 1% methanol/dichloromethane mixture as the eluant to afford 989 mg of 2-amino-4,5,6,7-tetrahydrobenzo[b]thiophene-3-carboxamide as an orange solid. Yield: 21%; m/z (ESI⁻) 195 (M⁻−H).

A mixture of 2-amino-4,5,6,7-tetrahydrobenzo[b]thiophene-3-carboxamide (0.1 g, 0.51 mmol) and triethylamine (0.11 mL, 0.76 mmol) in dichloromethane (10 mL) was treated with neat 2-chlorobenzoyl chloride (0.09 mL, 0.71 mmol) in a dropwise fashion. The reaction mixture was allowed to stir at room temperature for 24 h. The reaction mixture was washed with water, 2M hydrochloric acid, and a 5% aqueous sodium bicarbonate solution. The organic phase was dried over magnesium sulfate, filtered, and condensed in vacuo to afford a yellow oil. The oil was triturated with diethyl ether and the precipitate that formed was collected by filtration. The filtered solid was purified by column chromatography on silica using dichloromethane was the eluant and then recrystallized from methanol to afford 15 mg of 2-(2-chlorobenzamido)-4,5,6,7-tetrahydrobenzo[b]thiophene-3-carboxamide as a white solid. Yield 8%; m/z (ESI⁻) 333 (M⁻−H); ¹H NMR (500 MHz, CDCl₃) δ 12.71 (s, 1H), 7.75 (d, J=7.5 Hz, 1H), 7.46 (d, J=8.5 Hz, 1H), 7.42 (t, J=8 Hz, 1H), 7.36 (t, J=6 Hz, 1H), 5.76 (bs, 2H), 2.76-2.73 (m, 4H), 1.89-1.86 (m, 4H).

### Example 87

### N-(3-(4,5-Dimethylthiazol-2-yl)-6-ethyl-4,5,6,7-tetrahydrothieno[2,3-c]pyridin-2-yl)acetamide

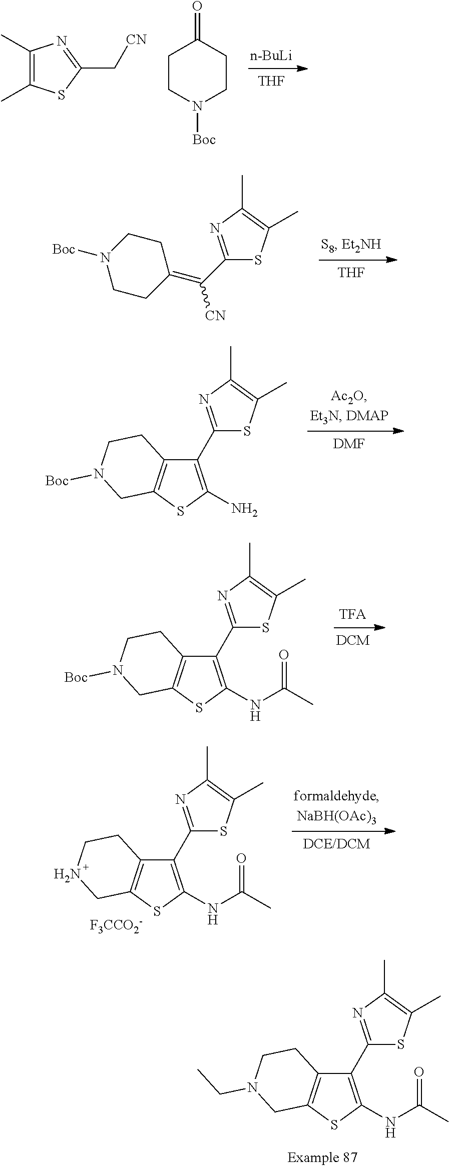

A solution of 2-(4,5-dimethylthiazol-2-yl)acetonitrile (2 g, 13.2 mmol) in anhydrous tetrahydrofuran (10 mL) was cooled to -70° C. and then treated with n-butyllithium (2.5M in hexane, 5.3 mL, 13.2 mmol) in a dropwise manner. The reaction mixture was allowed to stir at the reduced temperature for 20 min and then a solution of tert-butyl 4-oxopiperidine-1-carboxylate (2.62 g, 13.2 mmol) in dry tetrahydrofuran (15 mL) was added in a dropwise fashion. The reaction mixture was allowed to warm to room temperature and then stir for 1 h. The reaction mixture was diluted with a 1M hydrochloric acid solution (20 mL) and ethyl acetate (20 mL). The mixture was stirred vigorously for 10 min and then the organic phase was washed with brine, dried over magnesium sulfate, filtered, and condensed in vacuo. The crude product was recrystallized from hexanes to afford 3.6 g tert-butyl 4-(cyano(4,5-dimethylthiazol-2-yl)methylene)piperidine-1-carboxylate as a yellow crystalline solid. Yield: 82%; m/z (ESI) 356 (MNa⁺), 334 (MH⁺).

A mixture of tert-butyl 4-(cyano(4,5-dimethylthiazol-2-yl)methylene)piperidine-1-carboxylate (3.6 g, 10.8 mmol), sulfur (346 mg, 10.8 mmol), and diethylamine (4.1 mL, 39.5 mmol) in tetrahydrofuran (20 mL) was allowed to stir at room temperature. The progress of the reaction was monitored by thin layer chromatography. When the starting nitrile could no longer be detected, the precipitate that formed was collected by filtration, washed with diethyl ether, and allowed to dry in a stream of air to afford 2.5 g of tert-butyl 2-amino-3-(4,5-dimethylthiazol-2-yl)-4,5-dihydrothieno[2,3-c]pyridine-6(7H)-carboxylate. Yield: 64%; m/z (ESI⁺) 366 (MH⁺).

A mixture of 2-amino-3-(4,5-dimethylthiazol-2-yl)-4,5-dihydrothieno[2,3-c]pyridine-6(7H)-carboxylate (1 g, 2.74 mmol), N,N-dimethyl-4-aminopyridine (10 mg), triethylamine (1.2 mL, 8.21 mmol), and acetic anhydride (0.39 mL, 4.11 mmol) in N,N-dimethylformamide (7 mL) was allowed to stir at room temperature for 24 h. The reaction mixture was diluted with water and then extracted with ethyl acetate. The combined organic extracts were washed with 2M hydrochloric acid and brine. The organic phase was dried over magnesium sulfate, filtered, and condensed in vacuo. The crude product was purified by column chromatography on silica using an ethyl acetate/hexanes gradient (1:10-1:5) as the eluant to afford 0.6 g of tert-butyl 2-acetamido-3-(4,5-dimethylthiazol-2-yl)-4,5-dihydrothieno[2,3-c]pyridine-6(7H)-carboxylate. Yield: 54%; m/z (ESI⁺) 407 (WH⁺).

A solution of tert-butyl 2-acetamido-3-(4,5-dimethylthiazol-2-yl)-4,5-dihydrothieno[2,3-c]pyridine-6(7H)-carboxylate (600 mg, 1.47 mmol) in dichloromethane (5 mL) was treated with trifluoroacetic acid (0.55 mL, 7.36 mmol). The reaction mixture was allowed to stir at room temperature for 24 h and was then concentrated in vacuo. The product was recrystallized from ethyl acetate to afford 470 mg of 2-acetamido-3-(4,5-dimethylthiazol-2-yl)-4,5,6,7-tetrahydrothieno[2,3-c]pyridin-6-ium 2,2,2-trifluoroacetate. Yield: 76%; m/z (ESI⁺) 308 (M⁺).

A mixture of 2-acetamido-3-(4,5-dimethylthiazol-2-yl)-4,5,6,7-tetrahydrothieno[2,3-c]pyridin-6-ium 2,2,2-trifluoroacetate (0.2 g, 0.475 mmol) and acetaldehyde (0.043 mL, 0.760 mmol) in dichloroethane (5 mL) and dichloromethane (3 mL) was allowed to stir at room temperature for 1.5 h. The mixture was then treated with solid sodium triacetoxyborohydride (141 mg, 0.665 mmol) in one portion. The reaction mixture was allowed to stir at room temperature for 24 h. The reaction mixture was quenched through the addition of a 5% aqueous sodium bicarbonate solution. The mixture was extracted with chloroform and the combined organic extracts were dried over magnesium sulfate, filtered, and condensed in vacuo. The crude product was recrystallized from a mixture of hexanes and diethyl ether to afford 85mg of N-(3-(4,5-dimethylthiazol-2-yl)-6-ethyl-4,5,6,7-tetrahydrothieno[2,3-c]pyridin-2-yl)acetamide as a yellow crystalline solid. Yield: 53%; m/z (ESI⁺) 336 (MH⁺); ¹H NMR (500 MHz, CDCl₃) δ 12.83 (s, 1H); 3.64 (s, 2H), 2.91 (d, J=11.5 Hz, 2H), 2.89 (d, J=11.5 Hz, 2H), 2.38 (s, 3H), 2.37 (s, 3H), 2.27 (s, 3H), 1.21 (t, J=7 Hz, 3H)

### Example 88

### CFTR Folding and Trafficking Assay

CFTR transport assays. Either ΔF508 expressing CFBE410- lung cells were cultured in the presence or absence of reagent in 12 well Sarstedt dishes, harvested, lysed and the processing of CFTR from the band B to the band C glycoform detected by SDS-PAGE and immunoblotting using either monoclonal 3G11 or M3A7.

Quantitation of CFTR glycoforms: Immunoblot exposures were selected to allow visualization of CFTR recovery under identical protein loads in same SDS-PAGE for all treatments. Given the dynamic range, quantitation of the band B and C glycoforms was made by analysis of band intensities that were in the linear range. The x-ray films were exposed for increasing time and the different exposures were quantified using a FluroChemSP (Alpha Inotech) densitometer/software package. Where band B and C were quantified from different exposures, an internal reference was used to normalize the signal intensity. This method is described in more detail in Hutt et al. (2010).¹⁶ Reduced histone deacetylase 7 activity restores function to misfolded CFTR in cystic fibrosis, Nature Chemical Biology (2010), 6(1), 25-33, the contents of which are expressly incorporated by reference herein.

### Biological Activity of Selected Compounds:

### Immunoblot Analysis

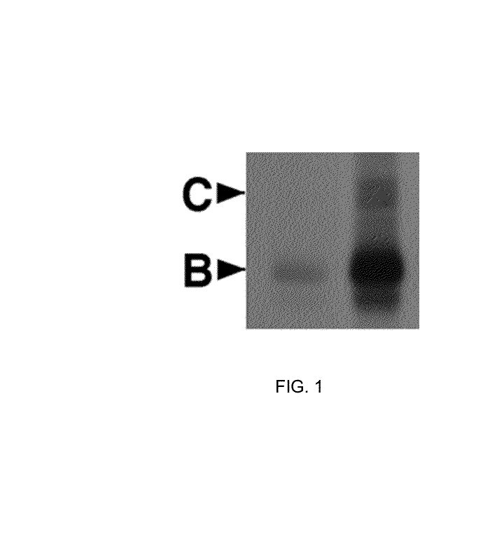

CFBE410- lung cells were cultured in the presence and absence of compound/example 2 as described by Hutt et al. (2010).¹⁶ Imunoblot analysis shown in FIG. 1 indicates enhanced levels of band B and C in the presence of compound 2.

### Example 89:CFTR Functional Assays

### CFTR Iodide Flux Assay:

Ninety six well microplates containing CFBE41^{o-} cells stably expressing ΔF508-CFTR and halide-sensitive YFP were incubated at 27° C. for 20 to 24 h. After incubation, cells were washed with PBS (containing 137 mM NaCl, 2.7 mM KCl, 8.1 mM Na₂HPO4, 1.5 mMKH₂PO₄, 1 mMCaCl₂, 0.5 mMMgCl₂) and stimulated for 20 min with forskolin and test compounds. Microplates were read using a plate reader equipped with excitation (HQ500/20×: 500 nm) and emission (HQ535/30M: 535 nm) filters for yellow fluorescent protein. Each assay consisted of a continuous 30-s fluorescence reading (5 points per second) with 3 s before and 27 s after injection of 165 ul of an iodide-containing solution (PBS with Cl⁻ replaced by I⁻). Final iodide concentration in the wells was 100 mM. These data were normalized to the initial background subtracted fluorescence.

### Monitoring of CFTR Function by Ussing Chamber Conductance:

CFBE41^{o-} cells stably expressing ΔF508 CFTR and primary ΔF/ΔF-HBE cells were cultured on permeable supports as described previously.¹⁷⁻¹⁹ Short-circuit currents (I_{sc}) were measured as previously described.^{20,21}

### Biological Activity of Select Compounds:

### Iodide Flux Assay

Table 6 shows percent quenching of yellow fluorescent protein (YFP) by select compounds described in the invention. Quenching values for the selected compounds were obtained at 10 μM. Quenching of YFP signal is an indirect measure of CFTR function.

TABLE 6

| Percent Quenching of YFP at 10 μM | Example/Compound Number |
|---|---|
| <15% | 1, 5, 8, 11, 12, 14, 19, 20, 21, 22, 23, |
| | 24, 25, 26, 32, 33, 34, 35, 37, 39, 41, 43, |
| | 45, 46, 47, 48, 49, 50, 52, 54, 57, 62, |
| | 63, 64, 65, 66, 67, 68, 71, 73, 74, 75, 76, |
| | 77, 78, 79, 80, 81, 82, 84, 85, 86, 87 |
| 15-20% | 9, 10, 44, 51, 56, 60, 61 |
| >20% | 2, 3, 4, 6, 7, 13, 15, 16, 17, 18, 36, |
| | 38, 40, 53, 55, 70 |

Ussing Chamber Conductance

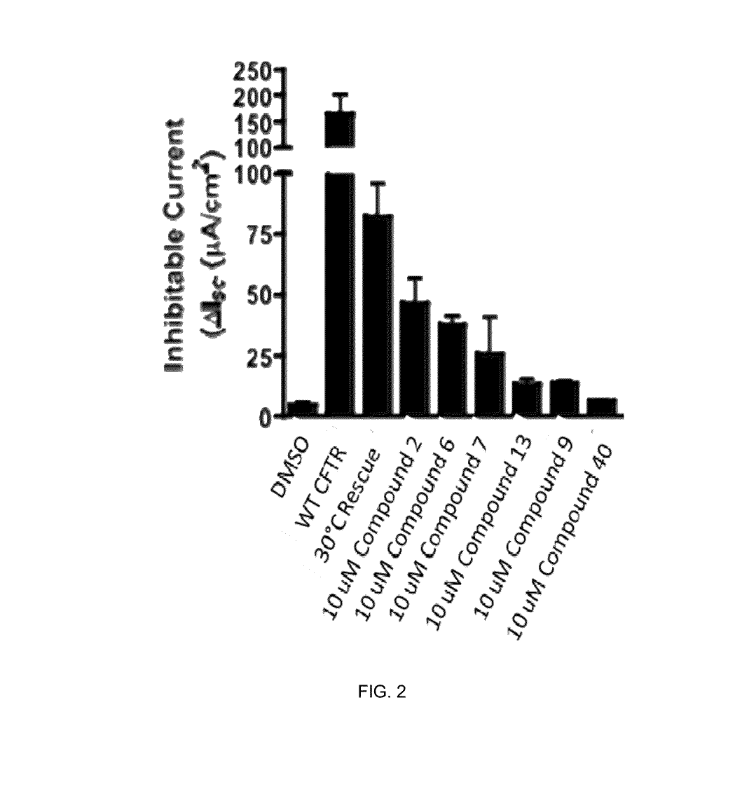

Compiled short circuit currents measured for compounds 2, 6, 7, 9, 13 and 40 are presented in FIG. 2.

### REFERENCES

1. Riordan, J. R. CFTR function and prospects for therapy. Annu. Rev. Biochem. 2008, 77, 701-26 .

2. Qu, B. H., Strickland, E. H. and Thomas, P. J. Localization and suppression of a kinetic defect in cystic fibrosis transmembrane conductance regulator folding. J. Biol. Chem. 1997, 272, 15739-44.

3. Boucher, R. C. Evidence for airway surface dehydration as the initiating event in CF airway disease. J. Intern. Med. 2007, 261, 5-16.

4. Balch, W. E., Morimoto, R. I., Dillin, A. and Kelly, J. W. Adapting proteostasis for disease intervention. Science. 2008, 319, 916-9.

5. Powers, E. T., Morimoto, R.I., Dillin, A., Kelly, J. W. and Balch, W. E. Biological and chemical approaches to diseases of proteostasis deficiency. Annu. Rev. Biochem.2009, 78, 959-91.

6. Hutt, D. M., Powers, E. T. and Balch, W. E. The proteostasis boundary in misfolding diseases of membrane traffic. FEBS Lett. 2009, 583, 2639-46.

7. Gregersen, N. Protein misfolding disorders: pathogenesis and intervention. J. Inherit. Metab. Dis. 2006, 29, 456-70.

8. Elkhol, Y. M. Studies with 2-Benzothiazolylacetonitrile: Synthesis of new 2-thienylbenzothiazoles and N-thieyl maleimide derivatives, Phosphorus, Sulfur and Silicon. 2002, 177, 115-122.

9. Sherida L., Mkrtchyan, A. P., Noravyan, A. S., and Petrosyan, V. M. Derivatives of condensed Thieno[2,3-d]pyrimidines. 15. Synthesis of 2-substituted 3-amino-6,6-dimethyl-5,6-dihydro-8H-pyrano[4′,3′:4,5]thieno[2,3-d]pyrimidin-4-ones, Chemistry of Heterocyclic Compounds. 2002, 38 2.

10. Johnson S. L., Chen L.-H., Barile E., Emdadi, A., Sabet M., Yuan, H., Wei J., Guiney, D., and Pellecchia, M. Structure—activity relationship studies of a novel series of anthrax lethal factor inhibitors Bioorg. & Med. Chem.2009, 17, 3352-3368.

11. Gewald, K., Schinke, E., and Bottcher, H. Chem. Ber.1966, 99, 94; Gewald, K. Heterocycles from CG-acid nitriles. VII. 2-Aminothiophene from a-oxo mercaptans and methylene-active nitriles. Chem. Ber. 1965-98(11), 3571.

12. Sabnis, R. W., Rangnekar, D. W. Synthesis of 3-Hetaryl-4,5,6,7-tetrahydrobenzo[b]thiophenes, J. Heterocyclic Chem. 1992, 29, 1027.

13. Romagnoli, R., Baraldi, P.G., Carrion, M. D., Cruz-Lopez, O., Cara, C. L., Tolomeo, M., Grimaudo, S., Di Cristina, A., Pipitone, M. R., Balzarini, J., Kandil, S., Brancale, A., Sarkar, T., andHamel, E. Synthesis and biological evaluation of 2-amino-3-(30,40,50-trimethoxybenzoyl)-6-substituted-4,5,6,7-tetrahydrothieno[2,3-c]pyridine derivatives as antimitotic agents and inhibitors of tubulin polymerization. Bioorg. & Med. Chem. Lett. 2008, 18, 5041-5045.

14. WO2009009550A1.

15. Fracchiolla, G., Laghezza, A., Piemontese, L., Carbonara, G., Lavecchia, A., Tortorella, P., Crestani, M., Novellino, E., and Loiodice, F. Synthesis, Biological Evaluation, and Molecular Modeling Investigation of Chiral Phenoxyacetic Acid Analogues with PPARa and PPARg Agonist Activity. ChemMedChem. 2007, 2, 641-654.

16. Hutt, D. M., Herman. D., Rodrigues, A. P. C., Noel, S., Pilewski, J. M., Matteson, J., Hoch, B., Kellner, W., Kelly, J.W., Schmidt, A., Thomas, P. J., Matsumura, Y., Skach, W. R., Gentzsch, M., Riordan, J. R., Sorscher, E. J., Okiyoneda, T., Yates, J. R. III, Lukacs, G. L., Frizzell, R. A., Manning, G., Gottesfeld, and J. M., Balch, W. E. Reduced histone deacetylase 7 activity restores function to misfolded CFTR in cystic fibrosis. Nature: Chemical Biology. 2010, 6(1), 25-33

17. Lei, D. C. et al. Episomal expression of wild-type CFTR corrects cAMP-dependent chloride transport in respiratory epithelial cells. Gene Ther.1996, 3, 427-436.

18. Devor, D.C., Bridges, R. J. & Pilewski, J. M. Pharmacological modulation of ion transport across wild-type and DeltaF508 CFTR-expressing human bronchial epithelia. Am. J. Physiol. Cell Physiol. 2000, 279, C461-C479.

19. Butterworth, M. B., Edinger, R. S., Johnson, J. P. & Frizzell, R. A. Acute ENaC stimulation by cAMP in a kidney cell line is mediated by exocytic insertion from a recycling channel pool. J. Gen. Physiol. 2005, 125, 81-101.

20. Gentzsch, M. et al. Endocytic trafficking routes of wild type and DeltaF508 cystic fibrosis transmembrane conductance regulator. Mol. Biol. Cell 2004, 15, 2684-2696.

21. Lahm, A. et al. Unraveling the hidden catalytic activity of vertebrate class IIa histone deacetylases. Proc. Natl. Acad. Sci. USA. 2007, 104, 17335-17340.

While this invention has been particularly shown and described with references to preferred embodiments thereof, it will be understood by those skilled in the art that various changes in form and details may be made therein without departing from the scope of the invention encompassed by the appended claims.

1-12. (canceled)

13. A method of treating a patient suffering from a condition associated with a dysfunction in proteostasis comprising administering an effective amount of a compound having the Formula (IIe), (IIf), (IIh), or (IIi): 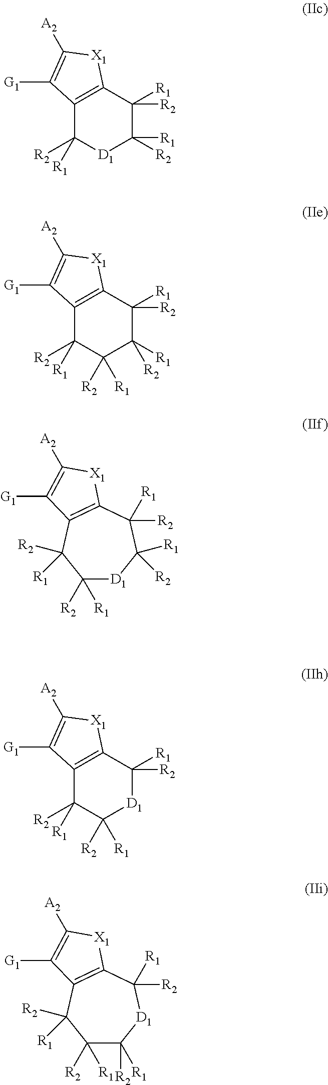
or a pharmaceutically acceptable salt, solvate, or prodrug of any of thereof; wherein:
X₁ is S;
D₁ is selected from the group consisting of N(Rₐ), N(ORₐ), N(Rₐ)(O), N(+)(Rₐ)₂, O and S;
A₂ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂,NR_{b}S(O)ₙR_{b}, NR_{b}S(O)ₙN(R_{b})₂ and OC(O)OR_{b};
G₁ is selected from the group consisting of hydrogen, optionally substituted 3- to 12-membered heterocyclic, optionally substituted heteroaryl, optionally substituted aryl, C(O)N(R_{b})₂, NR_{b}C(O)R_{b}, NR_{b}S(O)ₙR_{b}, and C(R_{b})₂OR_{b};
each of R₁ and R₂ are independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, SR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, S(O)ₙR_{b}, S(O)ₙNR_{b}R_{b}, OC(O)OR_{b} and (C═NR_{b})R_{b}; alternatively, two vicinal R₁ groups can be taken together with the carbon atoms to which they are attached to form a fused, optionally substituted cyclic group selected from the group consisting of optionally substituted C₄-C₈ cycloalkyl, optionally substituted C₄-C₈ cycloalkenyl, optionally substituted 4- to 8-membered heterocyclic, optionally substituted aryl and optionally substituted heteroaryl; yet alternatively, two geminal R₁ and R₂ groups can be taken together with the carbon to which they are attached to form a spiro, optionally substituted cyclic group selected from the group consisting of optionally substituted C₃-C₈ cycloalkyl, optionally substituted C₃-C₈ cycloalkenyl, optionally substituted 3- to 8-membered heterocyclic, optionally substituted aryl and optionally substituted heteroaryl, alternatively, two geminal R₁ and R₂ groups can be taken together with the carbon atom to which they are attached to form a carbonyl group;
Rₐ is selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl, C(O)R_{b}, C(O)C(O)R_{b}, C(O)NR_{b}R_{b}, O(R_{b}), and S(O)ₙR_{b};
each R_{b} is independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl; and
n is 0, 1 or 2.

14-27. (canceled)

28. The method of claim 13, wherein D₁ is N(Rₐ) and wherein each Rₐ is independently selected from the group consisting of hydrogen and optionally substituted C₁-C₁₀ alkyl.

29-32. (canceled)

33. The method of claim 28, wherein G₁ is an optionally substituted 5/6-membered fused heteroaryl.

34. The method of claim 33, wherein G₁ is benzothiazolyl, benzoxazolyl, benzimidazolyl, benzothiophenyl, and benzofuranyl, each optionally substituted.

35. A method of treating a patient suffering from a condition associated with a dysfunction in proteostasis comprising administering an effective amount of a compound having the Formula (IIIe) or (IIIf): 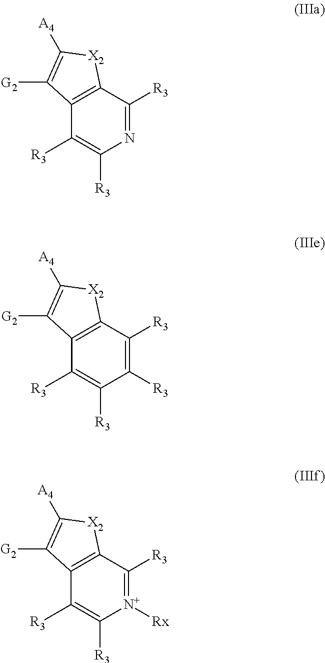
or a pharmaceutically acceptable salt, solvate, or prodrug of any thereof; wherein:
X₂ is S;
A₄ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{c}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂,NR_{b}S(O)ₙN(R_{b})₂, and OC(O)OR_{b};
G₂ is selected from the group consisting of optionally substituted 3- to 12-membered heterocyclic and optionally substituted heteroaryl, CON(R_{b})₂, NR_{b}C(O)R_{b}, NR_{b}S(O)ₙR_{b}, and C(R_{b})₂OR_{b};
each R₃ is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, SR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, S(O)ₙR_{b}, S(O)ₙNR_{b}R_{b}, OC(O)OR_{b} and (C═NR_{b})R_{b}; alternatively, two vicinal R₃ groups can be taken together with the carbon atoms to which they are attached to form a fused, optionally substituted cyclic group selected from the group consisting of optionally substituted C₄-C₈ cycloalkyl, optionally substituted C₄-C₈ cycloalkenyl, optionally substituted 4- to 8-membered heterocyclic, optionally substituted aryl and optionally substituted heteroaryl;
Rₐ is selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl, C(O)R_{b}, C(O)C(O)R_{b}, C(O)NR_{b}R_{b}, OR_{b}, and S(O)ₙR_{b};
each R_{b} is independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl; and
n is 0, 1 or 2.

36-46. (canceled)

47. The method of claim 35, wherein G₂ is an optionally substituted 5/6-membered fused heteroaryl.

48. The method of claim 47, wherein G₂ is benzothiazolyl, benzoxazolyl, benzimidazolyl, benzothiophenyl, and benzofuranyl, each optionally substituted.

49. A method of treating a patient suffering from a condition associated with a dysfunction in proteostasis comprising administering an effective amount of a compound having the Formula (IVa), (IVb) or (IVc): 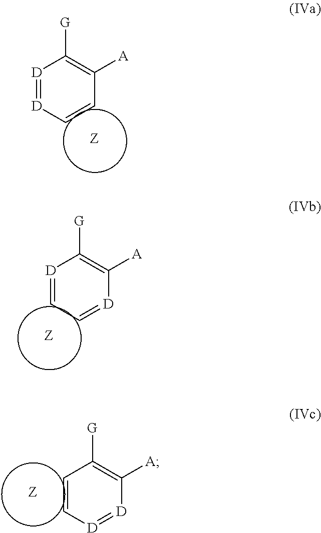
or a pharmaceutically acceptable salt, solvate, or prodrug of any of thereof; wherein:
the two D groups are each C(R), or alternatively, one of the D groups is C(Rₐ) and the other D group is N;
Ring Z is a monocyclic or polycyclic ring system fused to the six-membered aromatic ring containing the D groups, wherein Ring Z is a C₄-C₁₂ cycloalkyl, C₄-C₁₂ cycloalkenyl, 4- to 12-membered heterocyclic, aryl or heteroaryl, each optionally substituted;
A is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂ and OC(O)OR_{b};
G is selected from the group consisting of optionally substituted 3- to 12-membered heterocyclic, optionally substituted heteroaryl, CON(R_{b})₂, NR_{b}C(O)R_{b}, NR_{b}S(O)ₙR_{b}, and C(R_{b})₂OR_{b};
Rₐ is selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl, C(O)R_{b}, C(O)C(O)R_{b}, C(O)NR_{b}R_{b}, O(R_{b}), and S(O)ₙR_{b};
each R_{b} is independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl; and
n is 0, 1 or 2.

50-65. (canceled)

66. A method of treating a patient suffering from a condition associated with a dysfunction in proteostasis comprising administering an effective amount of a compound selected from the group consisting of:

| Compound | |
|---|---|
| Number | Compound |
| 1 | 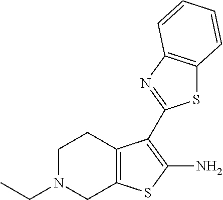 [SMILES: CCN1CCc2c(sc(N)c2-c2nc3ccccc3s2)C1] |
| 2 | 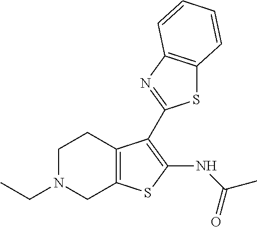 [SMILES: CCN1CCc2c(sc(NC(C)=O)c2-c2nc3ccccc3s2)C1] |
| 3 | 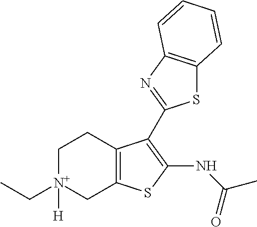 [SMILES: CC[NH+]1CCc2c(sc(NC(C)=O)c2-c2nc3ccccc3s2)C1] |
| 4 | 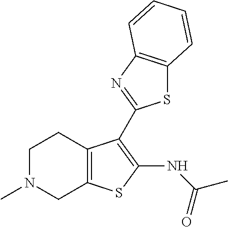 [SMILES: CC(=O)Nc1sc2c(c1-c1nc3ccccc3s1)CCN(C)C2] |
| 5 | 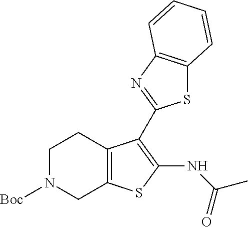 [SMILES: CC(=O)Nc1sc2c(c1-c1nc3ccccc3s1)CCN(C(=O)OC(C)(C)C)C2] |
| 6 | 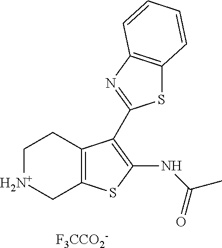 |
| 7 | 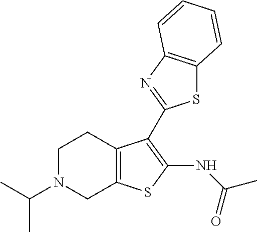 [SMILES: CC(=O)Nc1sc2c(c1-c1nc3ccccc3s1)CCN(C(C)C)C2] |
| 8 | 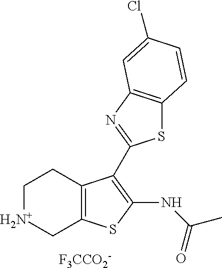 |
| 9 | 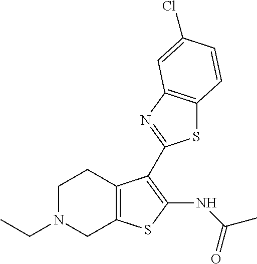 [SMILES: CCN1CCc2c(sc(NC(C)=O)c2-c2nc3cc(Cl)ccc3s2)C1] |
| 10 | 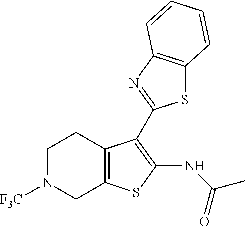 [SMILES: CC(=O)Nc1sc2c(c1-c1nc3ccccc3s1)CCN(C(F)(F)F)C2] |
| 11 | 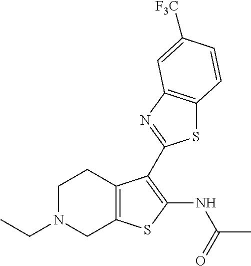 [SMILES: CCN1CCc2c(sc(NC(C)=O)c2-c2nc3cc(C(F)(F)F)ccc3s2)C1] |
| 12 | 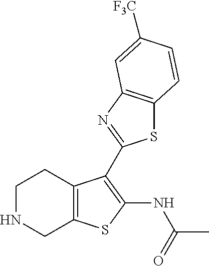 [SMILES: CC(=O)Nc1sc2c(c1-c1nc3cc(C(F)(F)F)ccc3s1)CCNC2] |
| 13 | 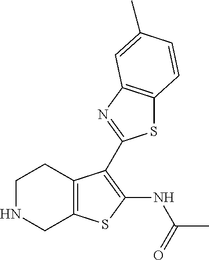 [SMILES: CC(=O)Nc1sc2c(c1-c1nc3cc(C)ccc3s1)CCNC2] |
| 14 | 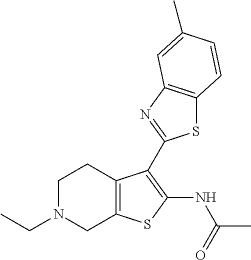 [SMILES: CCN1CCc2c(sc(NC(C)=O)c2-c2nc3cc(C)ccc3s2)C1] |
| 15 | 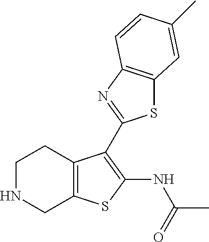 [SMILES: CC(=O)Nc1sc2c(c1-c1nc3ccc(C)cc3s1)CCNC2] |
| 16 | 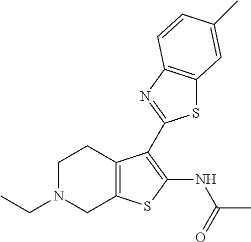 [SMILES: CCN1CCc2c(sc(NC(C)=O)c2-c2nc3ccc(C)cc3s2)C1] |
| 17 | 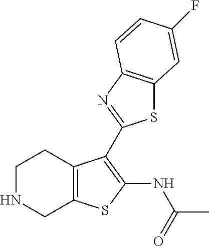 [SMILES: CC(=O)Nc1sc2c(c1-c1nc3ccc(F)cc3s1)CCNC2] |
| 18 | 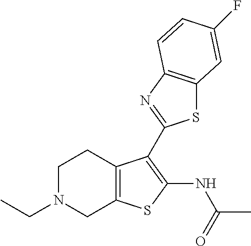 [SMILES: CCN1CCc2c(sc(NC(C)=O)c2-c2nc3ccc(F)cc3s2)C1] |
| 19 | 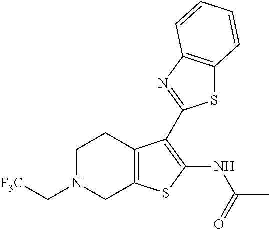 [SMILES: CC(=O)Nc1sc2c(c1-c1nc3ccccc3s1)CCN(CC(F)(F)F)C2] |
| 20 | 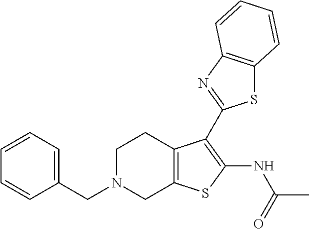 [SMILES: CC(=O)Nc1sc2c(c1-c1nc3ccccc3s1)CCN(Cc1ccccc1)C2] |
| 21 | 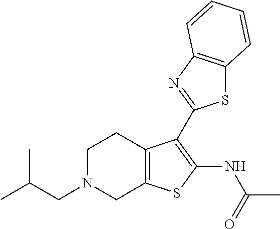 [SMILES: CC(=O)Nc1sc2c(c1-c1nc3ccccc3s1)CCN(CC(C)C)C2] |
| 22 | 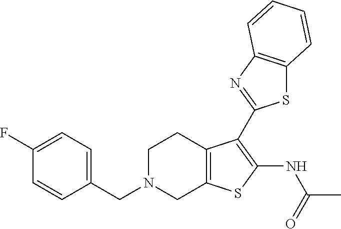 [SMILES: CC(=O)Nc1sc2c(c1-c1nc3ccccc3s1)CCN(Cc1ccc(F)cc1)C2] |
| 23 | 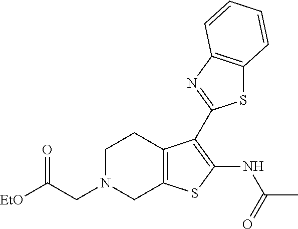 [SMILES: CCOC(=O)CN1CCc2c(sc(NC(C)=O)c2-c2nc3ccccc3s2)C1] |
| 24 | 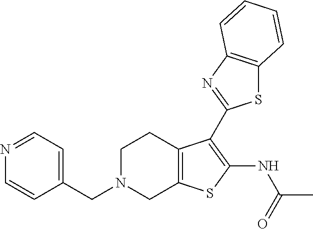 [SMILES: CC(=O)Nc1sc2c(c1-c1nc3ccccc3s1)CCN(Cc1ccncc1)C2] |
| 25 | 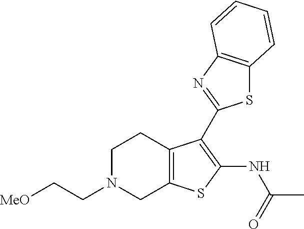 [SMILES: COCCN1CCc2c(sc(NC(C)=O)c2-c2nc3ccccc3s2)C1] |
| 26 | 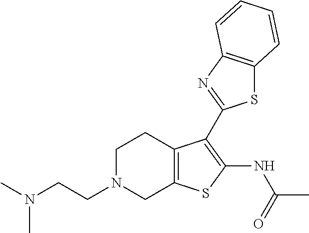 [SMILES: CC(=O)Nc1sc2c(c1-c1nc3ccccc3s1)CCN(CCN(C)C)C2] |
| 27 | 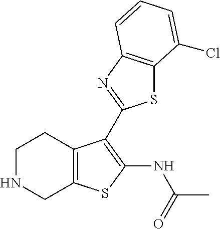 [SMILES: CC(=O)Nc1sc2c(c1-c1nc3cccc(Cl)c3s1)CCNC2] |
| 28 | 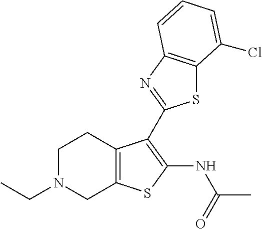 [SMILES: CCN1CCc2c(sc(NC(C)=O)c2-c2nc3cccc(Cl)c3s2)C1] |
| 29 | 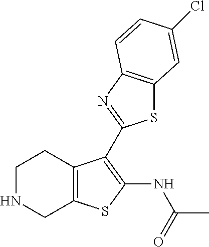 [SMILES: CC(=O)Nc1sc2c(c1-c1nc3ccc(Cl)cc3s1)CCNC2] |
| 30 | 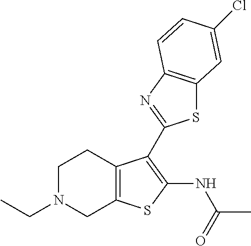 [SMILES: CCN1CCc2c(sc(NC(C)=O)c2-c2nc3ccc(Cl)cc3s2)C1] |
| 31 | 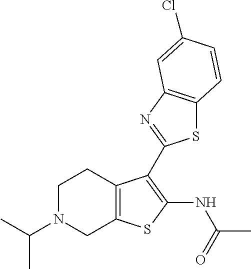 [SMILES: CC(=O)Nc1sc2c(c1-c1nc3cc(Cl)ccc3s1)CCN(C(C)C)C2] |
| 32 | 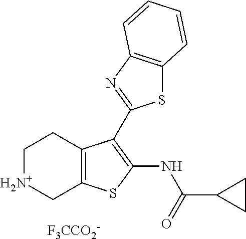 |
| 33 | 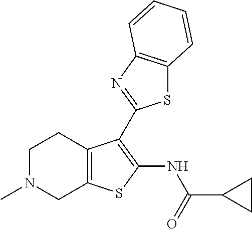 [SMILES: CN1CCc2c(sc(NC(=O)C3CC3)c2-c2nc3ccccc3s2)C1] |
| 34 | 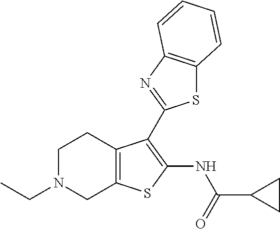 [SMILES: CCN1CCc2c(sc(NC(=O)C3CC3)c2-c2nc3ccccc3s2)C1] |
| 35 | 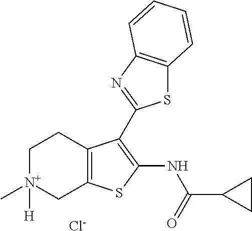 [SMILES: C[NH+]1CCc2c(sc(NC(=O)C3CC3)c2-c2nc3ccccc3s2)C1 ; [Cl-]] |
| 36 | 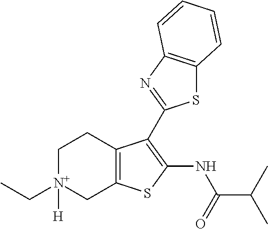 [SMILES: CC[NH+]1CCc2c(sc(NC(=O)C(C)C)c2-c2nc3ccccc3s2)C1] |
| 37 | 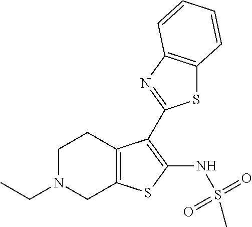 [SMILES: CCN1CCc2c(sc(NS(C)(=O)=O)c2-c2nc3ccccc3s2)C1] |
| 38 | 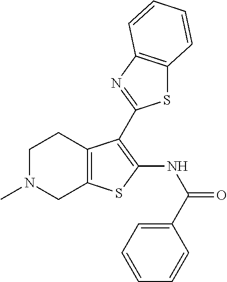 [SMILES: CN1CCc2c(sc(NC(=O)c3ccccc3)c2-c2nc3ccccc3s2)C1] |
| 39 | 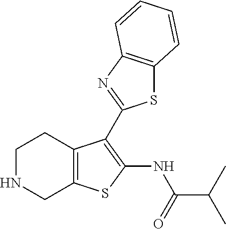 [SMILES: CC(C)C(=O)Nc1sc2c(c1-c1nc3ccccc3s1)CCNC2] |
| 40 | 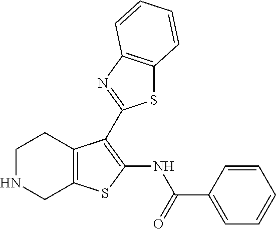 [SMILES: O=C(Nc1sc2c(c1-c1nc3ccccc3s1)CCNC2)c1ccccc1] |
| 41 | 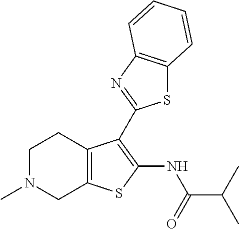 [SMILES: CC(C)C(=O)Nc1sc2c(c1-c1nc3ccccc3s1)CCN(C)C2] |
| 42 | 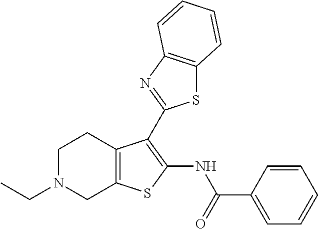 [SMILES: CCN1CCc2c(sc(NC(=O)c3ccccc3)c2-c2nc3ccccc3s2)C1] |
| 43 | 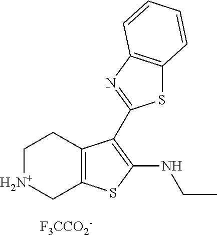 |
| 44 | 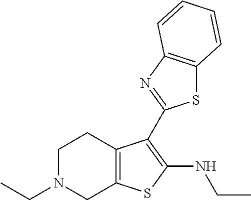 [SMILES: CCNc1sc2c(c1-c1nc3ccccc3s1)CCN(CC)C2] |
| 45 | 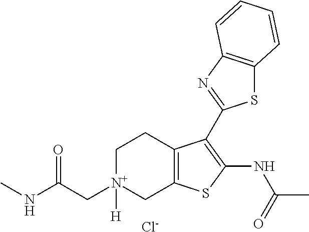 [SMILES: CNC(=O)C[NH+]1CCc2c(sc(NC(C)=O)c2-c2nc3ccccc3s2)C1 ; [Cl-]] |
| 46 | 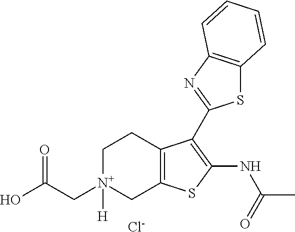 [SMILES: CC(=O)Nc1sc2c(c1-c1nc3ccccc3s1)CC[NH+](CC(=O)O)C2 ; [Cl-]] |
| 47 | 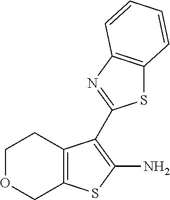 [SMILES: Nc1sc2c(c1-c1nc3ccccc3s1)CCOC2] |
| 48 | 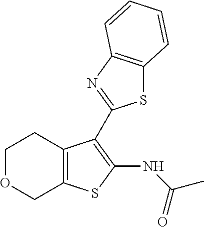 [SMILES: CC(=O)Nc1sc2c(c1-c1nc3ccccc3s1)CCOC2] |
| 51 | 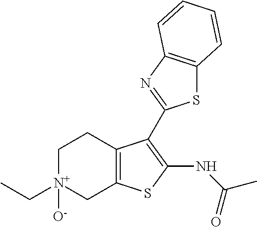 [SMILES: CC[N+]1([O-])CCc2c(sc(NC(C)=O)c2-c2nc3ccccc3s2)C1] |
| 52 | 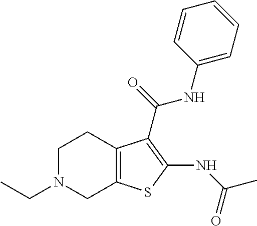 [SMILES: CCN1CCc2c(sc(NC(C)=O)c2C(=O)Nc2ccccc2)C1] |
| 53 | 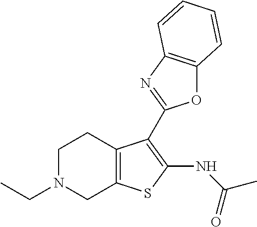 [SMILES: CCN1CCc2c(sc(NC(C)=O)c2-c2nc3ccccc3o2)C1] |
| 54 | 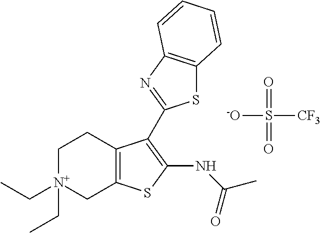 |
| 58 | 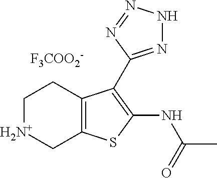 [SMILES: "F3COO2-" ; CC(=O)Nc1sc2c(c1-c1nn[nH]n1)CC[NH2+]C2] |
| 59 | 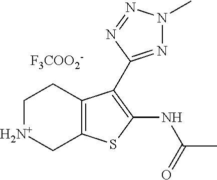 [SMILES: "F3COO2-" ; CC(=O)Nc1sc2c(c1-c1nnn(C)n1)CC[NH2+]C2] |
| 60 | 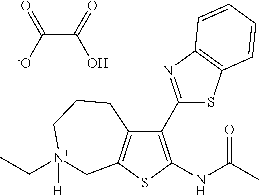 |
| 61 | 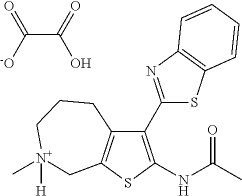 |
| 62 | 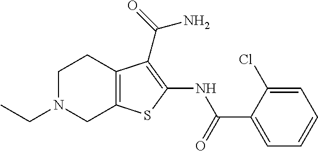 [SMILES: CCN1CCc2c(sc(NC(=O)c3ccccc3Cl)c2C(N)=O)C1] |
| 63 | 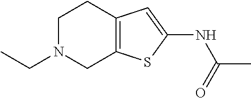 |
| 65 | 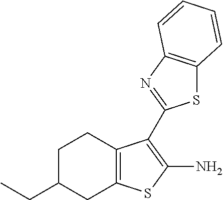 [SMILES: CCC1CCc2c(sc(N)c2-c2nc3ccccc3s2)C1] |
| 66 | 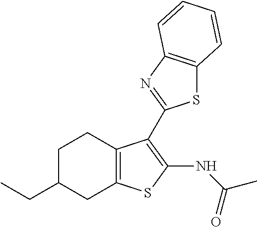 [SMILES: CCC1CCc2c(sc(NC(C)=O)c2-c2nc3ccccc3s2)C1] |
| 67 | 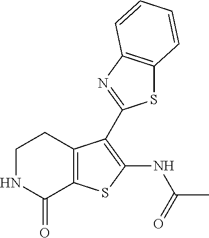 [SMILES: CC(=O)Nc1sc2c(c1-c1nc3ccccc3s1)CCNC2=O] |
| 68 | 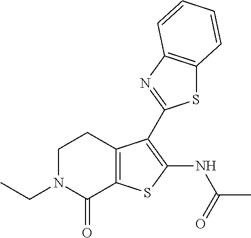 [SMILES: CCN1CCc2c(sc(NC(C)=O)c2-c2nc3ccccc3s2)C1=O] |
| 69 | 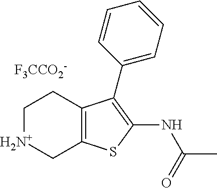 [SMILES: "F3CCO2-" ; CC(=O)Nc1sc2c(c1-c1ccccc1)CC[NH2+]C2] |
| 70 | 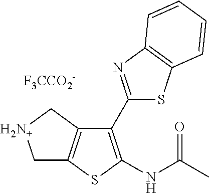 [SMILES: "F3CCO2-" ; CC(=O)Nc1sc2c(c1-c1nc3ccccc3s1)C[NH2+]C2] |
| 71 | 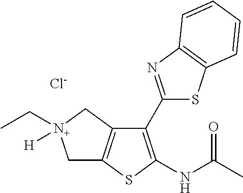 [SMILES: CC[NH+]1Cc2sc(NC(C)=O)c(-c3nc4ccccc4s3)c2C1 ; [Cl-]] |
| 72 | 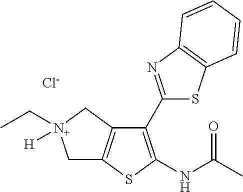 [SMILES: CC[NH+]1Cc2sc(NC(C)=O)c(-c3nc4ccccc4s3)c2C1 ; [Cl-]] |
| 77 | 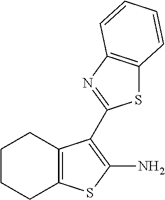 [SMILES: Nc1sc2c(c1-c1nc3ccccc3s1)CCCC2] |
| 78 | 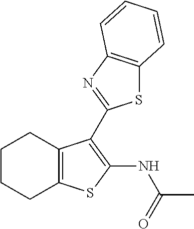 [SMILES: CC(=O)Nc1sc2c(c1-c1nc3ccccc3s1)CCCC2] |
| 80 | 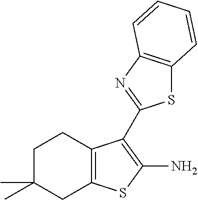 [SMILES: CC1(C)CCc2c(sc(N)c2-c2nc3ccccc3s2)C1] |
| 81 | 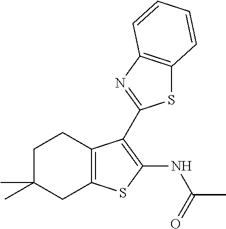 [SMILES: CC(=O)Nc1sc2c(c1-c1nc3ccccc3s1)CCC(C)(C)C2] |
| 82 | 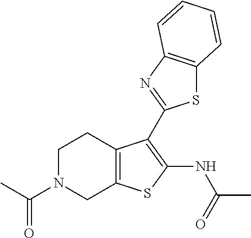 [SMILES: CC(=O)Nc1sc2c(c1-c1nc3ccccc3s1)CCN(C(C)=O)C2] |
| 83 | 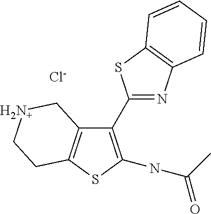 [SMILES: CC(=O)Nc1sc2c(c1-c1nc3ccccc3s1)C[NH2+]CC2 ; [Cl-]] |
| 84 | 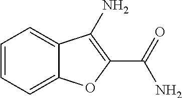 [SMILES: NC(=O)c1oc2ccccc2c1N] |
| 85 | 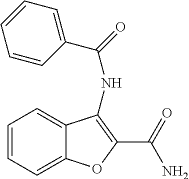 [SMILES: NC(=O)c1oc2ccccc2c1NC(=O)c1ccccc1] |
| 86 | 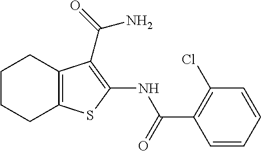 [SMILES: NC(=O)c1c(NC(=O)c2ccccc2Cl)sc2c1CCCC2] |
| 87 | 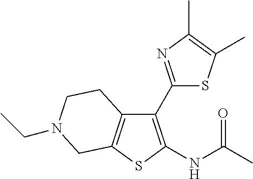 [SMILES: CCN1CCc2c(sc(NC(C)=O)c2-c2nc(C)c(C)s2)C1] |
| 88 | 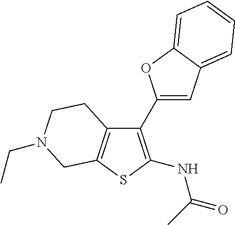 [SMILES: CCN1CCc2c(sc(NC(C)=O)c2-c2cc3ccccc3o2)C1] |
| 89 | 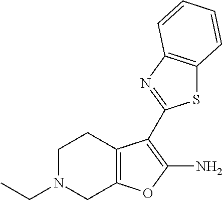 [SMILES: CCN1CCc2c(oc(N)c2-c2nc3ccccc3s2)C1] |
| 90 | 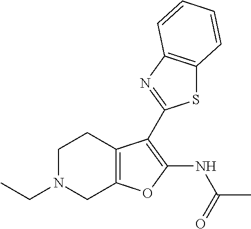 [SMILES: CCN1CCc2c(oc(NC(C)=O)c2-c2nc3ccccc3s2)C1] |
| 91 | 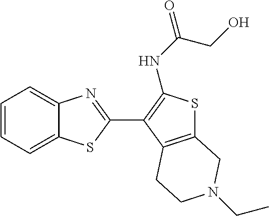 [SMILES: CCN1CCc2c(sc(NC(=O)CO)c2-c2nc3ccccc3s2)C1] |
| 92 | 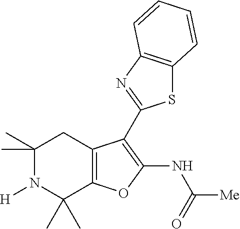 [SMILES: CC(=O)Nc1oc2c(c1-c1nc3ccccc3s1)CC(C)(C)NC2(C)C] |
| 93 | 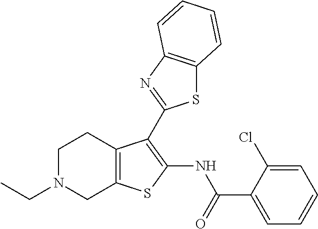 [SMILES: CCN1CCc2c(sc(NC(=O)c3ccccc3Cl)c2-c2nc3ccccc3s2)C1] |
| 94 | 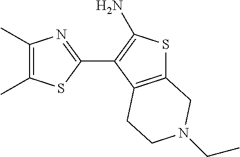 [SMILES: CCN1CCc2c(sc(N)c2-c2nc(C)c(C)s2)C1] |
| 95 | 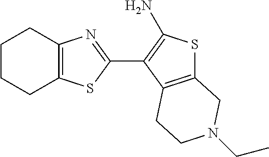 [SMILES: CCN1CCc2c(sc(N)c2-c2nc3c(s2)CCCC3)C1] |
| 96 | 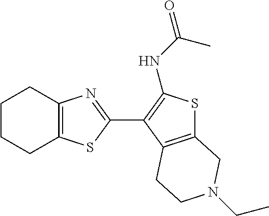 [SMILES: CCN1CCc2c(sc(NC(C)=O)c2-c2nc3c(s2)CCCC3)C1] |
| 97 | 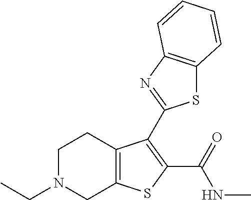 [SMILES: CCN1CCc2c(sc(C(=O)NC)c2-c2nc3ccccc3s2)C1] |
| 98 | 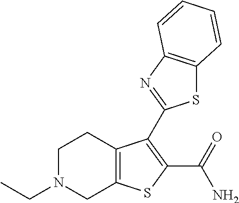 [SMILES: CCN1CCc2c(sc(C(N)=O)c2-c2nc3ccccc3s2)C1] |
| 99 | 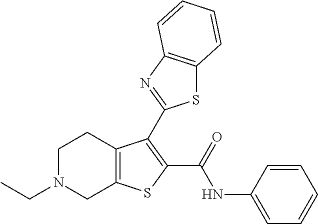 [SMILES: CCN1CCc2c(sc(C(=O)Nc3ccccc3)c2-c2nc3ccccc3s2)C1] |
| 100 | 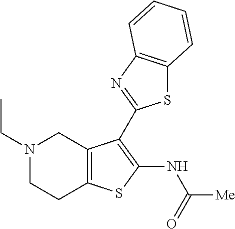 [SMILES: CCN1CCc2sc(NC(C)=O)c(-c3nc4ccccc4s3)c2C1] |
| 101 | 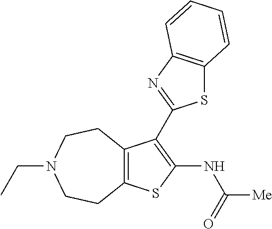 [SMILES: CCN1CCc2sc(NC(C)=O)c(-c3nc4ccccc4s3)c2CC1] |
| 102 | 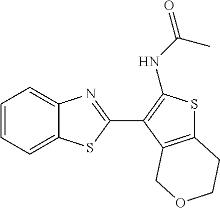 [SMILES: CC(=O)Nc1sc2c(c1-c1nc3ccccc3s1)COCC2] |
| 103 | 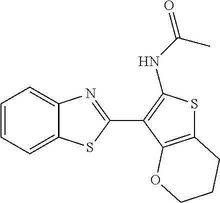 [SMILES: CC(=O)Nc1sc2c(c1-c1nc3ccccc3s1)OCCC2] |
| 104 | 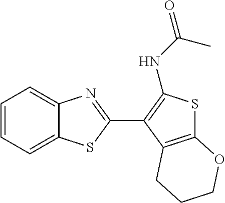 [SMILES: CC(=O)Nc1sc2c(c1-c1nc3ccccc3s1)CCCO2] |
| 105 | 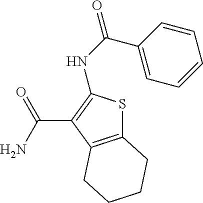 [SMILES: NC(=O)c1c(NC(=O)c2ccccc2)sc2c1CCCC2] |
| 106 | 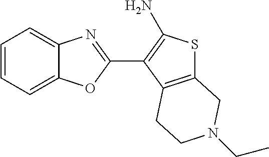 [SMILES: CCN1CCc2c(sc(N)c2-c2nc3ccccc3o2)C1] |
| 107 | 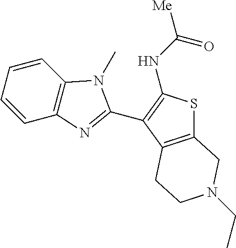 [SMILES: CCN1CCc2c(sc(NC(C)=O)c2-c2nc3ccccc3n2C)C1] |
| 108 | 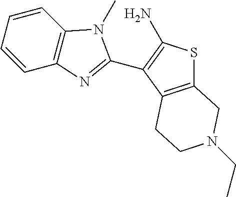 [SMILES: CCN1CCc2c(sc(N)c2-c2nc3ccccc3n2C)C1] |
| 109 | 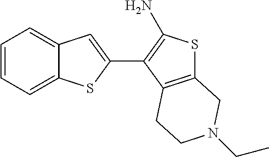 [SMILES: CCN1CCc2c(sc(N)c2-c2cc3ccccc3s2)C1] |
| 110 | 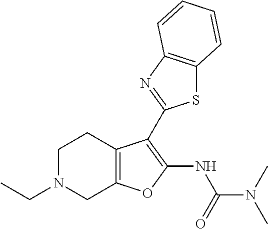 [SMILES: CCN1CCc2c(oc(NC(=O)N(C)C)c2-c2nc3ccccc3s2)C1] |
| 111 | 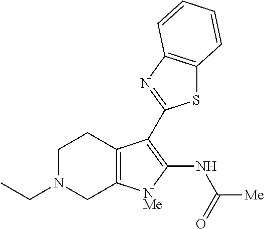 [SMILES: CCN1CCc2c(-c3nc4ccccc4s3)c(NC(C)=O)n(C)c2C1] |
| 112 | 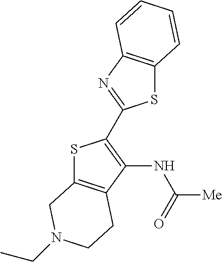 [SMILES: CCN1CCc2c(sc(-c3nc4ccccc4s3)c2NC(C)=O)C1] |
| 113 | 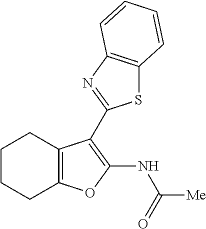 [SMILES: CC(=O)Nc1oc2c(c1-c1nc3ccccc3s1)CCCC2] |
| 114 | 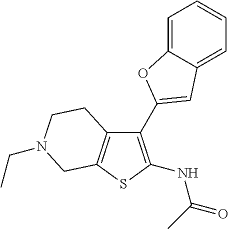 [SMILES: CCN1CCc2c(sc(NC(C)=O)c2-c2cc3ccccc3o2)C1] |
| 123 | 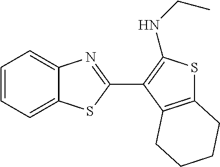 [SMILES: CCNc1sc2c(c1-c1nc3ccccc3s1)CCCC2] |
| 49 | 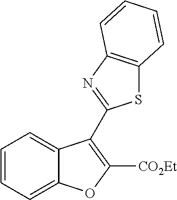 [SMILES: CCOC(=O)c1oc2ccccc2c1-c1nc2ccccc2s1] |
| 50 | 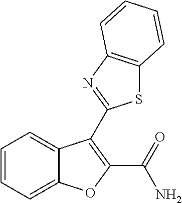 [SMILES: NC(=O)c1oc2ccccc2c1-c1nc2ccccc2s1] |
| 55 | 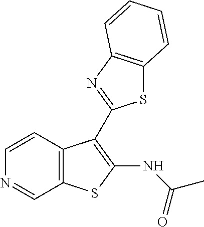 [SMILES: CC(=O)Nc1sc2cnccc2c1-c1nc2ccccc2s1] |
| 56 | 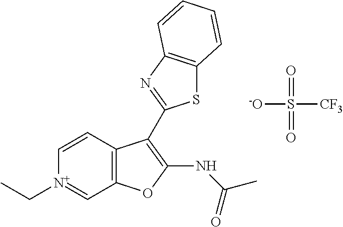 |
| 57 | 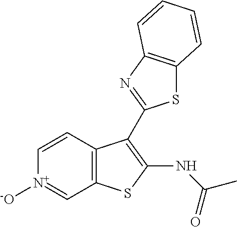 |
| 79 | 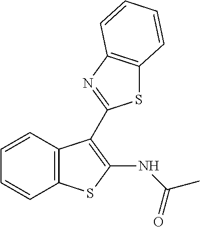 [SMILES: CC(=O)Nc1sc2ccccc2c1-c1nc2ccccc2s1] |
| 84 | 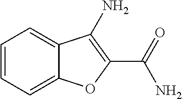 [SMILES: NC(=O)c1oc2ccccc2c1N] |
| 85 | 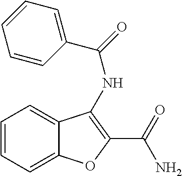 [SMILES: NC(=O)c1oc2ccccc2c1NC(=O)c1ccccc1] |
| 115 | 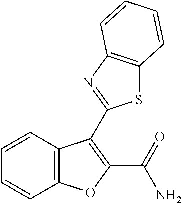 [SMILES: NC(=O)c1oc2ccccc2c1-c1nc2ccccc2s1] |
| 116 | 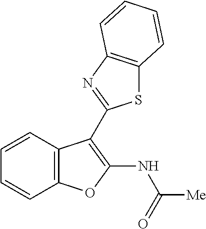 [SMILES: CC(=O)Nc1oc2ccccc2c1-c1nc2ccccc2s1] |
| 117 | 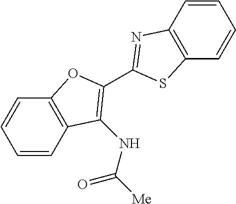 [SMILES: CC(=O)Nc1c(-c2nc3ccccc3s2)oc2ccccc12] |
| 118 | 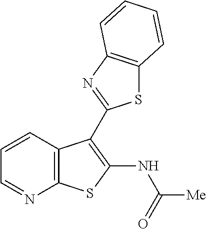 [SMILES: CC(=O)Nc1sc2ncccc2c1-c1nc2ccccc2s1] |
| 119 | 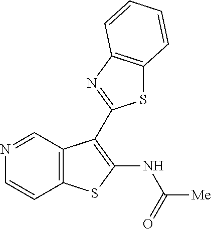 [SMILES: CC(=O)Nc1sc2ccncc2c1-c1nc2ccccc2s1] |
| 120 | 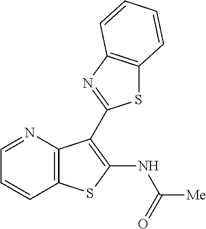 [SMILES: CC(=O)Nc1sc2cccnc2c1-c1nc2ccccc2s1] |
| Compound | |
|---|---|
| Number | Compound |
| 64 | 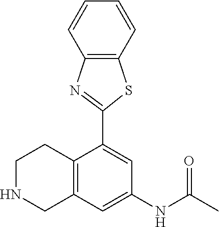 [SMILES: CC(=O)Nc1cc2c(c(-c3nc4ccccc4s3)c1)CCNC2] |
| 73 | 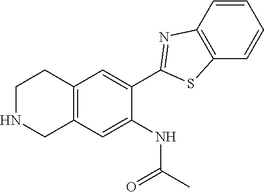 [SMILES: CC(=O)Nc1cc2c(cc1-c1nc3ccccc3s1)CCNC2] |
| 74 | 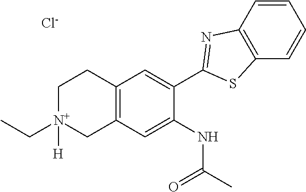 [SMILES: CC[NH+]1CCc2cc(-c3nc4ccccc4s3)c(NC(C)=O)cc2C1 ; [Cl-]] |
| 75 | 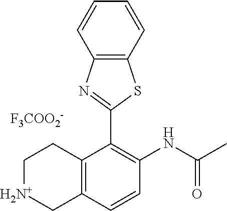 [SMILES: "F3COO2-" ; CC(=O)Nc1ccc2c(c1-c1nc3ccccc3s1)CC[NH2+]C2] |
| 76 | 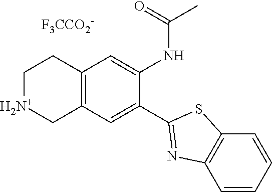 [SMILES: "F3CCO2-" ; CC(=O)Nc1cc2c(cc1-c1nc3ccccc3s1)C[NH2+]CC2] |
| 121 | 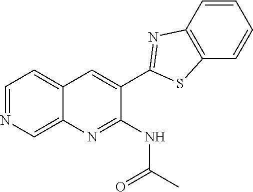 [SMILES: CC(=O)Nc1nc2cnccc2cc1-c1nc2ccccc2s1] |
| 122 | 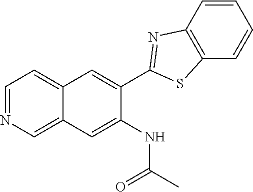 [SMILES: CC(=O)Nc1cc2cnccc2cc1-c1nc2ccccc2s1] |

67. A method of treating a patient suffering from a condition associated with a dysfunction in proteostasis comprising administering an effective amount of a compound represented by Formula (VII): 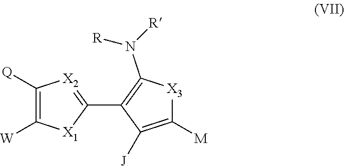
or a pharmaceutically acceptable salt, solvate, prodrug or N-oxide thereof, wherein:
X₁ and X₃ are each independently O, S or NR^{c}, where each R^{c} is independently H or C₁-C₆-alkyl;
X₂ is N or CR^{c};
J and M, together with the carbon atoms to which they are attached, form an optionally substituted 5 to 7 membered carbocyclic or heterocyclic ring, wherein the heterocyclic ring comprises one or two heteroatoms independently selected from nitrogen and oxygen and the remainder of the ring atoms are carbon;
Q and W, together with the carbon atoms to which they are attached, form an optionally substituted 5 to 7 membered carbocyclic ring; and
R and R′ are each independently hydrogen, C₁-C₆-alkyl, C₂-C₆-alkenyl, C₂-C₆-alkynyl, —C(O)-R^{d} or —SO₂-R^{d}, where R^{d} is C₁-C₆-alkyl, substituted or unsubstituted aryl, or substituted or unsubstituted aryl-C₁-C₆-alkyl.

68. The method of claim 67 wherein the compound is represented by Formula VIII: 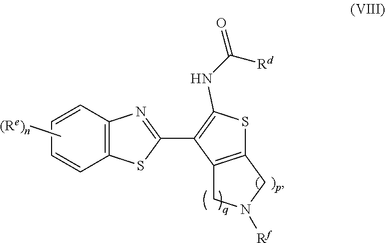
or a pharmaceutically acceptable salt, solvate, prodrug or N-oxide thereof, wherein:
R^{d} is C₁-C₆-alkyl, substituted C₁-C₆-alkyl, phenyl or halogen-substituted phenyl;
each R^{e} is C₁-C₆-alkyl, halogenated C₁-C₆-alkyl, OR^{d} or halogen;
R^{f} is hydrogen, C₁-C₆-alkyl or substituted C₁-C₆-alkyl;
n is 0 to 3;
p and q are each independently 1 to 3, provided that the sum of p and q is 2 to 4.

69. The method of claim 67 wherein the compound is represented by Formula (IX): 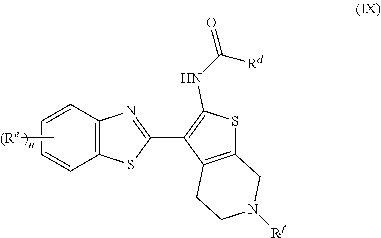
or a pharmaceutically acceptable salt, solvate, prodrug or N-oxide thereof, wherein:
R^{d} is C₁-C₆-alkyl, substituted C₁-C₆-alkyl, phenyl or halogen-substituted phenyl;
n is 0 or 1;
R^{e} is C₁-C₆-alkyl, halogenated C₁-C₆-alkyl or halogen; and
R^{f} is hydrogen, C₁-C₆-alkyl or substituted C₁-C₆-alkyl.

70. The method of claim 13, wherein the condition is associated with a dysfunction in the proteostasis of a protein selected from the group consisting of hexosamine A, cystic fibrosis transmembrane conductance regulator, aspartylglucsaminidase, a-galactosidase A, cysteine transporter, acid ceremidase, acid α-L-fucosidase, protective protein, cathepsin A, acid β-glucosidase, acid β-galactosidase, iduronate 2-sulfatase, α-L-iduronidase, galactocerebrosidase, acid α-mannosidase, acid β-mannosidase, arylsulfatase B, arylsulfatase A, N-acetylgalactosamine-6-sulfate sulfatase, acid β-galactosidase, N-acetylglucosamine-1-phosphotransferase, acid sphingmyelinase, NPC-1, acid α-glucosidase, β-hexosamine B, heparin N-sulfatase, α-N-acetylglucosaminidase, α-glucosaminide N-acetyltransferase, N-acetylglucosamine-6-sulfate sulfatase, α1 anti-trypsin, α-N-acetylgalactosaminidase, α-neuramidase, β-glucuronidase, β-hexosamine A and acid lipase, polyglutamine, α-synuclein, Aβ peptide, tau protein, hERG potassium channel, islet amyloid polypeptide, transthyretin Huntingtin, and superoxide dismutase.

71. The method of claim 13, wherein the condition is selected from the group consisting of Huntington's disease, Alzheimer's disease, Parkinson's disease, amyotrophic lateral sclerosis, diabetic retinopathy, diabetes, cancer and cystic fibrosis.

72. The method of claim 71, wherein the condition is cystic fibrosis.

73. (canceled)

74. A pharmaceutical composition for treating a condition associated with a dysfunction in proteostasis comprising an effective amount of a compound wherein the compound of Formula (I) represented by (IIc), (IIe), (IIf), (IIh) or (IIi): 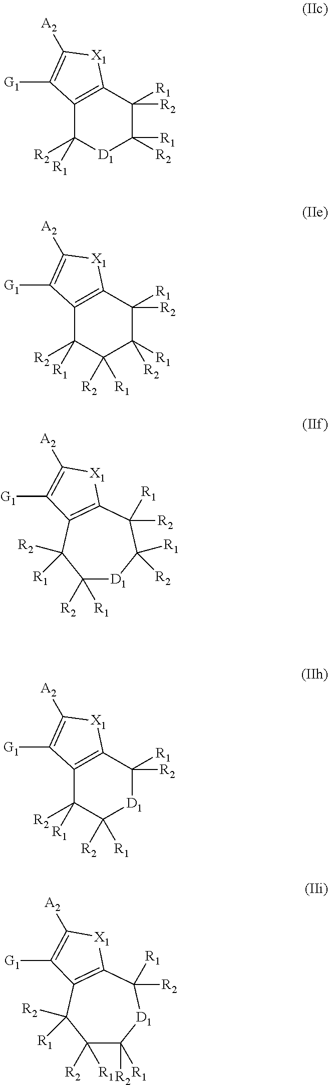
or a pharmaceutically acceptable salt, solvate, or prodrug of any of thereof; wherein:
X₁ is S;
D₁ is selected from the group consisting of N(Rₐ), N(ORₐ), N(Rₐ)(O), N(+)(Rₐ)₂, O and S;
A₂ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂,NR_{b}S(O)ₙR_{b}, NR_{b}S(O)ₙN(R_{b})₂ and OC(O)OR_{b};
G₁ is selected from the group consisting of hydrogen, optionally substituted 3- to 12-membered heterocyclic, optionally substituted heteroaryl, optionally substituted aryl, C(O)N(R_{b})₂, NR_{b}C(O)R_{b}, NR_{b}S(O)ₙR_{b}, and C(R_{b})₂OR_{b};
each of R₁ and R₂ are independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, SR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, S(O)ₙR_{b}, S(O)ₙNR_{b}R_{b}, OC(O)OR_{b} and (C═NR_{b})R_{b}; alternatively, two vicinal R₁ groups can be taken together with the carbon atoms to which they are attached to form a fused, optionally substituted cyclic group selected from the group consisting of optionally substituted C₄-C₈ cycloalkyl, optionally substituted C₄-C₈ cycloalkenyl, optionally substituted 4- to 8-membered heterocyclic, optionally substituted aryl and optionally substituted heteroaryl;
yet alternatively, two geminal R₁ and R₂ groups can be taken together with the carbon to which they are attached to form a spiro, optionally substituted cyclic group selected from the group consisting of optionally substituted C₃-C₈ cycloalkyl, optionally substituted C₃-C₈ cycloalkenyl, optionally substituted 3- to 8-membered heterocyclic, optionally substituted aryl and optionally substituted heteroaryl, alternatively, two geminal R₁ and R₂ groups can be taken together with the carbon atom to which they are attached to form a carbonyl group;
Rₐ is selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl, C(O)R_{b}, C(O)C(O)R_{b}, C(O)NR_{b}R_{b}, O(R_{b}), and S(O)ₙR_{b};
each R_{b} is independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl; and
n is 0, 1 or 2.

75. A pharmaceutical composition for treating a condition associated with a dysfunction in proteostasis comprising an effective amount of a compound represented by Formula (IIIa), (IIIe) or (IIf): 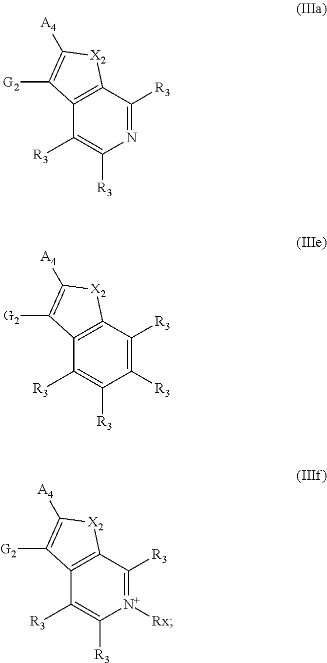
or a pharmaceutically acceptable salt, solvate, or prodrug of any of thereof; wherein:
X₂ is S;
A₄ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{c}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂,NR_{b}S(O)ₙN(R_{b})₂, and OC(O)OR_{b;}
G₂ is selected from the group consisting of optionally substituted 3- to 12-membered heterocyclic and optionally substituted heteroaryl, CON(R_{b})₂, NR_{b}C(O)R_{b}, NR_{b}S(O)ₙR_{b}, and C(R_{b})₂OR_{b};
each R₃ is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, SR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, S(O)ₙR_{b}, S(O)ₙNR_{b}R_{b}, OC(O)OR_{b} and (C═NR_{b})R_{b}; alternatively, two vicinal R₃ groups can be taken together with the carbon atoms to which they are attached to form a fused, optionally substituted cyclic group selected from the group consisting of optionally substituted C₄-C₈ cycloalkyl, optionally substituted C₄-C₈ cycloalkenyl, optionally substituted 4- to 8-membered heterocyclic, optionally substituted aryl and optionally substituted heteroaryl;
Rₐ is selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl, C(O)R_{b}, C(O)C(O)R_{b}, C(O)NR_{b}R_{b}, OR_{b}, and S(O)ₙR_{b};
each R_{b} is independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl; and
n is 0, 1 or 2.

76. A pharmaceutical composition for treating a condition associated with a dysfunction in proteostasis comprising an effective amount of a compound having the Formula (IVa), (IVb) or (IVc): 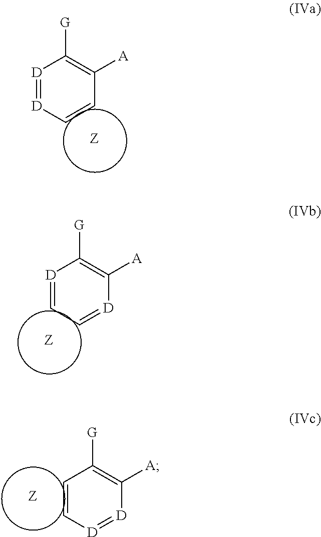
or a pharmaceutically acceptable salt, solvate, or prodrug of any of thereof; wherein:
the two D groups are each C(Rₐ), or alternatively, one of the D groups is C(Rₐ) and the other D group is N;
Ring Z is a monocyclic or polycyclic ring system fused to the six-membered aromatic ring containing the D groups, wherein Ring Z is a C₄-C₁₂ cycloalkyl, C₄-C₁₂ cycloalkenyl, 4- to 12-membered heterocyclic, aryl or heteroaryl, each optionally substituted;
A is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂ and OC(O)OR_{b};
G is selected from the group consisting of optionally substituted 3- to 12-membered heterocyclic, optionally substituted heteroaryl, CON(R_{b})₂, NR_{b}C(O)R_{b}, NR_{b}S(O)ₙR_{b}, and C(R_{b})₂OR_{b};
Rₐ is selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl, C(O)R_{b}, C(O)C(O)R_{b}, C(O)NR_{b}R_{b}, O(R_{b}), and S(O)ₙR_{b};
each R_{b} is independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl; and
n is 0, 1 or 2.

77-78. (canceled)

79. A compound having the Formula (IIe): 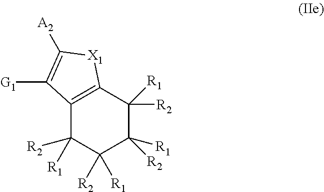
or a pharmaceutically acceptable salt, solvate, or prodrug of any of thereof; wherein:
X₁ is S;
A₂ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙR_{b}, NR_{b}S(O)ₙN(R_{b})₂ and OC(O)OR_{b};
G₁ is selected from the group consisting of optionally substituted 3- to 12-membered heterocyclic, optionally substituted heteroaryl, C(O)N(R_{b})₂, NR_{b}C(O)R_{b}, NR_{b}S(O)ₙR_{b}, and C(R_{b})₂OR_{b};
each of R₁ and R₂ are independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, SR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, S(O)ₙR_{b}, S(O)ₙNR_{b}R_{b}, OC(O)OR_{b} and (C═NR_{b})R_{b}; alternatively, two vicinal R₁ groups can be taken together with the carbon atoms to which they are attached to form a fused, optionally substituted cyclic group selected from the group consisting of optionally substituted C₄-C₈ cycloalkyl, optionally substituted C₄-C₈ cycloalkenyl, optionally substituted 4- to 8-membered heterocyclic, optionally substituted aryl and optionally substituted heteroaryl; yet alternatively, two geminal R₁ and R₂ groups can be taken together with the carbon to which they are attached to form a Spiro, optionally substituted cyclic group selected from the group consisting of optionally substituted C₃-C₈ cycloalkyl, optionally substituted C₃-C₈ cycloalkenyl, optionally substituted 3- to 8-membered heterocyclic, optionally substituted aryl and optionally substituted heteroaryl, alternatively, two geminal R₁ and R₂ groups can be taken together with the carbon atom to which they are attached to form a carbonyl group;
Rₐ is selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl, C(O)R_{b}, C(O)C(O)R_{b}, C(O)NR_{b}R_{b}, O(R_{b}), and S(O)ₙR_{b};
each R_{b} is independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl; and
n is 0, 1 or 2.

80-88. (canceled)

89. The compound of claim 79, wherein G₁ is an optionally substituted 5/6-membered fused heteroaryl.

90. The compound of claim 89, wherein G₁ is benzothiazolyl, benzoxazolyl, benzimidazolyl, benzothiophenyl, and benzofuranyl, each optionally substituted.

91. A compound having the Formula (IIa), (IIIe) or (IIIf): 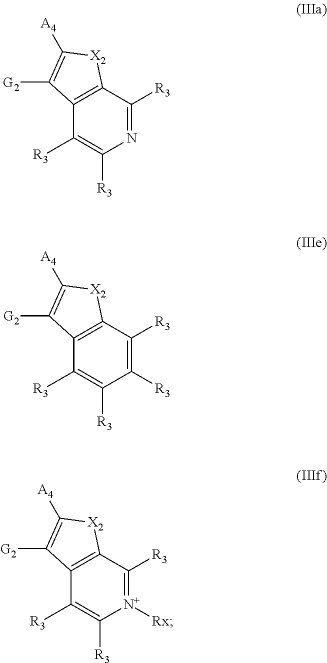
or a pharmaceutically acceptable salt, solvate, or prodrug of any of thereof; wherein:
X₂ is S;
A₄ is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{c}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂,NR_{b}S(O)ₙN(R_{b})₂, and OC(O)OR_{b;}
G₂ is selected from the group consisting of optionally substituted 3- to 12-membered heterocyclic and optionally substituted heteroaryl;
each R₃ is independently selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, SR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂, S(O)ₙR_{b}, S(O)ₙNR_{b}R_{b}, OC(O)OR_{b} and (C═NR_{b})R_{b}; alternatively, two vicinal R₃ groups can be taken together with the carbon atoms to which they are attached to form a fused, optionally substituted cyclic group selected from the group consisting of optionally substituted C₄-C₈ cycloalkyl, optionally substituted C₄-C₈ cycloalkenyl, optionally substituted 4- to 8-membered heterocyclic, optionally substituted aryl and optionally substituted heteroaryl;
Rₐ is selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl, C(O)R_{b}, C(O)C(O)R_{b}, C(O)NR_{b}R_{b}, OR_{b}, and S(O)ₙR_{b};
each R_{b} is independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl; and
n is 0, 1 or 2.

92-96. (canceled)

97. The compound of claim 91, wherein G₂ is an optionally substituted 5/6-membered fused heteroaryl.

98. The compound of claim 97, wherein G₂ is benzothiazolyl, benzoxazolyl, benzimidazolyl, benzothiophenyl, and benzofuranyl, each optionally substituted.

99. A compound having the Formula (IVa), (IVb) or (IVc): 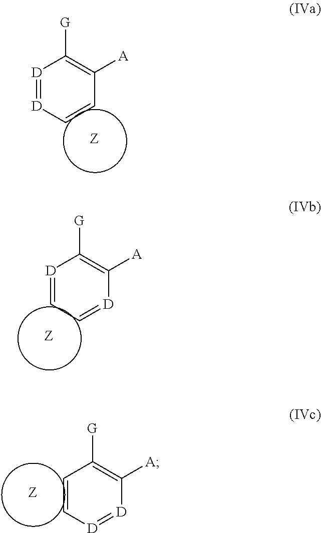
or a pharmaceutically acceptable salt, solvate, or prodrug of any of thereof; wherein:
the two D groups are each C(Rₐ), or alternatively, one of the D groups is C(Rₐ) and the other D group is N;
Ring Z is a monocyclic or polycyclic ring system fused to the six-membered aromatic ring containing the D groups, wherein Ring Z is a C₄-C₁₂ cycloalkyl, C₄-C₁₂ cycloalkenyl, 4- to 12-membered heterocyclic, aryl or heteroaryl, each optionally substituted;
A is selected from the group consisting of hydrogen, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl, optionally substituted heteroaryl, halo, OR_{b}, NR_{b}R_{b}, C(O)OR_{b}, NO₂, CN, C(O)R_{b}, C(O)C(O)R_{b}, C(O)NR_{b}R_{b}, NR_{b}C(O)R_{b}, NR_{b}S(O)ₙR_{b}, N(R_{b})COOR_{b}, NR_{b}C(O)C(O)R_{b}, NR_{b}C(O)R_{b}, NR_{b}C(O)N(R_{b})₂, NR_{b}S(O)ₙN(R_{b})₂ and OC(O)OR_{b;}
G is selected from the group consisting of optionally substituted 3- to 12-membered heterocyclic, optionally substituted heteroaryl, CON(R_{b})₂, NR_{b}C(O)R_{b}, NR_{b}S(O)ₙR_{b}, and C(R_{b})₂OR_{b};
Rₐ is selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl, C(O)R_{b}, C(O)C(O)R_{b}, C(O)NR_{b}R_{b}, O(R_{b}), and S(O)ₙR_{b};
each R_{b} is independently selected from the group consisting of H, optionally substituted C₁-C₁₀ alkyl, optionally substituted C₂-C₁₀ alkenyl, optionally substituted C₂-C₁₀ alkynyl, optionally substituted C₃-C₁₂ cycloalkyl, optionally substituted C₃-C₁₂ cycloalkenyl, optionally substituted heterocyclic, optionally substituted aryl and optionally substituted heteroaryl; and
n is 0, 1 or 2.

100-110. (canceled)

111. A compound selected from the compounds set forth in the tables below,
or a pharmaceutically acceptable salt, solvate or prodrug thereof:

| Compound | |
|---|---|
| Number | Compound |
| 1 | 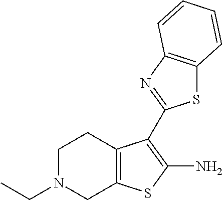 [SMILES: CCN1CCc2c(sc(N)c2-c2nc3ccccc3s2)C1] |
| 2 | 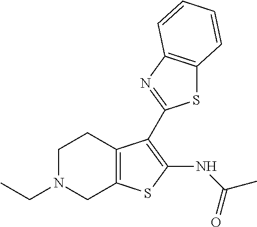 [SMILES: CCN1CCc2c(sc(NC(C)=O)c2-c2nc3ccccc3s2)C1] |
| 3 | 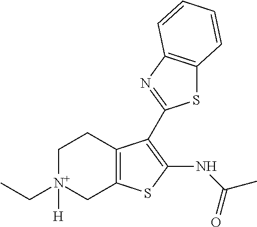 [SMILES: CC[NH+]1CCc2c(sc(NC(C)=O)c2-c2nc3ccccc3s2)C1] |
| 4 | 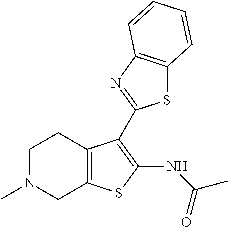 [SMILES: CC(=O)Nc1sc2c(c1-c1nc3ccccc3s1)CCN(C)C2] |
| 5 | 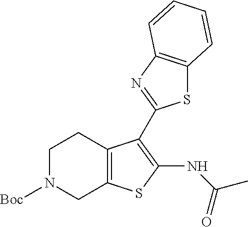 [SMILES: CC(=O)Nc1sc2c(c1-c1nc3ccccc3s1)CCN(C(=O)OC(C)(C)C)C2] |
| 6 | 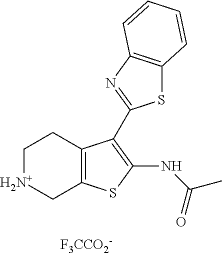 |
| 8 | 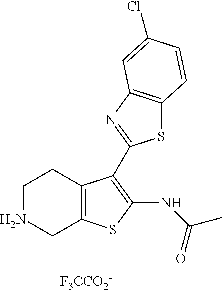 |
| 9 | 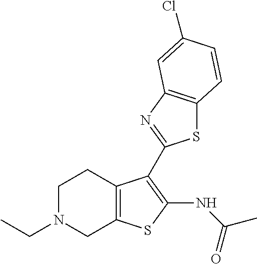 [SMILES: CCN1CCc2c(sc(NC(C)=O)c2-c2nc3cc(Cl)ccc3s2)C1] |
| 10 | 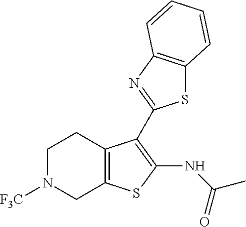 [SMILES: CC(=O)Nc1sc2c(c1-c1nc3ccccc3s1)CCN(C(F)(F)F)C2] |
| 11 | 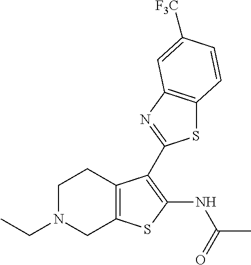 [SMILES: CCN1CCc2c(sc(NC(C)=O)c2-c2nc3cc(C(F)(F)F)ccc3s2)C1] |
| 12 | 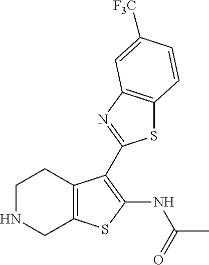 [SMILES: CC(=O)Nc1sc2c(c1-c1nc3cc(C(F)(F)F)ccc3s1)CCNC2] |
| 13 | 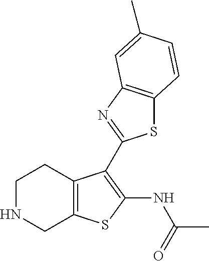 [SMILES: CC(=O)Nc1sc2c(c1-c1nc3cc(C)ccc3s1)CCNC2] |
| 14 | 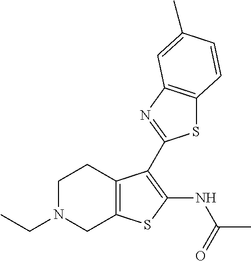 [SMILES: CCN1CCc2c(sc(NC(C)=O)c2-c2nc3cc(C)ccc3s2)C1] |
| 15 | 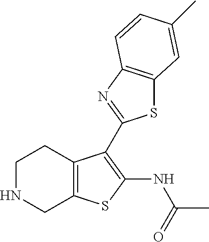 [SMILES: CC(=O)Nc1sc2c(c1-c1nc3ccc(C)cc3s1)CCNC2] |
| 16 | 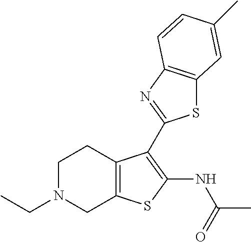 [SMILES: CCN1CCc2c(sc(NC(C)=O)c2-c2nc3ccc(C)cc3s2)C1] |
| 17 | 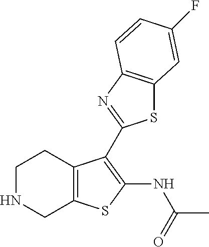 [SMILES: CC(=O)Nc1sc2c(c1-c1nc3ccc(F)cc3s1)CCNC2] |
| 18 | 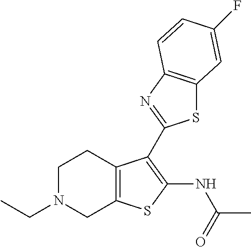 [SMILES: CCN1CCc2c(sc(NC(C)=O)c2-c2nc3ccc(F)cc3s2)C1] |
| 19 | 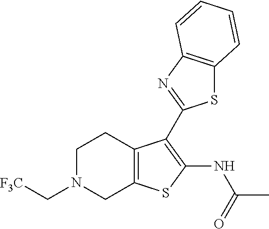 [SMILES: CC(=O)Nc1sc2c(c1-c1nc3ccccc3s1)CCN(CC(F)(F)F)C2] |
| 21 | 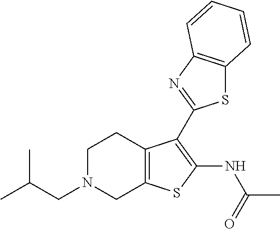 [SMILES: CC(=O)Nc1sc2c(c1-c1nc3ccccc3s1)CCN(CC(C)C)C2] |
| 22 | 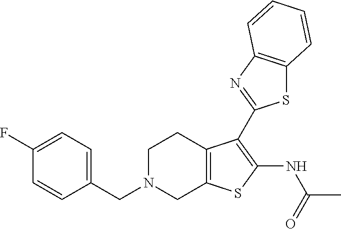 [SMILES: CC(=O)Nc1sc2c(c1-c1nc3ccccc3s1)CCN(Cc1ccc(F)cc1)C2] |
| 23 | 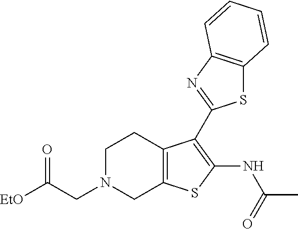 [SMILES: CCOC(=O)CN1CCc2c(sc(NC(C)=O)c2-c2nc3ccccc3s2)C1] |
| 24 | 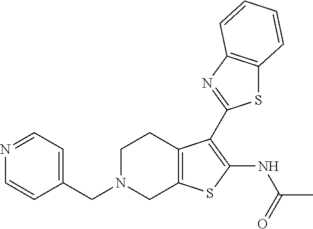 [SMILES: CC(=O)Nc1sc2c(c1-c1nc3ccccc3s1)CCN(Cc1ccncc1)C2] |
| 25 | 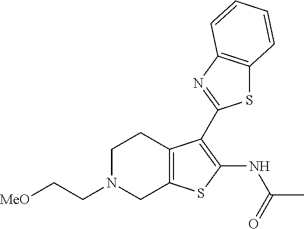 [SMILES: COCCN1CCc2c(sc(NC(C)=O)c2-c2nc3ccccc3s2)C1] |
| 26 | 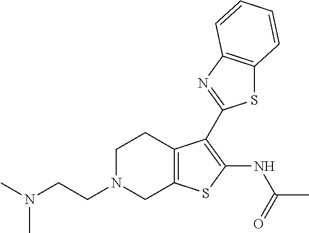 [SMILES: CC(=O)Nc1sc2c(c1-c1nc3ccccc3s1)CCN(CCN(C)C)C2] |
| 27 | 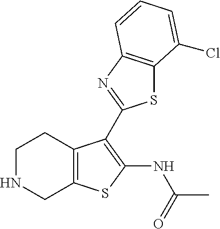 [SMILES: CC(=O)Nc1sc2c(c1-c1nc3cccc(Cl)c3s1)CCNC2] |
| 28 | 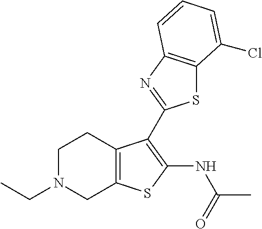 [SMILES: CCN1CCc2c(sc(NC(C)=O)c2-c2nc3cccc(Cl)c3s2)C1] |
| 29 | 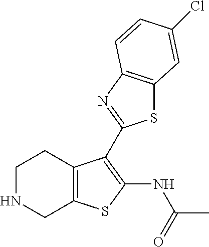 [SMILES: CC(=O)Nc1sc2c(c1-c1nc3ccc(Cl)cc3s1)CCNC2] |
| 30 | 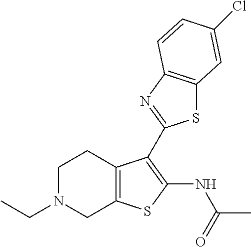 [SMILES: CCN1CCc2c(sc(NC(C)=O)c2-c2nc3ccc(Cl)cc3s2)C1] |
| 31 | 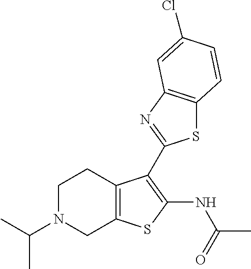 [SMILES: CC(=O)Nc1sc2c(c1-c1nc3cc(Cl)ccc3s1)CCN(C(C)C)C2] |
| 32 | 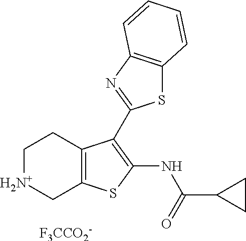 |
| 33 | 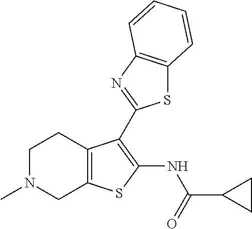 [SMILES: CN1CCc2c(sc(NC(=O)C3CC3)c2-c2nc3ccccc3s2)C1] |
| 34 | 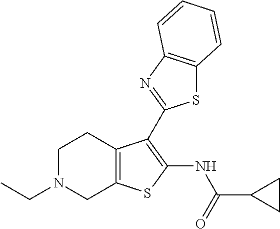 [SMILES: CCN1CCc2c(sc(NC(=O)C3CC3)c2-c2nc3ccccc3s2)C1] |
| 35 | 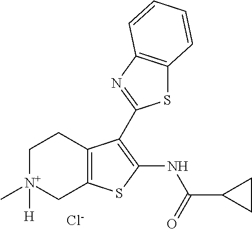 [SMILES: C[NH+]1CCc2c(sc(NC(=O)C3CC3)c2-c2nc3ccccc3s2)C1 ; [Cl-]] |
| 36 | 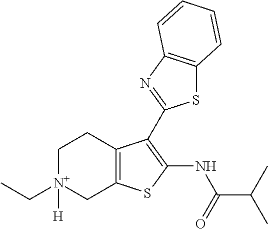 [SMILES: CC[NH+]1CCc2c(sc(NC(=O)C(C)C)c2-c2nc3ccccc3s2)C1] |
| 37 | 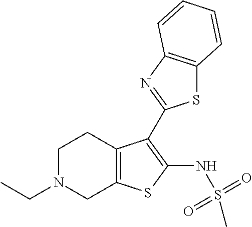 [SMILES: CCN1CCc2c(sc(NS(C)(=O)=O)c2-c2nc3ccccc3s2)C1] |
| 38 | 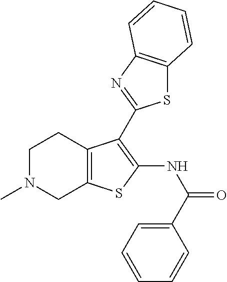 [SMILES: CN1CCc2c(sc(NC(=O)c3ccccc3)c2-c2nc3ccccc3s2)C1] |
| 39 | 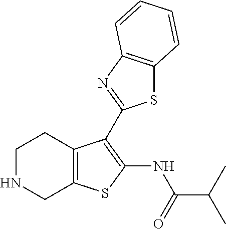 [SMILES: CC(C)C(=O)Nc1sc2c(c1-c1nc3ccccc3s1)CCNC2] |
| 40 | 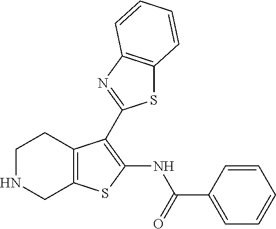 [SMILES: O=C(Nc1sc2c(c1-c1nc3ccccc3s1)CCNC2)c1ccccc1] |
| 41 | 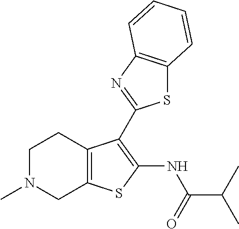 [SMILES: CC(C)C(=O)Nc1sc2c(c1-c1nc3ccccc3s1)CCN(C)C2] |
| 42 | 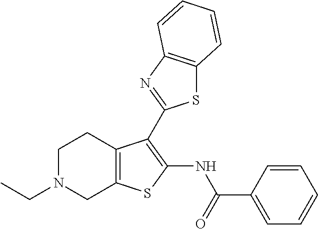 [SMILES: CCN1CCc2c(sc(NC(=O)c3ccccc3)c2-c2nc3ccccc3s2)C1] |
| 43 | 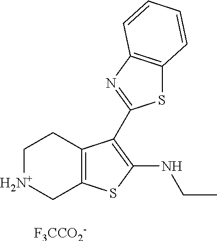 |
| 44 | 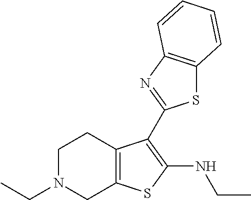 [SMILES: CCNc1sc2c(c1-c1nc3ccccc3s1)CCN(CC)C2] |
| 45 | 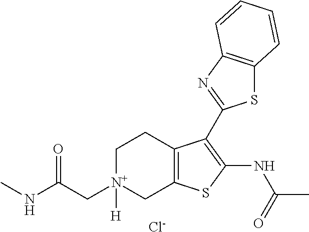 [SMILES: CNC(=O)C[NH+]1CCc2c(sc(NC(C)=O)c2-c2nc3ccccc3s2)C1 ; [Cl-]] |
| 46 | 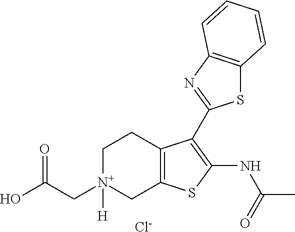 [SMILES: CC(=O)Nc1sc2c(c1-c1nc3ccccc3s1)CC[NH+](CC(=O)O)C2 ; [Cl-]] |
| 47 | 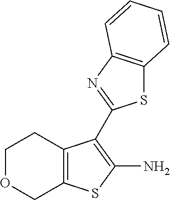 [SMILES: Nc1sc2c(c1-c1nc3ccccc3s1)CCOC2] |
| 48 | 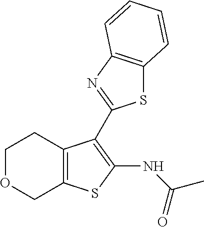 [SMILES: CC(=O)Nc1sc2c(c1-c1nc3ccccc3s1)CCOC2] |
| 51 | 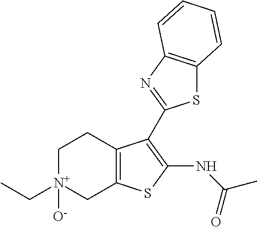 [SMILES: CC[N+]1([O-])CCc2c(sc(NC(C)=O)c2-c2nc3ccccc3s2)C1] |
| 52 | 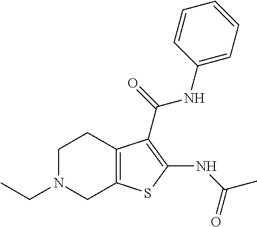 [SMILES: CCN1CCc2c(sc(NC(C)=O)c2C(=O)Nc2ccccc2)C1] |
| 53 | 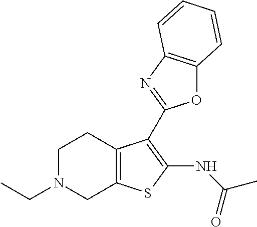 [SMILES: CCN1CCc2c(sc(NC(C)=O)c2-c2nc3ccccc3o2)C1] |
| 54 | 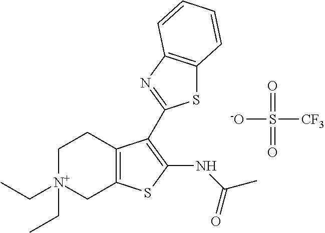 |
| 58 | 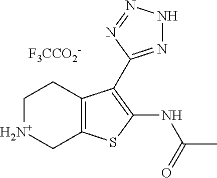 [SMILES: "F3CCO2-" ; CC(=O)Nc1sc2c(c1-c1nn[nH]n1)CC[NH2+]C2] |
| 59 | 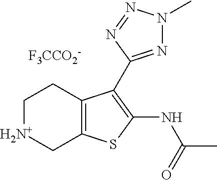 [SMILES: "F3CCO2-" ; CC(=O)Nc1sc2c(c1-c1nnn(C)n1)CC[NH2+]C2] |
| 60 | 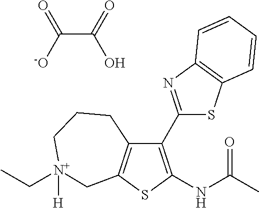 |
| 61 | 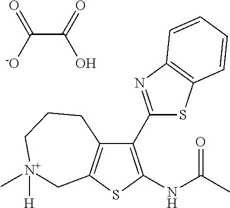 |
| 62 | 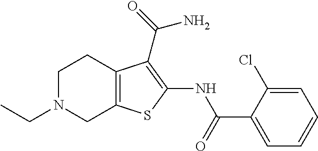 [SMILES: CCN1CCc2c(sc(NC(=O)c3ccccc3Cl)c2C(N)=O)C1] |
| 63 | 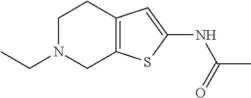 |
| 65 | 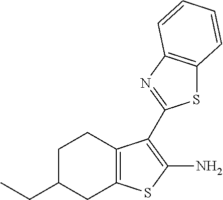 [SMILES: CCC1CCc2c(sc(N)c2-c2nc3ccccc3s2)C1] |
| 66 | 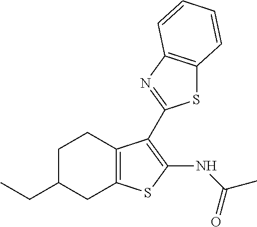 [SMILES: CCC1CCc2c(sc(NC(C)=O)c2-c2nc3ccccc3s2)C1] |
| 67 | 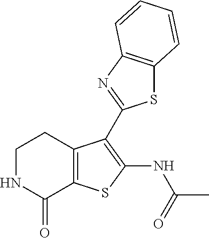 [SMILES: CC(=O)Nc1sc2c(c1-c1nc3ccccc3s1)CCNC2=O] |
| 68 | 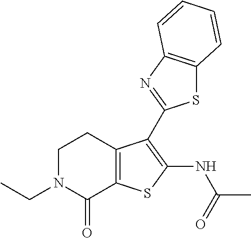 [SMILES: CCN1CCc2c(sc(NC(C)=O)c2-c2nc3ccccc3s2)C1=O] |
| 69 | 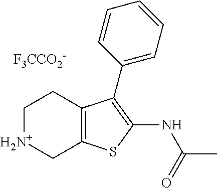 [SMILES: "F3CCO2-" ; CC(=O)Nc1sc2c(c1-c1ccccc1)CC[NH2+]C2] |
| 70 | 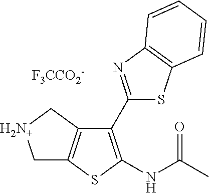 [SMILES: "F3CCO2-" ; CC(=O)Nc1sc2c(c1-c1nc3ccccc3s1)C[NH2+]C2] |
| 71 | 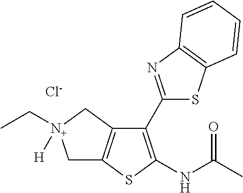 [SMILES: CC[NH+]1Cc2sc(NC(C)=O)c(-c3nc4ccccc4s3)c2C1 ; [Cl-]] |
| 72 | 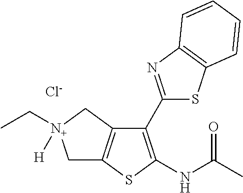 [SMILES: CC[NH+]1Cc2sc(NC(C)=O)c(-c3nc4ccccc4s3)c2C1 ; [Cl-]] |
| 77 | 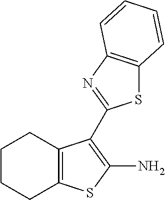 [SMILES: Nc1sc2c(c1-c1nc3ccccc3s1)CCCC2] |
| 78 | 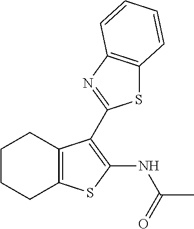 [SMILES: CC(=O)Nc1sc2c(c1-c1nc3ccccc3s1)CCCC2] |
| 80 | 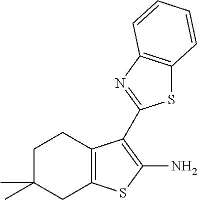 [SMILES: CC1(C)CCc2c(sc(N)c2-c2nc3ccccc3s2)C1] |
| 81 | 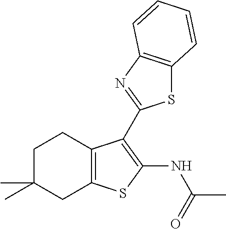 [SMILES: CC(=O)Nc1sc2c(c1-c1nc3ccccc3s1)CCC(C)(C)C2] |
| 82 | 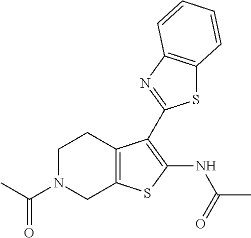 [SMILES: CC(=O)Nc1sc2c(c1-c1nc3ccccc3s1)CCN(C(C)=O)C2] |
| 83 | 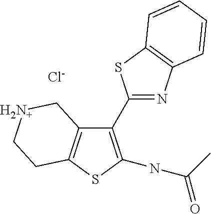 [SMILES: CC(=O)Nc1sc2c(c1-c1nc3ccccc3s1)C[NH2+]CC2 ; [Cl-]] |
| 84 | 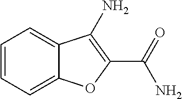 [SMILES: NC(=O)c1oc2ccccc2c1N] |
| 85 | 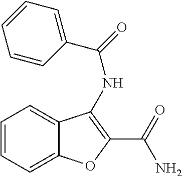 [SMILES: NC(=O)c1oc2ccccc2c1NC(=O)c1ccccc1] |
| 86 | 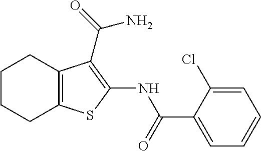 [SMILES: NC(=O)c1c(NC(=O)c2ccccc2Cl)sc2c1CCCC2] |
| 87 | 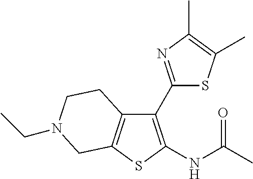 [SMILES: CCN1CCc2c(sc(NC(C)=O)c2-c2nc(C)c(C)s2)C1] |
| 88 | 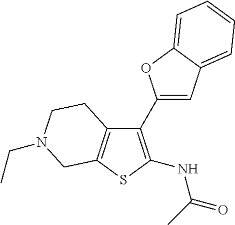 [SMILES: CCN1CCc2c(sc(NC(C)=O)c2-c2cc3ccccc3o2)C1] |
| 89 | 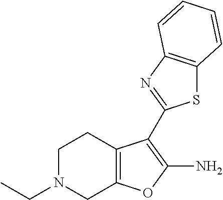 [SMILES: CCN1CCc2c(oc(N)c2-c2nc3ccccc3s2)C1] |
| 90 | 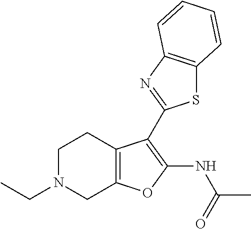 [SMILES: CCN1CCc2c(oc(NC(C)=O)c2-c2nc3ccccc3s2)C1] |
| 91 | 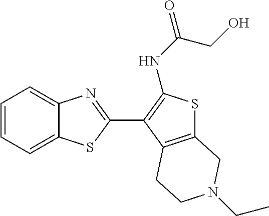 [SMILES: CCN1CCc2c(sc(NC(=O)CO)c2-c2nc3ccccc3s2)C1] |
| 92 | 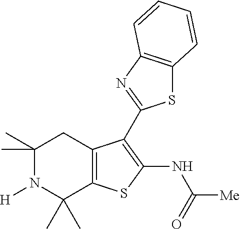 [SMILES: CC(=O)Nc1sc2c(c1-c1nc3ccccc3s1)CC(C)(C)NC2(C)C] |
| 93 | 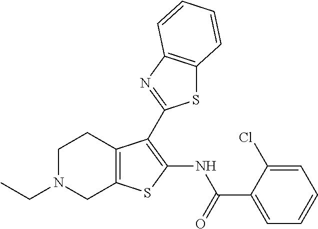 [SMILES: CCN1CCc2c(sc(NC(=O)c3ccccc3Cl)c2-c2nc3ccccc3s2)C1] |
| 94 | 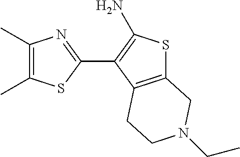 [SMILES: CCN1CCc2c(sc(N)c2-c2nc(C)c(C)s2)C1] |
| 95 | 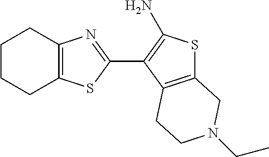 [SMILES: CCN1CCc2c(sc(N)c2-c2nc3c(s2)CCCC3)C1] |
| 96 | 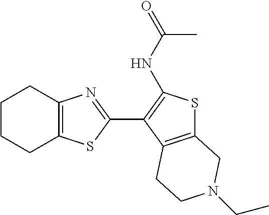 [SMILES: CCN1CCc2c(sc(NC(C)=O)c2-c2nc3c(s2)CCCC3)C1] |
| 97 | 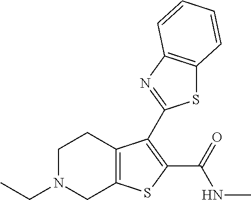 [SMILES: CCN1CCc2c(sc(C(=O)NC)c2-c2nc3ccccc3s2)C1] |
| 98 | 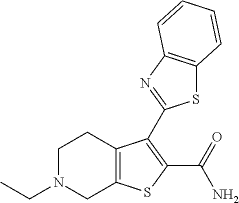 [SMILES: CCN1CCc2c(sc(C(N)=O)c2-c2nc3ccccc3s2)C1] |
| 99 | 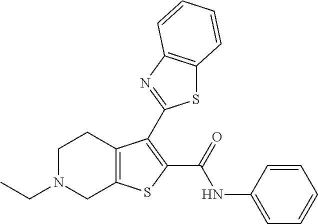 [SMILES: CCN1CCc2c(sc(C(=O)Nc3ccccc3)c2-c2nc3ccccc3s2)C1] |
| 100 | 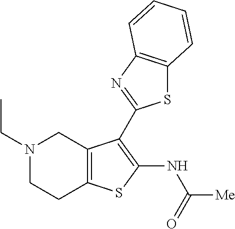 [SMILES: CCN1CCc2sc(NC(C)=O)c(-c3nc4ccccc4s3)c2C1] |
| 101 | 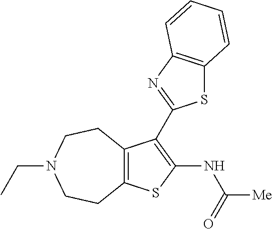 [SMILES: CCN1CCc2sc(NC(C)=O)c(-c3nc4ccccc4s3)c2CC1] |
| 102 | 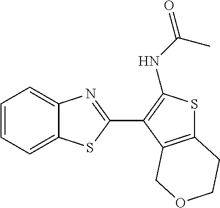 [SMILES: CC(=O)Nc1sc2c(c1-c1nc3ccccc3s1)COCC2] |
| 103 | 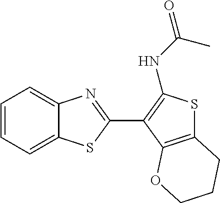 [SMILES: CC(=O)Nc1sc2c(c1-c1nc3ccccc3s1)OCCC2] |
| 104 | 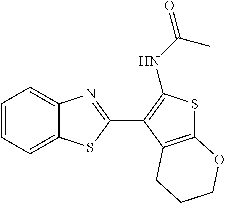 [SMILES: CC(=O)Nc1sc2c(c1-c1nc3ccccc3s1)CCCO2] |
| 105 | 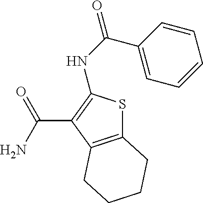 [SMILES: NC(=O)c1c(NC(=O)c2ccccc2)sc2c1CCCC2] |
| 106 | 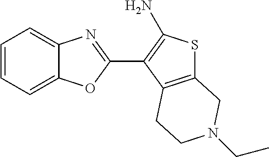 [SMILES: CCN1CCc2c(sc(N)c2-c2nc3ccccc3o2)C1] |
| 107 | 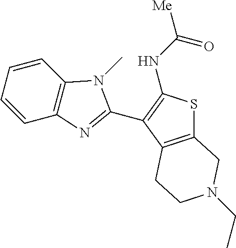 [SMILES: CCN1CCc2c(sc(NC(C)=O)c2-c2nc3ccccc3n2C)C1] |
| 108 | 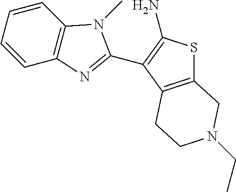 [SMILES: CCN1CCc2c(sc(N)c2-c2nc3ccccc3n2C)C1] |
| 109 | 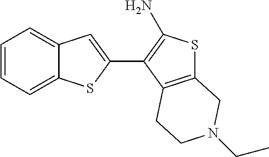 [SMILES: CCN1CCc2c(sc(N)c2-c2cc3ccccc3s2)C1] |
| 110 | 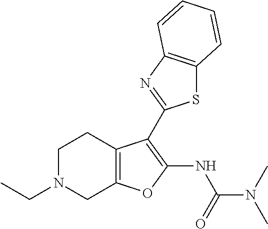 [SMILES: CCN1CCc2c(oc(NC(=O)N(C)C)c2-c2nc3ccccc3s2)C1] |
| 111 | 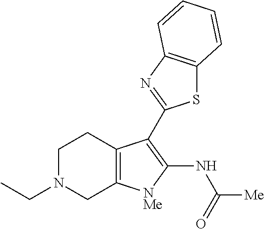 [SMILES: CCN1CCc2c(-c3nc4ccccc4s3)c(NC(C)=O)n(C)c2C1] |
| 112 | 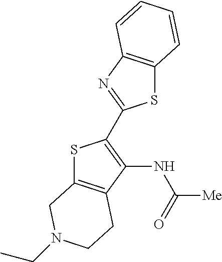 [SMILES: CCN1CCc2c(sc(-c3nc4ccccc4s3)c2NC(C)=O)C1] |
| 113 | 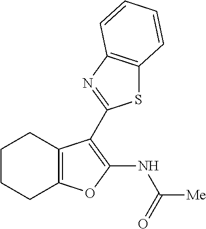 [SMILES: CC(=O)Nc1oc2c(c1-c1nc3ccccc3s1)CCCC2] |
| 114 | 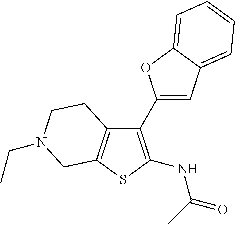 [SMILES: CCN1CCc2c(sc(NC(C)=O)c2-c2cc3ccccc3o2)C1] |
| 123 | 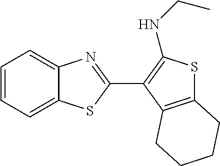 [SMILES: CCNc1sc2c(c1-c1nc3ccccc3s1)CCCC2] |
| Compound | |
|---|---|
| Number | Compound |
| 49 | 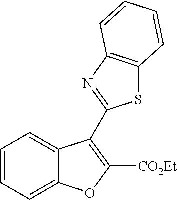 [SMILES: CCOC(=O)c1oc2ccccc2c1-c1nc2ccccc2s1] |
| 50 | 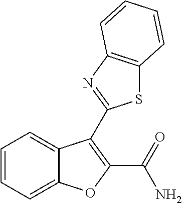 [SMILES: NC(=O)c1oc2ccccc2c1-c1nc2ccccc2s1] |
| 55 | 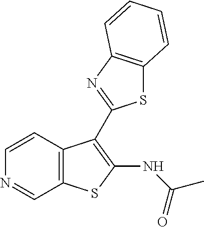 [SMILES: CC(=O)Nc1sc2cnccc2c1-c1nc2ccccc2s1] |
| 56 | 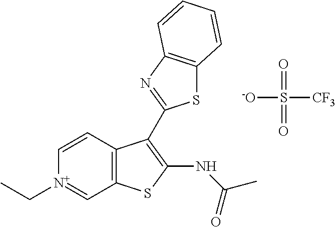 |
| 57 | 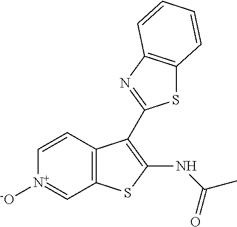 |
| 79 | 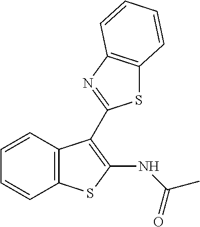 [SMILES: CC(=O)Nc1sc2ccccc2c1-c1nc2ccccc2s1] |
| 84 | 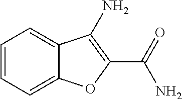 [SMILES: NC(=O)c1oc2ccccc2c1N] |
| 85 | 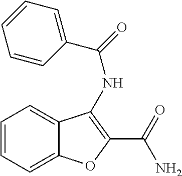 [SMILES: NC(=O)c1oc2ccccc2c1NC(=O)c1ccccc1] |
| 115 | 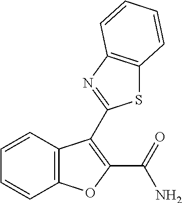 [SMILES: NC(=O)c1oc2ccccc2c1-c1nc2ccccc2s1] |
| 116 | 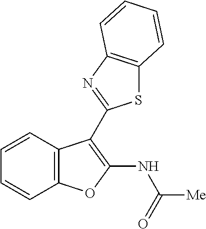 [SMILES: CC(=O)Nc1oc2ccccc2c1-c1nc2ccccc2s1] |
| 117 | 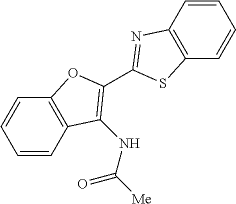 [SMILES: CC(=O)Nc1c(-c2nc3ccccc3s2)oc2ccccc12] |
| 118 | 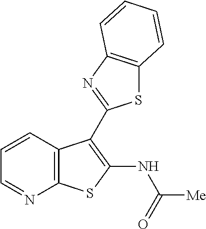 [SMILES: CC(=O)Nc1sc2ncccc2c1-c1nc2ccccc2s1] |
| 119 | 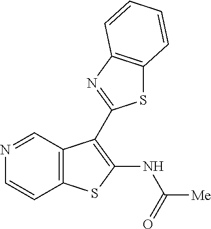 [SMILES: CC(=O)Nc1sc2ccncc2c1-c1nc2ccccc2s1] |
| 120 | 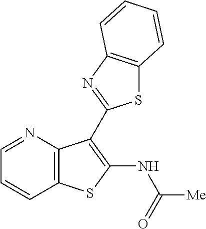 [SMILES: CC(=O)Nc1sc2cccnc2c1-c1nc2ccccc2s1] |
| 64 | 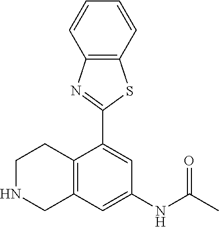 [SMILES: CC(=O)Nc1cc2c(c(-c3nc4ccccc4s3)c1)CCNC2] |
| 73 | 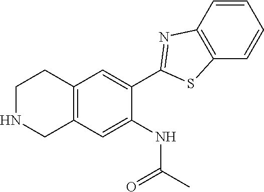 [SMILES: CC(=O)Nc1cc2c(cc1-c1nc3ccccc3s1)CCNC2] |
| 74 | 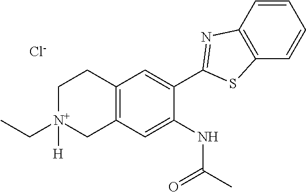 [SMILES: CC[NH+]1CCc2cc(-c3nc4ccccc4s3)c(NC(C)=O)cc2C1 ; [Cl-]] |
| 75 | 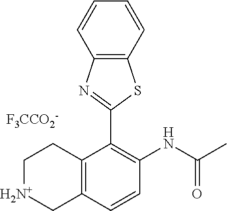 [SMILES: "F3CCO2-" ; CC(=O)Nc1ccc2c(c1-c1nc3ccccc3s1)CC[NH2+]C2] |
| 76 | 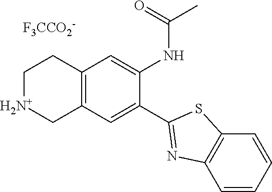 [SMILES: "F3CCO2-" ; CC(=O)Nc1cc2c(cc1-c1nc3ccccc3s1)C[NH2+]CC2] |
| 121 | 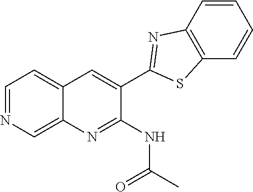 [SMILES: CC(=O)Nc1nc2cnccc2cc1-c1nc2ccccc2s1] |
| 122 | 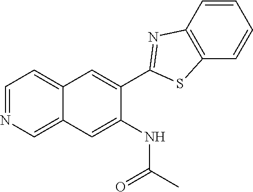 [SMILES: CC(=O)Nc1cc2cnccc2cc1-c1nc2ccccc2s1] |

112. The compound of claim 111, wherein the compound is selected from the group consisting of Compound 47, Compound 48, Compound 51, Compound 54, Compound 58, Compound 59, Compound 65, Compound 66, Compound 67, Compound 68, Compound 77, Compound 78, Compound 80, Compound 81, Compound 86, Compound 110, Compound 111, Compound 123, Compound 49, Compound 50, Compound 55, Compound 56, Compound 57, Compound 79, Compound 119, Compound 64, Compound 73, Compound 74, Compound 75, Compound 76, Compound 121 and Compound 122.
